(19) United States
(12) Patent Application Publication
(10) Pub. No.: US 2026/0053794 A1
(43) Pub. Date: Feb. 26, 2026

(54) SOMATOSTATIN RECEPTOR 2 AGONISTS AND USES THEREOF

(71) Applicant: Exelixis, Inc., Alameda, CA (US)

(72) Inventors: Dongcheng DAI, Shanghai (CN); Cui LI, Shanghai (CN); He ZHANG, Shanghai (CN)

(21) Appl. No.: 19/190,582

(22) Filed: Apr. 25, 2025

Related U.S. Application Data
(63) Continuation of application No. PCT/IB2023/060867, filed on Oct. 27, 2023.

(30) Foreign Application Priority Data
Oct. 28, 2022 (WO) PCT/CN2022/128301

(51) Int. Cl. A61K 31/4745; A61K 31/4436; A61K 31/444; A61K 31/4709; A61K 31/496; A61K 31/519; C07D 215/233; C07D 401/04; C07D 401/14; C07D 471/04; C07D 471/10; C07D 471/22; C07D 487/10; C07D 491/048; C07D 491/08; C07D 495/04; C07D 513/16; C07D 513/22

(52) U.S. Cl. CPC A61K 31/4745; A61K 31/4436; A61K 31/444; A61K 31/4709; A61K 31/496; A61K 31/519; C07D 215/233; C07D 401/04; C07D 401/14; C07D 471/04; C07D 471/10; C07D 471/22; C07D 487/10; C07D 491/048; C07D 491/08; C07D 495/04; C07D 513/16; C07D 513/22

(57) ABSTRACT

The subject matter described herein is directed to somatostatin receptor activating compounds, methods of making the compounds, pharmaceutical compositions, and their use in the treatment of diseases associated with somatostatin receptors.

### CROSS-REFERENCE TO RELATED APPLICATIONS

This patent application claims the benefit of and priority to PCT Application No. PCT/CN2022/128301, filed on Oct. 28, 2022, the entire contents of which are incorporated by reference herein.

### FIELD

The subject matter described herein is directed to somatostatin receptor activating compounds, methods of making the compounds, pharmaceutical compositions, and their use in the treatment of diseases associated with somatostatin receptors.

### BACKGROUND

Somatostatin (SST) is a peptide with numerous biofunctionalities including the modulation of secretion of growth hormone, insulin, glucagon, and gastric acid. Additionally, SST displays potent anti-proliferative effects.

The mechanism of action of somatostatin proceeds via high affinity membrane associated somatostatin receptors (SSTR). There are five pharmacologically distinct SSTRs (SSTR1-5) which are heterogeneously distributed. SSTR2 is of particular interest as it has been shown to mediate the inhibition of release of growth hormone from the anterior pituitary gland and glucagon from the pancreas. Growth hormone plays a causative role in diabetes associated complications such as diabetic retinopathy. Somatostatin's regulation of glucagon and growth hormone release posits the use of SSTR2 activators or agonists as treatment of diabetes and diabetes-related pathologies, including retinopathy, neuropathy, and nephropathy. In addition, somatostatin and SSTR2 have been implicated in a variety of other biological processes such as nociception, inflammation, and cell proliferation.

Therefore, the compounds and methods described herein may also be useful in the therapy of a variety of conditions which include diabetes, diarrhea, inflammatory bowel disease, irritable bowel syndrome, cancer, acromegaly, depression, chronic atrophic gastritis, Crohn's disease, ulcerative colitis, retinopathy, arthritis, restenosis, neuroendocrine tumors (NETs), and pain.

What is therefore needed and not effectively addressed by the art are compounds that act as agonists of SSTR2 that can be efficiently formulated and administered with desirable activity potentiating SSTR2. This deficiency is addressed by the subject matter described herein.

### BRIEF SUMMARY

In certain embodiments, the subject matter described herein is directed to a compound of Formula I, which includes Formulae Ia, Ia-1, Ia-2, Ia-3, Ia4, Ia4A, Ia-5, Ia-6, Ia-7, Ib, Ib-1a, Ib-1b, Ib-1c, Ib-1d, Ib-1e, Ib-2, Ib-3, Ic, Id, Ie, If, Ig, and I-2, or a pharmaceutically acceptable salt thereof.

In certain embodiments, the subject matter described herein is directed to a pharmaceutical composition comprising a compound of Formula I or a pharmaceutically acceptable salt thereof.

In certain embodiments, the subject matter described herein is directed to a method of treating a disease or disorder by administering a compound of Formula I or the pharmaceutical composition thereof.

In certain embodiments, the subject matter described herein is directed to a method of treating a subject afflicted with a disease associated with somatostatin, comprising administering to the subject a compound of Formula I or the pharmaceutical composition thereof.

In certain embodiments, the subject matter described herein is directed to a method of activating somatostatin receptors in a subject, comprising administering to the subject a compound of Formula I or the pharmaceutical composition thereof.

Other embodiments are also described.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literature, patents, and similar materials differs from or contradicts this application, including but not limited to defined terms, term usage, described techniques, or the like, this application controls.

### I. Definitions

As used in the present specification, the following words, phrases, and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ-Cᵥ” indicates that the following group has from u to v carbon atoms. For example, “C₁-C₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±50%. In certain other embodiments, the term “about” includes the indicated amount±20%. In certain other embodiments, the term “about” includes the indicated amount±10%. In other embodiments, the term “about” includes the indicated amount±5%. In certain other embodiments, the term “about” includes the indicated amount±1%. In certain other embodiments, the term “about” includes the indicated amount±0.5% and in certain other embodiments, 0.1%. Such variations are appropriate to perform the disclosed methods or employ the disclosed compositions. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁-C₂₀ alkyl), 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH), isobutyl (i.e., —CH₂CH(CH₃)₂) and tert-butyl (i.e., —C(CH)₃); and “propyl” includes n-propyl (i.e., —(CH₂)₂CH₃) and isopropyl (i.e., —CH(CH₃)₂).

The term “alkylene” by itself or as part of another substituent means a divalent radical derived from an alkane, such as, methylene —CH₂—, ethylene —CH₂CH₂—, and the like. As an example, a “hydroxy-methylene” refers to HO—CH₂—*, where * is the attachment point to the molecule.

Unless indicated explicitly otherwise, where combinations of groups are referred to herein as one moiety, e.g., arylalkyl or aralkyl, the last mentioned group contains the atom by which the moiety is attached to the rest of the molecule.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Amino” refers to the group —NR^{y}R^{z} wherein R^{y} and R^{z} are independently hydrogen, alkyl, cycloalkyl, heterocyclyl, aryl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₁-C₂₀ cycloalkyl), 3 to 12 ring carbon atoms (i.e., C₃-C₁₂ cycloalkyl), 3 to 10 ring carbon atoms (i.e., C₃-C₁₀ cycloalkyl), 3 to 8 ring carbon atoms (i.e., C₃-C₆ cycloalkyl), 3 to 7 ring carbon atoms (i.e., C₃-C₇ cycloalkyl), or 3 to 6 ring carbon atoms (i.e., C₃-C₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more (e.g., 1 to 6, or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like. “C₁-C₃ haloalkyl” and “halo-C₁-C₃ alkyl” are used interchangeably herein and refer to an alkyl chain having 1 to 3 carbon atoms, wherein one or more of the hydrogen atoms in the alkyl chain are replaced by a halogen. Further, C₁-C₃ fluoroalkyl (or fluoro-C₁-C₃ alkyl) refers to an alkyl chain having 1 to 3 carbon atoms, wherein one or more of the hydrogen atoms in the alkyl chain are replaced by fluoro. Non-limiting examples of haloalkyl groups include —CH₂CH₂CF₃, —CHF₂, —CF₃, —CH₂CHF₂, and —CH₂CF₃

“Haloalkoxy” refers to an alkoxy group as defined above, wherein one or more (e.g., 1 to 6, or 1 to 3) hydrogen atoms are replaced by a halogen. “C₁-C₃ haloalkoxy” and “halo-C₁-C₃ alkoxyl” are used interchangeably herein and refer to an alkoxy group having 1 to 3 carbon atoms in the alkyl unit of the alkoxy group, wherein one or more of the hydrogen atoms in the alkyl chain are replaced by a halogen. Non-limiting examples of haloalkoxy groups include —OCH₂CHF₂, —OCH₂CF₃, and —OCF₃.

“Hydroxyalkyl” or “hydroxyalkylene” and the like refers to an alkyl or alkylene group as defined above, wherein one or more (e.g., 1 to 6, or 1 to 3)hydrogen atoms are replaced by a hydroxy group. By way of example, the term “hydroxy-C₁-C₃ alkyl,” “C₁-C₃ hydroxyalkyl”, or “hydroxy-C₁-C₃ alkylene” refers to a one to three carbon alkyl chain where one or more hydrogens on any carbon is replaced by a hydroxy group, in particular, one hydrogen on one carbon of the chain is replaced by a hydroxy group.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₃₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems (9- to 10-membered heteroaryl), 6-10 membered ring systems (6- to 10-membered heteraryl), 5-10 membered ring systems (5- to 10-membered heteroaryl), 5-7 membered ring systems (5- to 7-membered heteroaryl), or 5-6 membered ring systems (5- to 6-membered heteroaryl), each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more (e.g., 1 to 3) oxo (═O) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂-C₂₀ heterocyclyl), 2 to 12 ring carbon atoms (i.e., C₂-C₁₂ heterocyclyl), 2 to 10 ring carbon atoms (i.e., C₂-C₁₀ heterocyclyl), 2 to 8 ring carbon atoms (i.e., C₂-C₈ heterocyclyl), 3 to 12 ring carbon atoms (i.e., C₃-C₁₂ heterocyclyl), 3 to 8 ring carbon atoms (i.e., C₃-C₈ heterocyclyl), or 3 to 6 ring carbon atoms (i.e., C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. When the heterocyclyl ring contains 4- or 6-ring atoms, it is also referred to herein as a 4- or 6-membered heterocyclyl. When the heterocyclyl ring contains 5- to 7-ring atoms, it is also referred to herein as a 5- to 7-membered heterocyclyl. When the heterocyclyl ring contains 5- to 10-ring atoms, it is also referred to herein as a 5- to 10-membered heterocyclyl. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

“(C₁-C₃ alkoxy)-C₁-C₃ alkyl” refers to an -alkyl-alkoxy group, wherein both the alkoxy unit and the alkyl unit each individually contain an alkyl chain having 1 to 3 carbon atoms.

“(C₁-C₃-alkoxy)-C₁-C₃ alkoxy” refers to an -alkoxy-alkoxy group, wherein both alkoxy units each individually contain an alkyl chain having 1 to 3 carbon atoms.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5, 1 to 4, or 1 to 3) hydrogen atoms on the designated atom or group may or may not be replaced by a moiety other than hydrogen.

The term “substituted” used herein means any of the above groups (i.e., alkyl, alkylene, alkoxy, haloalkyl, haloalkoxy, cycloalkyl, aryl, heterocyclyl, and/or heteroaryl) wherein at least one (e.g., 1 to 5, 1 to 4, or 1 to 3) hydrogen atom is replaced by a bond to a non-hydrogen atom such as, but not limited to alkyl, alkoxy, amino, aryl, aralkyl, carboxyl, carboxyl ester, cyano, cycloalkyl, halo, haloalkyl, haloalkoxy, hydroxyalkyl, heteroaryl, heterocyclyl, —NHNH₂, hydroxy, oxo, nitro, —S(O)OH, —S(O)₂OH, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5, 1 to 4, or 1 to 3) hydrogen atoms are independently replaced with deuterium, halo, cyano, nitro, oxo, alkyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NR^{g}S(═O)₁₋₂R^{g}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂₀R^{g}, —NR^{g}S(═O)₁₋₂NR^{g}R^{h}, ═NSO₂R^{g}, ═NOR^{g}, —S(═O)₁₋₂NR^{g}R^{h}, —SF₅, —SCF₃ or —OCF₃. In certain embodiments, “substituted” also means any of the above groups in which one or more (e.g., 1 to 5, 1 to 4, or 1 to 3) hydrogen atoms are replaced with —C(═O)R^{g}, —C(═O)ORs, —C(═O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkoxy, aryl, cycloalkyl, haloalkyl, heterocyclyl, and/or heteroaryl. In certain embodiments, “substituted” also means any of the above groups in which one or more (e.g., 1 to 5, 1 to 4, or 1 to 3) hydrogen atoms are replaced by a bond to an amino, cyano, hydroxyl, nitro, oxo, halo, alkyl, alkoxy, alkylamino, aryl, cycloalkyl, haloalkyl, heterocyclyl, N-heterocyclyl, heteroaryl, or two of R^{g} and R^{h} and Rⁱ are taken together with the atoms to which they are attached to form a heterocyclyl ring optionally substituted with oxo, halo or alkyl optionally substituted with oxo, halo, amino, hydroxyl, or alkoxy.

In certain embodiments described herein, the recitation “R¹ and Z together with the ring to which each is attached form a fused bicyclic ring” refers to a compound having the structure:

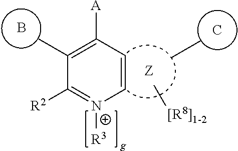

In certain embodiments, the recitation “R¹ and R^{C1} together with the ring to which each is attached form a fused tricyclic ring” refers to a compound having the structure:

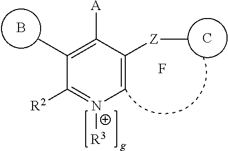

In certain embodiments, the recitation “R² and R^{B1} together with the ring to which each is attached form a fused tricyclic ring” refers to a compound having the structure:

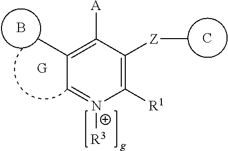

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to four. In certain embodiments, as used herein, the phrase “one or more” refers to one to three.

Any compound or structure given herein, is intended to represent unlabeled forms as well as isotopically labeled forms (isotopologues) of the compounds. These forms of compounds may also be referred to as and include “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism.” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium. Further, in some embodiments, the corresponding deuterated analog is provided.

In many cases, the compounds of this disclosure are capable of forming acid and/or base salts by virtue of the presence of amino and/or carboxyl groups or groups similar thereto.

Provided also are a pharmaceutically acceptable salt, isotopically enriched analog, deuterated analog, isomer (such as a stereoisomer), mixture of isomers (such as a mixture of stereoisomers), prodrug, and metabolite of the compounds described herein.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₃), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the an to comprise both amide and imidic acid tautomers. Thus, the amide containing compounds are understood to include their imidic acid tautomers. Likewise, the imidic acid containing compounds are understood to include their amide tautomers.

The present compounds, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present subject matter is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present subject matter contemplates various stereoisomers and mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Preparation, selection and use of prodrugs is discussed in T. Higuchi and V. Stella, “Pro-drugs as Novel Delivery Systems.” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

The term, “metabolite,” as used herein refers to a resulting product formed when a compound disclosed herein is metabolized. As used herein, the term “metabolized” refers to the sum of processes (including but not limited to hydrolysis reactions and reactions catalyzed by enzymes) by which a particular substance, such as a compound disclosed herein, is changed by an organism. For example, an aldehyde moiety (—C(O)H) of the compounds of the present subject matter may be reduced in vivo to a —CH₂OH moiety.

As used herein, the term “somatostatin receptor agonist” and the like refers to a compound that activates, increases, or modulates one or more of the biological activities of somatostatin receptors. The activity could increase, for example, at least about 5%, 10%, 15%, 20%, 25%, 30%, 35%, 40%, 45%, 50%, 55%, 60%, 65%, 70%, 75%, 80%, 85%, 95% or 100% of the activity of somatostatin receptor compared to an appropriate control. The increase can be a statistically significant increase.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a pyrukate kinase deficiency (PKD). The therapeutically effective amount may vary depending on the subject, and disease or condition being treated, the weight and age of the subject, the severity of the disease or condition, and the manner of administering, which can readily be determined by one of ordinary skill in the art.

Additional definitions may also be provided below as appropriate.

### II. Compounds

As described herein, in certain embodiments, the subject matter is directed to a compound of Formula I:

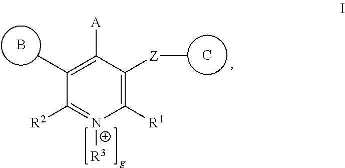

or a pharmaceutically acceptable salt thereof, wherein,
   g is 1 or 0;
Z is absent, —N(H)—C(═O)— or —C(═O)—NH—;
R¹ is hydrogen or halogen; or,
R¹ and Z together with the ring to which each is attached form a fused bicyclic ring, optionally substituted with one or two R⁸; or,
R¹ and R^{C1} together with the ring to which each is attached form a fused tricyclic ring;
Ring C is selected from the group consisting of 4- to 10-membered monocyclic or bicyclic fused heterocyclyl, 6- to 10-membered aryl, and 5- to 10-membered heteroaryl, each of which is substituted with R^{C1}, R^{C2} and R^{C3},
wherein said 4- to 10-membered monocyclic or bicyclic fused heterocyclyl or 5- to 10-membered heteroaryl each independently contains 1, 2, 3, or 4 ring heteroatoms selected from N, O, and S; and, wherein,
R^{C1} is hydrogen or can bind with R¹, R³ or R⁶;
R^{C2} and R^{C3} are each independently selected from the group consisting of hydrogen, halogen, cyano, C₁-C₆ alkyl, hydroxy, —C(═O)NH₂, —O—C₂-C₆alkenyl, —C₁-C₆alkoxy, —NH—(CH₂)₁₋₅—NH₂, —O-piperidinyl, —O—(CH₂)_{qC}—O—(CH₂)_{rC}—CH₃, wherein qC and rC are each independently an integer from 1 to 4,
R² is hydrogen or halogen; or,
R² and R^{B1} together with the ring to which each is attached form a fused tricyclic ring;
R³, if present, is a group covalently bound to R^{C1};
Ring B is phenyl or pyridinyl, each mono-substituted with R^{B1}, or di-substituted with R^{B1} and R^{B2};
   wherein, R^{B1} and R^{B2}, are each independently selected from the group consisting of hydrogen, cyano, C₁-C₆ alkyl, halogen, halo-C₁-C₆ alkyl, hydroxy-C₁-C₆alkyl, hydroxy,
   C₁-C₆ alkoxy, —O—C₂-C₆alkenyl, —NH—(CH₂)₁₋₅—NH₂, —O—(CH₂)_{q2}—O—(CH₂)ᵣ₂—CH₃, wherein q2 and r2 are each independently an integer from 1 to 4, —C(O)—NR^{Na}R^{Nb}, wherein each R^{Na} and R^{Nb} is each independently selected from the group consisting of hydrogen and C₁-C₆ alkyl;
      and,
A is selected from the group consisting of:
   i. —NR⁴R⁵, wherein
      R⁴ is optionally substituted C₁-C₆alkyl, —NH—C₁-C₆ alkyl-NHR^{4a}, and —C₁-C₆ alkyl-NHR^{4a}, wherein, R^{4a} is hydrogen or methyl; and,
      R⁵ is hydrogen or optionally substituted C₁-C₆ alkyl;
      wherein the optional substituents are selected from the group consisting of halogen and hydroxy;
   or,
      R⁵ and R^{B1} together with the N to which R⁵ is attached and Ring B to which R^{B1} is attached form a fused heterocyclyl;
   or,
      R⁵ and R^{C1} together with the N to which R⁵ is attached and Ring C to which R^{C1} is attached form a fused heterocyclyl;
   or,
      R⁵ and R⁸ together with the N to which R⁵ is attached and the ring to which R⁸ is attached form a fused heterocyclyl;
   ii. spirocyclic, substituted with —NH—R⁶ or R^{S}, wherein
      R^{S} is hydrogen or —C(═O)—C₁-C₆ alkyl;
and,
   iii. —O—(C₃-C₈ cycloalkyl), 5- to 11-membered heterocyclyl, or 5- to 6-membered heteroaryl, each substituted with —NH—R⁶, or substituted with R^{F} and R^{G}, wherein
      R^{F} is selected from the group consisting of hydroxyl, hydroxy-C₁-C₆ alkyl, nitro, —C(═O)H, —C₁-C₆alkoxy, —C(═NH)—NH₂, —NH—C(═NH)—NH₂, —C(═O)—C₁-C₆ alkyl, —(C═O)—O—C₁-C₆ alkyl and —(C₁-C₆alkyl)ₓ-NR^{F1}R^{F2}, wherein
         x is 0 or 1; and,
         R^{F1} and R^{F2} are each independently H, —(C═O)—O—C₁-C₆ alkyl, and C₁-C₆ alkyl;
      R^{G} is hydrogen or —C₁-C₆ alkoxy; or.
      R^{G} together with R^{C1} form a —O—(CH₂)ₖ—O—, wherein k is an integer from 1 to 5; or,
      R^{F} and R^{G} together form carboxy; and,
      R⁶ together with R^{C1} form:

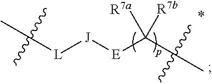

wherein,

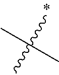

represents the attachment point of R⁶ to A;
p is an integer from 1 to 4;
R^{7a} and R^{7b} are, in each instance, independently selected from the group consisting of hydroxyl, optionally substituted —C₁-C₆ alkyl, —N₃, —NR^{a}R^{b}, wherein R^{a} and R^{b} are each independently H, or C₁-C₆ alkyl;
E is absent, —O— or —N(R^{b})—, wherein R^{b} is H or optionally substituted C₁-C₆alkyl;
J is —C(O)— or —C(R^{7a}R^{7b})—; and,
L is absent, —O— or —N(H)—,
   wherein the optional substituents are selected from the group consisting of halogen, —NH₂ and hydroxy;
provided that the compound is not one of:

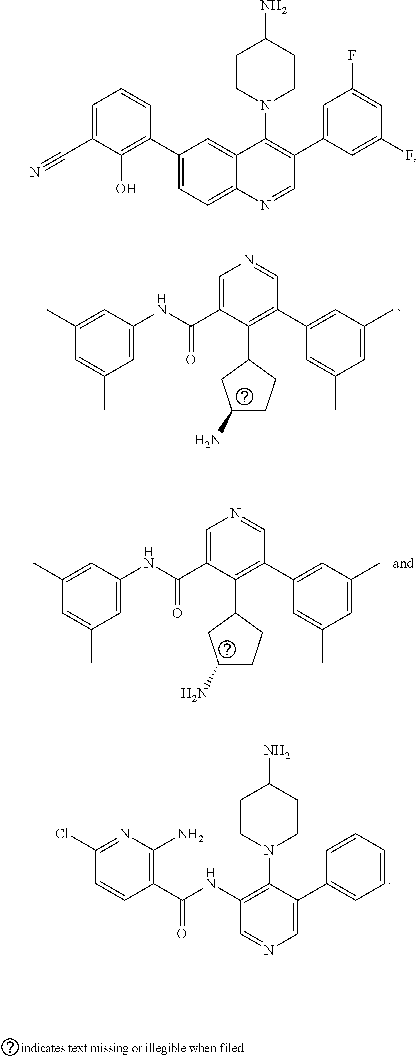

In certain above embodiments, useful compounds are those where Z is absent.

In certain above embodiments, useful compounds are those where Z is —N(H)—C(═O)— or —C(═O)—NH—, and y is 1.

In certain above embodiments, useful compounds are those where g is 0.

In certain above embodiments, useful compounds are those, having a structure of Formula Ia or Ib, wherein Z and R¹ together with the ring to which each is attached form a fused:

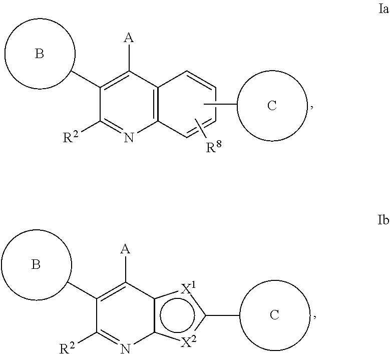

wherein, X¹ and X² are each independently selected from the group consisting of O, N, N—R⁸, S, and C—R⁸;
wherein, R⁸ is selected from the group consisting of hydrogen, optionally substituted C₁-C₆ alkyl, and —C(═O)OR^{8a}, wherein R^{8a} is hydrogen or C₁-C₆ alkyl, wherein the optional substituents are selected from the group consisting of halogen, —NH₂ and hydroxy.

In certain above embodiments, useful compounds are those having a structure of Formula Ia-1:

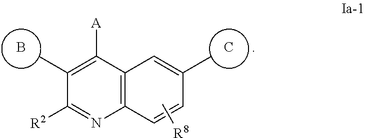

In certain above embodiments, useful compounds are those having a structure of Formula Ia-2:

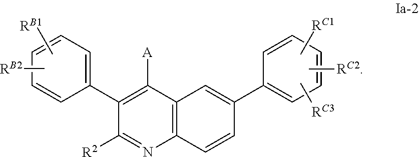

In certain above embodiments, useful compounds are those having a structure of Formula Ia-3:

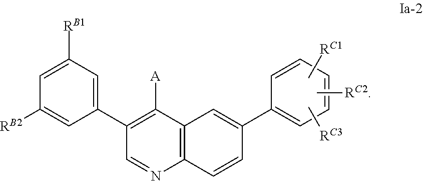

In certain above embodiments, useful compounds are those having a structure of Formula Ib-1:

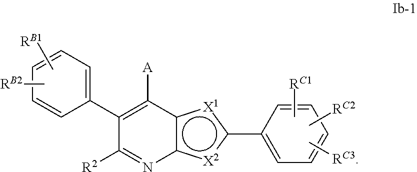

In certain above embodiments, useful compounds are those having a structure of Formula Ib-1a-Ib-1e:

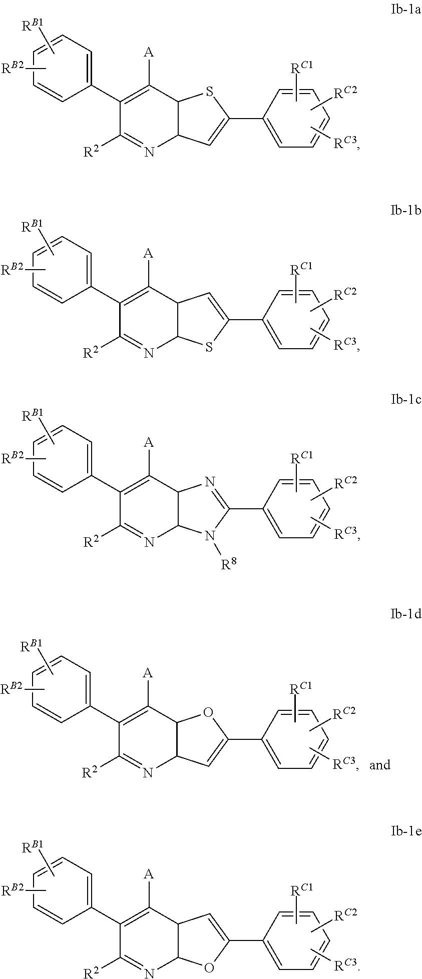

In certain above embodiments, useful compounds are those wherein the positions of R^{B1} and R^{B2} in each of Ib-1a -Ib-1e are:

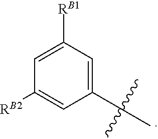

In certain above embodiments, useful compounds are those wherein, R^{B1} and R^{B2} are each independently selected from the group consisting of C₁-C₆ alkyl, halogen and C₁-C₆ alkoxy.

In certain above embodiments, useful compounds are those wherein the C₁-C₆ alkyl is methyl, the halogen is fluoro and the C₁-C₆alkoxy is methoxy.

In certain above embodiments, useful compounds are those wherein at least one of R^{B1} and R^{B2} is fluoro.

In certain above embodiments, useful compounds are those wherein both of R^{B1} and R^{B2} is fluoro.

In certain above embodiments, useful compounds are those wherein R^{C1} is hydrogen, and the positions of R^{C2} and R^{C3} are:

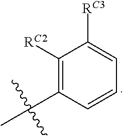

In certain above embodiments, useful compounds are those wherein,
R^{C2} is selected from the group consisting of hydroxy, C₁-C₆ alkoxy, —O-piperidinyl, —N—(CH₂)₃—NH₂, —O—C₂-C₆alkenyl, —O—(CH₂)_{q1}—O—(CH₂)ᵣ₁—CH₃, wherein q1 and r1 are each independently an integer from 1 to 4; and,
R^{C3} is selected from the group consisting of cyano and halogen.

In certain above embodiments, useful compounds are those wherein,
R^{C2} is selected from the group consisting of hydroxy, methoxy, —O—CH₂—CH═CH₂ and —O—CH₂—O—CH₃, and
R^{C3} is selected from the group consisting of cyano and bromo.

In certain above embodiments, useful compounds are those wherein R^{C1} is hydrogen, and the positions of R^{C2} and R^{C3} are:

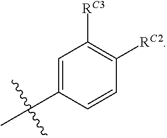

In certain above embodiments, useful compounds are those wherein,
R^{C2} and R^{C3} are each halogen.

In certain above embodiments, useful compounds are those wherein the halogen is fluoro.

In certain above embodiments, useful compounds are those wherein R^{C1} is hydrogen, and the positions of R^{C2} and R^{C1} are:

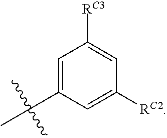

In certain above embodiments, useful compounds are those wherein,
R^{C2} and R^{C3} are each independently selected from the group consisting of hydroxy, C₁-C₆alkoxy, halogen, —(C═O)—NH₂, —O—C₂-C₆alkenyl and C₁-C₆alkyl.

In certain above embodiments, useful compounds are those wherein,
R^{C2} and R^{C3} are each independently selected from the group consisting of —(C═O)—NH₂, methoxy, fluoro and methyl.

In certain above embodiments, useful compounds are those wherein
Ring B is phenyl di-substituted with R^{B1} and R^{B2}; and,
Ring C is 4- to 10-membered monocyclic or bicyclic heterocyclyl.

In certain above embodiments, useful compounds are those wherein Ring C is a 9- to 10-membered bicyclic lactam or cyclic urea.

In certain above embodiments, useful compounds are those wherein Ring C is selected from the group consisting of:

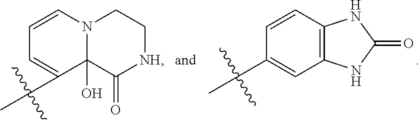 [SMILES: *C1=CC=CN2CCNC(=O)C12O ; "and" ; *c1ccc2[nH]c(=O)[nH]c2c1 ; "."]

In certain above embodiments, useful compounds are those having a structure of Formula Ic, wherein Z is absent and R¹ and R^{C1} together with the ring to which R¹ is attached and the ring to which R^{C1} is attached form a fused ring substituted with R⁸:

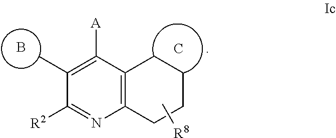

In certain above embodiments, useful compounds are those wherein R⁸ is hydrogen.

In certain above embodiments, useful compounds are those wherein Ring C is 8- to 10-membered heteroaryl substituted with R^{C2} and R^{C3}.

In certain above embodiments, useful compounds are those wherein Ring C is:

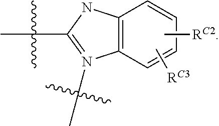

In certain above embodiments, useful compounds are those wherein each of R^{C2} and R^{C3} is fluor.

In certain above embodiments, useful compounds are those wherein Ring B is phenyl di-substituted with R^{B1} and R^{B2}.

In certain above embodiments, useful compounds are those wherein R^{B1} is halogen, and R^{B2} is halogen or C₁-C₆ alkyl.

In certain above embodiments, useful compounds are those wherein R^{B1} is fluor and R^{B2} is fluoro or methyl.

In certain above embodiments, useful compounds are those wherein the positions of R^{B1} and R^{B2} in the phenyl ring are:

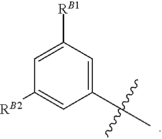

In certain above embodiments, useful compounds are those having a structure of Formula Id, wherein Z and R¹ and together with the ring to which each is attached form a fused ring:

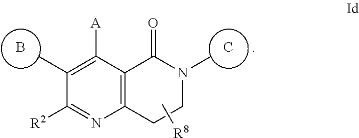

In certain above embodiments, useful compounds are those wherein R⁸ is hydrogen.

In certain above embodiments, useful compounds are those wherein Ring C is phenyl substituted with R^{C1}, R^{C2} and R^{C3}.

In certain above embodiments, useful compounds are those wherein R^{C1} is hydrogen, and the positions of R^{C2} and R^{C3} are:

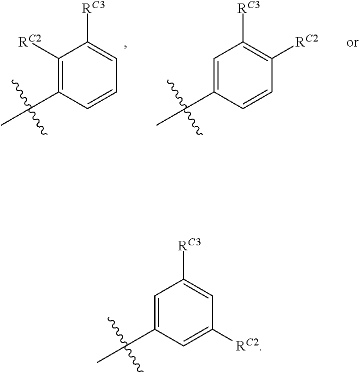

In certain above embodiments, useful compounds are those wherein, R^{C2} and R^{C3} are each independently selected from the group consisting of C₁-C₆ alkyl and halogen.

In certain above embodiments, useful compounds are those wherein the C₁-C₆ alkyl is methyl and the halogen is fluoro.

In certain above embodiments, useful compounds are those wherein Ring B is phenyl di-substituted with R^{B1} and R^{B2}.

In certain above embodiments, useful compounds are those wherein R^{B1} is halogen, and R^{B2} is halogen or C₁-C₆ alkyl.

In certain above embodiments, useful compounds are those wherein R^{B1} is fluoro and R^{B2} is fluoro or methyl.

In certain above embodiments, useful compounds are those wherein the positions of R^{B1} and R^{B2} in the phenyl are:

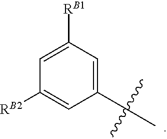

In certain above embodiments, useful compounds are those having a structure of Formula Ie, wherein R² and R^{B1} together with the ring to which R² is attached and the ring to which R^{B1} is attached form a fused ring:

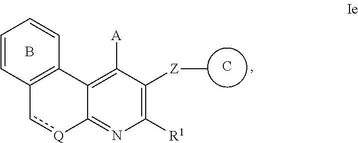

wherein,
   Q is O or CH₂, and [symbol] is a single bond; or Q is N and [symbol] is a double bond;
   R¹ is hydrogen or halogen; and,
   Ring B is phenyl substituted with R^{B2}.

In certain above embodiments, useful compounds are those wherein R¹ is hydrogen.

In certain above embodiments, useful compounds are those wherein Ring C is 8- to 10-membered heteroaryl substituted with R^{C1}, R^{C2} and R^{C3}.

In certain above embodiments, useful compounds are those wherein, R^{C1} is hydrogen, and Ring C is:

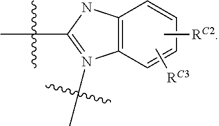

In certain above embodiments, useful compounds are those wherein R^{B2} is halogen.

In certain above embodiments, useful compounds are those wherein R^{B2} is fluoro.

In certain above embodiments, useful compounds are those wherein Q is N, and [symbol] is a double bond.

In certain above embodiments, useful compounds are those wherein Q is O, and [symbol] is a single bond.

In certain above embodiments, useful compounds are those wherein A is 5- to 11-membered heterocyclyl, or 5- to 6-membered heteroaryl, each substituted with —NH—R⁶; or with R^{F} and R^{G}.

In certain above embodiments, useful compounds are those wherein A has the structure:

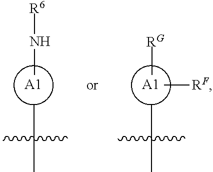

wherein,
   Ring A1 is 5- to 6-membered heteroaryl or 5- to 6-membered heterocyclyl.

In certain above embodiments, useful compounds are those wherein A has the structure:

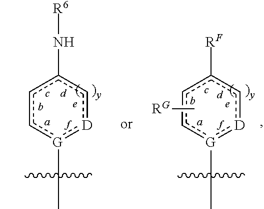

wherein,
   G is N; D is CH₂; y is 0 or 1; and each [symbol] is a single bond;
   G is C; D is CH₂; y is 1; and [symbol] ^{a} is a double bond, and the other [symbol] are each a single bond;
   G is N, D is CH₂; is 0; and [symbol] ^{b} and [symbol] ^{e} are each a double bond, and the other [symbol] are each a single bond;
   G is C; D is N or C—H; y is 1; and each [symbol] is a double bond.

In certain above embodiments, useful compounds are those wherein A is selected from the group consisting of:

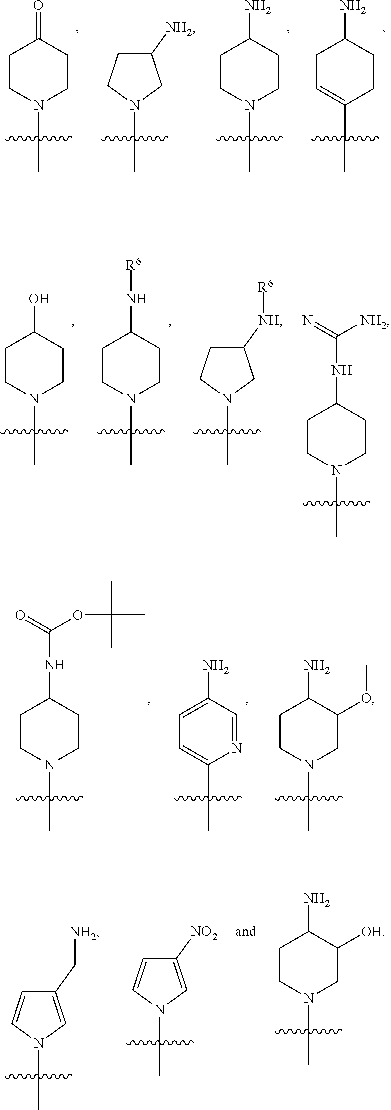

In certain above embodiments, useful compounds are those wherein A is a spirocyclic having the structure:

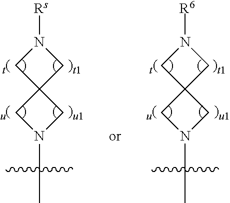

wherein, t, t1, u and u1 are each independently 1 or 2.

In certain above embodiments, useful compounds are those wherein A is selected from the group consisting of:

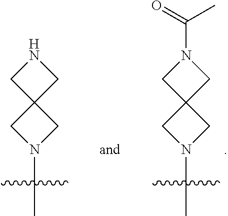 [SMILES: *N1CC2(CNC2)C1 ; *N1CC2(C1)CN(C(C)=O)C2]

In certain above embodiments, useful compounds are those wherein A is —O—(C₃-C₈ cycloalkyl) substituted with —NH—R⁶, or substituted with R^{F} and R^{G}.

In certain above embodiments, useful compounds are those wherein A has the structure:

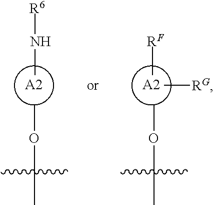

wherein,
   Ring A2 is C₄-C₆ cycloalkyl.

In certain above embodiments, useful compounds are those wherein A is selected from the group consisting of:

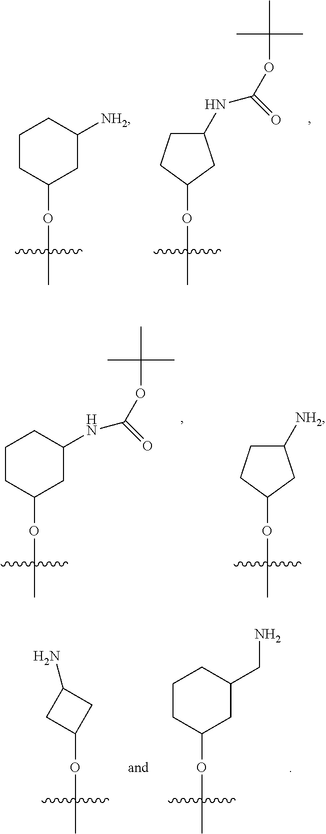 [SMILES: *OC1CCCC(N)C1 ; *OC1CCC(NC(=O)OC(C)(C)C)C1 ; *OC1CCCC(NC(=O)OC(C)(C)C)C1 ; *OC1CCC(N)C1 ; *OC1CC(N)C1 ; *OC1CCCC(CN)C1]

In certain above embodiments, useful compounds are those wherein
R⁶ together with R^{C1} form:

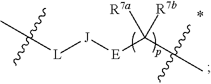

wherein,

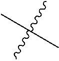

represents the attachment point of R⁶ to A;
p is an integer from 1 to 4;
R^{7a} and R^{7b} are, in each instance, independently selected from the group consisting of hydroxyl, optionally substituted —C₁-C₆ alkyl, —N₃, —NR^{a}R^{b}, wherein R^{a} an R^{b} are each independently H, or C₁-C₆ alkyl;
E is absent, —O— or —N(R^{b})—, wherein R^{b} is H or optionally substituted C₁-C₆alkyl;
J is —C(O)— or —C(R^{7a}R^{7b})—; and,
L is absent, —O— or —N(H)—.

In certain above embodiments, useful compounds are those wherein R⁶ together with R^{C1} form:

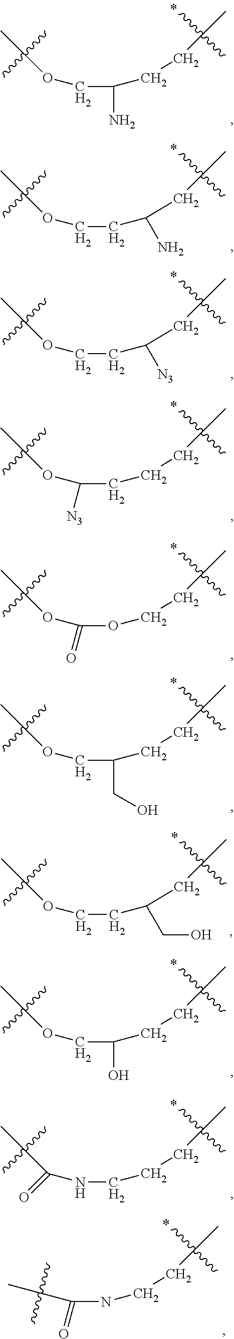

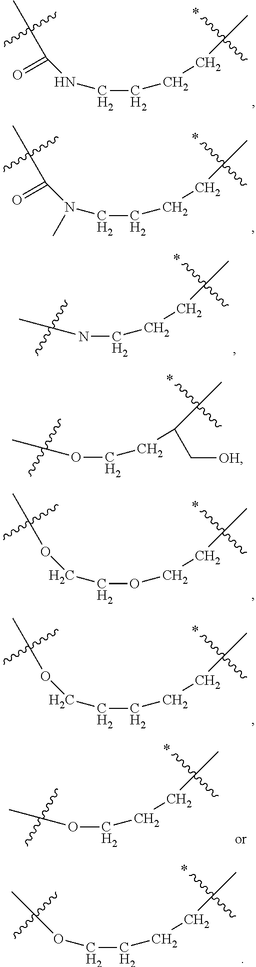

In certain above embodiments, useful compounds are those having a structure of Formula Ia-4:

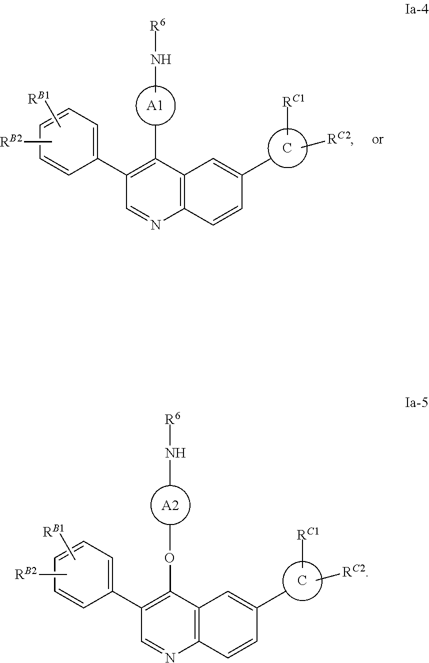

In certain above embodiments, useful compounds arm those having a structure of Formula Ia-4A:

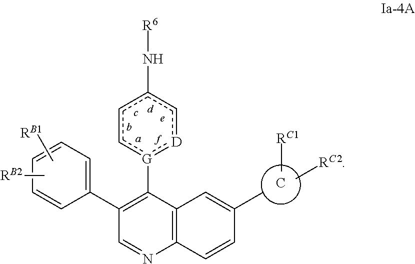

In certain above embodiments, useful compounds are those wherein Ring C to which R^{C1} is attached has the structure:

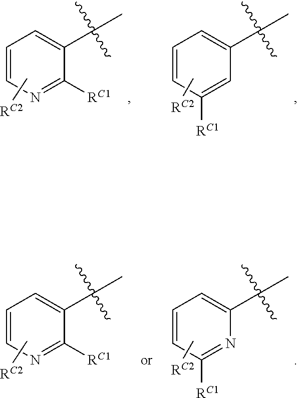

In certain above embodiments, useful compounds are those wherein Ring C to which R^{C1} is attached has the structure:

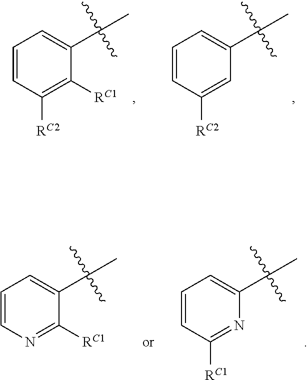

In certain above embodiments, useful compounds are those wherein R^{C2} is cyano or —(C═O)—NH₂.

In certain above embodiments, useful compounds are those having a structure of Formulae Ia-6, Ia-7, Ib-2 and Ib-3:

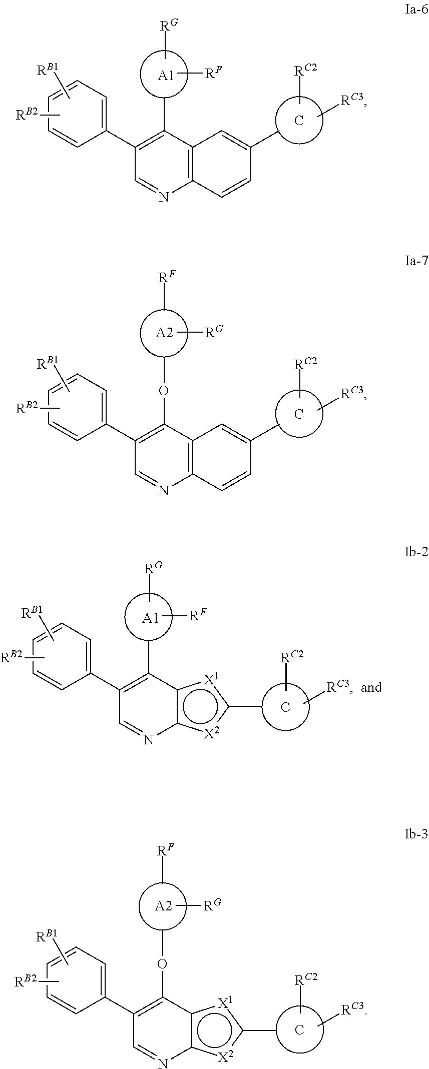

In certain above embodiments, useful compounds are those wherein the positions of R^{B1} and R^{B2} in each of Ia-6, Ia-7, Ib-2 and Ib-3 are:

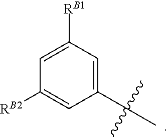

In certain above embodiments, useful compounds are those wherein, R^{B1} an R^{B2} are each independently selected from the group consisting of C₁-C₆ alkyl, halogen and C₁-C₆ alkoxy.

In certain above embodiments, useful compounds are those wherein the C₁-C₆ alkyl is methyl, the halogen is fluoro and the C₁-C₆ alkoxy is methoxy.

In certain above embodiments, useful compounds are those wherein at least one of R^{B1} and R^{B2} is fluoro.

In certain above embodiments, useful compounds are those wherein both of R^{B1} and R^{B2} is fluoro.

In certain above embodiments, useful compounds are those wherein the positions of R^{C2} and R^{C3} are:

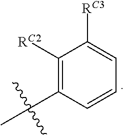

In certain above embodiments, useful compounds are those wherein,
R^{C2} is selected from the group consisting of hydroxy. C₁-C₆alkoxy, —O-piperidinyl, —N—(CH₂)₃—NH₂, —O—C₂-C₆alkenyl, —O—(CH₂)_{q}—O—(CH₂)ᵣ₁—CH₃, wherein q1 and r1 are each independently an integer from 1 to 4; and,
R^{C3} is selected from the group consisting of cyano and halogen.

In certain above embodiments, useful compounds are those wherein,
R^{C2} is selected from the group consisting of hydroxy, methoxy, —O—CH₂—CH═CH₂ and —O—CH₂—O—CH₃, and
R^{C3} is selected from the group consisting of cyano and bromo.

In certain above embodiments, useful compounds are those wherein R^{C1} is hydrogen, and the positions of R^{C2} and R^{C3} are:

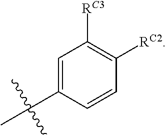

In certain above embodiments, useful compounds are those wherein,
R^{C2} and R^{C3} are each halogen.

In certain above embodiments, useful compounds are those wherein the halogen is fluoro.

In certain above embodiments, useful compounds are those wherein R^{C1} is hydrogen, and the positions of R^{C2} and R^{C3} are:

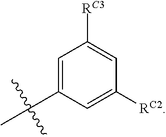

In certain above embodiments, useful compounds are those wherein,
R^{C2} and R^{C3} are each independently selected from the group consisting of hydroxy, C₁-C₆ alkoxy, halogen, —(C═O)—NH₂, —O—C₂-C₆ alkenyl and C₁-C₆ alkyl.

In certain above embodiments, useful compounds are those wherein,
R^{C2} and R^{C3} are each independently selected from the group consisting of —(C═O)—NH₂, methoxy, fluoro and methyl.

In certain above embodiments, useful compounds are those wherein A is —NR⁴R⁵.

In certain above embodiments, useful compounds are those wherein
R⁴ is optionally substituted C₁-C₆ alkyl, —C₁-C₆ alkyl-NH—CH₃, C₁-C₆ alkyl-NH₂, —NH—C₁-C₆ alkyl-NH—CH₃, —NH—C₁-C₆ alkyl-NH₂ and,
R⁵ is hydrogen or optionally substituted C₁-C₆ alkyl.

In certain above embodiments, useful compounds are those wherein R⁴ is —(CH₂)₃—NH—CH₃; and R⁵ is hydrogen.

In certain above embodiments, useful compounds are those having a structure of Formula If, wherein R⁵ and R^{B1} together with the N to which R⁵ is attached and Ring B to which R^{B1} is attached form a fused ring:

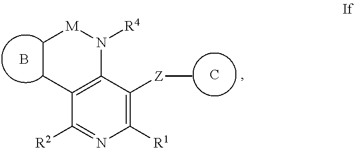

wherein,
   M is a carbonyl or C—R^{M1}R^{M2}, wherein R^{M1} and R^{M2} are each independently selected from the group consisting of hydrogen, optionally substituted C₁-C₆ alkyl, and C₁-C₆ alkyl-NH₂, wherein, the optional substituents are selected from the group consisting of halogen and hydroxy.

In certain above embodiments, useful compounds are those wherein
M is CH₂;
R¹ and R² are each hydrogen; and,
R⁴ is C₁-C₆alkyl-NHR^{4a}.

In certain above embodiments, useful compounds are those wherein Z is —C(═O)—NH—.

In certain above embodiments, useful compounds are those wherein
Ring B is phenyl monosubstituted with R^{B1}, or disubstituted with R^{B1} and R^{B2}.

In certain above embodiments, useful compounds are those wherein R^{B1} and R^{B2} are each fluoro.

In certain above embodiments, useful compounds are those wherein Ring C is 4- to 10-membered monocyclic or bicyclic fused heterocyclyl or 5- to 10-membered heteroaryl, each substituted with R^{C1}, R^{C2} and R^{C1}.

In certain above embodiments, useful compounds are those wherein Ring C is 6- to 10-membered aryl substituted with R^{C1}, R^{C2} and R^{C3}.

In certain above embodiments, useful compounds are those having a structure of Formula Ig, wherein Z is absent, and R⁵ and R^{C1} together with the N to which R⁵ is attached and Ring C to which R^{C1} is attached form a fused ring:

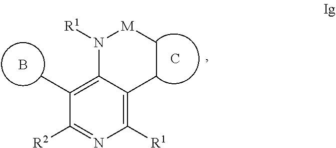

wherein,
   M is a carbonyl, or C—R^{M1}R^{M2}, wherein R^{M1} and R^{M2} are each independently selected from the group consisting of hydrogen, optionally substituted C₁-C₆ alkyl, and C₁-C₆alkyl-NH₂, wherein, the optional substituent is halogen or hydroxy.

In certain above embodiments, useful compounds are those wherein
M is carbonyl;
R¹ and R² are each hydrogen; and,
R⁴ is C₁-C₆ alkyl-NHR^{4a}.
In certain above embodiments, useful compounds are those wherein
Ring B is phenyl monosubstituted with R^{B1}, or disubstituted with R^{B1} and R^{B2}.

In certain above embodiments, useful compounds are those wherein R^{B1} and R^{B2} are each fluoro.

In certain above embodiments, useful compounds are those wherein Ring C is 4- to 10-membered monocyclic or bicyclic fused heterocyclyl or 5- to 10-membered heteroaryl, each substituted with R^{C1}, R^{C2} and R^{C3}.

In certain above embodiments, useful compounds are those wherein g is 1. These compounds will have a counter ion such as a halo.

In certain above embodiments, useful compounds are those wherein R^{C1} together with R³ form:

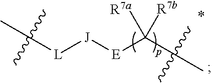

wherein,

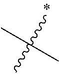

represents the attachment point of R⁶ to A;
p is an integer from 1 to 4;
R^{7a} and R^{7b} are, in each instance, independently selected from the group consisting of hydroxyl, optionally substituted —C₁-C₆alkyl, —N₃, —NR^{a}R^{b}, wherein R^{a} and R^{b} are each independently H, or C₁-C₆ alkyl:
E is absent, —O— or —N(R^{b})—, wherein R^{b} is H or optionally substituted C₁-C₆alkyl;
J is —C(O)— or —C(R^{7a}R^{7b})—; and,
L is absent, —O— or —N(H)—.

In certain above embodiments, useful compounds are those having a structure of Formula I-2:

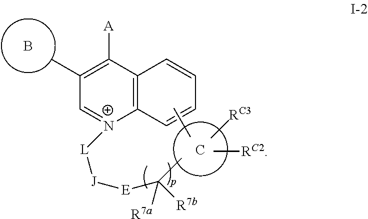

In certain above embodiments, useful compounds are those wherein -L-J-E-(CR^{7a}R^{7b})ₚ— form:

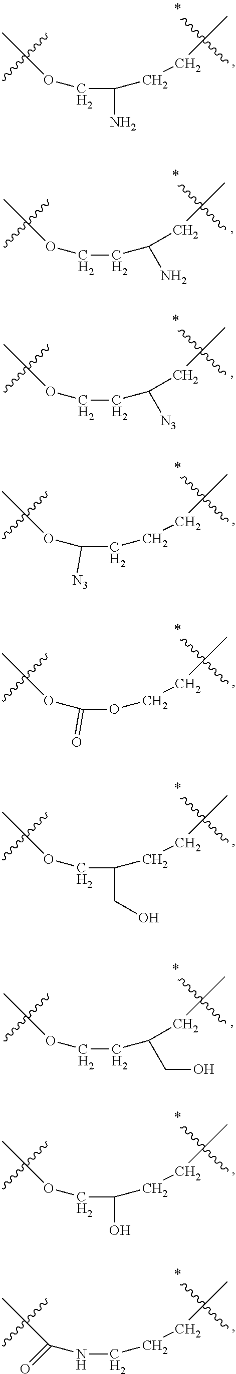

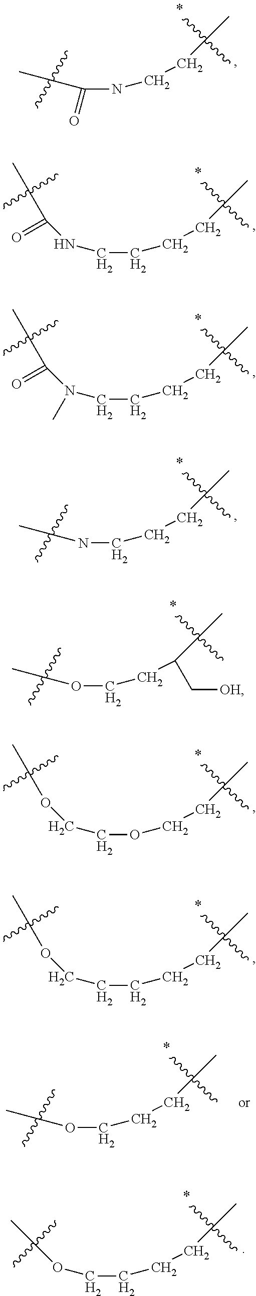

In certain above embodiments, useful compounds are those wherein Ring B is phenyl di-substituted with R^{B1} and R^{B2}.

In certain above embodiments, useful compounds are those wherein R^{B1} is halogen, and R^{B2} is halogen or C₁-C₆ alkyl.

In certain above embodiments, useful compounds are those wherein R^{B1} is fluoro, and R^{B2} is fluoro or methyl.

In certain above embodiments, useful compounds are those wherein the positions of R^{B1} and R^{B2} in the phenyl ring are:

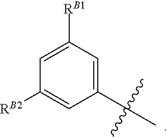

In certain above embodiments, useful compounds are those wherein Ring C to which R^{C1} is attached has the structure:

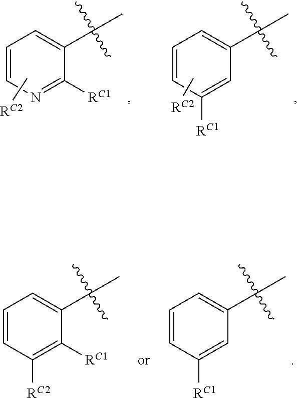

In certain above embodiments, useful compounds are those wherein R^{C2} is cyano.

In certain above embodiments, useful compounds are those wherein A has the structure:

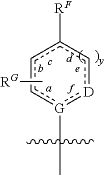

wherein,
   G is N; D is CH₂; y is 0 or 1; and each [symbol] is a single bond;
   G is C; D is CH₂; y is 1; and [symbol] ^{a} is a double bond, and the other [symbol] are each a single bond;
   G is N, D is CH₂; y is 0; and [symbol] ^{b} and [symbol] ^{e} are each a double bond, and the other [symbol] are each a single bond;
   G is C; D is N or C—H; y is 1; and each [symbol] is a double bond.

In certain above embodiments, useful compounds are those wherein A is selected from the group consisting of:

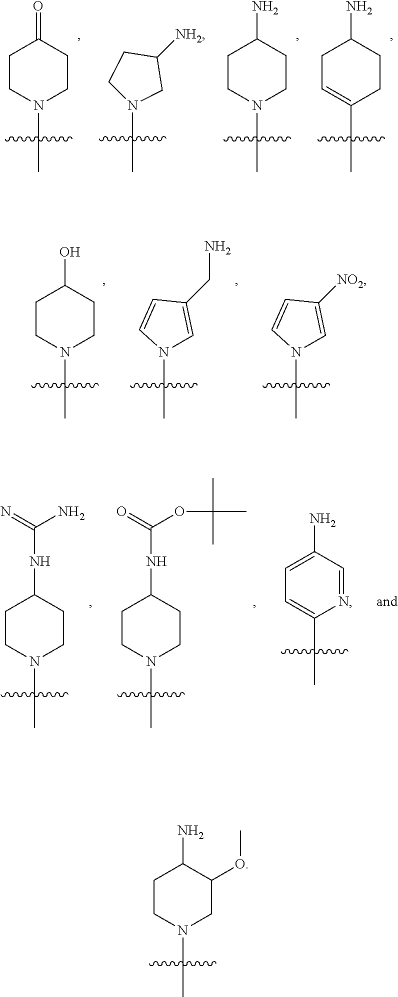

In certain above embodiments, useful compounds are those wherein A has the structure:

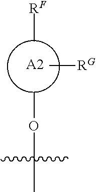

wherein, Ring A2 is C₄-C₆ cycloalkyl.

In certain above embodiments, useful compounds are those wherein A is selected from the group consisting of:

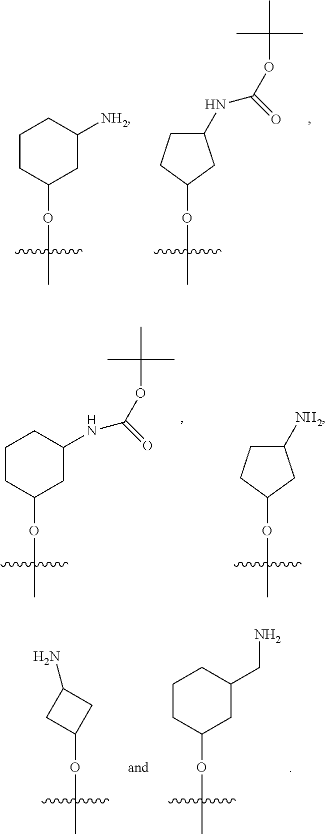 [SMILES: *OC1CCCC(N)C1 ; *OC1CCC(NC(=O)OC(C)(C)C)C1 ; *OC1CCCC(NC(=O)OC(C)(C)C)C1 ; *OC1CCC(N)C1 ; *OC1CC(N)C1 ; *OC1CCCC(CN)C1]

In certain above embodiments, useful compounds are those shown in Table 1, or pharmaceutically acceptable salt thereof.

In certain embodiments, the subject matter described herein is directed to a pharmaceutical composition comprising a compound of any above embodiment, or pharmaceutically acceptable salt thereof, and a pharmaceutically acceptable excipient.

In certain embodiments, the subject matter described herein is directed to a method of treating a subject afflicted with a disease associated with somatostatin, comprising administering to the subject a compound of any above embodiment or a pharmaceutical composition thereof.

In certain embodiments, the subject matter described herein is directed to a method of treating a subject afflicted with a disease wherein the disease is selected from the group consisting of diabetes, diarrhea, inflammatory bowel disease, irritable bowel syndrome, cancer, acromegaly, depression, chronic atrophic gastritis, Crohn's disease, ulcerative colitis, retinopathy, arthritis, restenosis, neuroendocrine tumors (NETs), and pain.

In certain embodiments, the subject matter described herein is directed to a method of activating somatostatin receptor in a subject, comprising administering to the subject a compound of any above embodiment or pharmaceutical composition thereof.

In certain embodiments, the subject matter described herein is directed to compounds shown in Table 1.

TABLE 1

| Cpd. No. | Structure | Cpd. No. | Structure |
|---|---|---|---|
| 1 | 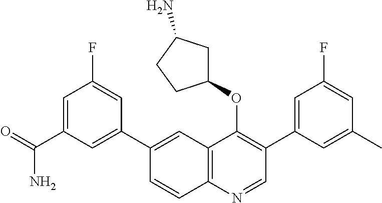 [SMILES: Cc1cc(F)cc(-c2cnc3ccc(-c4cc(F)cc(C(N)=O)c4)cc3c2O[C@H]2CC[C@H](N)C2)c1] | 83 | 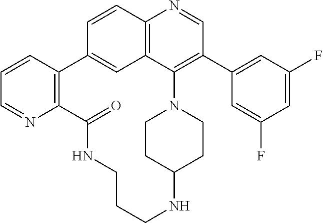 [SMILES: O=C1NCCCNC2CCN(CC2)c2c(-c3cc(F)cc(F)c3)cnc3ccc(cc23)-c2cccnc21] |
| 2 | 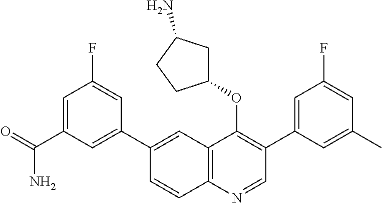 [SMILES: Cc1cc(F)cc(-c2cnc3ccc(-c4cc(F)cc(C(N)=O)c4)cc3c2O[C@@H]2CC[C@H](N)C2)c1] | 84 | 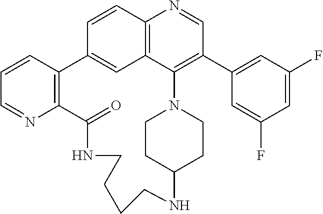 [SMILES: O=C1NCCCCNC2CCN(CC2)c2c(-c3cc(F)cc(F)c3)cnc3ccc(cc23)-c2cccnc21] |
| 3 | 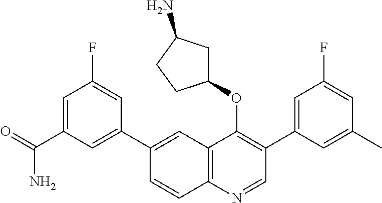 [SMILES: Cc1cc(F)cc(-c2cnc3ccc(-c4cc(F)cc(C(N)=O)c4)cc3c2O[C@H]2CC[C@@H](N)C2)c1] | 85 | 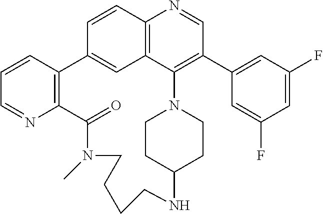 [SMILES: CN1CCCCNC2CCN(CC2)c2c(-c3cc(F)cc(F)c3)cnc3ccc(cc23)-c2cccnc2C1=O] |
| 4 | 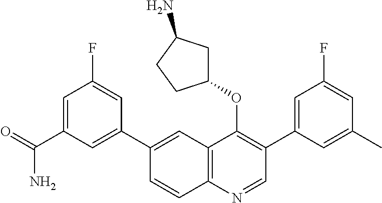 [SMILES: Cc1cc(F)cc(-c2cnc3ccc(-c4cc(F)cc(C(N)=O)c4)cc3c2O[C@@H]2CC[C@@H](N)C2)c1] | 86 | 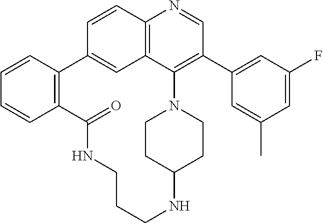 [SMILES: Cc1cc(F)cc(-c2cnc3ccc4cc3c2N2CCC(CC2)NCCCNC(=O)c2ccccc2-4)c1] |
| 5 | 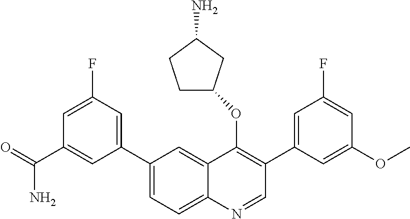 [SMILES: COc1cc(F)cc(-c2cnc3ccc(-c4cc(F)cc(C(N)=O)c4)cc3c2O[C@@H]2CC[C@H](N)C2)c1] | 87 | 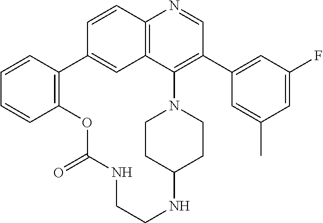 [SMILES: Cc1cc(F)cc(-c2cnc3ccc4cc3c2N2CCC(CC2)NCCNC(=O)Oc2ccccc2-4)c1] |
| 6 | 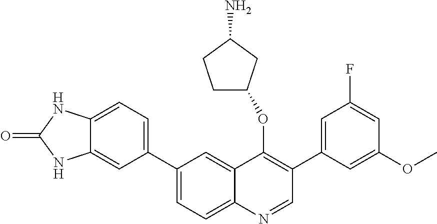 [SMILES: COc1cc(F)cc(-c2cnc3ccc(-c4ccc5[nH]c(=O)[nH]c5c4)cc3c2O[C@@H]2CC[C@H](N)C2)c1] | 88* | 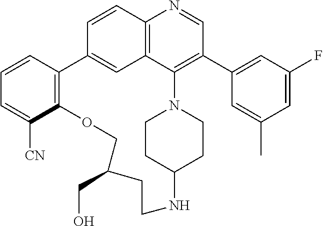 |
| 7 | 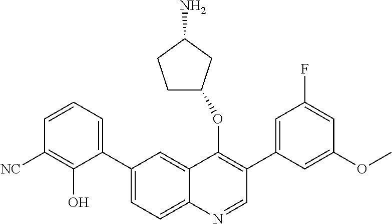 [SMILES: COc1cc(F)cc(-c2cnc3ccc(-c4cccc(C#N)c4O)cc3c2O[C@@H]2CC[C@H](N)C2)c1] | 89* | 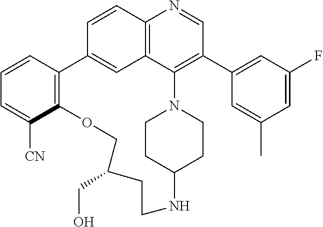 |
| 8 | 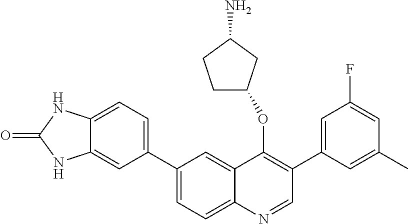 [SMILES: Cc1cc(F)cc(-c2cnc3ccc(-c4ccc5[nH]c(=O)[nH]c5c4)cc3c2O[C@@H]2CC[C@H](N)C2)c1] | 90* | 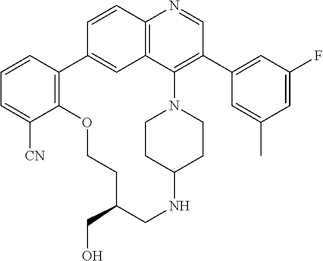 [SMILES: Cc1cc(F)cc(-c2cnc3ccc4cc3c2N2CCC(CC2)NC[C@@H](CO)CCOc2c(C#N)cccc2-4)c1] |
| 9 | 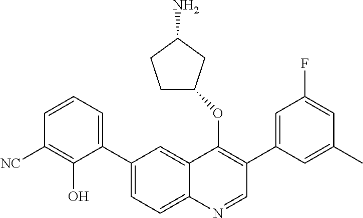 [SMILES: Cc1cc(F)cc(-c2cnc3ccc(-c4cccc(C#N)c4O)cc3c2O[C@@H]2CC[C@H](N)C2)c1] | 91* | 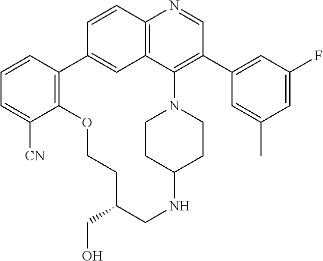 [SMILES: Cc1cc(F)cc(-c2cnc3ccc4cc3c2N2CCC(CC2)NC[C@H](CO)CCOc2c(C#N)cccc2-4)c1] |
| 10 | 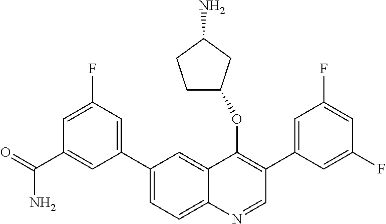 [SMILES: NC(=O)c1cc(F)cc(-c2ccc3ncc(-c4cc(F)cc(F)c4)c(O[C@@H]4CC[C@H](N)C4)c3c2)c1] | 92 | 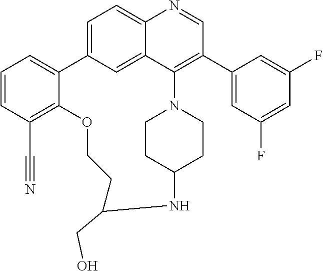 [SMILES: N#Cc1cccc2c1OCCC(CO)NC1CCN(CC1)c1c(-c3cc(F)cc(F)c3)cnc3ccc-2cc13] |
| 11 | 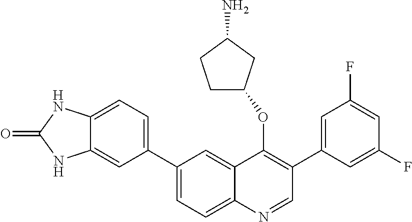 [SMILES: N[C@H]1CC[C@@H](Oc2c(-c3cc(F)cc(F)c3)cnc3ccc(-c4ccc5[nH]c(=O)[nH]c5c4)cc23)C1] | 93 | 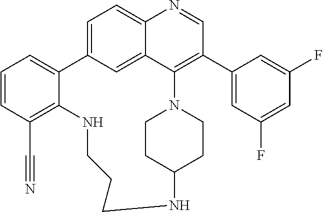 [SMILES: N#Cc1cccc2c1NCCCNC1CCN(CC1)c1c(-c3cc(F)cc(F)c3)cnc3ccc-2cc13] |
| 12 | 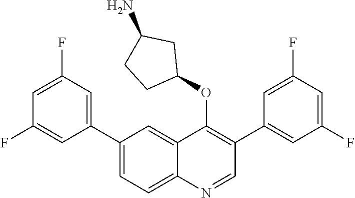 [SMILES: N[C@@H]1CC[C@H](Oc2c(-c3cc(F)cc(F)c3)cnc3ccc(-c4cc(F)cc(F)c4)cc23)C1] | 94 | 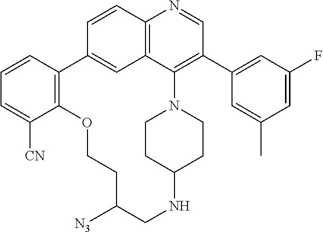 [SMILES: Cc1cc(F)cc(-c2cnc3ccc4cc3c2N2CCC(CC2)NCC(N=[N+]=[N-])CCOc2c(C#N)cccc2-4)c1] |
| 13 | 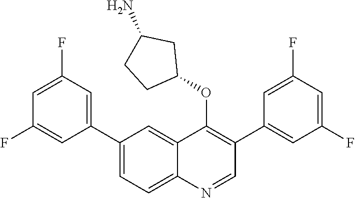 [SMILES: N[C@H]1CC[C@@H](Oc2c(-c3cc(F)cc(F)c3)cnc3ccc(-c4cc(F)cc(F)c4)cc23)C1] | 95 | 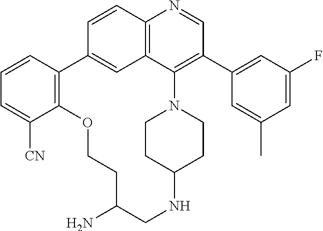 [SMILES: Cc1cc(F)cc(-c2cnc3ccc4cc3c2N2CCC(CC2)NCC(N)CCOc2c(C#N)cccc2-4)c1] |
| 14 | 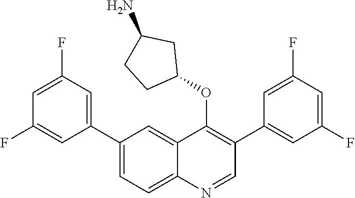 [SMILES: N[C@@H]1CC[C@@H](Oc2c(-c3cc(F)cc(F)c3)cnc3ccc(-c4cc(F)cc(F)c4)cc23)C1] | 96* | 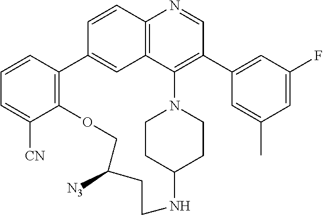 [SMILES: Cc1cc(F)cc(-c2cnc3ccc4cc3c2N2CCC(CC2)NCC[C@@H](N=[N+]=[N-])COc2c(C#N)cccc2-4)c1] |
| 15 | 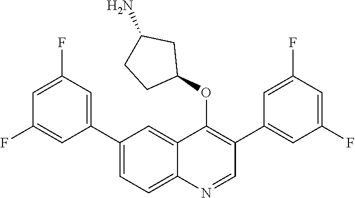 [SMILES: N[C@H]1CC[C@H](Oc2c(-c3cc(F)cc(F)c3)cnc3ccc(-c4cc(F)cc(F)c4)cc23)C1] | 97* | 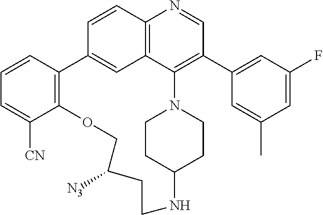 [SMILES: Cc1cc(F)cc(-c2cnc3ccc4cc3c2N2CCC(CC2)NCC[C@H](N=[N+]=[N-])COc2c(C#N)cccc2-4)c1] |
| 16 | 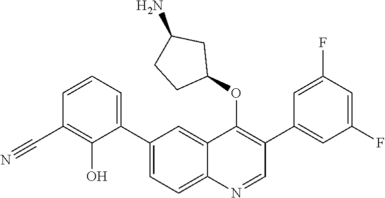 [SMILES: N#Cc1cccc(-c2ccc3ncc(-c4cc(F)cc(F)c4)c(O[C@H]4CC[C@@H](N)C4)c3c2)c1O] | 98* | 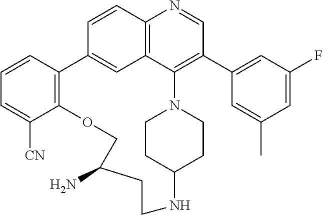 [SMILES: Cc1cc(F)cc(-c2cnc3ccc4cc3c2N2CCC(CC2)NCC[C@@H](N)COc2c(C#N)cccc2-4)c1] |
| 17 | 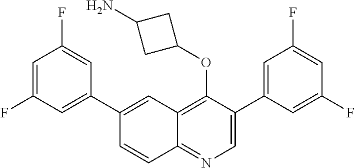 [SMILES: NC1CC(Oc2c(-c3cc(F)cc(F)c3)cnc3ccc(-c4cc(F)cc(F)c4)cc23)C1] | 99* | 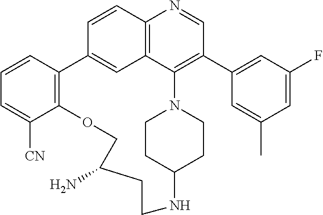 [SMILES: Cc1cc(F)cc(-c2cnc3ccc4cc3c2N2CCC(CC2)NCC[C@H](N)COc2c(C#N)cccc2-4)c1] |
| 18 | 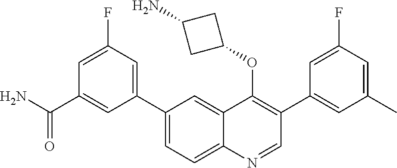 [SMILES: Cc1cc(F)cc(-c2cnc3ccc(-c4cc(F)cc(C(N)=O)c4)cc3c2O[C@H]2C[C@@H](N)C2)c1] | 100* | 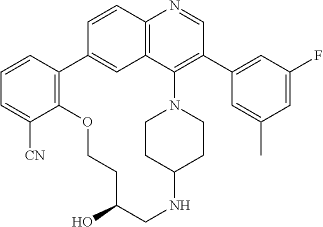 [SMILES: Cc1cc(F)cc(-c2cnc3ccc4cc3c2N2CCC(CC2)NC[C@@H](O)CCOc2c(C#N)cccc2-4)c1] |
| 19 | 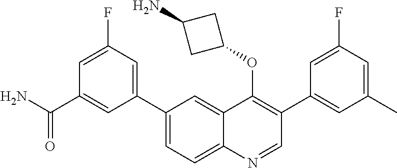 [SMILES: Cc1cc(F)cc(-c2cnc3ccc(-c4cc(F)cc(C(N)=O)c4)cc3c2O[C@H]2C[C@H](N)C2)c1] | 101* | 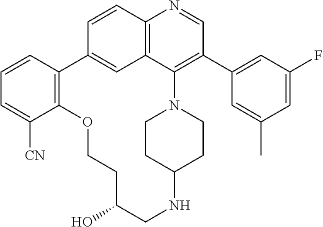 [SMILES: Cc1cc(F)cc(-c2cnc3ccc4cc3c2N2CCC(CC2)NC[C@H](O)CCOc2c(C#N)cccc2-4)c1] |
| 20 | 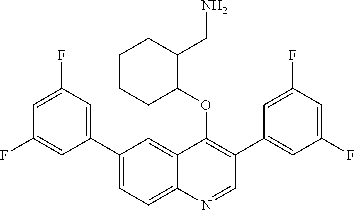 [SMILES: NCC1CCCCC1Oc1c(-c2cc(F)cc(F)c2)cnc2ccc(-c3cc(F)cc(F)c3)cc12] | 102* | 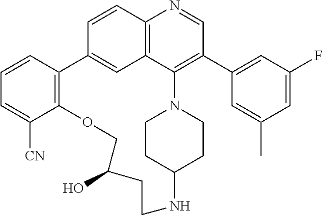 [SMILES: Cc1cc(F)cc(-c2cnc3ccc4cc3c2N2CCC(CC2)NCC[C@@H](O)COc2c(C#N)cccc2-4)c1] |
| 21 | 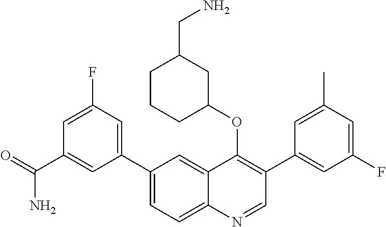 [SMILES: Cc1cc(F)cc(-c2cnc3ccc(-c4cc(F)cc(C(N)=O)c4)cc3c2OC2CCCC(CN)C2)c1] | 103* | 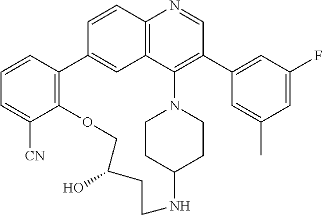 [SMILES: Cc1cc(F)cc(-c2cnc3ccc4cc3c2N2CCC(CC2)NCC[C@H](O)COc2c(C#N)cccc2-4)c1] |
| 22 | 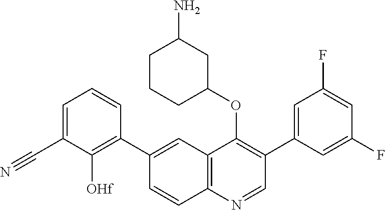 | 104 | 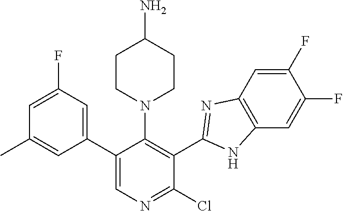 [SMILES: Cc1cc(F)cc(-c2cnc(Cl)c(-c3nc4cc(F)c(F)cc4[nH]3)c2N2CCC(N)CC2)c1] |
| 23 | 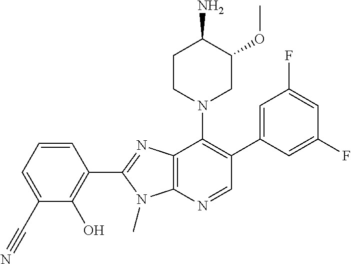 [SMILES: CO[C@@H]1CN(c2c(-c3cc(F)cc(F)c3)cnc3c2nc(-c2cccc(C#N)c2O)n3C)CC[C@H]1N] | 105 | 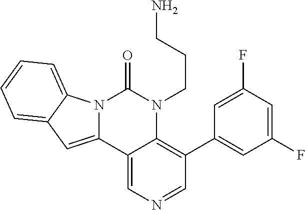 [SMILES: NCCCn1c(=O)n2c3ccccc3cc2c2cncc(-c3cc(F)cc(F)c3)c21] |
| 24 | 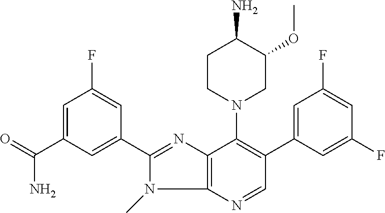 [SMILES: CO[C@@H]1CN(c2c(-c3cc(F)cc(F)c3)cnc3c2nc(-c2cc(F)cc(C(N)=O)c2)n3C)CC[C@H]1N] | 106 | 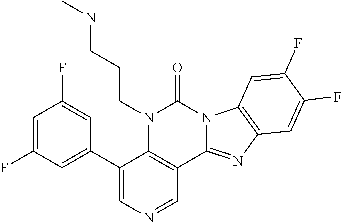 [SMILES: CNCCCn1c(=O)n2c3cc(F)c(F)cc3nc2c2cncc(-c3cc(F)cc(F)c3)c21] |
| 25 | 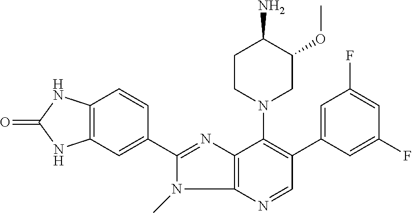 [SMILES: CO[C@@H]1CN(c2c(-c3cc(F)cc(F)c3)cnc3c2nc(-c2ccc4[nH]c(=O)[nH]c4c2)n3C)CC[C@H]1N] | 107 | 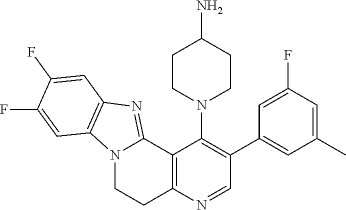 [SMILES: Cc1cc(F)cc(-c2cnc3c(c2N2CCC(N)CC2)-c2nc4cc(F)c(F)cc4n2CC3)c1] |
| 26 | 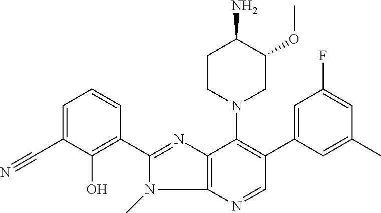 [SMILES: CO[C@@H]1CN(c2c(-c3cc(C)cc(F)c3)cnc3c2nc(-c2cccc(C#N)c2O)n3C)CC[C@H]1N] | 108 | 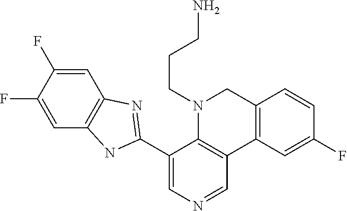 [SMILES: NCCCN1Cc2ccc(F)cc2-c2cncc(-c3nc4cc(F)c(F)cc4[nH]3)c21] |
| 27 | 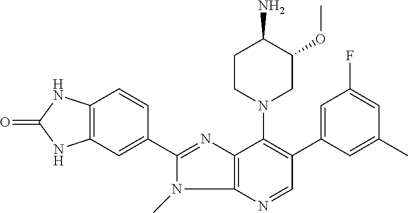 [SMILES: CO[C@@H]1CN(c2c(-c3cc(C)cc(F)c3)cnc3c2nc(-c2ccc4[nH]c(=O)[nH]c4c2)n3C)CC[C@H]1N] | 109 | 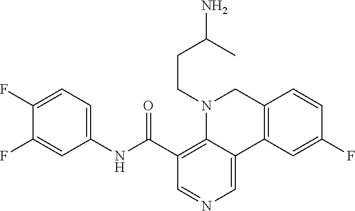 [SMILES: CC(N)CCN1Cc2ccc(F)cc2-c2cncc(C(=O)Nc3ccc(F)c(F)c3)c21] |
| 28 | 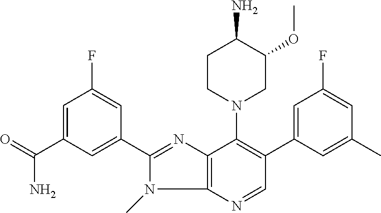 [SMILES: CO[C@@H]1CN(c2c(-c3cc(C)cc(F)c3)cnc3c2nc(-c2cc(F)cc(C(N)=O)c2)n3C)CC[C@H]1N] | 110 | 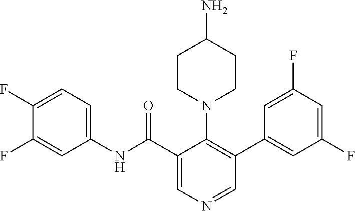 [SMILES: NC1CCN(c2c(C(=O)Nc3ccc(F)c(F)c3)cncc2-c2cc(F)cc(F)c2)CC1] |
| 29 | 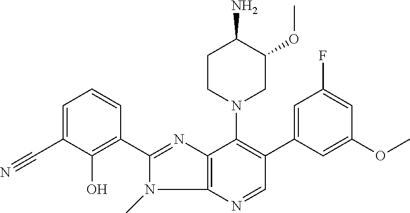 [SMILES: COc1cc(F)cc(-c2cnc3c(nc(-c4cccc(C#N)c4O)n3C)c2N2CC[C@@H](N)[C@H](OC)C2)c1] | 111 | 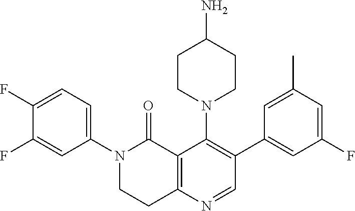 [SMILES: Cc1cc(F)cc(-c2cnc3c(c2N2CCC(N)CC2)C(=O)N(c2ccc(F)c(F)c2)CC3)c1] |
| 30 | 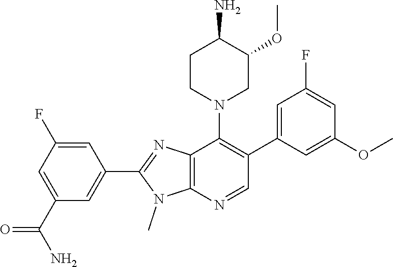 [SMILES: COc1cc(F)cc(-c2cnc3c(nc(-c4cc(F)cc(C(N)=O)c4)n3C)c2N2CC[C@@H](N)[C@H](OC)C2)c1] | 112 | 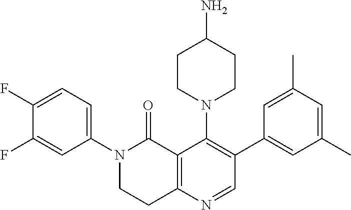 [SMILES: Cc1cc(C)cc(-c2cnc3c(c2N2CCC(N)CC2)C(=O)N(c2ccc(F)c(F)c2)CC3)c1] |
| 31 | 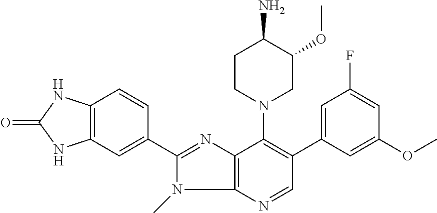 [SMILES: COc1cc(F)cc(-c2cnc3c(nc(-c4ccc5[nH]c(=O)[nH]c5c4)n3C)c2N2CC[C@@H](N)[C@H](OC)C2)c1] | 113 | 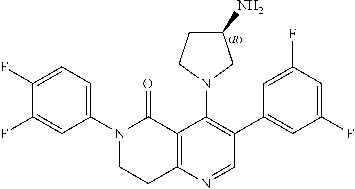 |
| 32 | 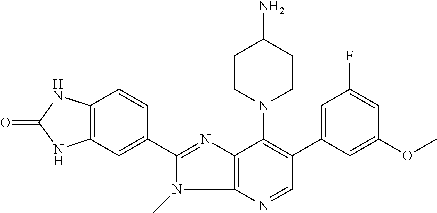 [SMILES: COc1cc(F)cc(-c2cnc3c(nc(-c4ccc5[nH]c(=O)[nH]c5c4)n3C)c2N2CCC(N)CC2)c1] | 114 | 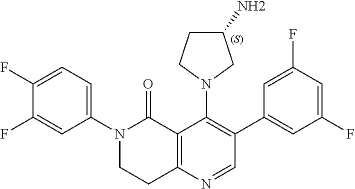 |
| 33 | 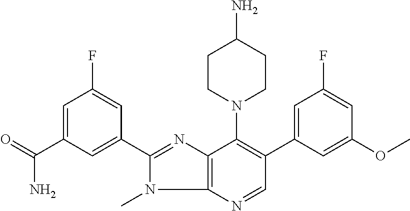 [SMILES: COc1cc(F)cc(-c2cnc3c(nc(-c4cc(F)cc(C(N)=O)c4)n3C)c2N2CCC(N)CC2)c1] | 115 | 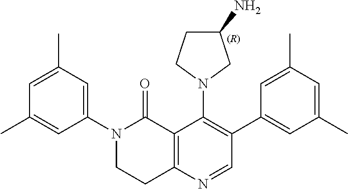 |
| 34 | 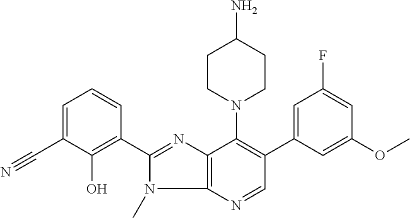 [SMILES: COc1cc(F)cc(-c2cnc3c(nc(-c4cccc(C#N)c4O)n3C)c2N2CCC(N)CC2)c1] | 116 | 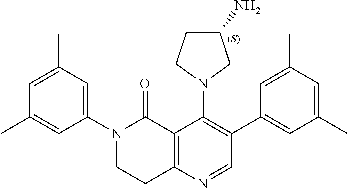 |
| 35 | 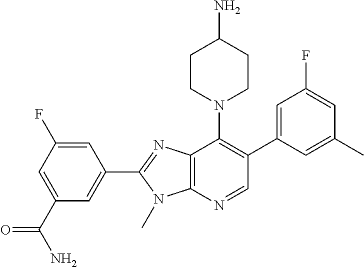 [SMILES: Cc1cc(F)cc(-c2cnc3c(nc(-c4cc(F)cc(C(N)=O)c4)n3C)c2N2CCC(N)CC2)c1] | 117 | 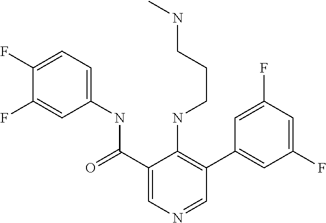 [SMILES: CNCCCNc1c(C(=O)Nc2ccc(F)c(F)c2)cncc1-c1cc(F)cc(F)c1] |
| 36 | 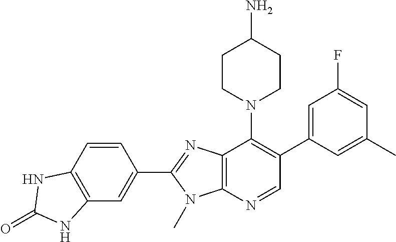 [SMILES: Cc1cc(F)cc(-c2cnc3c(nc(-c4ccc5[nH]c(=O)[nH]c5c4)n3C)c2N2CCC(N)CC2)c1] | 118 | 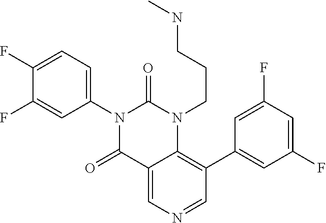 [SMILES: CNCCCn1c(=O)n(-c2ccc(F)c(F)c2)c(=O)c2cncc(-c3cc(F)cc(F)c3)c21] |
| 37 | 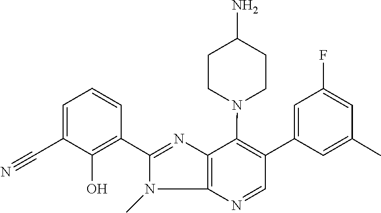 [SMILES: Cc1cc(F)cc(-c2cnc3c(nc(-c4cccc(C#N)c4O)n3C)c2N2CCC(N)CC2)c1] | 119 | 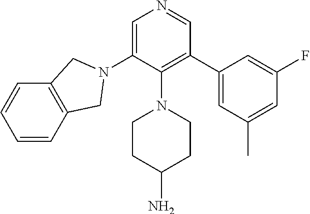 [SMILES: Cc1cc(F)cc(-c2cncc(N3Cc4ccccc4C3)c2N2CCC(N)CC2)c1] |
| 38 | 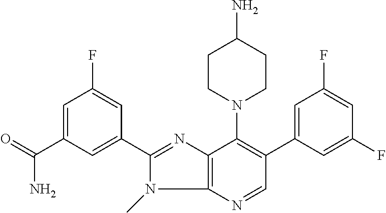 [SMILES: Cn1c(-c2cc(F)cc(C(N)=O)c2)nc2c(N3CCC(N)CC3)c(-c3cc(F)cc(F)c3)cnc21] | 120 | 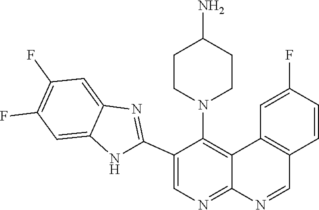 [SMILES: NC1CCN(c2c(-c3nc4cc(F)c(F)cc4[nH]3)cnc3ncc4ccc(F)cc4c23)CC1] |
| 39 | 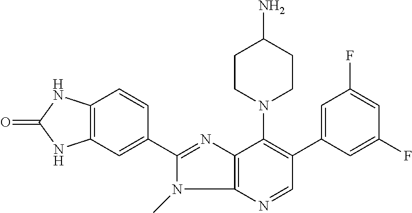 [SMILES: Cn1c(-c2ccc3[nH]c(=O)[nH]c3c2)nc2c(N3CCC(N)CC3)c(-c3cc(F)cc(F)c3)cnc21] | 121 | 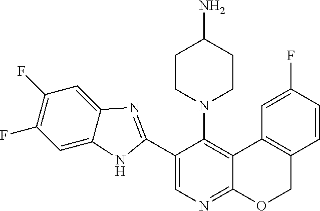 [SMILES: NC1CCN(c2c(-c3nc4cc(F)c(F)cc4[nH]3)cnc3c2-c2cc(F)ccc2CO3)CC1] |
| 40 | 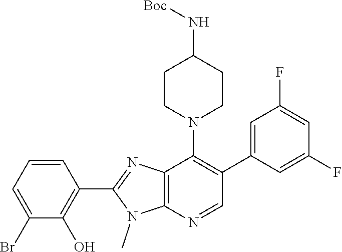 [SMILES: Cn1c(-c2cccc(Br)c2O)nc2c(N3CCC(NC(=O)OC(C)(C)C)CC3)c(-c3cc(F)cc(F)c3)cnc21] | 122 | 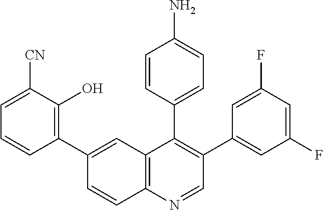 [SMILES: N#Cc1cccc(-c2ccc3ncc(-c4cc(F)cc(F)c4)c(-c4ccc(N)cc4)c3c2)c1O] |
| 41 | 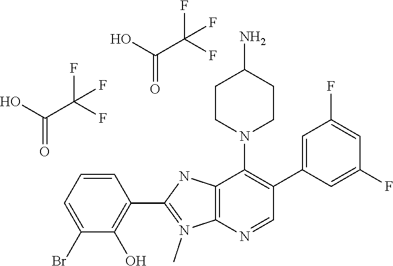 | 123 | 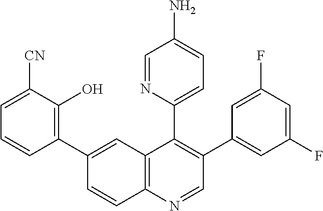 [SMILES: N#Cc1cccc(-c2ccc3ncc(-c4cc(F)cc(F)c4)c(-c4ccc(N)cn4)c3c2)c1O] |
| 42 | 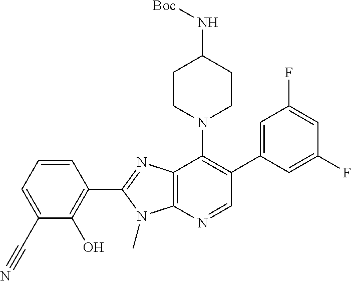 [SMILES: Cn1c(-c2cccc(C#N)c2O)nc2c(N3CCC(NC(=O)OC(C)(C)C)CC3)c(-c3cc(F)cc(F)c3)cnc21] | 124 | 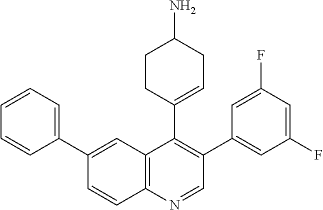 [SMILES: NC1CC=C(c2c(-c3cc(F)cc(F)c3)cnc3ccc(-c4ccccc4)cc23)CC1] |
| 43 | 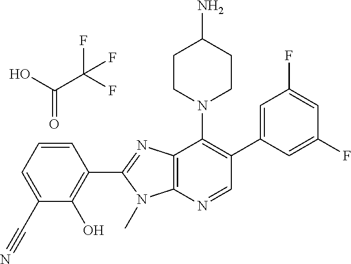 | 125 | 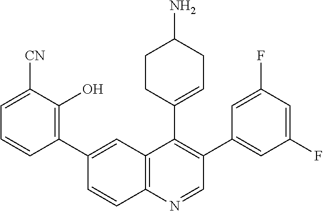 [SMILES: N#Cc1cccc(-c2ccc3ncc(-c4cc(F)cc(F)c4)c(C4=CCC(N)CC4)c3c2)c1O] |
| 44 | 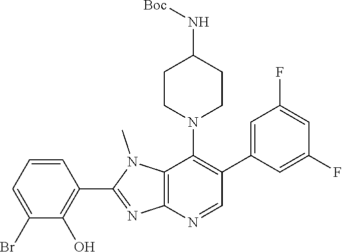 [SMILES: Cn1c(-c2cccc(Br)c2O)nc2ncc(-c3cc(F)cc(F)c3)c(N3CCC(NC(=O)OC(C)(C)C)CC3)c21] | 127 | 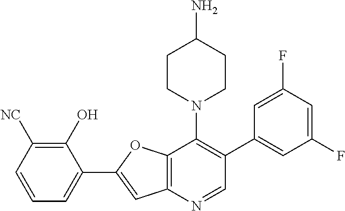 [SMILES: N#Cc1cccc(-c2cc3ncc(-c4cc(F)cc(F)c4)c(N4CCC(N)CC4)c3o2)c1O] |
| 45 | 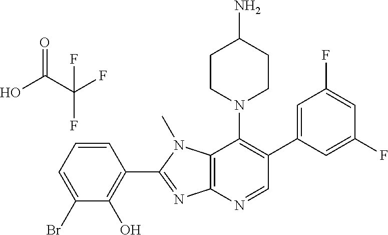 | 128 | 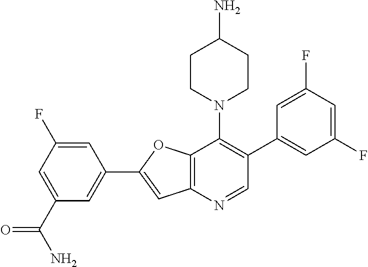 [SMILES: NC(=O)c1cc(F)cc(-c2cc3ncc(-c4cc(F)cc(F)c4)c(N4CCC(N)CC4)c3o2)c1] |
| 46 | 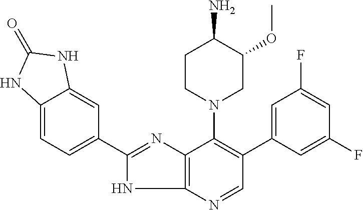 [SMILES: CO[C@@H]1CN(c2c(-c3cc(F)cc(F)c3)cnc3[nH]c(-c4ccc5[nH]c(=O)[nH]c5c4)nc23)CC[C@H]1N] | 129 | 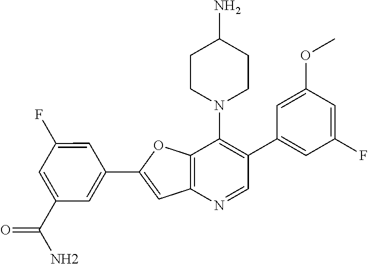 [SMILES: COc1cc(F)cc(-c2cnc3cc(-c4cc(F)cc(C(N)=O)c4)oc3c2N2CCC(N)CC2)c1] |
| 47 | 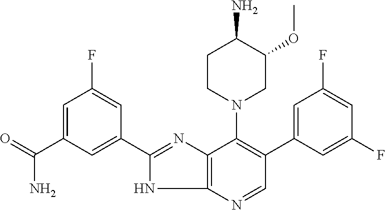 [SMILES: CO[C@@H]1CN(c2c(-c3cc(F)cc(F)c3)cnc3[nH]c(-c4cc(F)cc(C(N)=O)c4)nc23)CC[C@H]1N] | 130 | 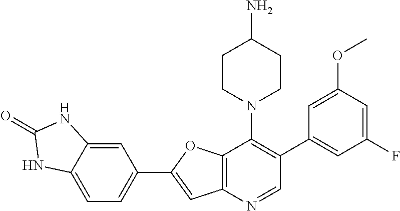 [SMILES: COc1cc(F)cc(-c2cnc3cc(-c4ccc5[nH]c(=O)[nH]c5c4)oc3c2N2CCC(N)CC2)c1] |
| 48 | 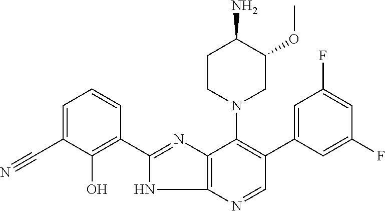 [SMILES: CO[C@@H]1CN(c2c(-c3cc(F)cc(F)c3)cnc3[nH]c(-c4cccc(C#N)c4O)nc23)CC[C@H]1N] | 131 | 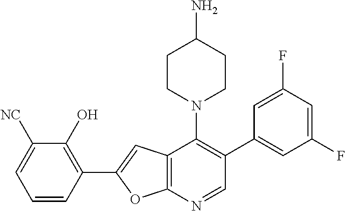 [SMILES: N#Cc1cccc(-c2cc3c(N4CCC(N)CC4)c(-c4cc(F)cc(F)c4)cnc3o2)c1O] |
| 49 | 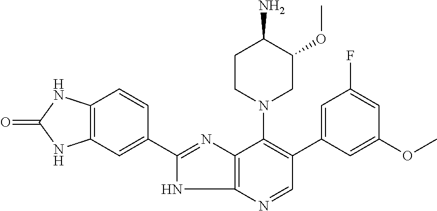 [SMILES: COc1cc(F)cc(-c2cnc3[nH]c(-c4ccc5[nH]c(=O)[nH]c5c4)nc3c2N2CC[C@@H](N)[C@H](OC)C2)c1] | 132 | 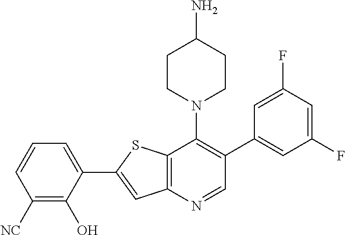 [SMILES: N#Cc1cccc(-c2cc3ncc(-c4cc(F)cc(F)c4)c(N4CCC(N)CC4)c3s2)c1O] |
| 50 | 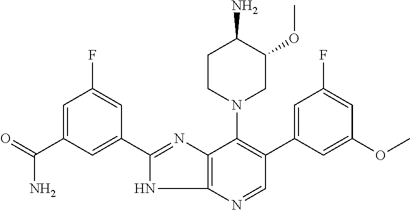 [SMILES: COc1cc(F)cc(-c2cnc3[nH]c(-c4cc(F)cc(C(N)=O)c4)nc3c2N2CC[C@@H](N)[C@H](OC)C2)c1] | 133 | 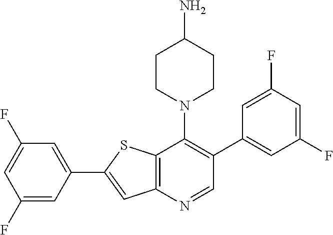 [SMILES: NC1CCN(c2c(-c3cc(F)cc(F)c3)cnc3cc(-c4cc(F)cc(F)c4)sc23)CC1] |
| 51 | 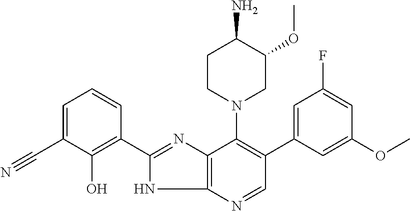 [SMILES: COc1cc(F)cc(-c2cnc3[nH]c(-c4cccc(C#N)c4O)nc3c2N2CC[C@@H](N)[C@H](OC)C2)c1] | 134 | 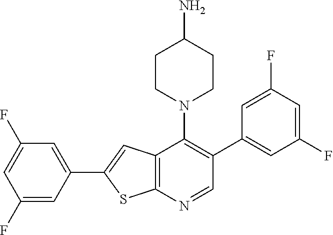 [SMILES: NC1CCN(c2c(-c3cc(F)cc(F)c3)cnc3sc(-c4cc(F)cc(F)c4)cc23)CC1] |
| 52 | 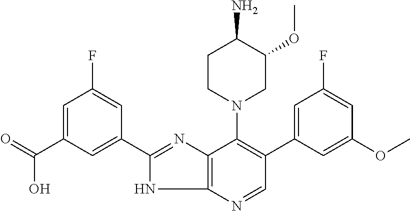 [SMILES: COc1cc(F)cc(-c2cnc3[nH]c(-c4cc(F)cc(C(=O)O)c4)nc3c2N2CC[C@@H](N)[C@H](OC)C2)c1] | 135 | 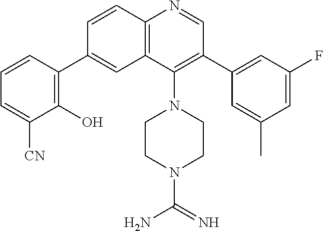 [SMILES: Cc1cc(F)cc(-c2cnc3ccc(-c4cccc(C#N)c4O)cc3c2N2CCN(C(=N)N)CC2)c1] |
| 53 | 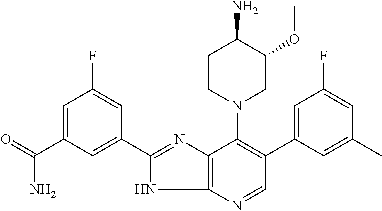 [SMILES: CO[C@@H]1CN(c2c(-c3cc(C)cc(F)c3)cnc3[nH]c(-c4cc(F)cc(C(N)=O)c4)nc23)CC[C@H]1N] | 136 | 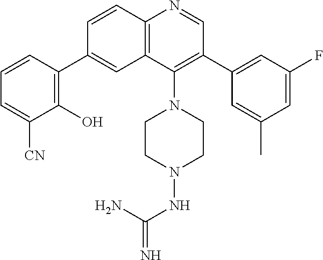 [SMILES: Cc1cc(F)cc(-c2cnc3ccc(-c4cccc(C#N)c4O)cc3c2N2CCN(NC(=N)N)CC2)c1] |
| 54 | 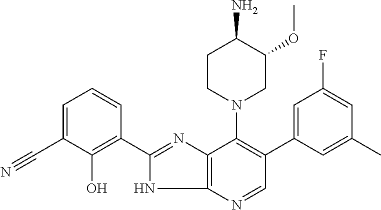 [SMILES: CO[C@@H]1CN(c2c(-c3cc(C)cc(F)c3)cnc3[nH]c(-c4cccc(C#N)c4O)nc23)CC[C@H]1N] | 137 | 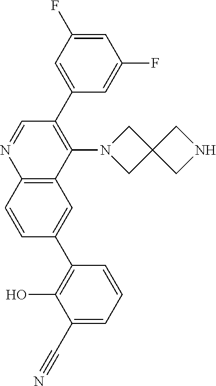 [SMILES: N#Cc1cccc(-c2ccc3ncc(-c4cc(F)cc(F)c4)c(N4CC5(CNC5)C4)c3c2)c1O] |
| 55 | 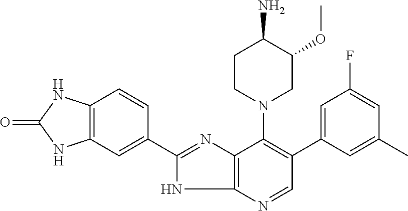 [SMILES: CO[C@@H]1CN(c2c(-c3cc(C)cc(F)c3)cnc3[nH]c(-c4ccc5[nH]c(=O)[nH]c5c4)nc23)CC[C@H]1N] | 138 | 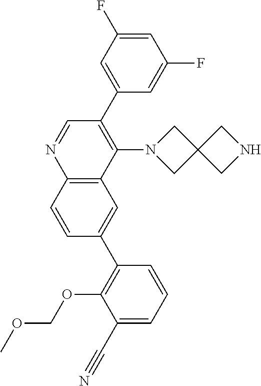 [SMILES: COCOc1c(C#N)cccc1-c1ccc2ncc(-c3cc(F)cc(F)c3)c(N3CC4(CNC4)C3)c2c1] |
| 56 | 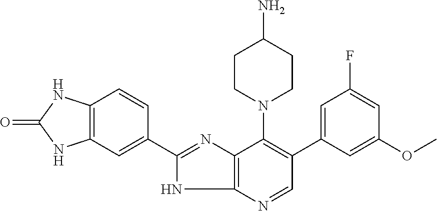 [SMILES: COc1cc(F)cc(-c2cnc3[nH]c(-c4ccc5[nH]c(=O)[nH]c5c4)nc3c2N2CCC(N)CC2)c1] | 139 | 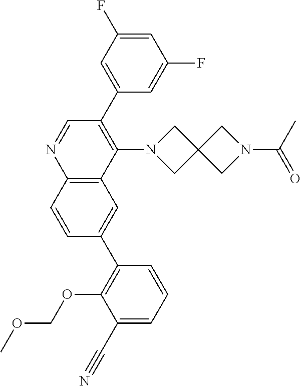 [SMILES: COCOc1c(C#N)cccc1-c1ccc2ncc(-c3cc(F)cc(F)c3)c(N3CC4(CN(C(C)=O)C4)C3)c2c1] |
| 57 | 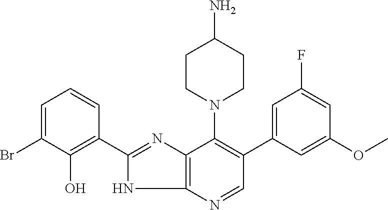 [SMILES: COc1cc(F)cc(-c2cnc3[nH]c(-c4cccc(Br)c4O)nc3c2N2CCC(N)CC2)c1] | 140 | 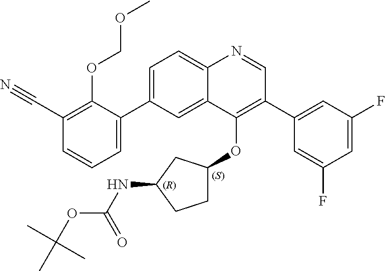 |
| 58 | 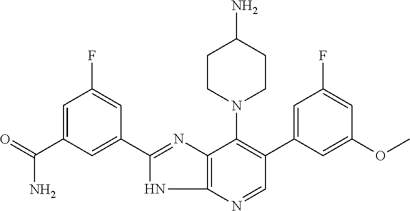 [SMILES: COc1cc(F)cc(-c2cnc3[nH]c(-c4cc(F)cc(C(N)=O)c4)nc3c2N2CCC(N)CC2)c1] | 141 | 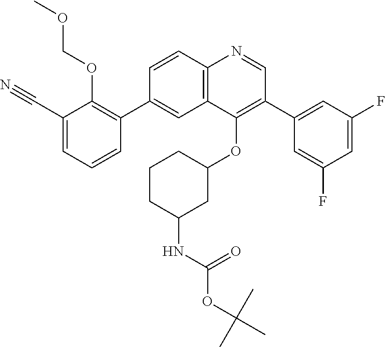 [SMILES: COCOc1c(C#N)cccc1-c1ccc2ncc(-c3cc(F)cc(F)c3)c(OC3CCCC(NC(=O)OC(C)(C)C)C3)c2c1] |
| 59 | 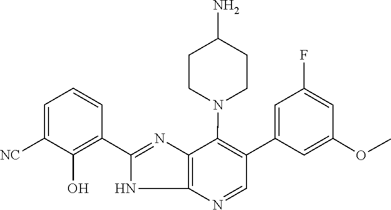 [SMILES: COc1cc(F)cc(-c2cnc3[nH]c(-c4cccc(C#N)c4O)nc3c2N2CCC(N)CC2)c1] | 142 | 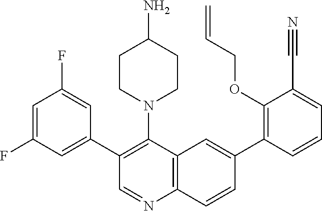 [SMILES: C=CCOc1c(C#N)cccc1-c1ccc2ncc(-c3cc(F)cc(F)c3)c(N3CCC(N)CC3)c2c1] |
| 60 | 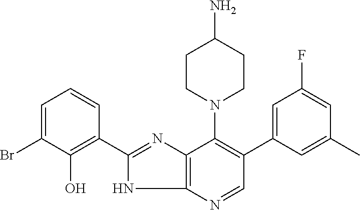 [SMILES: Cc1cc(F)cc(-c2cnc3[nH]c(-c4cccc(Br)c4O)nc3c2N2CCC(N)CC2)c1] | 143 | 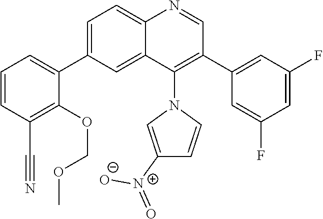 [SMILES: COCOc1c(C#N)cccc1-c1ccc2ncc(-c3cc(F)cc(F)c3)c(-n3ccc([N+](=O)[O-])c3)c2c1] |
| 61 | 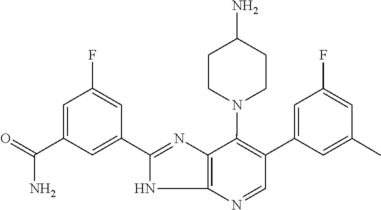 [SMILES: Cc1cc(F)cc(-c2cnc3[nH]c(-c4cc(F)cc(C(N)=O)c4)nc3c2N2CCC(N)CC2)c1] | 144 | 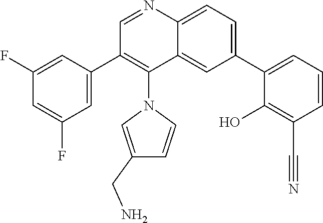 [SMILES: N#Cc1cccc(-c2ccc3ncc(-c4cc(F)cc(F)c4)c(-n4ccc(CN)c4)c3c2)c1O] |
| 62 | 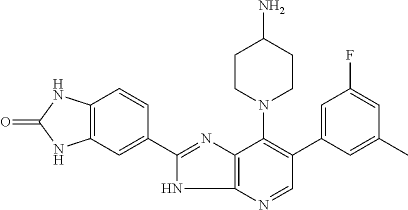 [SMILES: Cc1cc(F)cc(-c2cnc3[nH]c(-c4ccc5[nH]c(=O)[nH]c5c4)nc3c2N2CCC(N)CC2)c1] | 145 | 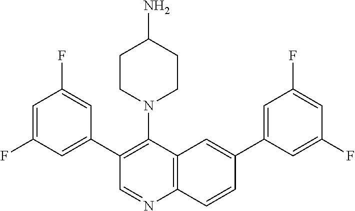 [SMILES: NC1CCN(c2c(-c3cc(F)cc(F)c3)cnc3ccc(-c4cc(F)cc(F)c4)cc23)CC1] |
| 63 | 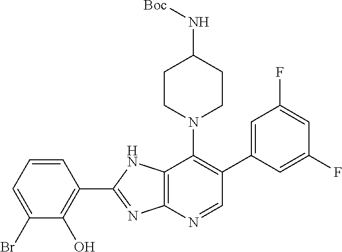 [SMILES: CC(C)(C)OC(=O)NC1CCN(c2c(-c3cc(F)cc(F)c3)cnc3nc(-c4cccc(Br)c4O)[nH]c23)CC1] | 146 | 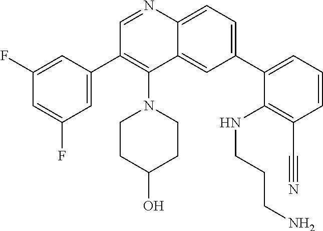 [SMILES: N#Cc1cccc(-c2ccc3ncc(-c4cc(F)cc(F)c4)c(N4CCC(O)CC4)c3c2)c1NCCCN] |
| 64 | 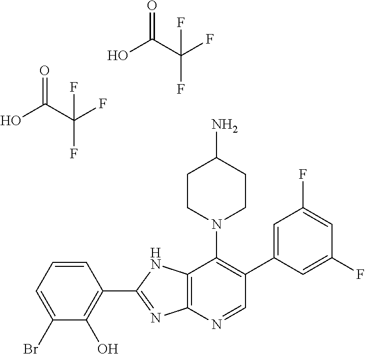 [SMILES: O=C(O)C(F)(F)F ; NC1CCN(c2c(-c3cc(F)cc(F)c3)cnc3nc(-c4cccc(Br)c4O)[nH]c23)CC1 ; O=C(O)C(F)(F)F] | 147 | 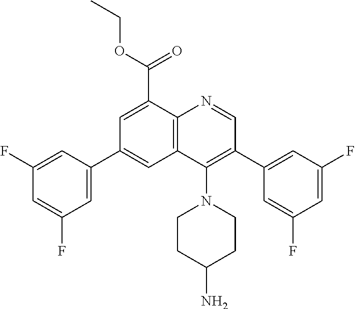 [SMILES: CCOC(=O)c1cc(-c2cc(F)cc(F)c2)cc2c(N3CCC(N)CC3)c(-c3cc(F)cc(F)c3)cnc12] |
| 65 | 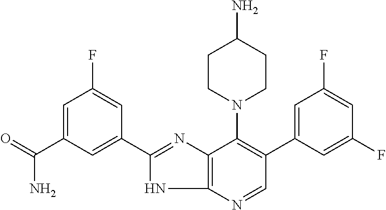 [SMILES: NC(=O)c1cc(F)cc(-c2nc3c(N4CCC(N)CC4)c(-c4cc(F)cc(F)c4)cnc3[nH]2)c1] | 148 | 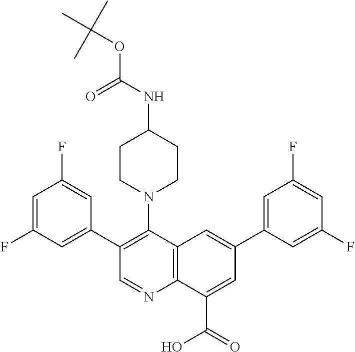 [SMILES: CC(C)(C)OC(=O)NC1CCN(c2c(-c3cc(F)cc(F)c3)cnc3c(C(=O)O)cc(-c4cc(F)cc(F)c4)cc23)CC1] |
| 66 | 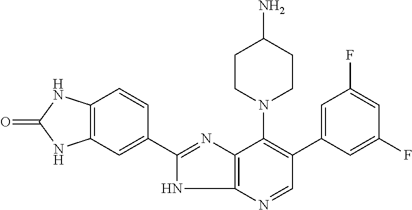 [SMILES: NC1CCN(c2c(-c3cc(F)cc(F)c3)cnc3[nH]c(-c4ccc5[nH]c(=O)[nH]c5c4)nc23)CC1] | 149 | 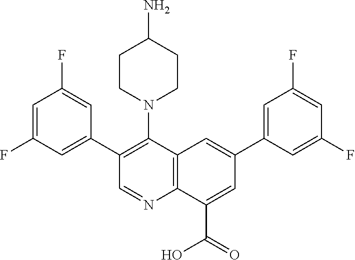 [SMILES: NC1CCN(c2c(-c3cc(F)cc(F)c3)cnc3c(C(=O)O)cc(-c4cc(F)cc(F)c4)cc23)CC1] |
| 67 | 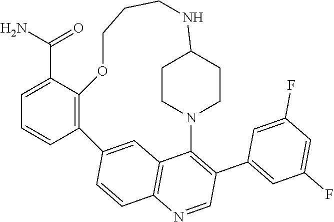 [SMILES: NC(=O)c1cccc2c1OCCCNC1CCN(CC1)c1c(-c3cc(F)cc(F)c3)cnc3ccc-2cc13] | 150 | 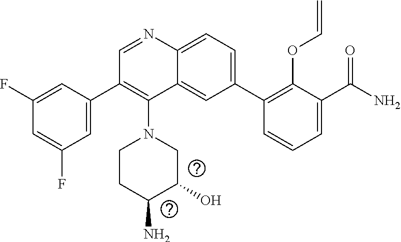 [SMILES: C=COc1c(C(N)=O)cccc1-c1ccc2ncc(-c3cc(F)cc(F)c3)c(N3CC[C@H](N)[C@@H](O)C3)c2c1] |
| 68 | 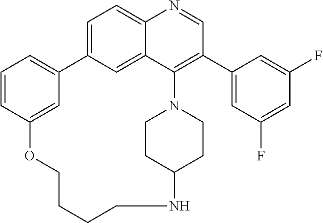 [SMILES: Fc1cc(F)cc(-c2cnc3ccc4cc3c2N2CCC(CC2)NCCCCOc2cccc-4c2)c1] | 151 | 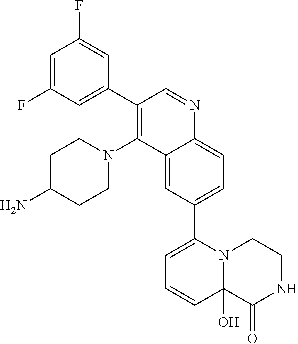 [SMILES: NC1CCN(c2c(-c3cc(F)cc(F)c3)cnc3ccc(C4=CC=CC5(O)C(=O)NCCN45)cc23)CC1] |
| 69 | 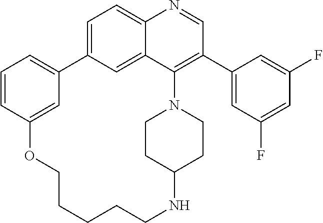 [SMILES: Fc1cc(F)cc(-c2cnc3ccc4cc3c2N2CCC(CC2)NCCCCCOc2cccc-4c2)c1] | 152 | 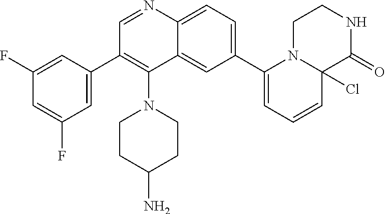 [SMILES: NC1CCN(c2c(-c3cc(F)cc(F)c3)cnc3ccc(C4=CC=CC5(Cl)C(=O)NCCN45)cc23)CC1] |
| 70 | 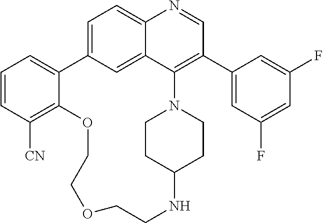 [SMILES: N#Cc1cccc2c1OCCOCCNC1CCN(CC1)c1c(-c3cc(F)cc(F)c3)cnc3ccc-2cc13] | 153 | 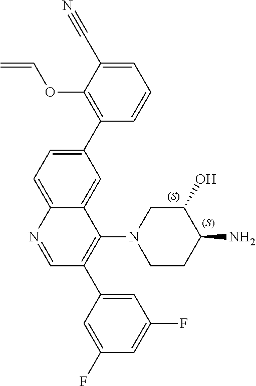 |
| 71 | 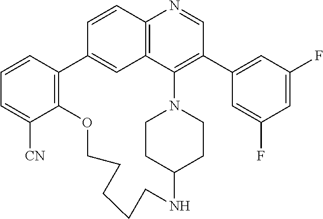 [SMILES: N#Cc1cccc2c1OCCCCCNC1CCN(CC1)c1c(-c3cc(F)cc(F)c3)cnc3ccc-2cc13] | 154 | 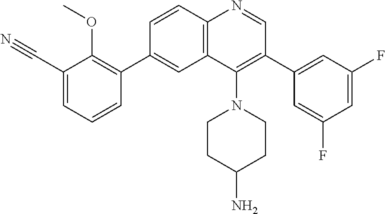 [SMILES: COc1c(C#N)cccc1-c1ccc2ncc(-c3cc(F)cc(F)c3)c(N3CCC(N)CC3)c2c1] |
| 72 | 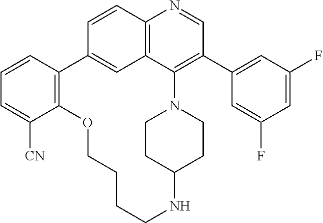 [SMILES: N#Cc1cccc2c1OCCCCNC1CCN(CC1)c1c(-c3cc(F)cc(F)c3)cnc3ccc-2cc13] | 155 | 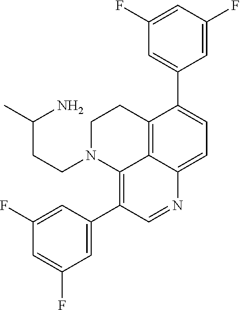 [SMILES: CC(N)CCN1CCc2c(-c3cc(F)cc(F)c3)ccc3ncc(-c4cc(F)cc(F)c4)c1c23] |
| 73 | 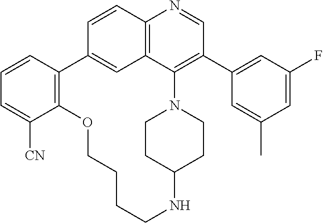 [SMILES: Cc1cc(F)cc(-c2cnc3ccc4cc3c2N2CCC(CC2)NCCCCOc2c(C#N)cccc2-4)c1] | 156 | 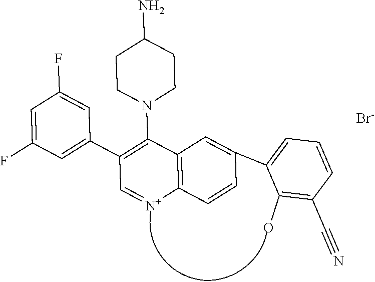 [SMILES: N#Cc1cccc(-c2ccc3[nH+]cc(-c4cc(F)cc(F)c4)c(N4CCC(N)CC4)c3c2)c1O ; [Br-]] |
| 74 | 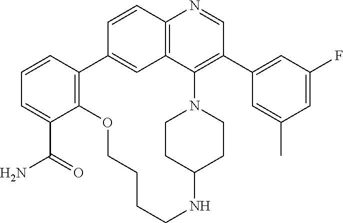 [SMILES: Cc1cc(F)cc(-c2cnc3ccc4cc3c2N2CCC(CC2)NCCCCOc2c(C(N)=O)cccc2-4)c1] | 157 | 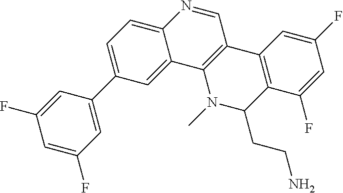 [SMILES: CN1c2c(cnc3ccc(-c4cc(F)cc(F)c4)cc23)-c2cc(F)cc(F)c2C1CCN] |
| 75 | 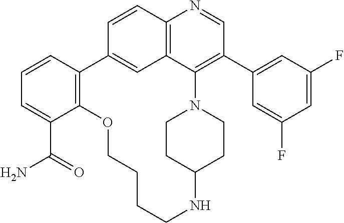 [SMILES: NC(=O)c1cccc2c1OCCCCNC1CCN(CC1)c1c(-c3cc(F)cc(F)c3)cnc3ccc-2cc13] | 158 | 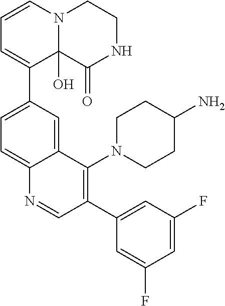 [SMILES: NC1CCN(c2c(-c3cc(F)cc(F)c3)cnc3ccc(C4=CC=CN5CCNC(=O)C45O)cc23)CC1] |
| 76 | 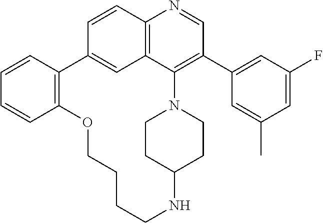 [SMILES: Cc1cc(F)cc(-c2cnc3ccc4cc3c2N2CCC(CC2)NCCCCOc2ccccc2-4)c1] | 159 | 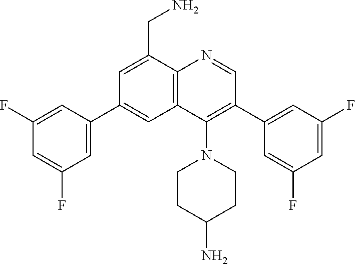 [SMILES: NCc1cc(-c2cc(F)cc(F)c2)cc2c(N3CCC(N)CC3)c(-c3cc(F)cc(F)c3)cnc12] |
| 77 | 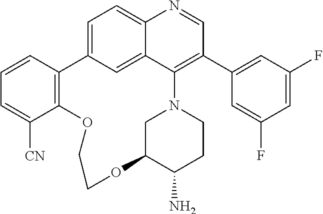 [SMILES: N#Cc1cccc2c1OCCO[C@H]1CN(CC[C@@H]1N)c1c(-c3cc(F)cc(F)c3)cnc3ccc-2cc13] | 160 | 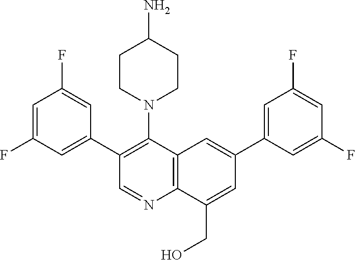 [SMILES: NC1CCN(c2c(-c3cc(F)cc(F)c3)cnc3c(CO)cc(-c4cc(F)cc(F)c4)cc23)CC1] |
| 78 | 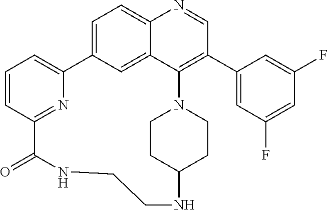 [SMILES: O=C1NCCNC2CCN(CC2)c2c(-c3cc(F)cc(F)c3)cnc3ccc(cc23)-c2cccc1n2] | 161 | 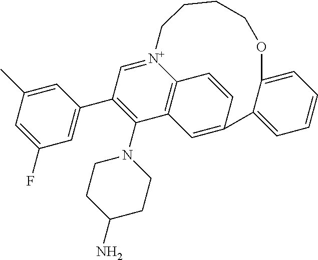 [SMILES: Cc1cc(F)cc(-c2c[n+]3c4ccc(cc4c2N2CCC(N)CC2)-c2ccccc2OCCCC3)c1] |
| 79 | 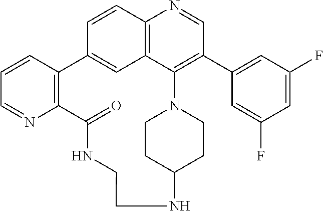 [SMILES: O=C1NCCNC2CCN(CC2)c2c(-c3cc(F)cc(F)c3)cnc3ccc(cc23)-c2cccnc21] | 162 | 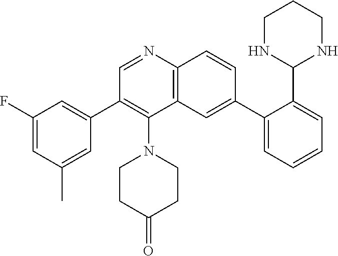 [SMILES: Cc1cc(F)cc(-c2cnc3ccc(-c4ccccc4C4NCCCN4)cc3c2N2CCC(=O)CC2)c1] |
| 80 | 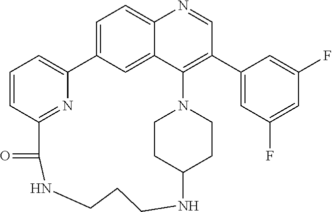 [SMILES: O=C1NCCCNC2CCN(CC2)c2c(-c3cc(F)cc(F)c3)cnc3ccc(cc23)-c2cccc1n2] | 163 | 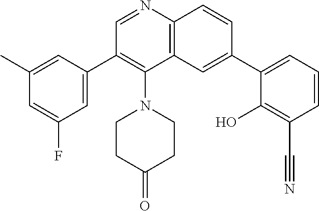 [SMILES: Cc1cc(F)cc(-c2cnc3ccc(-c4cccc(C#N)c4O)cc3c2N2CCC(=O)CC2)c1] |
| 81 | 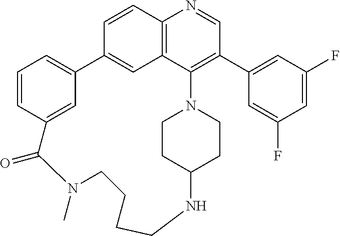 [SMILES: CN1CCCCNC2CCN(CC2)c2c(-c3cc(F)cc(F)c3)cnc3ccc(cc23)-c2cccc(c2)C1=O] | 164 | 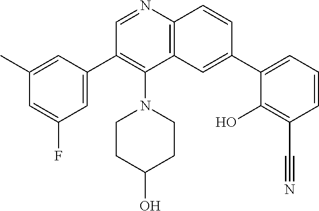 [SMILES: Cc1cc(F)cc(-c2cnc3ccc(-c4cccc(C#N)c4O)cc3c2N2CCC(O)CC2)c1] |
| 82 | 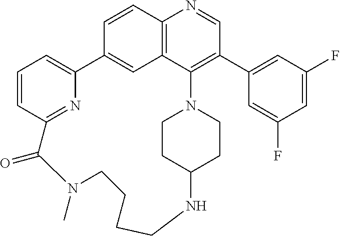 [SMILES: CN1CCCCNC2CCN(CC2)c2c(-c3cc(F)cc(F)c3)cnc3ccc(cc23)-c2cccc(n2)C1=O] | 165 | 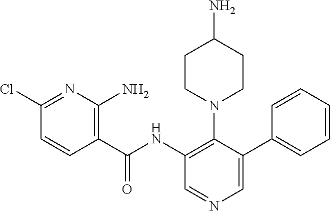 [SMILES: Nc1nc(Cl)ccc1C(=O)Nc1cncc(-c2ccccc2)c1N1CCC(N)CC1] |

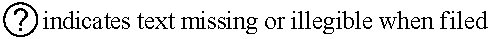

*indicates that stereochemistry is arbritrarily assigned

### III. Pharmaceutical Compositions and Modes of Administration

Compounds provided herein are usually administered in the form of pharmaceutical compositions. Thus, provided herein are also pharmaceutical compositions that comprise one or more of the compounds described herein or a pharmaceutically acceptable salt, a stereoisomer, or a mixture of stereoisomers thereof and one or more pharmaceutically acceptable vehicles selected from carriers, adjuvants and excipients. Suitable pharmaceutically acceptable vehicles may include, for example, inert solid diluents and filers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, a stereoisomer, or a mixture of stereoisomers thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10/6 by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, a stereoisomer, or a mixture of stereoisomers thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770, 4,326,525, 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, a stereoisomer, or a mixture of stereoisomers thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

### IV. Methods of Treatment

The methods described herein may be applied to cell populations in vivo or ex vivo. “In vivo” means within a living individual, as within an animal or human. In this context, the methods described herein may be used therapeutically in an individual. “Er vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In certain embodiments, the methods of administering and treating described herein further comprise co-administration of one or more additional pharmaceutically active compounds.

In a combination therapy, the pharmaceutically active compounds can be administered at the same time, in the same formulation, or at different times. Such combination therapy comprises co-administration of a compound of Formula I or a pharmaceutically acceptable salt thereof with at least one additional pharmaceutically active compound. Combination therapy in a fixed dose combination therapy comprises co-administration of a compound of Formula I or a pharmaceutically acceptable salt thereof with at least one additional pharmaceutically active compound in a fixed-dose formulation. Combination therapy in a free dose combination therapy comprises co-administration of a compound of Formula I or a pharmaceutically acceptable salt thereof and at least one additional pharmaceutically active compound in free doses of the respective compounds, either by simultaneous administration of the individual compounds or by sequential use of the individual compounds over a period of time.

### V. Methods of Preparing Compounds of Formula I and Pharmaceutically Acceptable Salts Thereof

The starting materials and reagents used in preparing the compounds described herein are either available from commercial suppliers such as Sigma-Aldrich Chemical Co., (Milwaukee, Wis.), Bachem (Torrance, Calif.), or are prepared by methods known to those skilled in the art following procedures set forth in references such as Fieser and Fieser's Reagents for Organic Synthesis, Volumes 1-17 (John Wiley and Sons. 1991); Rodd's Chemistry of Carbon Compounds, Volumes 1-5 and Supplementals (Elsevier Science Publishers, 1989); Organic Reactions, Volumes 140 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds may be prepared singly or as compound libraries comprising at least 2, for example 5 to 1,000 compounds, or 10 to 100 compounds. Libraries of compounds of Formula I may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or pharmaceutically acceptable salts thereof.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

The following examples are offered by way of illustration and not by way of limitation.

### EXAMPLES

### 1. Synthetic Examples

### Example A

### Synthesis of the Common Intermediate Compound 1-3 (6-bromo-4-chloro-3-iodoquinoline)

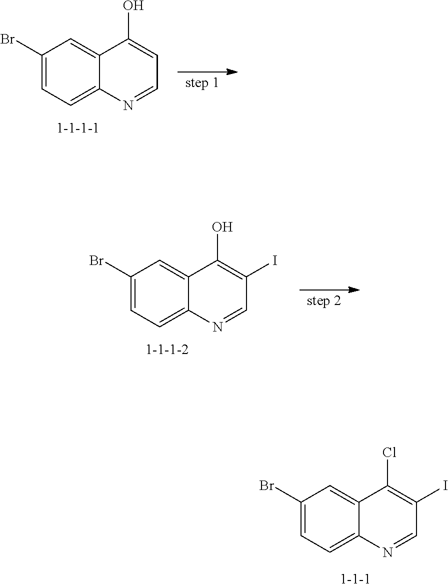 [SMILES: Oc1ccnc2ccc(Br)cc12 ; Oc1c(I)cnc2ccc(Br)cc12 ; Clc1c(I)cnc2ccc(Br)cc12]

### Step 1: Synthesis of 6-bromo-3-iodoquinolin-4-ol (Compound 1-1-1-2)

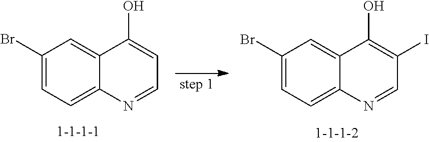 [SMILES: Oc1ccnc2ccc(Br)cc12 ; Oc1c(I)cnc2ccc(Br)cc12]

A mixture of compound 1-1-1-1 (8 g, 35.71 mmol) and 2-iodocyclopentane-1,3-dione (NIS, 8.00 g, 35.71 mmol) in acetic acid (100 mL) at 25° C. was stirred at 60° C. for 5 h. The reaction was monitored by LCMS, which showed the reaction was completed. The reaction mixture was filtered and the precipitated solid was washed with PE (50 mL×3) and dried under vacuum to afford title compound 6-bromo-3-iodo-quinolin-4-ol (compound 1-1-1-2, 11.20 g, 32.00 mmol, 89.63% yield) as white solid. MS (m/z) 349.9 (M+H)⁺.

### Step 2: Synthesis of 6-bromo-4-chloro-3-iodoquinoline (Compound 1-1-1)

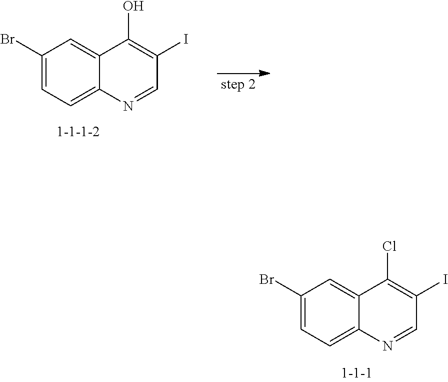 [SMILES: Oc1c(I)cnc2ccc(Br)cc12 ; Clc1c(I)cnc2ccc(Br)cc12]

A solution of compound 1-1-1-2 (10 g, 28.58 mmol) in phosphorus oxychloride (20 mL) was stirred at 100° C. for 2 h. The reaction mixture was concentrated to dryness. Added dichloromethane (100 mL) into the residue, lots of solid was observed then filtered. The filter cake was washed with 20 mL of dichloromethane, dried in vacuum to give the compound 1-1-1(6.00 g, 15.39 mmol, 53.87% yield, 94.52% purity) as yellow solid. MS (m/z) 367.90 (M+H)⁺.

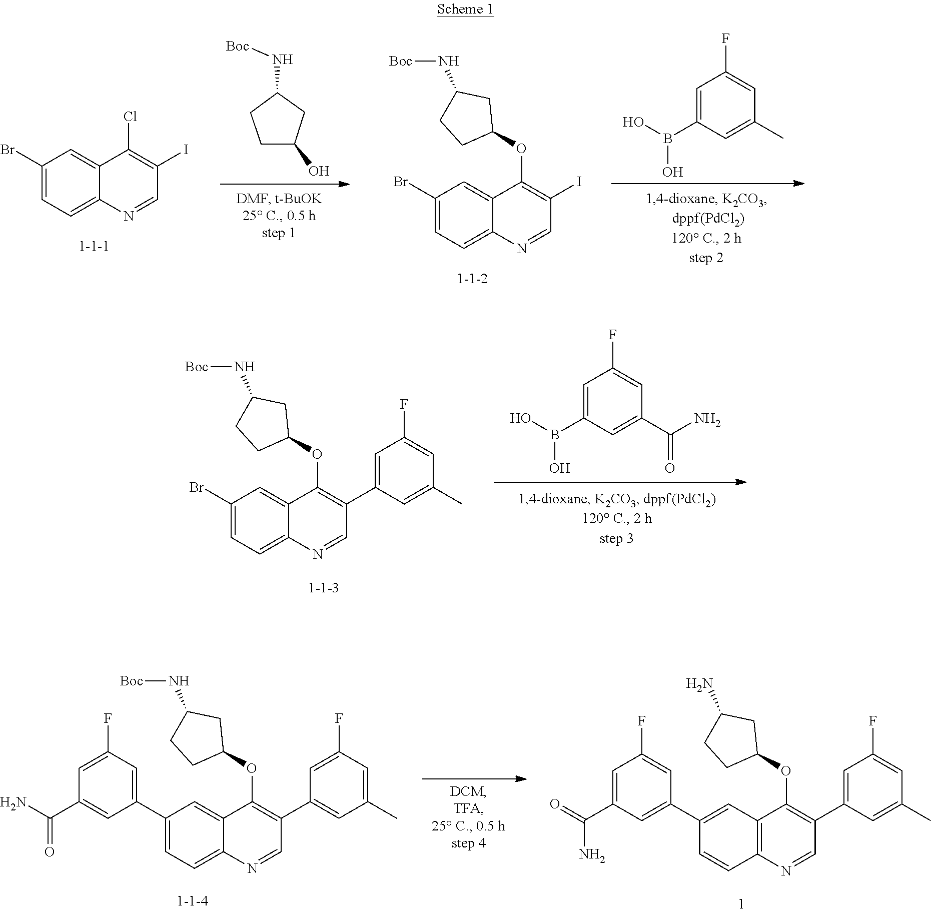

### Step 1: tert-butyl ((1S,3S)-3-((6-bromo-3-iodoquinolin-4-yl)oxy)cyclopentyl)carbamate (Compound 1-1-2)

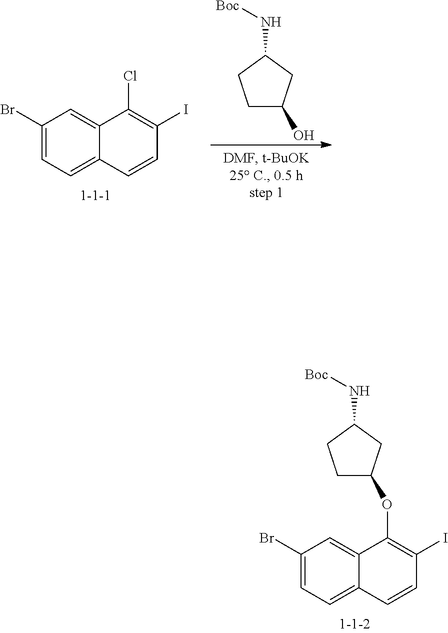 [SMILES: Clc1c(I)ccc2ccc(Br)cc12 ; CC(C)(C)OC(=O)N[C@H]1CC[C@H](O)C1 ; CC(C)(C)OC(=O)N[C@H]1CC[C@H](Oc2c(I)ccc3ccc(Br)cc23)C1]

To a solution of 6-bromo-4-chloro-3-iodo-quinoline (compound 1-1-1, 150 mg, 407.17 umol) in N,N-dimethylformamide (5 mL) was added tert-butyl N-[(1S,3S)-3-hydroxycyclopentyl]carbamate (98.34 mg, 488.60 mol) at 25° C., then the resulting mixture was stirred at 25° C. for 1 min. Potassium tert-butoxide (137.07 mg, 1.22 mmol) was added at 25° C., the resulting mixture was then stirred at 25° C. for 30 min. The reaction mixture was diluted with water and ethyl acetate. Then it was separated and water phase was extracted with ethyl acetate (20 mL×2). Combined all organic phases and dried over anhydrous Na₂SO₄, filtered and concentrated under reduced pressure, The crude product was obtained and purified by silica gel column (100-200 mesh) chromatography purification eluted with PE:EtOAc=4:1 to give the tert-butyl ((1S,3S)-3-((6-bromo-3-iodoquinolin-4-yl)oxy)cyclopentyl)carbamate (compound 1-1-2, 150.00 mg.) as white solid. Note product was not pure enough after purification and to be used into next step without further purification. MS (m/z) 349.9 (M+H)⁺.

### Step 2: tert-butyl ((1S,3S)-3-((6-bromo-3-(3-fluoro-5-methylphenyl)quinolin-4-yl)oxy)cyclopentyl)carbamate (compound 1-1-3)

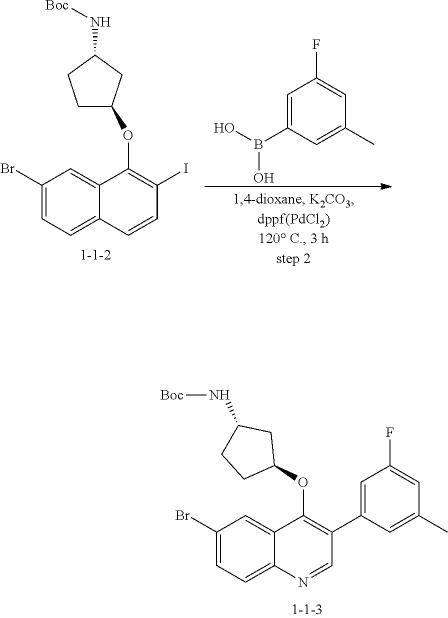 [SMILES: CC(C)(C)OC(=O)N[C@H]1CC[C@H](Oc2c(I)ccc3ccc(Br)cc23)C1 ; Cc1cc(F)cc(B(O)O)c1 ; Cc1cc(F)cc(-c2cnc3ccc(Br)cc3c2O[C@H]2CC[C@H](NC(=O)OC(C)(C)C)C2)c1]

To a solution of compound 1-1-2 (150 mg, 281.95 μmol) and (3-fluoro-5-methylphenyl)boronic acid (118.89 mg, 699.55 μmol) in mixed solvent of 1,4-dioxane (10 mL) and H₂O (1 mL) was added PdCl2(dppf) (102.66 mg, 139.91 μmol) and K₂CO₃ (290.05 mg, 2.10 mmol) at 25° C., the resulting mixture was stirred at 120° C. for 3 hr in N₂. The progress of the reaction was monitored by LCMS, and showed it worked. The reaction mixture was diluted with water and ethyl acetate. Then it was separated, the water phase was extracted with water (50 mL*2). The organic phase was washed by brine, dried over anhydrous Na₂SO₄, filtered, and concentrated to give the residue, which was purified by chromatography with PE:EtOAc=3/2 to give title compound 1-1-3 (70 mg) as yellow oil. MS (m/z) 352.3 (M+H)⁺.

### Step 3: tert-butyl ((1S,3S)-3-((6-(3-carbamoyl-5-fluorophenyl)-3-(3-fluoro-5-methylphenyl)quinolin-4-yl)oxy)cyclopentyl)carbamate (Compound 1-1-4)

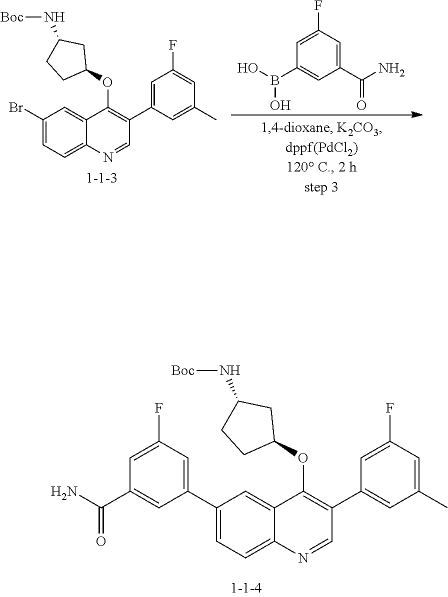 [SMILES: Cc1cc(F)cc(-c2cnc3ccc(Br)cc3c2O[C@H]2CC[C@H](NC(=O)OC(C)(C)C)C2)c1 ; NC(=O)c1cc(F)cc(B(O)O)c1 ; Cc1cc(F)cc(-c2cnc3ccc(-c4cc(F)cc(C(N)=O)c4)cc3c2O[C@H]2CC[C@H](NC(=O)OC(C)(C)C)C2)c1]

To a solution of compound 1-1-3 (70.00 mg, 135.81 μmol) and (3-carbamoyl-5-fluoro-phenyl) boronic acid (24.85 mg, 135.81 μmol) in Dioxane (5 mL) and H₂O (0.5 mL) was added PdCl2(dppf) (19.93 mg, 27.16 μmol) and K2CO3 (56.31 mg, 407.44 μmol), then the resulting mixture was degassed with N2, then stirred at 120° C. for 2 hr under N₂ condition. The progress of the reaction was monitored by LCMS, and showed it worked. The reaction mixture was diluted with water and ethyl acetate. Then it was separated, the water phase was extracted with water (50 mL*2). The organic phase was washed by brine, dried over anhydrous Na₂SO₄, filtered, and concentrated to give the residue, which was purified by chromatography with PE/EtOAc from 5/1 to 1/1 to give the compound 1-14 (40.00 mg) as yellow oil. MS (m/z) 391.1 (M+H)⁺.

### Step 4: 3-(4-(((1S,3S)-3-aminocyclopentyl)oxy)-3-(3-fluoro-5-methylphenyl)quinolin-6-yl)-5-fluorobenzamide (Compound 1)

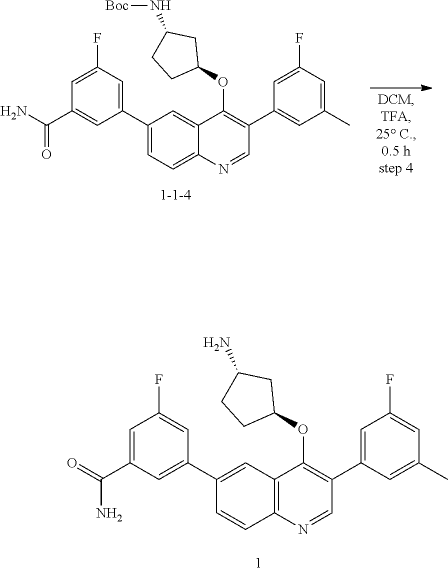 [SMILES: Cc1cc(F)cc(-c2cnc3ccc(-c4cc(F)cc(C(N)=O)c4)cc3c2O[C@H]2CC[C@H](NC(=O)OC(C)(C)C)C2)c1 ; Cc1cc(F)cc(-c2cnc3ccc(-c4cc(F)cc(C(N)=O)c4)cc3c2O[C@H]2CC[C@H](N)C2)c1]

To a solution of compound 1-14 (40 mg, 69.73 μmol) in dichloromethane (3 mL) was added trifluoroacetic acid (1 mL) at 25° C., the resulting mixture was then stirred at 25° C. for 0.5 hr. The reaction solution was diluted with 20 mL of dichloromethane, then the organic phase was concentrated to remove solvent. This above operation was repeated three times to make sure excess TFA was gone. The crude product was purified by Prep-HPLC (Prep-C18, 5 μM OBD, 19×250 mm, Column, Waters; gradient elution of 40% MeCN in water to 55% MeCN in water over a 11 min period, where both solvents contain 10 mmol/L NH₄HCO₃) to provide compound 1 (4.10 mg, 12.15% yield) as a white solid.

Compounds 1-11 were synthesized according to the 4-step procedure described in Example 1. Analytical data for compounds 1-11 are provided below.

| Cpd. No. | Structure | Analytical Data |
|---|---|---|
| 1 | 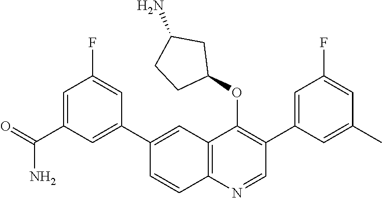 [SMILES: Cc1cc(F)cc(-c2cnc3ccc(-c4cc(F)cc(C(N)=O)c4)cc3c2O[C@H]2CC[C@H](N)C2)c1] | 1H-NMR (400 MHz, DMSO-D6): δ 8.88 (s, 1H), 8.38 (d, J = 1.6 Hz, 1H),8.22-8.16 (m, 3H), 7.89-7.86 (m, 1H), 7.71 (dd, J = 9.6, 1.4 Hz, 1H), 7.33 (t, J = 9.2 Hz, 2H), 7.16 (d, J = 9.6 Hz, 1H), 4.59 (d, J = 3,3 Hz, 1H), 3,80-3,73 (m, 1H), 2.41 (s, 3H), 2.16 (dd, J = 14.2, 7.8 Hz, 1H), 1.98 (dt, J = 21.4, 8.0 Hz, 1H), 1.76-1.71 (m, 2H), 1.61-1.44 (m, 2H). MS (m/z) 474.2 (M + H)⁺ |
| 2 | 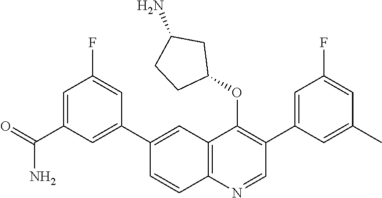 [SMILES: Cc1cc(F)cc(-c2cnc3ccc(-c4cc(F)cc(C(N)=O)c4)cc3c2O[C@@H]2CC[C@H](N)C2)c1] | 1H NMR (400 MHz, DMSO-d6): δ 9.37 (s, 1H), 9.06 (s, 1H), 8.73-8.62 (m, 4H), 8.38 (d, J = 10.2 Hz, 1H), 8.21- 8.13 (m, 2H), 7.87-7.83 (m, 2H), 7.64 (d, J = 10.2 Hz, 1H), 4.86 (s, 1H), 3.67 (d, J = 5.5 Hz, 1H), 2.91 (s, 3H), 2.49- 2.44 (m, 1H), 2.23-1.99 (m, 4H), 1.70 (s, 1H). MS (m/z) 474.2 (M + H)⁺. |
| 3 | 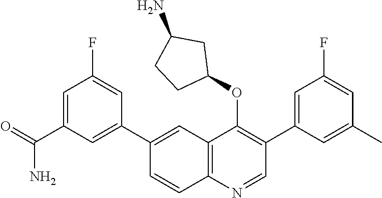 [SMILES: Cc1cc(F)cc(-c2cnc3ccc(-c4cc(F)cc(C(N)=O)c4)cc3c2O[C@H]2CC[C@@H](N)C2)c1] | 1H NMR (400 MHz, DMSO-d6): δ 8.89 (d, J = 5.8 Hz, 1 H), 8.60-8.49 (m, 1H), 8.27-8.15 (m, 4H), 7.91 (d, J = 9.3 Hz, 1H), 7.79-7.66 (m, 2H), 7.40-7.36 (m, 2H), 7.17 (d, J = 9.9 Hz, 1H), 4,39 (s, 1H), 3.19 (t, J = 7.0 Hz, 1H), 2,44 (d, J = 4.1 Hz, 3H), 2.02-1.95 (m, 1H), 1.80-1.45 (m, 5H). MS (m/z) 474.2 (M + H)+. |
| 4 | 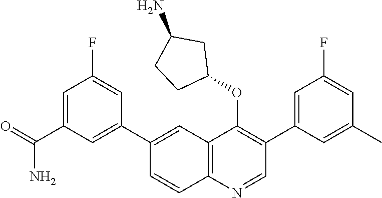 [SMILES: Cc1cc(F)cc(-c2cnc3ccc(-c4cc(F)cc(C(N)=O)c4)cc3c2O[C@@H]2CC[C@@H](N)C2)c1] | 1H NMR (400 MHz, DMSO-d6): δ 9.01 (s, 1H), 8.47 (d, J = 1.9 Hz, IH), 8.34-8.19 (m, 3H), 7.92 (dt, J = 9.4, 1.9 Hz, 1H), 7.78-7.75 (m, 1H), 7.38-7.35 (m, 2H), 7.22 (d, J = 9,6 Hz, 1H), 4.71 (d, J = 3.8 Hz, 1H), 3.69-3.61 (m, 1H), 2.46 (s, 3H), 2.24 (q, J = 7.2 Hz, 1H), 2.05 (dt, J = 21.8, 8.2 Hz, 1H), 1.82-1.77 (m, 2H), 1.67-1.50 (m, 2H). MS (m/z) 474.2 (M + H)⁺. |
| 5 | 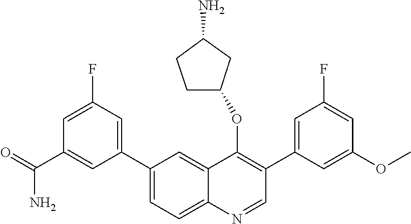 [SMILES: COc1cc(F)cc(-c2cnc3ccc(-c4cc(F)cc(C(N)=O)c4)cc3c2O[C@@H]2CC[C@H](N)C2)c1] | 1H NMR (400 MHz, DMSO-d6): δ 8.94-8.89 (m, 1H), 8.54-8.50 (m, 1H), 8.26-8.17 (m, 4H), 7.88 (dt, J = 9.5, 2.0 Hz, 1H), 7.75 (dq, J = 9.5, 1.2 Hz, 1H), 7.66 (s, 1H), 7.17- 7.11 (m, 2H), 7.01-6.95 (m, 1H), 4.44-4.39 (m, 1H), 3.87 (s, 3H), 2.52 (s, 1H), 2.22-2.14 (m, 1H), 1.88-1.70 (m, 4H), 1.59-1.52 (m, 1H). MS (m/z) 490.2 (M + H)⁺. |
| 6 | 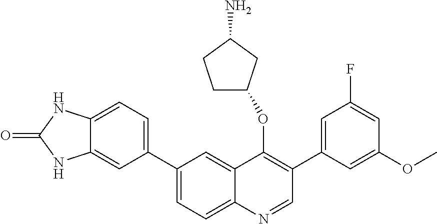 [SMILES: COc1cc(F)cc(-c2cnc3ccc(-c4ccc5[nH]c(=O)[nH]c5c4)cc3c2O[C@@H]2CC[C@H](N)C2)c1] | 1H NMR (400 MHz, DMSO-d6): δ 8.84 (d, J = 8.8 Hz, 1H), 8.38 (d, J = 12.4 Hz, 1H), 8.10 (d, J = 1.1 Hz, 2H), 7.43-7.36 (m, 2H), 7.16-7.07 (m, 3H), 6.97 (dt, J = 11.1, 2,3 Hz, 1H), 4.41-4.36 (m, 1H), 3.87 (s, 3H), 2.16-2.09 (m, 1H), 1.90-1.70 (m, 4H), 1.58-1.49 (m, 1H), 1.33-1.15 (m, 1H). MS (m/z) 485.2 (M + H)⁺. |
| 7 | 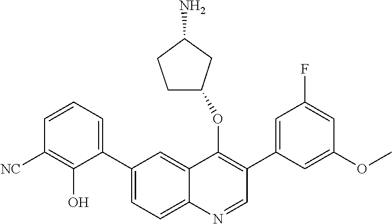 [SMILES: COc1cc(F)cc(-c2cnc3ccc(-c4cccc(C#N)c4O)cc3c2O[C@@H]2CC[C@H](N)C2)c1] | 1H NMR (400 MHz, DMSO-d6): δ 8.91 (d, J = 22.5 Hz, 1H), 8.65 (d, J = 46.7 Hz, 1H), 8.11-8.07 (m, 1H), 7.92 (dd, J = 8.7, 2.1 Hz, 1H), 7.68-7.55 (m, 2H), 7.17-7.11 (m, 2H), 6.99-6.91 (m, 2H), 4.37 (t, J = 2.9 Hz, 1H), 3.88-3.85 (m, 3H), 2.68-2.62 (m, 1H), 2.16-2.09 (m, 1H), 1.91-1.75 (m, 4H), 1.61-1.45 (m, 1H). MS (m/z) 470.2 (M + H)⁺. |
| 8 | 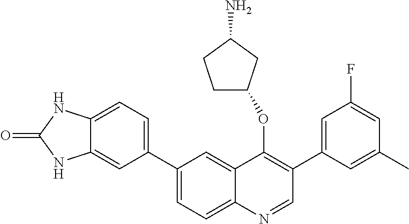 [SMILES: Cc1cc(F)cc(-c2cnc3ccc(-c4ccc5[nH]c(=O)[nH]c5c4)cc3c2O[C@@H]2CC[C@H](N)C2)c1] | 1H-NMR (400 MHz, DMSO-D6) δ 10.82 (s, 1H), 8.85 (s, 1H), 8.35 (s, 1H), 8.10 (s, 2H), 7.42-7.09 (m, 7H), 4.36 (s, 1H), 3.09-3.29 (1H), 2.44 (s, 3H), 2.19-2.12 (m, 1H), 1.87- 1.70 (m, 4H), 1.57-1.48 (m, 1H). MS (m/z) 469.2 (M + H)⁺. |
| 9 | 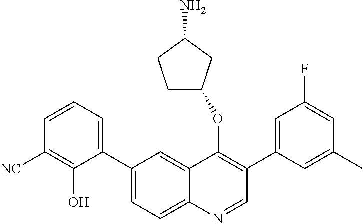 [SMILES: Cc1cc(F)cc(-c2cnc3ccc(-c4cccc(C#N)c4O)cc3c2O[C@@H]2CC[C@H](N)C2)c1] | 1H-NMR (400 MHz, DMSO-D6) δ 9.44 (s, 1H), 8.80 (s, 1H), 7.98 (d, J = 8.8 Hz, 1H), 7.91 (dd, J = 8.8, 1.9 Hz, 1H), 7.61 (dd, J = 7.4, 1,9 Hz, 1H), 7.44 (s, 1H), 7,39 (d, J = 9,9 Hz, 1H), 7.22 (dd, J = 7.7, 1.6 Hz, 1H), 7.15 (d, J = 9.6 Hz, 1H), 6.32 (t, J = 7.4 Hz, 1H), 4.29 (t, J = 5.8 Hz, 1H), 2.43 (s, 3H), 2.15-1.91 (m, 5H), 1.65-1.56 (m, 1H), 1.23 (s, 1H). MS (m/z) 454.2 (M + H)⁺. |
| 10 | 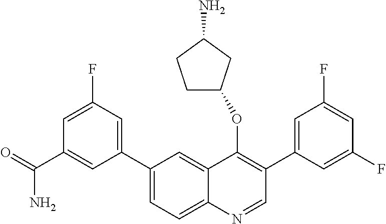 [SMILES: NC(=O)c1cc(F)cc(-c2ccc3ncc(-c4cc(F)cc(F)c4)c(O[C@@H]4CC[C@H](N)C4)c3c2)c1] | 1H-NMR (400 MHz, DMSO-D6) δ 8.95 (s, 1H), 8.48 (d, J = 1.6 Hz, 1H), 8.26 (dd, J = 8.8, 2.2 Hz, 1H), 8.22-8.20 (m, 2H), 7.96-7.87 (m, 1H), 7.76 (d, J = 9.3 Hz, 1H), 7.51- 7.49 (m, 2H), 7.45-7.40 (m, 1H),4.44-4.38 (m, 1H), 2.68- 2,61 (m, 1H), 2.47 (t, J = 1.9 Hz, 1H), 2.33 (t, J = 1.8 Hz, 1 H), 2.24 ( td, J = 14.2, 7.3 Hz, 1H), 2.03-1.97 (m, 1H), 1.88-1.73 (m, 2H), 1.60-1.54 (m, 1H). MS (m/z) 478.2 (M + H)⁺. |
| 11 | 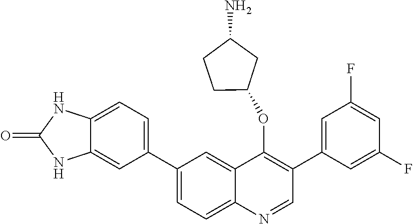 [SMILES: N[C@H]1CC[C@@H](Oc2c(-c3cc(F)cc(F)c3)cnc3ccc(-c4ccc5[nH]c(=O)[nH]c5c4)cc23)C1] | 1H-NMR (400 MHz, DMSO-D6) δ 8.87 (s, 1H), 8.36 (s, 1H), 8.12 (s,2H), 7.49 (d, J = 6.3 Hz, 2H), 7.42 (d, J = 7.4 Hz, 3H), 7.08 (d, J = 8.2 Hz, 1H), 4.37 (s, 1H), 3.37-3.32 (m, 1H), 2.69-2.63 (m, 1H),2.33 (s, 1H), 2.23 (s, 1H), 1.89- 1.77 (m, 3H), 1.56 (s, 1H). MS (m/z) 473.2 (M + H)⁺. |

### Example 2

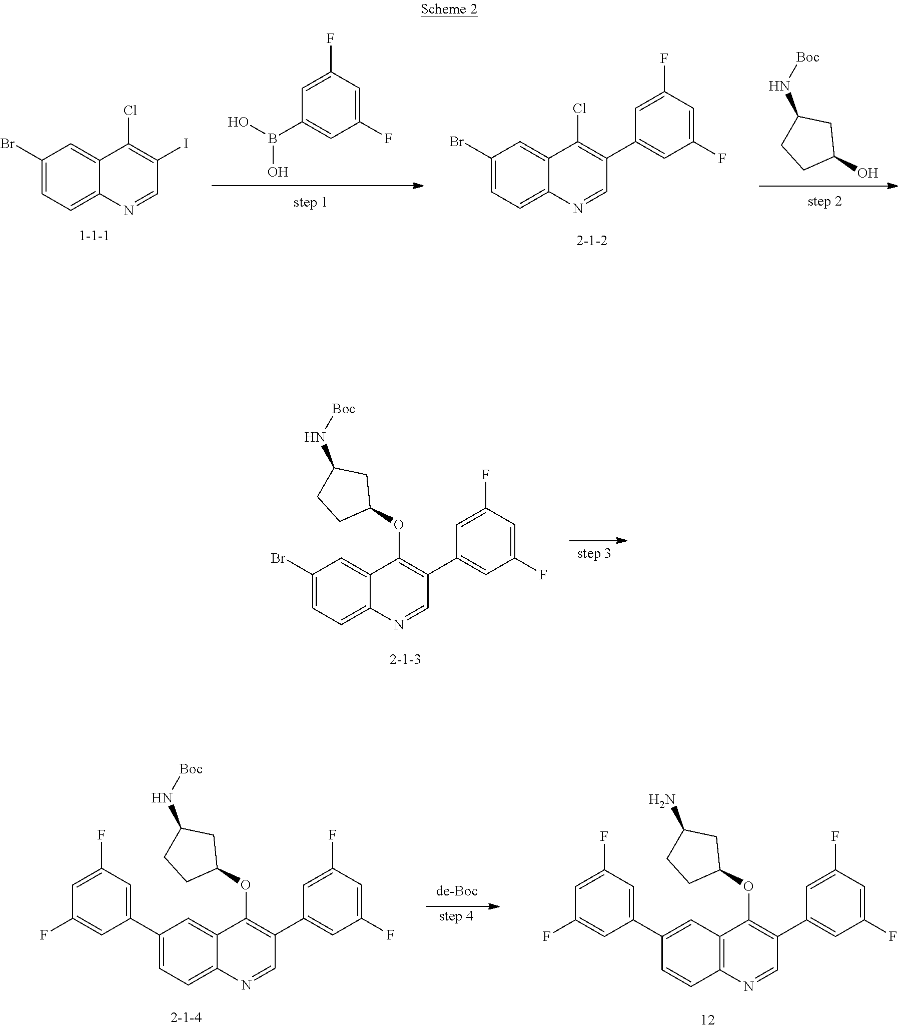

### Step 1: Synthesis of 6-bromo-4-chloro-3-(3,5-difluorophenyl)quinoline (Compound 2-1-2)

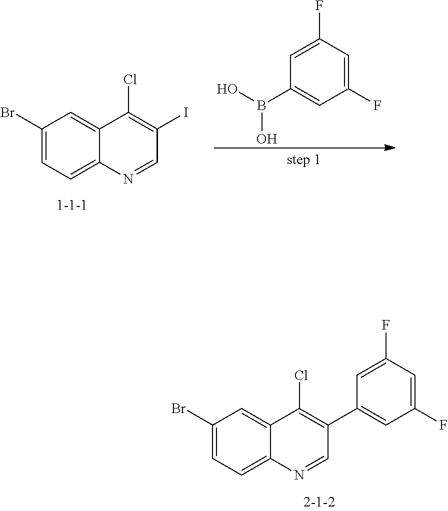 [SMILES: Clc1c(I)cnc2ccc(Br)cc12 ; OB(O)c1cc(F)cc(F)c1 ; Fc1cc(F)cc(-c2cnc3ccc(Br)cc3c2Cl)c1]

To a solution of compound 1-1-1 (2 g, 5.43 mmol) and (3,5-difluorophenyl)boronic acid (1.97 g, 12.49 mmol) in 1,4-dioxane (15 mL) was added PdCl₂(dppf) (39.83 mg, 54.29 μmol) and K₂CO₃ (2.25 g, 16.29 mmol) at 25° C. then the resulting mixture was degassed and stirred at 120° C. for 16 h under N₂ condition. The reaction was monitored by LCMS and TLC, which showed the reaction was completed. The reaction mixture was diluted with water (30 mL) then was extracted with EA (50 mL×3). The combined organic layer was dried over Na2SO4 and concentrated to give the crude material. The crude product was purified by silica gel column (100-200 mesh) chromatography purification eluted with Petroleum ether:Ethyl acetate (from 100:0 to 95:5) to obtain compound 2-1-2 (1.40 g, 2.70 mmol, 49.68% yield, 68.31% purity) as a white solid. MS (m/z) 356.10 (M+H)⁺.

### Step 2: Synthesis of tert-butyl ((1R,3S)-3-((6-bromo-3-(3,5-difluorophenyl) quinolin-4-yl)oxy)cyclopentyl)carbamate (Compound 2-1-3)

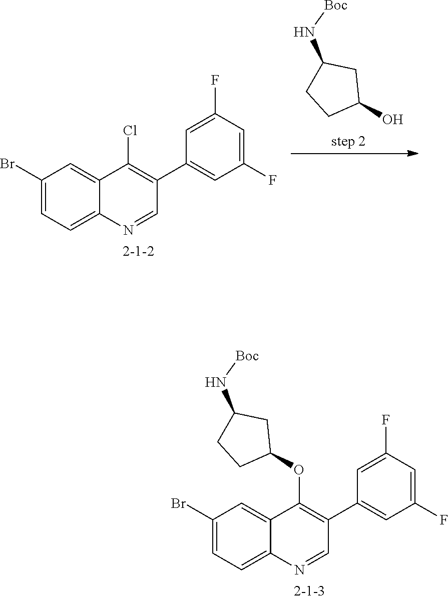 [SMILES: Fc1cc(F)cc(-c2cnc3ccc(Br)cc3c2Cl)c1 ; CC(C)(C)OC(=O)N[C@@H]1CC[C@H](O)C1 ; CC(C)(C)OC(=O)N[C@@H]1CC[C@H](Oc2c(-c3cc(F)cc(F)c3)cnc3ccc(Br)cc23)C1]

To the solution of compound 2-1-2 (176.18 mg, 496.86 μmol) and tert-butyl N-[(1R,3S)-3-hydroxycyclopentyl]carbamate (100 mg, 496.86 μmol) in N,N-Dimethylformamide (5 mL) was added Potassium tert-butoxide (167.26 mg, 1.49 mmol) at 25° C., and the resulting mixture was stirred at 25° C. for 3 h. The reaction mixture was added water (50 mL) then was extracted with EA (50 mL×3). The combined organic layer was dried over Na₂SO₄ and concentrated. The crude product was purified by silicagel column (100-200 mesh) chromatography purification eluted with Petroleum ether:Ethyl acetate (from 100:0 to 80:20) to obtain compound 2-1-3 (42.00 mg, 63.37 μmol, 12.75% yield, 78.37% purity) as white oil. MS (m/z) 519.1 (M+H)⁺.

### Synthesis of tert-butyl ((2-hydroxycyclohexyl)methyl)carbamate (Compound 2-9-ammonia)

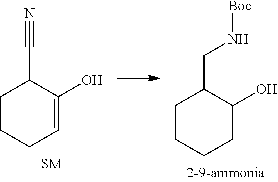 [SMILES: N#CC1CCCC=C1O ; CC(C)(C)OC(=O)NCC1CCCCC1O]

To a solution of compound SM (130 mg, 1.06 mmol) in methanol (4 mL) was added Raney Ni (185.87 mg) and Boc₂O (230.38 mg, 1.06 mmol), then the resulting mixture was degassed with hydrogen completely. After degassing, the reaction mixture was stirred at 50° C. for 2 hr under H₂ condition. The reaction solution was filtered to collect reaction liquid, which was concentrated to give the crude material (compound 2-9-ammonia, 150 mg, crude), which was to be used into next step without purification.

### Step 3: Synthesis of tert-butyl ((1R,3S)-3-((3,6-bis(3,5-difluorophenyl)quinolin-4-yl)oxy)cyclopentyl)carbamate (Compound 2-14)

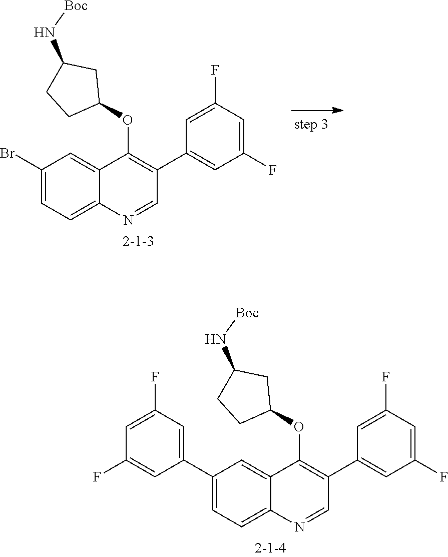 [SMILES: CC(C)(C)OC(=O)N[C@@H]1CC[C@H](Oc2c(-c3cc(F)cc(F)c3)cnc3ccc(Br)cc23)C1 ; CC(C)(C)OC(=O)N[C@@H]1CC[C@H](Oc2c(-c3cc(F)cc(F)c3)cnc3ccc(-c4cc(F)cc(F)c4)cc23)C1]

To a solution of compound 2-1-3 (42 mg, 80.87 μmol) and (3,5-difluorophenyl)boronic acid (12.77 mg, 80.87 μmol) in 1,4-dioxane (3 mL) and Water (300 μL) was added cyclopenta-1,3-dien-1-yl(diphenyl)phosphane;iron;palladium(2+);dichloride (29.67 mg, 40.43 μmol) and potassium carbonate (33.53 mg, 242.60 μmol) at 25° C., then the resulting mixture was stirred at 120° C. for 2 h in N₂. The reaction mixture was diluted with water (30 mL) and extracted with EtOAc (30 mL×3). The combined organic layers were dried over Na₂SO₄ and concentrated to obtain crude product. The crude product was purified by silica gel column (100-200 mesh) chromatography purification eluted with Petroleum ether:Ethyl acetate (from 100:0 to 70:30) to obtain compound 2-1-4 (20 mg, 11.60 μmol, 14.34% yield) as yellow solid. MS (m/z) 553.3 (M+H)⁺.

### Step 4: Synthesis of (1R,3S)-3-((3,6-bis(3,5-difluorophenyl)quinolin-4-yl)oxy) cyclopentanamine (Compound 12)

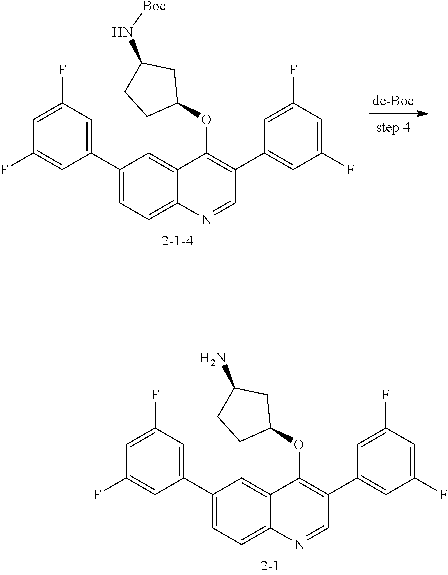 [SMILES: CC(C)(C)OC(=O)N[C@@H]1CC[C@H](Oc2c(-c3cc(F)cc(F)c3)cnc3ccc(-c4cc(F)cc(F)c4)cc23)C1 ; N[C@@H]1CC[C@H](Oc2c(-c3cc(F)cc(F)c3)cnc3ccc(-c4cc(F)cc(F)c4)cc23)C1]

To a solution of compound 2-14 (20 mg, 0.036 mmol) in dichloromethane (3 mL) was added Trifluoroacetic acid (1 mL) at 25° C., the resulting mixture was stirred at 25° C. for 0.5 h. LCMS showed the reaction was completed. The mixture was distilled under reduced pressure to remove the solvent, which was purified by Prep-HPLC to obtain compound 12 (9.3 mg, 55% yield) as white solid.

Compounds 12-22 were synthesized according to the 4-step procedure described in Example 2. Analytical data for compounds 12-22 are provided below.

| Cpd. No. | Structure | Analytical Data |
|---|---|---|
| 12 | 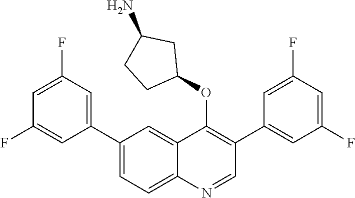 [SMILES: N[C@@H]1CC[C@H](Oc2c(-c3cc(F)cc(F)c3)cnc3ccc(-c4cc(F)cc(F)c4)cc23)C1] | 1H NMR (400 MHz, DMSO-d6): δ ppm 8.90 (d, J = 5.8 Hz, 1H), 8.46 (s, 1H), 8.23-8.12 (m, 2H), 7.67-7.59 (m, 2H), 7.49- 7.46 (m, 2H), 7.39-7.32 (m, 2H), 4.38 (d, J = 22.5 Hz, IH), 2.68- 2.62 (m, 2H), 2.33 (t, J = 1.8 Hz, IH), 2.03-1.92 (m, 1H), 1.69 (s, 2H), 1.48(d, J = 21.4 Hz, 2H), 1.19 (d, J = 34.6 Hz, 1H). MS (m/z) 453.2 (M + H)⁺. |
| 13 | 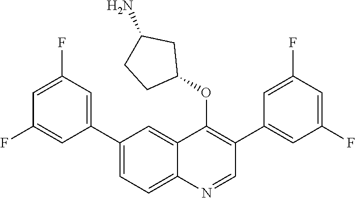 [SMILES: N[C@H]1CC[C@@H](Oc2c(-c3cc(F)cc(F)c3)cnc3ccc(-c4cc(F)cc(F)c4)cc23)C1] | 1H NMR (400 MHz, DMSO-d6): δ ppm 8.88-8.83 (m, 1H), 8.49 (d, J = 1.6 Hz, 1H), 8.20-8.14 (m, 2H), 7.47-7.42 (m, 2H), 7.42-7.34(m, 2H), 7.16-7.03 (m, 2H), 5.34 (t, J = 4.5 Hz, 1H), 4.99 (d, J =14.8 Hz, 1H), 3.92-3.81 (m, 1H), 3.55-3.47 (m, 1H), 2.43-2.33 (m, 1H), 2.21-2.16 (m, IH), 2.13-1,98 (m, 2H), 1.94- 1.83 (m, 2H), 1.73-1.58 (m, 2H). MS (m/z) 453.2 (M + H)⁺. |
| 14 | 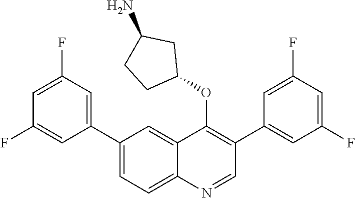 [SMILES: N[C@@H]1CC[C@@H](Oc2c(-c3cc(F)cc(F)c3)cnc3ccc(-c4cc(F)cc(F)c4)cc23)C1] | 1H NMR (400 MHz, DMSO-d6): δ ppm 8.90 (s, 1H), 8.35 (s,1H), 8.15 (dd, J = 18.4, 8.8 Hz, 2H), 7.57 (d, J = 8.5 Hz, 2H), 7.45(d, J = 6.6 Hz, 2H), 7.39-7.30 (m, 2H), 4.57-4.54 (m, 1H), 3.49 (s, 1H), 2.65 (s, 1H), 2.31 (s, 1H), 1.93-1.72 (m, 3H), 1.65 (s, 1H), 1.32-1.13 (m, 2H). MS (m/z) 453.2 (M + H)⁺. |
| 15 | 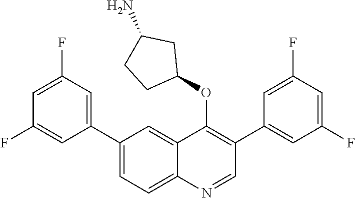 [SMILES: N[C@H]1CC[C@H](Oc2c(-c3cc(F)cc(F)c3)cnc3ccc(-c4cc(F)cc(F)c4)cc23)C1] | 1H NMR (400 MHz, DMSO-d6): δ ppm 8.95 (s, 1H), 8.38 (d, J = 1.9 Hz, 1H), 8.23-8.16 (m, 2H), 7.83 (s, 2H), 7.67-7.61 (m, 2H), 7.50-7.32 (m, 4H), 4.65 (t, J = 2.5 Hz, 1H), 3.63 (d, J = 2.2 Hz, 1H), 2.18 (dd, J = 14,4, 7.8 Hz, 1H), 2.01 (dt, J = 21.3, 8.0 Hz, 1H), 1.85-1.63 (m, 3H), 1.57-1.49 (m, 1H). MS (m/z) 453.2 (M + H)⁺. |
| 16 | 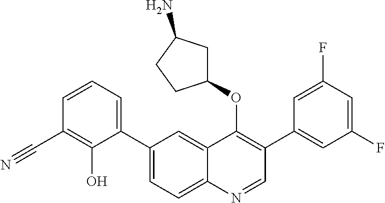 [SMILES: N#Cc1cccc(-c2ccc3ncc(-c4cc(F)cc(F)c4)c(O[C@H]4CC[C@@H](N)C4)c3c2)c1O] | 1H NMR (400 MHz, DMSO-d6): δ ppm 9.351 (s,1H), 8.835 (s, 1H ), 8.009 (d, J = 8.8 Hz, 1H), 7.938 (d, J = 9.2 Hz, 1H), 7.618 (d, J = 9.2 Hz, 1H ), 7.514 (d, J = 6.8 Hz, 2H), 7.349- 7.396 (m, 1H), 7.272 (d, J = 8.0 Hz, 1H ), 6.396-6.403 (m, 1H), 4.301 (s, 1H), 1.965-2.040 (m, 6H), 1.650-1.670(m, 1H). MS (m/z) 458.1 (M + H)⁺. |
| 17 | 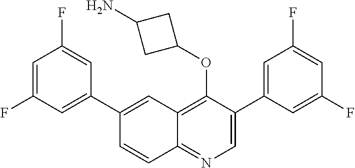 [SMILES: NC1CC(Oc2c(-c3cc(F)cc(F)c3)cnc3ccc(-c4cc(F)cc(F)c4)cc23)C1] | 1H NMR (400 MHz, DMSO-d6): δ ppm 8.90 (d, J = 5.8 Hz, 1H), 8.42 (t, J = 2.5 Hz, 1H), 8.17 (dd, J = 20.9, 8.8 Hz, 2H), 7.61 (d, J = 6.9 Hz, 2H), 7.45-7.32 (m, 4H), 4.68-4.03 (m, 1H), 3.53-3.49 (m, 2H), 2.80-2.72 (m, 1H), 2.36-2.30 (m, 2H), 2.03-1.82 (m, 2H). MS (m/z) 439.1 (M + H)⁺. |
| 18 | 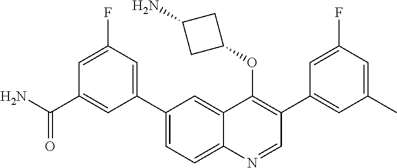 [SMILES: Cc1cc(F)cc(-c2cnc3ccc(-c4cc(F)cc(C(N)=O)c4)cc3c2O[C@H]2C[C@@H](N)C2)c1] | 1H-NMR (400 MHz, DMSO-D₆) δ 8.75 (s, 1H), 8.36 (d, J = 1.6 Hz, 1H), 8.16-8.03 (m, 4H), 7.75 (dt, J = 9.4, 1.9 Hz, 1H), 7.63-7.60 (m, 1H), 7.53 (s, 1H), 7.22-7.18 (m, 2H), 7.04 (d, J = 9.9 Hz, 1H), 3.93-3.84 (m, 1H), 2.56-2.47 (m, 1H), 2.32 (s, 3H), 2.18-2.12 (m, 2H), 1.76-1.69 (m, 2H). MS (m/z) 460.2 (M + H)⁺. |
| 19 | 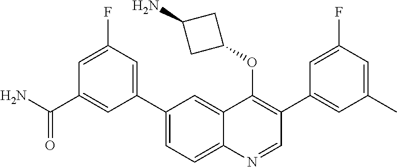 [SMILES: Cc1cc(F)cc(-c2cnc3ccc(-c4cc(F)cc(C(N)=O)c4)cc3c2O[C@H]2C[C@H](N)C2)c1] | 1H-NMR (400 MHz, DMSO-D6) δ 8.85 (s, 1H), 8.50-8.46 (m, 1H), 8.29 (d, J = 13.2 Hz, 1H), 8.21-8.13 (m, 3H), 7.86 (d, J = 9.3 Hz, 1H), 7.73-7.65 (m, 2H), 7.31 (d, J = 14.3 Hz, 2H), 7.14 (d, J = 9.6 Hz, 1H), 4.61-4.55 (m, 1H), 3.49-3.43 (m, 1H), 2.41 (s, 3H), 2.34-2.21 (m, 2H), 1.75-1.68 (m, 2H). MS (m/z) 460.2 (M + H)⁺. |
| 20 | 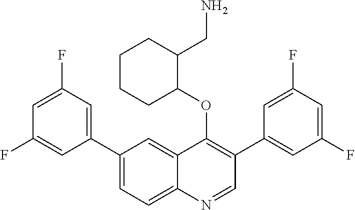 [SMILES: NCC1CCCCC1Oc1c(-c2cc(F)cc(F)c2)cnc2ccc(-c3cc(F)cc(F)c3)cc12] | 1H-NMR (400 MHz, DMSO-D6): δ 8.66 (dd, J = 40.1, 1.6 Hz, 1H), 8.31 (d, J = 18.7 Hz, 1H), 8.08 (dq, J = 8.8, 1.9 Hz, 1H), 7.92 (dd, J = 8.8, 2.2 Hz, 1H), 7.69-7.64 (m, 2H), 7.32-7.02 (m, 4H), 4.53 (dd, J = 202.8, 4.4 Hz, 1H), 3.53 (s, 2H), 3.07-2.86 (m, 1H), 2.76-2.61 (m, 1H), 1.55-1.39 (m, 4H), 1.23-0.89 (m, 5H). 19F-NMR (376 MHz, DMSO-D6): δ −109.1 (d, J = 30.3 Hz, 2F), −109.8 (d, J = 26.0 Hz, 2F). MS (m/z) 481.0 (M + H)⁺. |
| 21 | 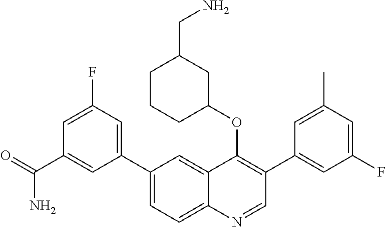 [SMILES: Cc1cc(F)cc(-c2cnc3ccc(-c4cc(F)cc(C(N)=O)c4)cc3c2OC2CCCC(CN)C2)c1] | 1H-NMR (400 MHz, DMSO-D6) δ 9.08 (d, J = 8.2 Hz, 1H), 8.58 (d, J = 1.9 Hz, 1H), 8.38 (dd, J = 9.1, 1.9 Hz, 1H), 8.28 (d, J = 8.8 Hz, 1H), 8.18 (s, 1H), 7.92 (d, J = 9,6 Hz, IH), 7.78 (d, J = 9.3 Hz, 1H), 7.38 (d, J = 12. 1 Hz, 2H), 7.24 (d, J = 9.6 Hz, 1H), 3.58 (s, 2H), 2.61 (d, J = 5.2 Hz, 2H), 2.45 (s, 3H), 1.89 (s, IH), 1.78-1.75 (m, 1H), 1.67 (d, J = 3.0 Hz, 1H), 1.55 (d, J = 7.7 Hz, 1H), 1.45-1.16 (m, 4H), 0.87-0.76 (m, 2H). MS (m/z) 502.2 (M + H)⁺. |
| 22 | 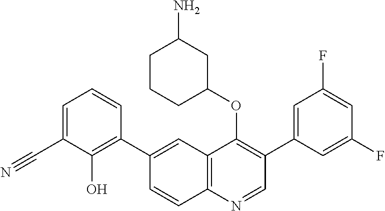 [SMILES: N#Cc1cccc(-c2ccc3ncc(-c4cc(F)cc(F)c4)c(OC4CCCC(N)C4)c3c2)c1O] | 1H NMR (400 MHz, DMSO-d6): δ ppm 8.972-8.964 (d, J = 3.2 Hz, 1H), 8.313-8.308 (d, J = 2.0 Hz, 1H), 8.189- 8.168 (d, J = 8.4 Hz, 1H), 8.006-7.980 (dd, J1 = 8.8, J2 = 2.0 Hz, 1H), 7.755-7.735 (d, J = 8.0 Hz, 2H), 7.487-7.461 (dd, J1 = 8.4, J2 = 2,0 Hz, 2H), 7.412- 7.360 (m, 1H), 7.242-7.203 (t, J = 7.6 Hz, 1H), 3.925 (s, 1H), 2.857-2.798 (m, 1H), 2.098-2.066 (d, J = 12.8 Hz, 1H), 1.777-1.656 (m, 3H), 1.468-1.233 (m, 4H). MS (m/z) 472.2 (M + H)⁺. |

### Example 3

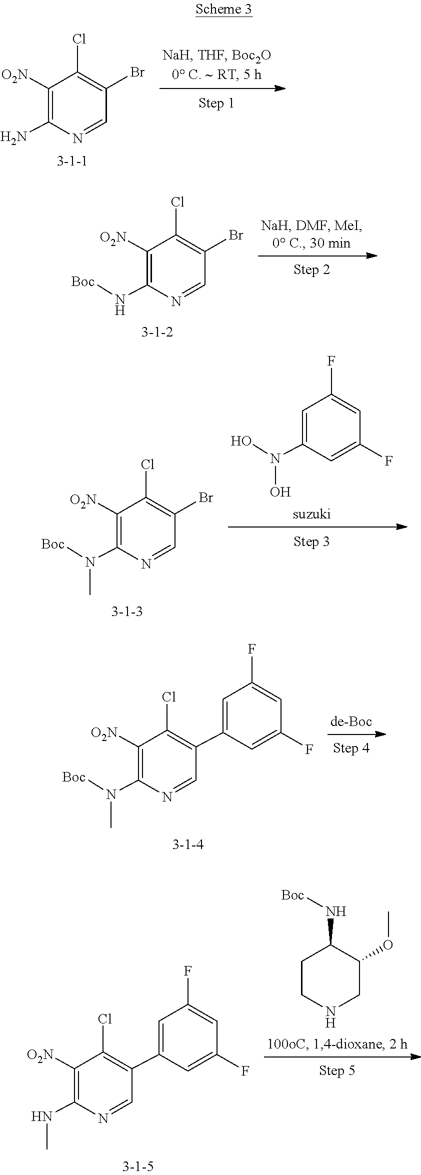

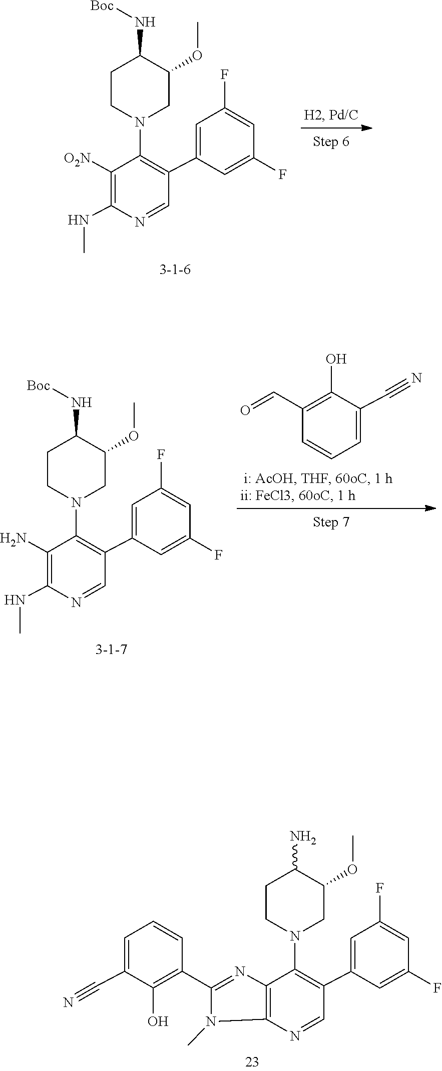 [SMILES: CNc1ncc(-c2cc(F)cc(F)c2)c(N2CC[C@@H](NC(=O)OC(C)(C)C)[C@H](OC)C2)c1[N+](=O)[O-] ; CNc1ncc(-c2cc(F)cc(F)c2)c(N2CC[C@@H](NC(=O)OC(C)(C)C)[C@H](OC)C2)c1N ; N#Cc1cccc(C=O)c1O ; CO[C@@H]1CN(c2c(-c3cc(F)cc(F)c3)cnc3c2nc(-c2cccc(C#N)c2O)n3C)CCC1N]

### Step 1: Synthesis of tert-butyl (5-bromo-4-chloro-3-nitropyridin-2-yl) carbamate (Compound 3-1-2)

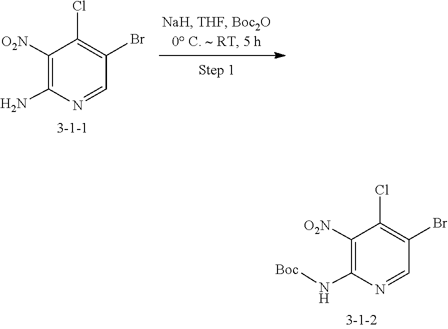 [SMILES: Nc1ncc(Br)c(Cl)c1[N+](=O)[O-] ; CC(C)(C)OC(=O)Nc1ncc(Br)c(Cl)c1[N+](=O)[O-]]

To the solution of compound 3-1-1 (3000 mg, 12.0 mmol) in THF (30.0 mL) was added NaH 60% (576 mg, 14.4 mmol) at 0° C., and then the reaction mixture was stirred at 0° C. for 1h. Boc₂O (3140 mg, 2.4 mmol) was added to reaction mixture. The reaction was stirred for 1 h and detected by LCMS, and 80% product was detected in LCMS. The reaction mixture was poured into water and extracted with EA (200 mL×2), combined organic phase, dried over, concentrated and purification by Flash (PE:EA=3:1) to give compound 3-1-2 (3680 mg) as yellow solid. MS (m/z) 295.9 (M+H−55)⁺.

### Step 2:Synthesis of tert-butyl (5-bromo-4-chloro-3-nitropyridin-2-yl) (methyl)carbamate (Compound 3-1-3)

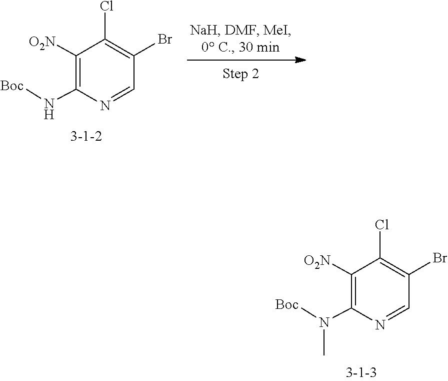 [SMILES: CC(C)(C)OC(=O)Nc1ncc(Br)c(Cl)c1[N+](=O)[O-] ; CN(C(=O)OC(C)(C)C)c1ncc(Br)c(Cl)c1[N+](=O)[O-]]

To the solution of compound 3-1-2 (216 mg, 8.0 mmol) in DMF (20.0 mL) was added NaH 60% (480 mg, 12.0 mmol) at 0° C., and then the reaction mixture was stirred at 0° C. for 1 h. MeI (1704 mg, 3.0 mmol) was added to reaction mixture. The reaction was stirred for 1 h and detected by LCMS, and 80% product was detected in LCMS. The reaction mixture was poured into water and extracted with EA (100 mL×2), combined organic phase, dried over, concentrated and purification by Flash (PE:EA=3:1) to give compound 3-1-3 (2400 mg) as yellow solid. MS (m/z) 309.9 (M+H−55)⁺.

1H NMR (400 MHz, DMSO-d6): 9.05 (s, 1H), 3.21 (s, 3H), 1.35 (s, 9H).

### Step 3: Synthesis of tert-butyl (4-chloro-5-(3-fluoro-5-methylphenyl)-3-nitropyridin-2-yl)(methyl)carbamate (Compound 3-1-4)

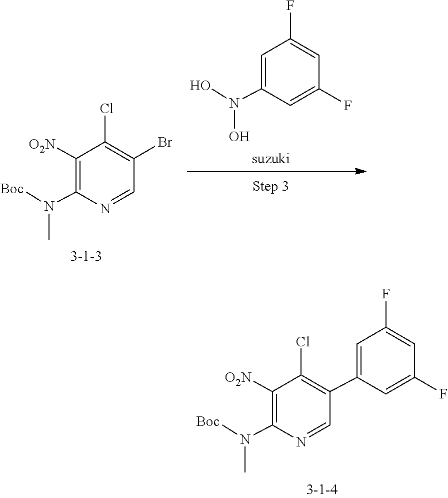 [SMILES: CN(C(=O)OC(C)(C)C)c1ncc(Br)c(Cl)c1[N+](=O)[O-] ; ON(O)c1cc(F)cc(F)c1 ; CN(C(=O)OC(C)(C)C)c1ncc(-c2cc(F)cc(F)c2)c(Cl)c1[N+](=O)[O-]]

Added compound 3-1-3 into a 100 mL flask and dissolved in 1,4-dioxane (20 mL), and then added (3,5-difluorophenyl)boronic acid (488.47 mg, 3.09 mmol), dipotassium carbonate (1.07 g, 7.73 mmol) to the solution, the solids were dissolved completely by ultrasound and then added [1,1′-Bis(diphenylphosphino)ferrocene]dichloropalladium(II) (188.62 mg, 257.78 μmol) and H₂O (2 mL) at 25° C. And the reaction solution was stirred in an oil bath with a N₂ ball at 110° C. for 5 hr. The resulting solution liquid was spun dry, and then extracted with EA (60 mL) and H₂O (20 mL) for three times. The combined organic layer was dried over Na₂SO₄ and concentrated. The crude product was purified by silica gel chromatography (100-200) eluted with PE:EtOAc=93:7 to give two peaks, and to give product compound 3-14 (425.00 mg, 1.01 mmol, 39.18% yield). MS (m/z) 344.0 (M+H)⁺.

### Step 4: Synthesis of 4-chloro-5-(3-fluoro-5-methylphenyl)-N-methyl-3-nitropyridin-2-amine (Compound 3-1-5)

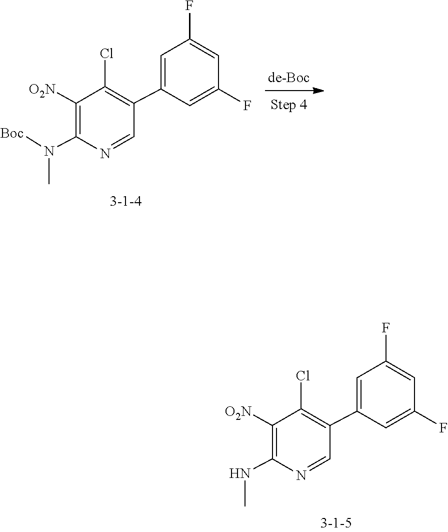 [SMILES: CN(C(=O)OC(C)(C)C)c1ncc(-c2cc(F)cc(F)c2)c(Cl)c1[N+](=O)[O-] ; CNc1ncc(-c2cc(F)cc(F)c2)c(Cl)c1[N+](=O)[O-]]

A solution of compound 3-1-4 (2400 mg, 5.5 mmol) in dichloromethane (15 mL) was added 2,2,2-trifluoroacetic acid (4.25 g, 37.30 mmol, 2.86 mL) at 25° C., the resulting mixture was stirred at 25° C. for 2 h in N₂. The reaction solution was diluted with 20 mL dichloromethane, the organic phase was concentrated, and the above operation was repeated three times. The crude used for the next step without further purification. The compound 3-1-5 (2.00 g, crude) was obtained as yellow oil. MS (m/z) 300.0 (M+H)⁺.

### Step 5: Synthesis of tert-butyl ((3R,4R)-1-(5-(3,5-difluorophenyl)-2-(methylamino)-3-nitropyridin-4-yl)-3-methoxypiperidin-4-yl)carbamate (Compound 3-1-6)

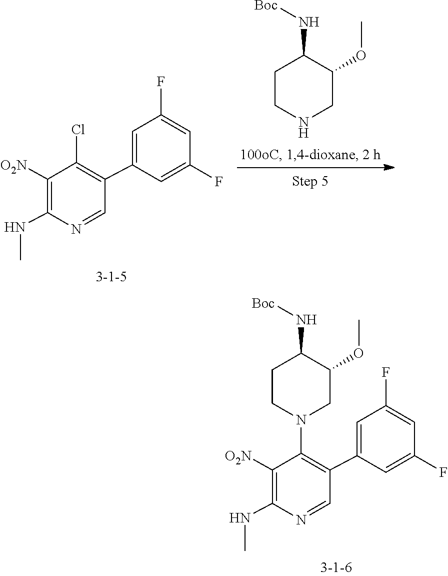 [SMILES: CNc1ncc(-c2cc(F)cc(F)c2)c(Cl)c1[N+](=O)[O-] ; CO[C@@H]1CNCC[C@H]1NC(=O)OC(C)(C)C ; CNc1ncc(-c2cc(F)cc(F)c2)c(N2CC[C@@H](NC(=O)OC(C)(C)C)[C@H](OC)C2)c1[N+](=O)[O-]]

A solution of compound 3-1-5 (2000 mg, 4.7 mmol) and tert-butyl N-[(3R,4R)-3-methoxy-4-piperidyl] carbamate (1285.4 mg, 5.6 mmol) in 1,4-dioxane (16 mL) was added DIPEA (1800 mg, 14 mmol) at 25° C., the resulting mixture was stirred at 100° C. for 5 hr in N₂. The reaction mixture was added water (50 mL). And then it was extracted with ethyl acetate (50 mL*2). Combined all organic phases and dried over anhydrous Na₂SO₄. Concentrated the organic phase to obtain a crude product. The crude product was purified by silica gel chromatography eluted with PE:EtOAc=5:1 to give compound 3-1-6 (1500.00 mg, 2.5 mmol, 74.10% yield, 82.76% purity) as yellow solid. MS (m/z) 494.3 (M+H)⁺.

### Step 6: Synthesis of tert-butyl ((3R,4R)-1-(3-amino-5-(3,5-difluorophenyl)-2-(methylamino)pyridin-4-yl)-3-methoxypiperidin-4-yl) carbamate (Compound 3-1-7)

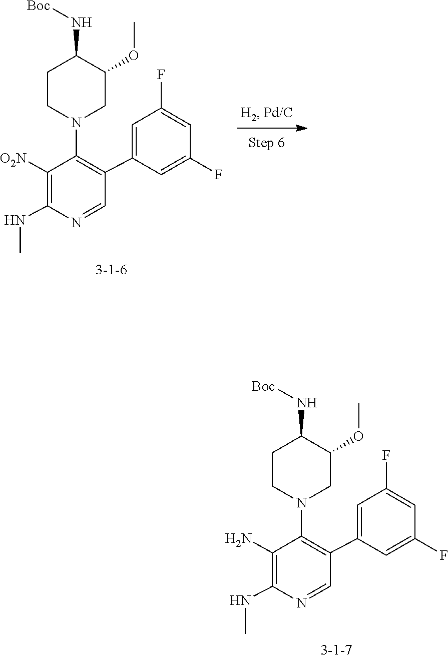 [SMILES: CNc1ncc(-c2cc(F)cc(F)c2)c(N2CC[C@@H](NC(=O)OC(C)(C)C)[C@H](OC)C2)c1[N+](=O)[O-] ; CNc1ncc(-c2cc(F)cc(F)c2)c(N2CC[C@@H](NC(=O)OC(C)(C)C)[C@H](OC)C2)c1N]

To a solution of compound 3-1-6 (tert-butyl N-[(3R,4R)-1-[5-(3-fluoro-5-methoxy-phenyl)-2-(methylamino)-3-nitro-4-pyridyl]-3-methoxy-4-piperidyl]carbamate) (740 mg, 1.32 mmol) in methanol (15 mL) was added Pd/C (320.00 mg, 131.74 μmol, 312.19 μL, 5% purity) under N₂. The suspension was degassed under vacuum and purged with H₂ several times. The mixture was stirred under H₂ balloon at 25° C. for 1 h. The suspension was filtered through Celite and filter cake was washed with MeOH (10 mL×2). The combined filtrates were concentrated to dryness to give the product compound 3-1-7 (tert-butyl N-[(3R,4R)-1-[3-amino-5-(3-fluoro-5-methoxy-phenyl)-2-(methylamino)-4-pyridyl]-3-methoxy-4-piperidyl]carbamate) (510.00 mg, 1.07 mmol, 81.40% yield) as purple solid. MS (m/z) 464.3 (M+H)⁺.

### Step 7: Synthesis of 3-(7-((3R,4R)-4-amino-3-methoxypiperidin-1-yl)-6-(3,5-difluorophenyl)-3-methyl-3H-imidazo[4,5-b]pyridin-2-yl)-2-hydroxybenzonitrile (Compound 23)

To a solution of compound 3-1-7 (tert-butyl N-[(3R,4R)-1-[3-amino-5-(3-fluoro-5-methoxy-phenyl)-2-(methylamino)-4-pyridyl]-3-methoxy-4-piperidyl]carbamate) (100 mg, 199.77 μmol) and 3-formyl-2-hydroxy-benzonitrile (35.27 mg, 239.72 μmol) in tetrahydrofuran (4 mL) was added ferric trichloride (97.21 mg, 599.30 μmol) and acetic acid (3.60 mg, 59.93 μmol) at 25° C., the resulting mixture was stirred at 60° C. for 2 hr. The reaction was complete detected by TLC and LC-MS. TLC and LCMS showed the reaction was completed. Then concentrated to give the crude material, the crude product was purified by Prep-HPLC to give the product compound 23 (38.10 mg).

Compounds 23-34 were synthesized according to the 7-step procedure described in Example 3. Analytical data for compounds 23-34 are provided below.

| Cpd. No. | Structure | Analytical Data |
|---|---|---|
| 23 | 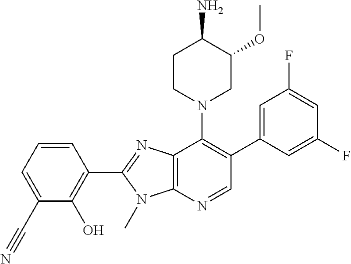 [SMILES: CO[C@@H]1CN(c2c(-c3cc(F)cc(F)c3)cnc3c2nc(-c2cccc(C#N)c2O)n3C)CC[C@H]1N] | 1H NMR (400 MHz, DMSO-d6): δ ppm 7.98 (s, 1H), 7.44 (dd, J = 7.3, 1.8 Hz, 1H), 7.32 (dd, J = 7.7, 1.9 Hz, 1H), 7.24-7.19 (m, 3H), 6.16 (t, J = 7.6 Hz, 1H), 4.29-4.26 (m, 1H), 3,73 (s, 3H), 3.53-3.43 (m, 1H), 3,22 (s, 5H), 3.05- 2.89 (m, 3H), 2.75-2.62 (m, 2H), 1.81-1.78 (m, 1H), 1.46- 1.37 (m, 1H). 19F-NMR (376 MHz, DMSO-D6): δ ppm −109.5 (t, J = 13.0 Hz,2 F). MS (m/z) 491.2 (M + H)⁺. |
| 24 | 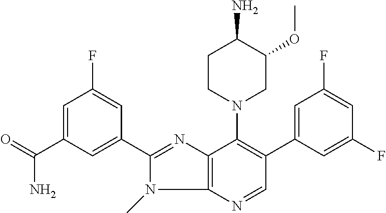 [SMILES: CO[C@@H]1CN(c2c(-c3cc(F)cc(F)c3)cnc3c2nc(-c2cc(F)cc(C(N)=O)c2)n3C)CC[C@H]1N] | 1H NMR (400 MHz, DMSO-d6): δ ppm 8.27 (t, J = 13.5 Hz, 1H), 8.07 (s, 1H), 7.96-7.86 (m, 2H), 7.72 (d, J = 12.4 Hz, 1H), 7.27-7.24 (m, 3H), 4.27 (s, 1H), 3.96 (d, J = 14,8 Hz, 3H), 3.63 (d, J = 26.7 Hz, 1H), 3.23 (d, J = 12.9 Hz, 3H), 3.09 (d, J = 29.1 Hz, 2H), 2.72-2.67 (m, 1H), 2.54 (s, 4H), 1.77 (s, 1H), 1.32 (s, 1H). 19F-NMR (376 MHz, DMSO-D6): δ ppm −109.5 (s, 2F), −111.9 (t, J = 10.8 Hz, 1F). MS (m/z) 511.3 (M + H)⁺. |
| 25 | 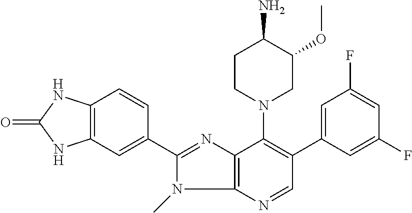 [SMILES: CO[C@@H]1CN(c2c(-c3cc(F)cc(F)c3)cnc3c2nc(-c2ccc4[nH]c(=O)[nH]c4c2)n3C)CC[C@H]1N] | 1H NMR (400 MHz, DMSO-D6): δ ppm 8.02 (s, 1H), 7.53-7.46 (m, 2H), 7.27-7.20 (m, 3H), 7.12 (d, J = 8,0 Hz, 1H), 4.24-4.22 (m, 1H), 3.88 (s, 3H), 3.59 (d, J = 13.2 Hz, 1H), 3.21 (d, J = 7.4 Hz, 3H), 3.16-3.04 (m, 2H), 2.75-2.61 (m, 3H), 2.33 (q, J = 1.9 Hz, 1H), 1.77 (d, J = 8.2 Hz, 1H), 1.39-1.34 (m, 1H). 19F-NMR (376 MHz, DMSO-D6): δ ppm −109.6 (t, ]δ = 8.7 Hz, 2F). MS (m/z) 506.2 (M + H)⁺. |
| 26 | 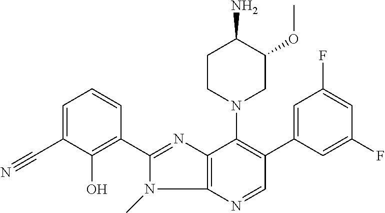 | 1H-NMR NMR (400 MHz, DMSO-D6) δ 7.91 (s, 1H), 7.48-7.46 (d, J = 7.4 Hz, 1H), 7.34-7.32 (d, J = 7.8 Hz, 1H)), 7.09 (s, J = 0.6 Hz, 1H), 7.05-7.02 (d. J = 9.9 Hz, 1H), 6.98-6.95 (d, J = 9.8 Hz, 1H),6.23-6.20 (t, J = 6.3 Hz), 4.16-4.14 (d, J = 9.9 Hz, 1H), 3.71 (s, 3H), 3.60-3.57 (d, J = 13.0 Hz, 1H), 3.10 (s, 3H), 3.02-2.97 (t, J = 11.8Hz, 1H), 2.90-2.83 (m, 1H), 2.64-2.61 (m, 1H), 2.50- 2.48 (m, 1H), 2.35 (s, 3H), 1.77-1.75 (d, J = 8.5 Hz, 1H), 1.43-1.39 (dd, J = 12.4,4.2 Hz, 1H). MS (m/z) 487.4 (M + H)⁺. |
| 27 | 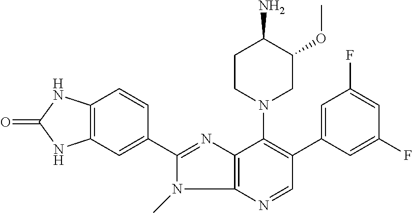 | 1H-NMR (400 MHz, DMSO-D6) δ 7.97 (d, J = 1.6 Hz, 1H), 7.52-7.46 (m, 2H), 7.16-7.10 (m, 3H), 7.01 (d, J = 9.6 Hz, 1H), 4.11 (d, J = 10.7 Hz, 1H), 3.87 (s, 3H), 3.68 (d, J = 12.1 Hz, 1H), 3.08 (dd, J =25.1, 12.0 Hz, 4H), 2.90-2.85 (m, 1H), 2.63 (q, J = 11.3 Hz, 2H), 2.36 (d, J = 27.5 Hz, 3H), 1.69 (d. J = 8.5 Hz, 1H), 1.28-1.23 (m, 1H). MS (m/z) 502.3 (M + H)⁺. |
| 28 | 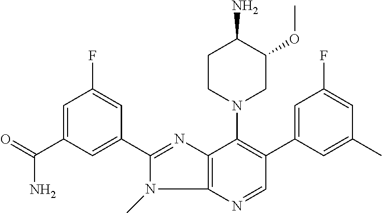 [SMILES: CO[C@@H]1CN(c2c(-c3cc(C)cc(F)c3)cnc3c2nc(-c2cc(F)cc(C(N)=O)c2)n3C)CC[C@H]1N] | 1H-NMR (400 MHz, DMSO-D6) δ 8.25-8.19 (m, 2H), 8.00 (d, J = 18.1 Hz, 1H), 7.95-7.82 (m, 2H), 7.68 (d. J = 14.3 Hz, 1H), 7.16-6.97 (m, 3H), 4.14 (t, J = 12.6 Hz, 1H), 3.90 (d, J = 18,1 Hz, 3H), 3.66 (t, J = 14.3 Hz, 1H), 3.13-3.03 (m, 4H), 2.88-2.82 (m, 1H), 2.38 (d, J = 17.3 Hz, 3H), 1.69 (s, 2H), 1.27-1.19 (m, 2H). MS (m/z) 507.4 (M + H)⁺. |
| 29 | 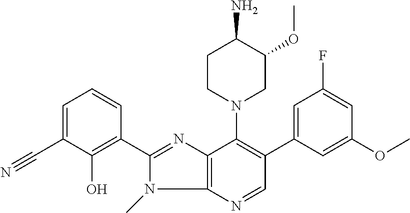 [SMILES: COc1cc(F)cc(-c2cnc3c(nc(-c4cccc(C#N)c4O)n3C)c2N2CC[C@@H](N)[C@H](OC)C2)c1] | 1H-NMR (400 MHz, DMSO-D6) δ 7.94 (d, J = 1.4 Hz, 1H), 7.48 (s, 1H), 7.33 (s, 1H), 6.85 (d, J = 5.6 Hz, 2H), 6.77 (dt, J = 11,0, 2.2 Hz, 1H), 6.20 (s,1H), 4.25 (d, J = 14.5 Hz, 1H), 3.79 (s,3H), 3.71 (d, J = 2.1 Hz, 3H), 3.58 (d, J = 12.4 Hz, 1H), 3.17 (s,3H), 3.05-2.88 (m, 2H), 2.71-2.50 (m, 2H), 2.48 (d, J = 1.9 Hz, 1H), 2.29 (dt, J = 3.7, 1.8 Hz, 1H), 1.79 (d, J = 12.3 Hz, 1H), 1.43 (q, J = 13.7 Hz, 1H). MS (m/z) 503.4 (M + H)⁺. |
| 30 | 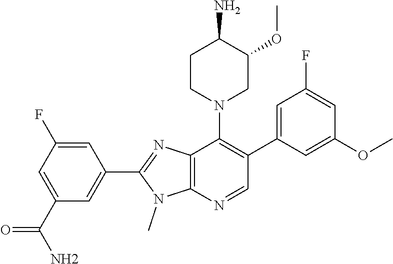 [SMILES: COc1cc(F)cc(-c2cnc3c(nc(-c4cc(F)cc(C(N)=O)c4)n3C)c2N2CC[C@@H](N)[C@H](OC)C2)c1] | 1H-NMR (400 MHz, DMSO-D6) δ 8.28 (t, J = 1.4 Hz, 1H), 8.23 (s, 1H), 8.08 (s, 1H), 8.05-8.00 (m, 1H), 7.97- 7.94 (m, 1H), 7.92-7.89 (m, 1H), 7.71 (s, 1H), 6.92-6,83 (m, 4H), 4.47-4.44 (m, 1H), 3.95 (s, 3H), 3.84 (s, 3H), 3.63 (d, J = 13.5 Hz, 1H), 3.49 (td, J = 10.0, 4.1 Hz, 1H), 3.17- 3.05 (m, 3H), 2.80-2.65 (m, 2H), 1.90 (d, J = 8.8 Hz, 1H), 1.57-1.50 (m, 1H), 1.19 (d, J = 34.4 Hz, 1H). MS (m/z) 523.2 (M + H)⁺. |
| 31 | 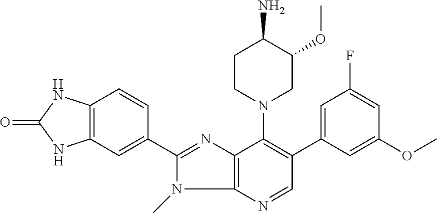 [SMILES: COc1cc(F)cc(-c2cnc3c(nc(-c4ccc5[nH]c(=O)[nH]c5c4)n3C)c2N2CC[C@@H](N)[C@H](OC)C2)c1] | 1H-NMR (400 MHz, DMSO-D6) δ 7.91 (s, 1H), 7.48- 7.46 (d, J = 7.4 Hz, 1H), 7.34-7.32 (d, J = 7.8 Hz, 1H)), 7.09 (s, J = 0.6 Hz,1H), 7.05-7.02 (d, J = 9.9 Hz, 1H), 6.98-6.95 (d, J = 9.8 Hz, 1H), 6.23-6.20 (t, J = 6.3 Hz), 4.16-4.14 (d. J = 9.9 Hz, 1H), 3.71 (s, 3H), 3.60-3.57 (d, J = 13.0 Hz, 1H), 3.10 (s, 3H), 3.02-2.97 (t, J = 11.8 Hz, 1H), 2.90-2.83 (m, 1H), 2.64-2.61 (m, 1H), 2.50-2.48 (m, 1H), 2.35 (s, 3H), 1.77-1.75 (d, J = 8.5 Hz, 1H), 1.43-1.39 (dd, J = 12.4, 4.2 Hz, 1H). MS (m/z) 518.2 (M + H)⁺. |
| 32 | 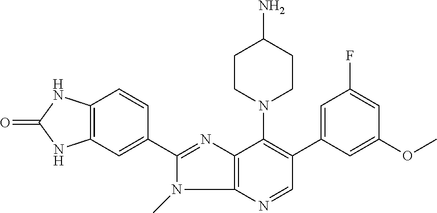 [SMILES: COc1cc(F)cc(-c2cnc3c(nc(-c4ccc5[nH]c(=O)[nH]c5c4)n3C)c2N2CCC(N)CC2)c1] | 1H NMR (400 MHz, DMSO-d6): δ ppm 10.911 (s, 1H), 8.404 (s, 1H), 7.994 (s,1H), 7.516-7.492 (dd, J1 = 1.6 Hz, J2 = 8 Hz, 1H), 7.460 (s,1H), 7.126-7.106 (d, J = 8 Hz, 1H), 6.911-6.891 (m, 2H), 6.823-6.785 (m, 1H), 3.867 (s, 3H), 3.835 (s, 3H), 3.821-3.787 (d, J = 13.6 Hz, 3H), 3.119-3.018 (m, 4H), 1.797-1.769 (d, J = 11.2 Hz, 2H), 1.513-1.432 (dd, J1 = 10.8 Hz, J2 = 22.4 Hz, 2H). MS (m/z) 488.2 (M + H)⁺. |
| 33 | 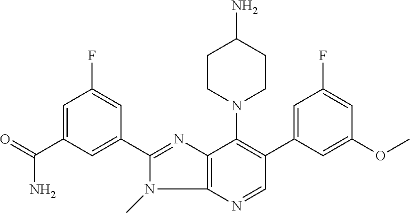 [SMILES: COc1cc(F)cc(-c2cnc3c(nc(-c4cc(F)cc(C(N)=O)c4)n3C)c2N2CCC(N)CC2)c1] | 1H NMR (400 MHz, DMSO-d6): 8.398 (s, 1H), 8.312 (s, 1H), 8.241 (s, 1H), 7.944-7.876 (dd, J1 = 8.8 Hz, J2 = 17.2 Hz, 2H), 7.719 (s, 1H), 6.914-6,894 (m, 2H), 6.835-6.802 (m, 1H), 3.920 (s, 3H), 3.837-3.814 (m, 6H), 3.123-3.043 (dd, J1 = 12 Hz, J2 = 24.4 Hz, 4H), 1.815- 1.785 (d, J = 12 Hz), 1.539-1.452 (dd, J1 = 12 Hz, J2 = 22.4 Hz, 2H). MS (m/z) 493.2 (M + H)⁺. |
| 34 | 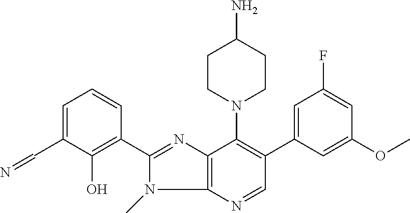 [SMILES: COc1cc(F)cc(-c2cnc3c(nc(-c4cccc(C=N)c4O)n3C)c2N2CCC(N)CC2)c1] | 1H NMR (400 MHz, DMSO-d6): 7.935 (s, 2H), 7.393- 7.326 (m, 2H), 6.844-6.761 (m, 3H), 6.214-6.178 (t, J = 7.2 Hz, 1H), 3.805 (s, 3H), 3.703-3.683 (m, 5H), 3.000-2.941] (t, J = 12 Hz, 2H), 2.891-2.848 (t, J = 7.2 Hz, 1H), 1.675-1.649 (m, 2H), 1.468-1.391 (m, 2H). MS (m/z) 473.0 (M + H)⁺. |

### Example 4

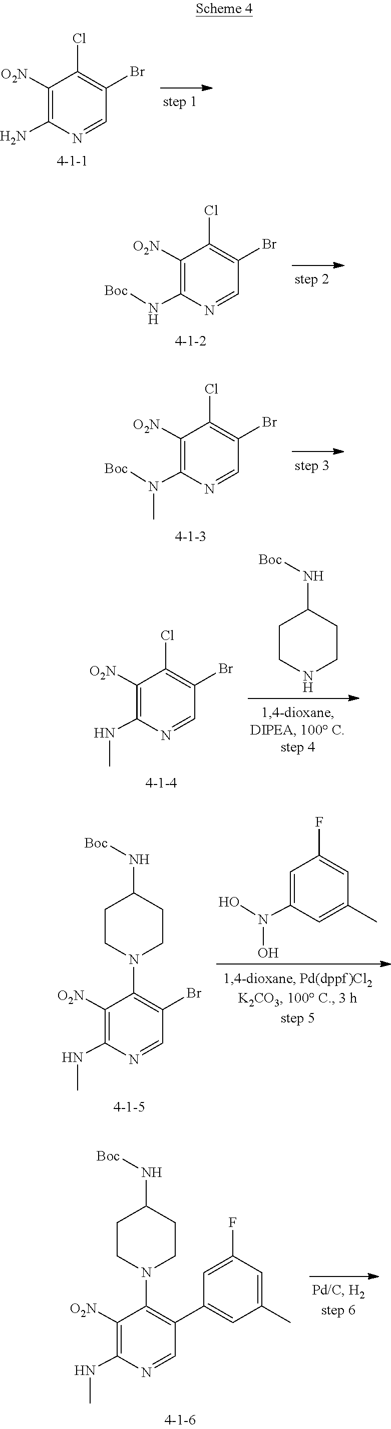

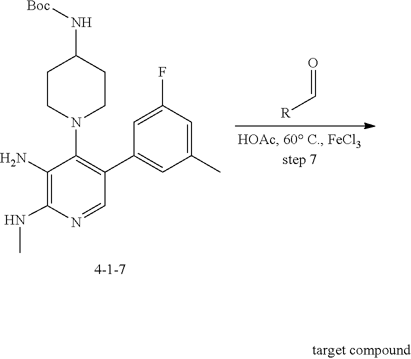

### Step 1: Synthesis of tert-butyl (5-bromo-4-chloro-3-nitropyridin-2-yl) carbamate (Compound 4-1-2)

To the solution of compound 4-1-1 (3 g, 11.88 mmol) in THF (40 mL) was added NaH 60% (950 mg, 14.26 mmol) at 0° C., then the reaction mixture was stirred at 0° C. for 1 h. (Boc)₂O (3.11 g, 14.26 mmol) was added to reaction mixture. The reaction was stirred for Ib and detected by LCMS. The reaction mixture was poured into water and extracted with EA (200 mL×3), combined organic phase, dried over, concentrated and purification by Flash (PE:EA=3:1) to give compound 4-1-2 (3.2 g) as yellow solid. MS (m/z) 295.9 (M+H−55.

### Step 2: Synthesis of tert-butyl (5-bromo-4-chloro-3-nitropyridin-2-yl) (methyl)carbamate (Compound 4-1-3)

To the solution of compound 4-1-2 (3.2 g, 9.07 mmol) in DMF (30.0 mL) was added NaH 60% (544 mg, 3.0 mmol) at 0° C., and then the reaction mixture was stirred at 0° C. for 1 h. MeI (1.9 g, 3.0 mmol) was added to reaction mixture. The reaction was stirred for 1 h and detected by LCMS. The reaction mixture was poured into water and extracted with EA (200 mL×3), combined organic phase, dried over, concentrated and purification by Flash (PE:EA=3:1) to give compound 4-1-3 (1.5 g) as yellow solid. MS (m/z) 309.9 (M+H−55)⁺.

### Step 3: Synthesis of 5-bromo-4-chloro-N-methyl-3-nitropyridin-2-amine (Compound 4-1-4)

To the solution of compound 4-1-3 (1.5 g, 4.09 mmol) in DCM (15 mL) was added TFA (5 mL) at 25° C. and then the reaction mixture was stirred at 25° C. for 1 h and detected by LCMS. The reaction mixture was concentrated to give the crude product compound 4-1-4 for next step. MS (m/z) 266.0 (M+H)⁺.

### Step 4: Synthesis of tert-butyl (1-(5-bromo-2-(methylamino)-3-nitropyridin-4-yl)piperidin-4-yl)carbamate (Compound 4-1-5)

To the solution of compound 4-1-4 (1 g, 3.75 mmol) was added tert-butyl piperidin-4-ylcarbamate (0.9 g, 4.5 mmol) and DIPEA (1.45 g, 11.25 mmol) in 1,4-dioxane (10.0 mL) at 25° C. The reaction was warmed to 80° C. stirred for 21. The reaction was detected by LCMS, reaction is ok. LCMS show the reaction was worked complete. The solution was cooled to 0° C., and lots of solid was observed. After filter, compound 4-1-5 (1.5 g, 93% yield) was obtained as a yellow solid. MS (m/z) 430.0 (M+H)⁺.

### Step 5: Synthesis of tert-butyl (1-(5-(3-fluoro-5-methylphenyl)-2-(methylamino)-3-nitropyridin-4-yl) piperidin-4-yl) carbamate (Compound 4-1-6)

To a solution of compound 4-1-5 (700 mg, 1.63 μmol, 1 eq), (3-fluoro-5-methylphenyl) boronic acid (377 mg, 2.45 mmol, 1.5 eq) and K₂CO₃ (676 mg, 4.89 mmol, 3 eq) in the mixture of dioxane (15 mL) and H₂O (1 mL) was added PdCl₂(dppf) (117 mg, 0.161 mmol) under N₂. The suspension was degassed under vacuum and purged with N₂ several times. Then the reaction mixture was stirred at 110° C. for 2 h. The reaction mixture was diluted with water (20 mL) and extracted with EA (40 mL×3). The combined organic extract was washed with brine (15 mL), dried over Na₂SO₄, filtered and concentrated to give the crude material, which was purified by chromatography through a Redi-Sep pre-packed silica gel column (12 g), eluting with a gradient of 0% to 10% MeOH in DCM to give compound 4-1-6 (545 mg, 72.9% yield) as white solid. MS (m/z) 460.2 (M+H)⁺.

### Step 6: Synthesis of tert-butyl (1-(3-amino-5-(3-fluoro-5-methylphenyl)-2-(methylamino)pyridin-4-yl)piperidin-4-yl)carbamate (Compound 4-1-7)

To a solution of compound 4-1-6 (545 mg, 1.19 mmol, 1 eq) in THF (20 mL) was added Pd/C (126.3 mg, 1.19 mmol) the suspension was degassed under vacuum and purged with H₂ several times. Then the reaction mixture was stirred at rt for 2h. TLC and LCMS showed the reaction was complete. The reaction mixture was filtered and concentrated to give compound 4-1-7 (420 mg, 82.4% yield) as oil. MS (m/z) 430.2 (M+H)⁺.

### Step 7: Synthesis of 3-(7-(4-aminopiperidin-1-yl)-6-(3-fluoro-5-methylphenyl)-3-methyl-3H-imidazo[4,5-b]pyridin-2-yl)-5-fluorobenzamide (Compound 35)

To a solution of compound 4-1-7 (100 mg, 0.233 mmol, 1 eq) and 3-fluoro-5-formylbenzamide (47 mg, 0.280 mmol, 1.2 eq) in THF (10 mL) was added AcOH (0.05 mL). The reaction mixture was stirred at 60° C. for 2 h. After 1 hour, FeCl₃ (37.3 mg, 0.233 mmol, 1 eq) was added. The reaction mixture was stirred at 60° C. for 2h. TLC and LCMS showed the reaction was complete. The reaction mixture was diluted with water (30 mL) and extracted with EA (50 mL×4). The combined organic extract concentrated to give the crude material, the crude material was purified by silica gel chromatography eluted with DCM:MeOH (from 100:1 to 1:1) to give crude material, then the crude material was purified by Prep-HPLC to give compound 35 (19.8 mg) as white solid.

### Synthesis of Compound 42 and Compound 43

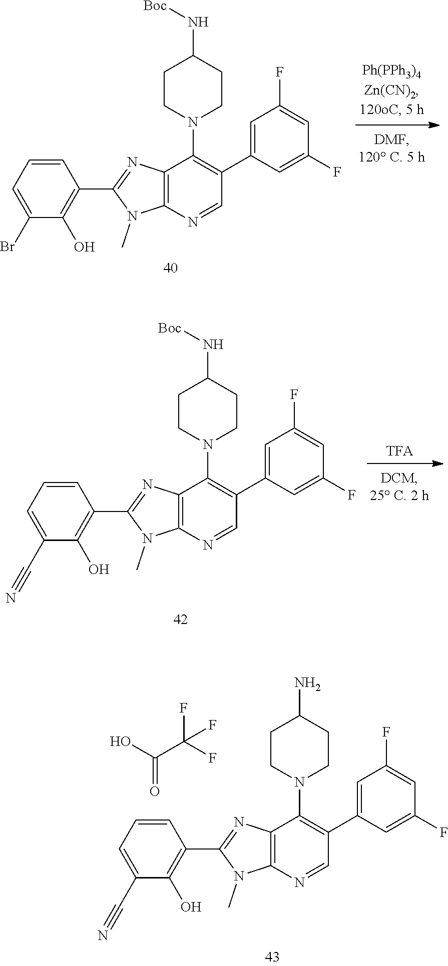

The compound 40 (61 mg, 0.10 mmol, 1.0 equiv), Zn(CN)₂ (26 mg, 0.22 mmol, 2.2 equiv), Pd(PPh₃)₄ (14 mg, 0.02 mmol, 20 mol %) were suspended with DMF (2.0 mL) at protected by N₂, and the reaction mixture was stirred at 120° C. for 5 h. The reaction mixture was purification by HPLC to give compound 42 (27 mg) as white solid.

To the solution of compound 42 (22 mg, 0.06 mmol) in DCM (2.0 mL) was added TFA (0.5 mL) at 25° C., and then the reaction mixture was stirred at 25° C. for 2 h and detected by LCMS, 100% product in LCMS. The reaction mixture was concentrated and freeze-dried to give compound 43 (16.5 mg) as two TFA salt white solid.

### Synthesis of 3-fluoro-5-formylbenzamide (Compound 4-1-aldehyde)

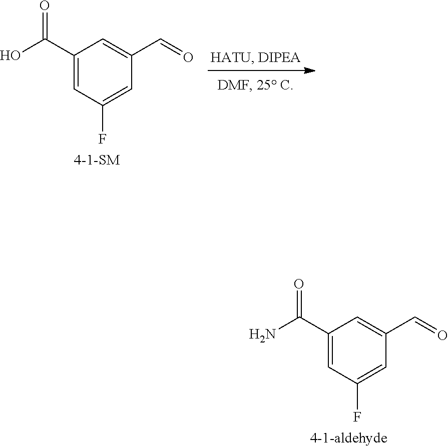 [SMILES: O=Cc1cc(F)cc(C(=O)O)c1 ; NC(=O)c1cc(F)cc(C=O)c1]

To a solution of 3-fluoro-5-formylbenzoic acid (compound 4-1-SM, 100 mg, 0.60 mmol, 1.0 eq) and NH₄Cl (65 mg, 1.19 mmol, 1.2 eq) in DMF (2 mL) was added DIPEA (230 mg, 1.79 mmol, 3.0 eq) and HATU (270 mg, 0.71 mmol, 1.2 eq). The mixture was stirred at 25° C. for 2 h. The mixture was extracted with water and ethyl acetate, the aqueous phase was extracted with ethyl acetate, the combined organic layers were washed with brine and dried with Na₂SO₄, and the organic layers were concentrated under reduced pressure to obtain a crude product, which was purified by silica gel column chromatography DCM:MeOH from 0% to 11% to give the product 3-fluoro-5-formylbenzamide (compound 4-1-aldehyde, 35 mg) as white solid. MS (m/z) 168.1 (M+H)⁺.

Compounds 3543 were synthesized according to the 7-step procedure described in Example 4. Analytical data for compounds 3543 are provided below.

| Cpd. No. | Structure | Analytical Data |
|---|---|---|
| 35 | 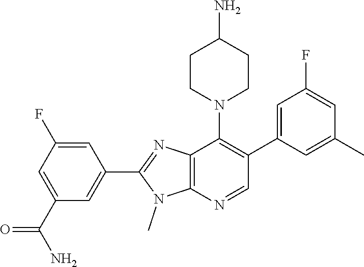 [SMILES: Cc1cc(F)cc(-c2cnc3c(nc(-c4cc(F)cc(C(N)=O)c4)n3C)c2N2CCC(N)CC2)c1] | 1H NMR (400 MHz, DMSO-d6): δ ppm 8.386 (s, 1H), 8.289 (s, 1H), 8.237 (s, 1H), 7.943-7.920 (d, J = 9.2 Hz, 1H), 7.898- 7.874 (d, J = 9.6 Hz, 1H), 7.710 (s, 1H), 7.168 (s, 1H), 7.125-7.102 (d, J = 9.2 Hz, 1H), 7.028- 7.004 (d, J = 9.6 Hz, 1H), 3.920 (s, 3H), 3.818- 3.786 (d, J = 12.8 Hz, 3H), 3.116-3.055 (t, J = 12 Hz, 4H), 2.405 (s, 3H)), 1.795-1.767 (d, J = 11.2 Hz, 2H), 1.484-1.461 (d, J = 9.2 Hz, 2H). MS (m/z) 477.2 (M + H)⁺. |
| 36 | 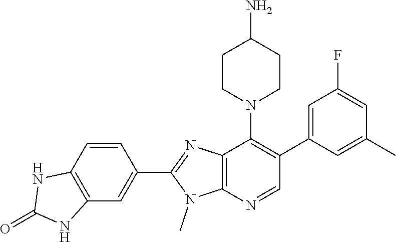 [SMILES: Cc1cc(F)cc(-c2cnc3c(nc(-c4ccc5[nH]c(=O)[nH]c5c4)n3C)c2N2CCC(N)CC2)c1] | 1H NMR (400 MHz, DMSO-d6): δ ppm 11.016-10.795 (m, 1H), 8.393 (s, 1H), 7.978 (s, 1H), 7.512-7.492 (d, J = 8 Hz, 1H), 7.460 (s, 1H), 7.167 (s, 1H), 7.125-7.105 (d, J = 8 Hz, 2H), 7.018-6.993 (d, J = 10 Hz, 1H), 3.867 (s, 3H), 3.792-3.762 (d, J = 12 Hz, 3H), 3.113-3.051 (t, J = 12.8 Hz, 4H), 2.403 (s, 3H), 1.792-1.758 (d, J = 13.6 Hz, 2H), 1.503-1.4159 (m, 2H). MS (m/z) 472.2 (M + H)⁺. |
| 37 | 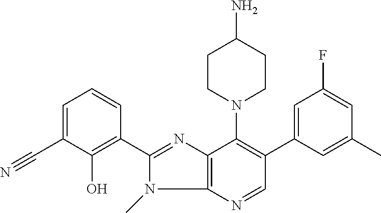 [SMILES: Cc1cc(F)cc(-c2cnc3c(nc(-c4cccc(C#N)c4O)n3C)c2N2CCC(N)CC2)c1] | 1H NMR (400 MHz, DMSO-d6): 6 ppm 8.198 (s, 1H) 7.939 (s, 1H), 7.509-7.486 (m, 1H), 7.406-7.382 (m, 1H), 7.106 (s, 1H), 7.064-7.039 (d, J = 10 Hz, 1H), 6.995- 6.969 (d, J = 10.4 Hz, 1H), 6.330-6.293 (t, J = 7.6 Hz, 1H), 3.726 (s, 3H), 3.700-3.667 (d, J = 13.2 Hz, 3H), 3,018-2.960 (t, J = 12 Hz, 4H), 2.368 (s, 3H), 1.717- 1.689 (d, J = 11.2 Hz, 2H), 1.500-1.409 (m, 2H). MS (m/z) 457.2 (M + H)⁺. |
| 38 | 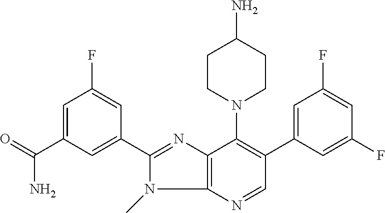 [SMILES: Cn1c(-c2cc(F)cc(C(N)=O)c2)nc2c(N3CCC(N)CC3)c(-c3cc(F)cc(F)c3)cnc21] | 1H NMR (400 MHz, DMSO-d6): 8.379 (s, 1H), 8.313- 8.239 (s, 1H ), 8.073 (s, 1H ), 7.952-7.874 (m, 2H), 7.714 (s,1H), 7.252-7.230 (d, J = 8.8 Hz, 3H), 3.925 (s, 3H), 3.811-3.779 (d, J = 12.8 Hz, 4H ), 3.143-3.083 (t, J = 12.0 Hz, 3H), 1.815-1.786 (d, J = 11.6 Hz, 2H), 1.511-1.479 (d, J = 12,8 Hz, 2H). MS (m/z) 481.2 (M + H)⁺. |
| 39 | 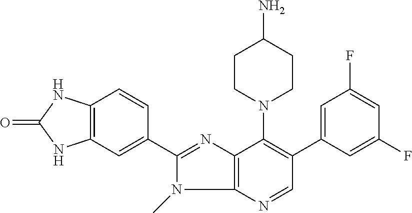 [SMILES: Cn1c(-c2ccc3[nH]c(=O)[nH]c3c2)nc2c(N3CCC(N)CC3)c(-c3cc(F)cc(F)c3)cnc21] | 1H NMR (400 MHz, DMSO-d6): δ ppm 10.892 (s, 1H), 8.412 (s, 1H), 8.016 (s, 1H), 7.520-7.465 (m, 2H), 7.244- 7.187 (m, 3H), 7.127-7.107 (d, J = 8 Hz, 1H), 3.872 (s, 3H), 3.780-3.747 (d, J = 13.2 Hz, 2H), 3.135-3.082 (t, J = 11.6 Hz, 3H), 3.031-2.984 (m, 2.H), 1.799-1.770 (d, J = 11.6 Hz, 2.H), 1.510-1.426 (dd, J1 = 22.8 Hz, J2 = 10 Hz, 2H). MS (m/z) 476.3 (M + H)⁺. |
| 40 | 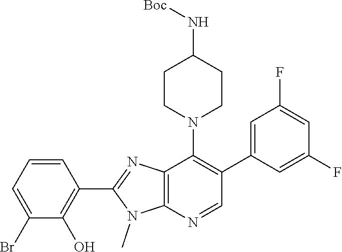 [SMILES: Cn1c(-c2cccc(Br)c2O)nc2c(N3CCC(NC(=O)OC(C)(C)C)CC3)c(-c3cc(F)cc(F)c3)cnc21] | 1H NMR (400 MHz, CDCl₃): δ ppm 13.98 (s, 1H), 8.09 (s, 1H), 7.85-7.82 ( dd, J1 = 1.6 Hz, J2 =8.0Hz, 1H ), 7.68-7.66 ( dd, J1 = 1.6 Hz, J2 = 8.0 Hz, 1H ), 7.05- 7.01 (m, 2H), 6.93-6.89 (1, J = 9.6 Hz, 1H), 6.84-6.78 (m, 1H), 4.51 (s, 1H), 4.16 (s, 3H), 3.75-3.72 (m, 2H), 3,70 (s, 1H), 3.25-3.18 (m, 2H), 1.98-1.96 (m, 2H), 1.52 (s, 9H), 1.48-1.50 (m, 2H). MS (m/z) 614.2 (M + H)⁺. |
| 41 | 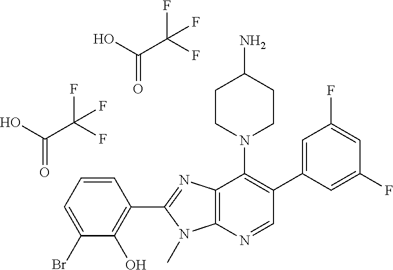 | 1H NMR (400 MHz, DMSO-d6): δ ppm 13.22 (s, 1H), 8.14 (s, 1H), 8.01-7,98 ( dd, J1 = 1,6 Hz, J2 = 8.0 Hz, 1H), 7.91 (s, 2H), 7.78-7.76 ( dd, J1 = 1.6 Hz, J2 = 8.0 Hz, 1H ), 7.29-7.24 (m, 3H), 7.05-7.01 (t, J = 9.6 Hz, 1H), 4.04 (s, 3H), 3.75-3.72 (m, 2H), 3.28-3.27 (m, 1H), 3.17-3.11 (m, 2H), 1.92-1.89 (m, 2H), 1.27-1.25 (m, 2H). MS (m/z) 514.1 (M + H)⁺. |
| 42 | 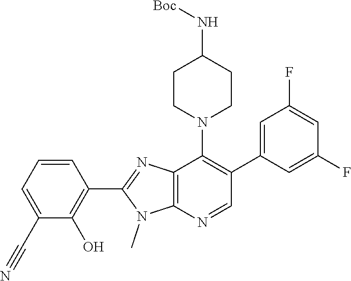 [SMILES: Cn1c(-c2cccc(C#N)c2O)nc2c(N3CCC(NC(=O)OC(C)(C)C)CC3)c(-c3cc(F)cc(F)c3)cnc21] | 1H NMR (400 MHz, DMSO-d6): δ ppm 14.11 (s, 1H), 8.32-8.29 ( dd, J1 = 1.2 Hz, J2 = 8.0 Hz, 1H ), 8.13 (s, 1H), 7.90-7.88 ( dd, J1 = 1.2 Hz, J2 = 8.0 Hz, 1H ), 7.26-7.19 (m, 4H), 6.97-6.95 (m, 1H), 4.06 (s, 3H), 3.69-3.48 (m, 2H), 3.46-3.45 (m, 1H), 3.15-3.09 (m, 2H), 1.74-1.72 (m, 2H), 1.47-1.44 (m, 2H), 1.37 (s, 9H). MS (m/z) 561.0 (M + H)⁺. |
| 43 | 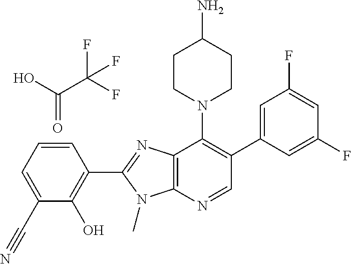 | 1H NMR (400 MHz, DMSO-d63): δ ppm 14.11 (s, 1H), 8.33-8.31 (dd, J₁ = 1.2 Hz, J2 = 8.0 Hz, 1H ), 8.16 (s, 1H), 7.92-7.86 ( m, 3H ), 7.54-7.52 (m, 1H), 7.30-7.20 (m, 4H), 4.08 (s, 3H), 3.69-3.48 (m, 2H), 3.46-3.45 (m, 1H), 3.15-3.09 (m, 2H), 1.61-1.56 (m, 2H), 1.47-1.44 (m, 2H). MS (m/z) 461.2 (M + H)⁺. |

### Example 5

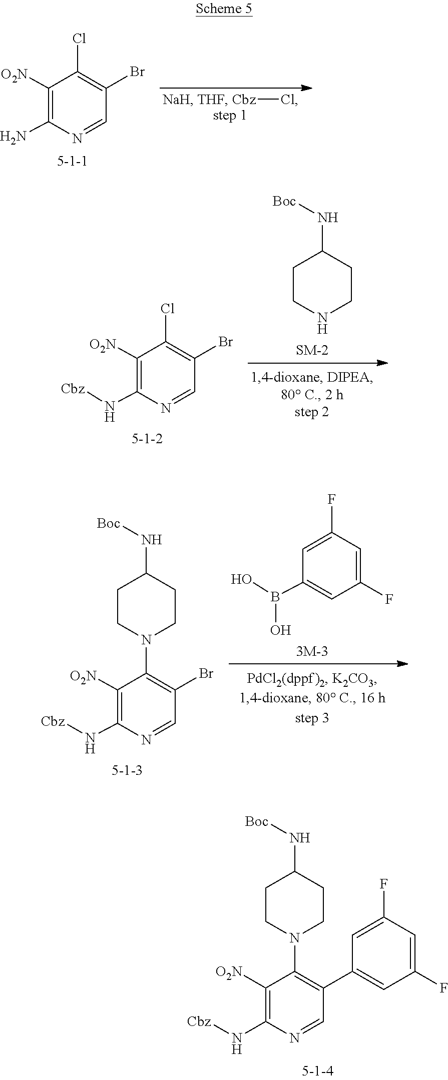

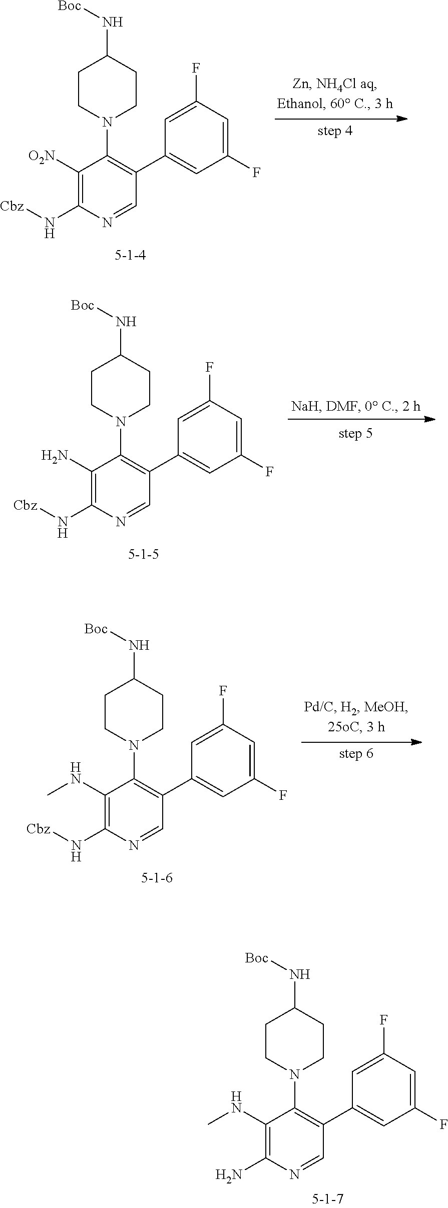 [SMILES: CC(C)(C)OC(=O)NC1CCN(c2c(-c3cc(F)cc(F)c3)cnc(NC(=O)OCc3ccccc3)c2[N+](=O)[O-])CC1 ; CC(C)(C)OC(=O)NC1CCN(c2c(-c3cc(F)cc(F)c3)cnc(NC(=O)OCc3ccccc3)c2N)CC1 ; CNc1c(NC(=O)OCc2ccccc2)ncc(-c2cc(F)cc(F)c2)c1N1CCC(NC(=O)OC(C)(C)C)CC1 ; CNc1c(N)ncc(-c2cc(F)cc(F)c2)c1N1CCC(NC(=O)OC(C)(C)C)CC1]

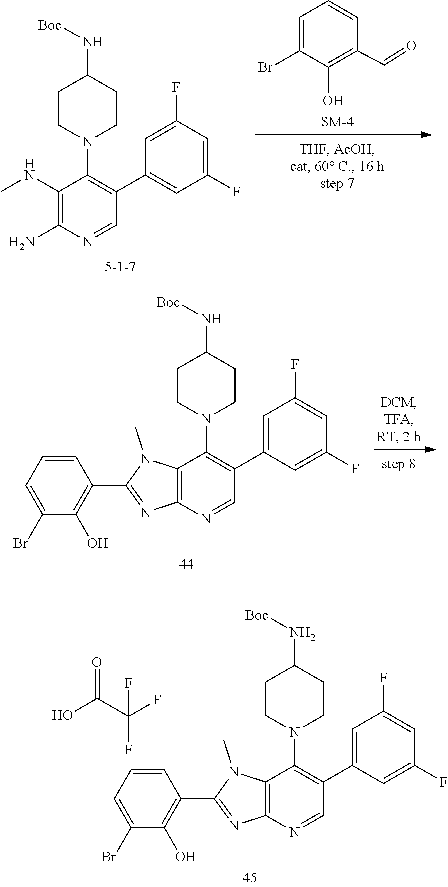

### Step 1: Synthesis of Compound 5-1-2

To the solution of compound 5-1-1 (750 mg, 3.0 mmol) in THE (15.0 mL) was added NaH 60% (144 mg, 3.6 mmol) at 0° C. and then the reaction mixture was stirred at 0° C. for 1 h. Cbz-Cl (614 mg, 0.6 mmol) was added to reaction mixture. The reaction was stirred for 1 h and detected by LCMS, and 80% product was detected in LCMS. The reaction mixture was poured into water and extracted with EA (50 mL×2), combined organic phase, dried over, concentrated and purification by Flash (PE:EA=3:1) to give compound 5-1-2 (510 mg) as yellow solid. MS (m/z) 386.1 (M+H)⁺.

### Step 2: Synthesis of Compound 5-1-3

The compound 5-1-2 (425 mg, 1.1 mmol), SM-2 (250 mg, 1.2 mmol) and DIPEA (429 mg, 3.3 mmol) were dissolved in 1,4-dioxane (10.0 mL) at it. The reaction was warmed to 80° C. and stirred for 2h. LCMS showed the reaction was completed. The reaction mixture was concentrated and purification by Flash (PE:EA=1:1) to give compound 5-1-3 (280 mg) as yellow solid. MS (m/z) 550.2 (M+H)⁺.

### Step 3: Synthesis of Compound 5-1-4

The compound 5-1-3 (200 mg, 0.36 mmol, 1.0 equiv), SM-3 (68 mg, 0.44 mmol, 1.2 equiv). Na₂CO₃ (134 mg, 1.08 mmol, 3.0 equiv), PdCl₂[dppf] (52 mg, 0.07 mmol, 20 mol %) were suspended with 1,4-dioxane (6.0 mL) and Water (2.0 mL) at protected by N₂, and the reaction mixture was stirred at 110° C. for 5 h. The reaction was detected by LCMS, and 70% product was detected. The reaction mixture was purification by flash (PE:EA=3:1-1:1) to give compound 5-1-4 (140 mg) as yellow solid. MS (m/z) 584.2 (M+H)⁺.

### Step 4: Synthesis of Compound 5-1-5

To a mixture of compound 5-1-4 (58 mg, 0.10 mmol), Zn (50 mg) in EtOH (3.0 mL) and NH₄Cl aq (1.0 mL) at 25° C. The reaction mixture was stirred for 3 h at 50° C. The mixture was detected by LCMS, reaction was complete 95%. The reaction was poured into water, extracted with EA (20 mL×2), combined organic phase, dried over, concentrated to give compound 5-1-5 (74 mg) as gray solid. MS (m/z) 554.2 (M+H)⁺.

### Step 5: Synthesis of Compound 5-1-6

To the solution of compound 5-1-5 (74 mg, 0.13 mmol) in DMF (3.0 mL) was added NaH 60/6 (5.2 mg, 0.13 mmol) at 0° C., and then the reaction mixture was stirred at 0° C. for 1 h. MeI (19 mg, 0.13 mmol) was added to reaction mixture. The reaction was stirred for 1 h and detected by LCMS, and 80% product was detected in LCMS. The reaction mixture was poured into water and extracted with EA (50 mL×2), combined organic phase, dried over, concentrated and purification by Flash (PE:EA=3:1) to give compound 5-1-6 (52 mg) as yellow solid. MS (m/z) 568.2 (M+H)⁺.

### Step 6: Synthesis of Compound 5-1-7

To a mixture of compound 5-1-6 (52 mg) in MeOH (5 mL) at 25° C. Pd/C (100 mg) was added to reaction mixture. The reaction mixture was stirred for 3 h at 25° C. H₂ protect. The mixture was detected by LCMS, reaction was complete 95%. The reaction was filtered, concentrated and purification by flash to give compound 5-1-7 (42 mg) as gray solid. MS (m/z) 434.2 (M+H)⁺.

### Step 7: Synthesis of Compound 44

The compound 5-1-7 (42 mg, 0.10 mmol), SM-4 (24 mg, 0.12 mmol), AcOH (15 mg, cat.) was dissolved in THF (5.0 mL) at 25° C. The reaction was warmed to 60° C. stirred for 16 h. The reaction was detected by LCMS, reaction is ok. The reaction mixture was concentrated and purification by HPLC (0.5% FA) to give compound 44 (21 mg) as white solid. 7.3 mg was delivered.

### Step 8: Synthesis of Compound 45

The compound 44 (20 mg) was dissolved in DCM (2.0 mL) at 25° C. TFA (0.5 mL) was added to reaction, and then the reaction mixture was stirred at 25° C. for 2 h. The reaction mixture was concentrated and freeze-dried to give compound 45 (18.3 mg) as one TFA salt white solid.

Compounds 44 and 45 were synthesized according to the 7- or 8-step procedure described in Example 5. Analytical data for compounds 44 and 45 are provided below.

| Cpd. No. | Structure | Analytical Data |
|---|---|---|
| 44 | 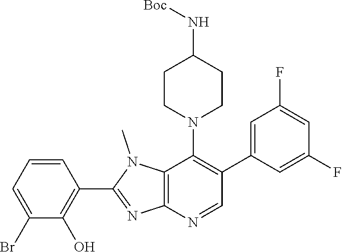 [SMILES: Cn1c(-c2cccc(Br)c2O)nc2ncc(-c3cc(F)cc(F)c3)c(N3CCC(NC(=O)OC(C)(C)C)CC3)c21] | 1H NMR (400 MHz, DMSO-d6): δ ppm 13.32 (s, 1H), 8.10 (s, 1H), 8.00-7.98 ( d, J = 8.0 Hz, 1H ), 7.77-7.75 ( d, J = 8.0 Hz, 1H ), 7.25-7.22 ( m, 3H ), 7.03-7.95 (m, 2H), 4.03 (s, 3H), 3.69-3.66 (m, 2H), 3.44-3.42 (m, 1H), 3.15-3.08 (m, 2H), 1.73-1.71 (m, 2H), 1.44-1.41 (m, 2H), 1.37 (s, 9H). MS (m/z) 614.2 (M + H)⁺. |
| 45 | 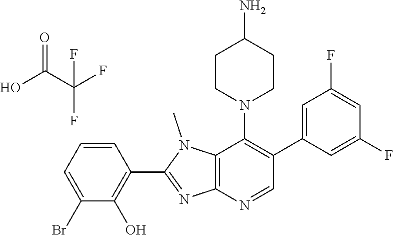 | 1H NMR (400 MHz, DMSO-d6): δ ppm 13.52 (s, 1H), 8.14 (s. 1H ), 8.01-7.99 (m, 1H), 7.88-7.86 ( m, 3H ), 7.78-7.76 (m, 1H), 7.29-7.24 (m, 3H), 7.05-7.01 (m, 1H), 4.04 (s, 3H), 3.81-3.75 (m, 2H), 3.27-3.26 (m, 1H), 3.17-3.11 (m, 2H), 1.91-1.88 (m, 2H), 1.59-1.56 (m, 2H). MS (m/z) 514.1 (M + H)⁺. |

### Example 6

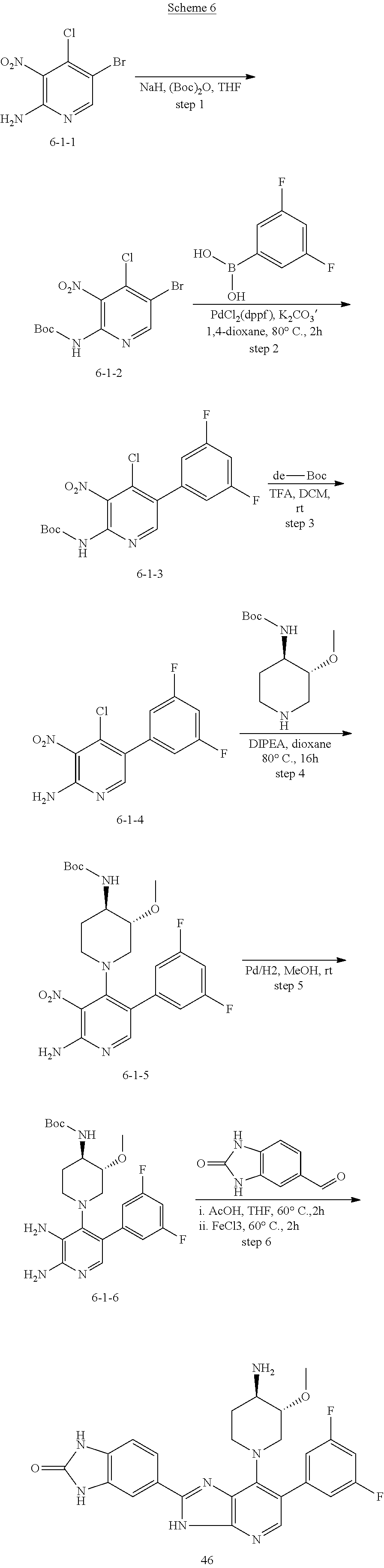

### Step 1: Synthesis of tert-butyl (5-bromo-4-chloro-3-nitropyridin-2-yl)carbamate (Compound 6-1-2)

The compound 6-1-1 (3 g, 11.88 mmol) was dissolved in THF (40 mL) at 25° C. The reaction was cold to 0° C. NaH 60% (950 mg, 14.26 mmol) was added to reaction, then the reaction mixture was stirred at 0° C. for 1 h. (Boc)₂O (3.11 g, 14.26 mmol) was added to reaction mixture. The reaction was stirred for 1 h. The reaction mixture was poured into water and extracted with EA (200 mL-3), combined organic phase, dried over, concentrated and purification by Flash (PE:EA=3:1) to give compound 6-1-2 (3.2 g) as yellow solid. MS (m/z) 295.9 (M+H−55)⁺.

### Step 2: Synthesis of tert-butyl (4-chloro-5-(3,5-difluorophenyl)-3-nitropyridin-2-yl) carbamate (Compound 6-1-3)

To a solution of compound 6-1-2 (1500 mg, 4.27 mmol, 1 eq), (3,5-difluorophenyl) boronic acid (740 mg, 4.70 mmol, 1.1 eq) and K₂CO₃ (1770 mg, 12.82 mmol, 3 eq) in the mixture of 1,4-Dioxane (20 mL) and Water (2 mL) was added PdCl₂(dppf) (310 mg, 0.43 mmol, 0.1 eq). The suspension was degassed under vacuum and purged with Nitrogen several times. The mixture was stirred at 80° C. for 2 h under Nitrogen. The reaction was monitored by LC-MS and TLC, which showed the reaction was complete. The reaction liquid was extracted with Water (10 mL) and Ethyl acetate (10 mL×3) and the combined organic phase dried with Na₂SO₄. The organic phase was dried with silica gel by rotary evaporator and was purified by flash chromatography Petroleum ether:Ethyl acetate from 0 to 10% to obtain the liquid product that was dried by rotary evaporator to obtain crude tert-butyl (4-chloro-5-(3,5-difluorophenyl)-3-nitropyridin-2-yl)carbamate (compound 6-1-3, 1216 mg). MS (m/z) 408.1 (M+Na)⁺, 330.0 (M+H−55)⁺.

### Step 3: Synthesis of 4-chloro-5-(3,5-difluorophenyl)-3-nitropyridin-2-amine (Compound 6-1-4)

To a solution of compound 6-1-3 (600 mg, 1.56 mmol, 1 eq) in Dichloromethane (2.00 mL) was added Trifluoroacetic acid (4 mL). The mixture was stirred for 2 h at 25° C. The reaction was monitored by LCMS and TLC, which showed the reaction was complete. The reaction liquid was dried by rotary evaporator to obtain the product in the form of trifluoroacetate as a yellow oil, which was dried with Dichloromethane by rotary evaporator to obtain crude 4-chloro-5-(3,5-difluorophenyl)-3-nitropyridin-2-amine (compound 6-14). MS (m/z) 286.0 (M+H)⁺.

### Step 4: Synthesis of tert-butyl ((3R,4R)-1-(2-amino-5-(3,5-difluorophenyl)-3-nitropyridin-4-yl)-3-methoxypiperidin-4-yl)carbamate (Compound 6-1-5)

To a solution of compound 6-1-4 (350 mg, 1.23 mmol, 1 eq) in 1,4-dioxane (3.00 mL) was added tert-butyl ((3R,4R)-3-methoxypiperidin-4-yl) carbamate (283 mg, 1.23 mmol, 1 eq) and DIPEA (476 mg, 3.68 mmol, 3 eq). The mixture was stirred for 16 h at 80° C. The reaction was monitored by LCMS and TLC, which showed the reaction was complete. The reaction liquid was extracted with Water (10 mL) and Ethyl acetate (10 mL*3) and the organic phase dried with Na₂SO₄. The organic phase was dried with silica gel by rotary evaporator and was purified by flash chromatography Petroleum ether:Ethyl acetate from 0 to 20% to obtain the liquid product that was dried by rotary evaporator to obtain crude tert-butyl((3R,4R)-1-(2-amino-5-(3,5-difluorophenyl)-3-nitropyridin-4-yl)-3-methoxypiperidin-4-yl)carbamate (compound 6-1-5, 336 mg). MS (m/z) 480.2 (M+H)⁺, 502.2 (M+Na)⁺.

### Step 5: Synthesis of tert-butyl ((3R,4R)-1-(2,3-diamino-5-(3,5-difluorophenyl)pyridin-4-yl)-3-methoxypiperidin-4-yl)carbamate (Compound 6-1-6)

To a solution of compound 6-1-5 (336 mg, 0.70 mmol, 1 eq) in methanol (5.00 mL) was added Pd/C (15 mg, 0.14 mmol, 0.2 eq). The mixture was stirred for 2 h at 25° C. under Hydrogen. The reaction was monitored by LCMS and TLC, which showed the reaction was complete. The reaction liquid was filtered to obtain the filtrate that was dried by rotary evaporator to obtain crude tert-butyl ((3R,4R)-1-(2,3-diamino-5-(3,5-difluorophenyl)pyridin-4-yl)-3-methoxypiperidin-4-yl)carbamate (compound 6-1-6, 122 mg). MS (n/z) 450.2 (M+H)⁻.

### Step 6: Synthesis of 5-(7-((3R,4R)-4-amino-3-methoxypiperidin-1-yl)-6-(3,5-difluorophenyl)-3H-imidazo[4,5-b]pyridin-2-yl)-1,3-dihydro-2H-benzo[d]imidazol-2-one (Compound 46)

To a solution of compound 6-1-6 (60 mg, 0.13 mmol, 1 eq) and 2-oxo-2,3-dihydro-1H-benzo[d]imidazole-5-carbaldehyde (26 mg, 0.16 mmol, 1.2 eq) in THE (2.00 mL) was added AcOH (0.80 mL), which was stirred for 2 h at 60° C. Then, the mixture was added FeCl₃ (32 mg, 0.20 mmol, 1.5 eq), which was stirred for 2 h at 60° C. The reaction was monitored by LCMS and TLC, which showed the reaction was complete. The reaction liquid was dried with silica gel by rotary evaporator and was purified by flash chromatography DCM:Methanol from 0 to 20% to obtain the liquid product that was dried by rotary evaporator to give the crude product. The crude product was purified by PREP-HPLC to obtain 5-(7-((3R,4R)-4-amino-3-methoxypiperidin-1-yl)-6-(3,5-difluorophenyl)-3H-imidazo[4,5-b]pyridin-2-yl)-1,3-dihydro-2H-benzo[d]imidazol-2-one (compound 46, 22.9 mg, 35% yield) as white solid.

Compounds 46-66 were synthesized according to the 6-step procedure described in Example 6. Analytical data for compounds 46-66 are provided below.

| Cpd. | | |
|---|---|---|
| No. | Structure | Analytical Data |
| 46 | 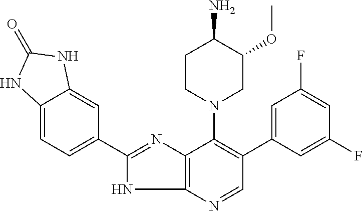 [SMILES: CO[C@@H]1CN(c2c(-c3cc(F)cc(F)c3)cnc3[nH]c(-c4ccc5[nH]c(=O)[nH]c5c4)nc23)CC[C@H]1N] | 1H NMR (400 MHz, DMSO-d6): δ ppm 10.947 (s, 1H), 8.326 (s, 1H), 7.938 (s, 1H), 7.853-7.809 (t, J = 8Hz, 2H), 7.254-7.195 (dd, J1 = 14.4Hz, J2 = 16.8Hz, 4H), 7.079-7.058 (d, J = 8.4Hz, 1H), 4.203-4.174 ( d, J = 11.6Hz, 1H), 3.617-3.584 ( d, J = 13.2Hz, 2H), 3.262 (s, 3H), 3.121-3.062 (t, J = 12.4, 2H), 2.800-2.672 ( m, 3H), 1.821-1.793 (d, J = 11.2, 1H), 1.463-1.373 (m, 1H). MS (m/z) 492.2 (M + H)⁺. |
| 47 | 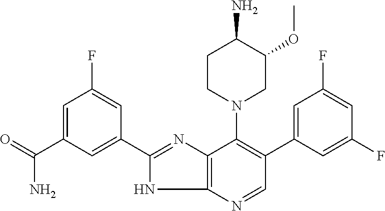 [SMILES: CO[C@@H]1CN(c2c(-c3cc(F)cc(F)c3)cnc3[nH]c(-c4cc(F)cc(C(N)=O)c4)nc23)CC[C@H]1N] | 1H NMR (400 MHz, DMSO-d6): δ ppm 8.632 (s, 1H), 8.380 (s, 1H), 8.213 (s, 1H), 8.138-8.114 (d, J = 9.6Hz, 1H), 8.004 (s, 1H), 7.816-7.792 (d, J = 9.6Hz, 1H), 7.655 (s, 1H), 7.252-7.231 (d, J = 8.4Hz, 3H), 4.324-4.293 (d, J = 12.4Hz, 1H), 3.587-3.553 (d, J = 13.6Hz, 2H), 3.309 (s, 3H), 3.246-3.202 (m, 2H), 3.104- 3.042 (t, J = 12.8Hz, 1H), 2.793-2.720 (m, 2H), 1.843-1.808 (d, J = 14Hz, 1H), 1.464-1.365 (dd, J1 = 26, J2 = 26.4, 1H). MS (m/z) 497.2 (M + H)⁺. |
| 48 | 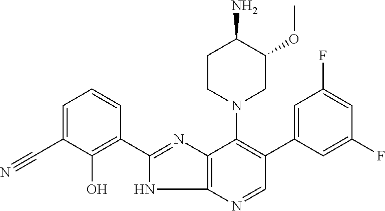 [SMILES: CO[C@@H]1CN(c2c(-c3cc(F)cc(F)c3)cnc3[nH]c(-c4cccc(C#N)c4O)nc23)CC[C@H]1N] | 1H NMR (400 MHz, DMSO-d6): δ ppm 8.216-8.175 (t, J = 16.4Hz, 2H), 7.959 (s, 1H), 7.412-7.393 (d, J = 7.6Hz, 1H), 7.291-7.200 (m, 4H), 6.451-6.413 (d, J = 15.2Hz, 1H), 4.073- 3.999 (m, 1H), 3.595-3.566 (d, J = 11.6Hz, 2H), 3.243 (s, 3H), 3.183-3.155 (m, 2H), 3.100-3.036 (m, 2H), 2.783-2.727 (t, J = 12.4Hz, 1H), 1.989-1.917 (m, 1H), 1.626-1.570 (m, 1H). MS (m/z) 477.2 (M + H)⁺. |
| 49 | 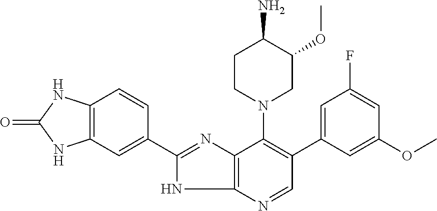 [SMILES: COc1cc(F)cc(-c2cnc3[nH]c(-c4ccc5[nH]c(=O)[nH]c5c4)nc3c2N2CC[C@@H](N)[C@H](OC)C2)c1] | 1H NMR (400 MHz, DMSO-d6):δ ppm 11.031-11.011 (d, J1 = 8.0Hz, 1H), 8.337 (s, 3H), 7.950-7.891 (m, 2H), 7.838 (S, 1H), 7106-7.087 (d, J₁ = 2.0 Hz, J₂ = 7.6 Hz, 1H), 6.934-6.917 (m, 2H), 6.864-6.838 (d, J = 10.4 Hz, 1H), 4.412 (s, 1H), 3.847 (s, 3H), 3.780-3.753 (m, 2H), 3.293 (s, 3H), 3.147-3.047 (m, 2H), 2.841-2.783 (m, 1H), 2.050-2.024 (d, J = 10.4 Hz, 1H), 1.651-1.608 (d, J = 17.2 Hz, 1H). 19F NMR (376 MHz, DMSO-d6); δ ppm −111.257(s, 1F). MS (m/z) 504.3 (M + H)⁺. |
| 50 | 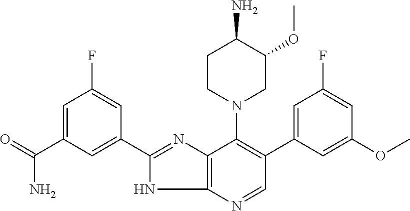 [SMILES: COc1cc(F)cc(-c2cnc3[nH]c(-c4cc(F)cc(C(N)=O)c4)nc3c2N2CC[C@@H](N)[C@H](OC)C2)c1] | 1H NMR (400 MHz, DMSO-d6):δ ppm 8.616 (s, 1H), 8.320- 8.097 (m, 3H), 7.987 (s, 1H), 7.815-7.786 (d, J = 11.6 Hz, 1H), 7.658 (s, 1H), 6.913-6.895 (d, J = 7.2 Hz, 2H), 6.831-6.802 (d, J = 11.6Hz, 1H), 4.317 (s, 1H), 3.834 (s, 3H), 3.683-3.547 (m, 4H), 3.283-3.234 (m, 8H). MS (m/z) 509.1 (M + H)⁺. |
| 51 | 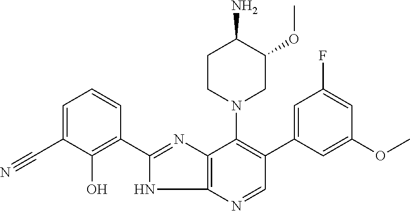 [SMILES: COc1cc(F)cc(-c2cnc3[nH]c(-c4cccc(C#N)c4O)nc3c2N2CC[C@@H](N)[C@H](OC)C2)c1] | 1H NMR (400 MHz, DMSO-d6): δ ppm 8.414-8.390 (m, 1H), 8.090 (s, 1H), 8.040 (s, 2H), 7.871-7.848 (m, 1H), 7.225-7.186(m, 1H), 6.973-6.866 (m, 3H), 3.981-3.883 (m, 4H), 3.846 (s, 3H), 3.303-3.171(m, 3H), 3.133(s, 3H), 2.771- 2.671 (m, 1H), 2.048-2.016 (d, J = 12.8 Hz, 1H), 1.689- 1.641 (m, 1H). MS (m/z) 489.0 (M + H)⁺. |
| 52 | 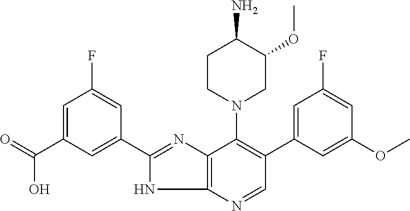 [SMILES: COc1cc(F)cc(-c2cnc3[nH]c(-c4cc(F)cc(C(=O)O)c4)nc3c2N2CC[C@@H](N)[C@H](OC)C2)c1] | 1H NMR (400 MHz, DMSO-d6): δ ppm 8.637 (s, 1H), 7.984 (s, 1H), 7.945-7.925 (d, J = 8Hz, 1H), 7.633-7.609 (d, J = 9.6Hz, 1H), 6.922-6.903 (m, 2H), 6.841-6.803 (m, 1H), 4.310 (s, 1H), 3.839 (s, 3H), 3.722 (s, 1H), 3.422 (m, 2H), 3.167-3.019 (m, 3H), 2.745-2.670 (m, 1 H), 2.324-2.281 (m, 2H), 2.058 (s, 2H), 1.642-1.504 (m, 2H). MS (m/z) 510.2 (M + H)⁺. |
| 53 | 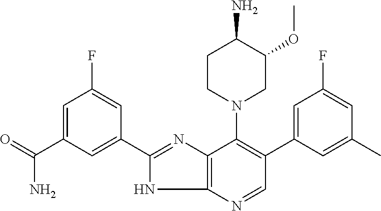 [SMILES: CO[C@@H]1CN(c2c(-c3cc(C)cc(F)c3)cnc3[nH]c(-c4cc(F)cc(C(N)=O)c4)nc23)CC[C@H]1N] | 1H NMR (400 MHz, DMSO-d6): 8.689 (s, 1H), 8.208-8.187 (d, J = 8.4Hz, 5H ), 8.020 (s, 1H ), 7.877-7.853 (d, J = 9.6Hz, 1H), 7.691 (s, 2H), 7.176-7.136 (d, J = 16.0Hz, 1H), 7.085-7.061 (d, J = 9.6Hz, 1H), 4.438(s, 2H ), 3.592-3.488(m, 2H), 3.298 (s, 3H), 3.132-3.077 (m, 2H), 2.843-2.788 (t, J = 11.0Hz, 1H), 2.424(s, 3H), 2.003-1.975(d, J = 11.2Hz, 1H), 1.633-1.604(m, 1H). MS (m/z) 493.2 (M + H)⁺. |
| 54 | 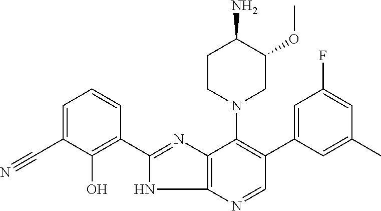 [SMILES: CO[C@@H]1CN(c2c(-c3cc(C)cc(F)c3)cnc3[nH]c(-c4cccc(C#N)c4O)nc23)CC[C@H]1N] | 1H NMR (400 MHz, DMSO-d6): δ ppm 8.217-8.151 (m, 2H), 7.931 (s, 1H), 7.420-7.403 (d, J = 6.8 Hz, 1H), 7.183- 7.125 (m, 2H), 7,027-7.000 (d, J = 10.8 Hz, 1H), 6.455 (s, 1H), 3.669-3.631 (m, 1H), 3.224-3.047 (m, 8H), 2.725-2.665 (m, 1H), 2.333 (s, 3H), 2.248 (s, 1H), 1.997-1.815 (m, 2H), 1.653- 1.597 (m, 1H). MS (m/z) 473.1 (M + H)⁺. |
| 55 | 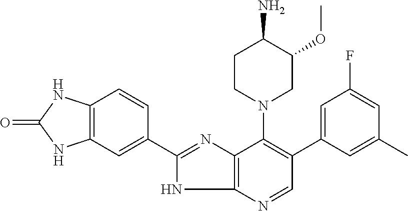 [SMILES: CO[C@@H]1CN(c2c(-c3cc(C)cc(F)c3)cnc3[nH]c(-c4ccc5[nH]c(=O)[nH]c5c4)nc23)CC[C@H]1N] | 1H NMR (400 MHz, DMSO-d6):δ ppm 10.987-10.953 (d, J = 13.6Hz, 2H), 8.051 (s, 3H), 7.943 (s, 1H), 7.866-7.846 (d, J = 80Hz, 1H), 7.806 (s, 1H), 7.170-7.031 (m, 4H), 4.381-4.242 (m, 2H), 3.385-3.252 (m, 1H), 3.207 (s, 3H), 3.207-3.204 (m, 2H), 2.512-2.503 (t, J = 1.6Hz, 1H), 2.413 (d, 3H), 1.987-1.966 (m, 1H), 1.604-1.487 (m, 1H). 19F NMR (376 MHz, DMSO-d6):δ ppm −113.677(s, 1F). MS (m/z) 488.2 (M + H)⁺. |
| 56 | 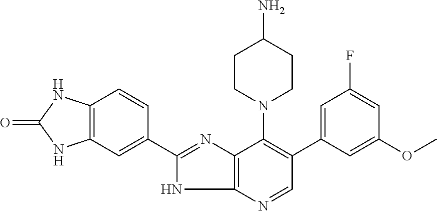 [SMILES: COc1cc(F)cc(-c2cnc3[nH]c(-c4ccc5[nH]c(=O)[nH]c5c4)nc3c2N2CCC(N)CC2)c1] | 1H NMR (400 MHz, MeOD-d4):δ ppm 7.828-7.763( m, 3H), 7.089-7.069( d, J = 8Hz, 1H), 6.785- 6.749 (m, 2H), 6.634- 6.596(m, 1H), 3.916-3.883(d, J = 13.2Hz, 2H), 3.764(s, 3H), 3.164-3.094(m, 3H), 1.882-1.851(d, J = 12.4Hz, 2H), 1.719-1.629(m, 2H). MS (m/z) 474.0 (M + H)⁺. |
| 57 | 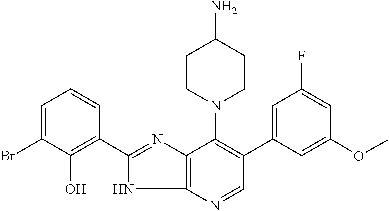 [SMILES: COc1cc(F)cc(-c2cnc3[nH]c(-c4cccc(Br)c4O)nc3c2N2CCC(N)CC2)c1] | 1H NMR (400 MHz, DMSO-d6):δ ppm 14.170, (s, 1H), 8.162-8.123 (t, J = 8Hz, 1H), 8.048 (s, 1H), 7.706-7.685 (d, J = 8Hz, 1H), 6.988-9.903 (m, 3H), 6.855-6.817 (m, 1H), 3.842 (s, 3H), 3.778-3.745 (d, J = 13.2Hz, 2H), 3.286-3.258 (m, 2H), 3.145-3.086 (t, J = 12.4Hz, 2H), 1.932-1.906 (d, J = 10.4Hz, 2H), 1.639-1.580 (dd, J1 = 3.1Hz, J2 = 21.2Hz, 2H). MS (m/z) 512.1 (M + H)⁺. |
| 58 | 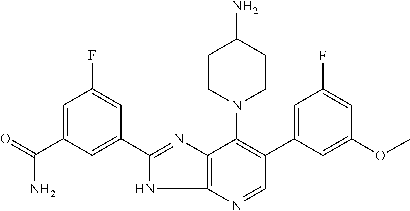 [SMILES: COc1cc(F)cc(-c2cnc3[nH]c(-c4cc(F)cc(C(N)=O)c4)nc3c2N2CCC(N)CC2)c1] | 1H NMR (400 MHz, DMSO-d6): 1H NMR (400 MHz, DMSO-d6): δ ppm 8.587-8.581 (t, J = 1.2Hz, 1H), 8.336 (s, 1H), 8.182 (s, 1H), 8.138-8.105 (m, 1H), 7.985(s, 1H), 7.817- 7.785 (m, 1H), 7.669 (s, 1H), 6.924-6.919 (dd, J1 = 2Hz, J2 = 8.4Hz, 2H), 6.825-6.786 (m, 1H), 3.859 (s, 1H), 3.837 (s, 3H), 3.152-3.092 (t, J = 12Hz, 4H), 1.857-1.830 (d, J = 10.85Hz, 2H), 1.589-1.512 (dd, J1 = 10.4Hz, J2 = 21.2Hz, 2H). MS (m/z) 479.2 (M + H)⁺. |
| 59 | 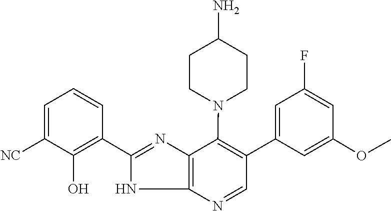 [SMILES: COc1cc(F)cc(-c2cnc3[nH]c(-c4cccc(C#N)c4O)nc3c2N2CCC(N)CC2)c1] | 1H NMR (400 MHz, DMSO-d6): 8.218-8.179 (m, 2H), 7.954 (s, 1H), 7.433-7.411 (dd, J1 = 2Hz, J2 = 7.6Hz, 1H), 6.939-6.911 (m, 2H), 6.808-6.711 (m, 1H), 6.494-6.459 (t, J = 7.2Hz, 1H), 3.836 (s, 3H), 3.670-3.640 (d, J = 12Hz, 3H), 3.123-3.064 (t, J = 12Hz, 3H), 1.908-1.881 (d, J = 10.8Hz, 2H), 1.646-1.559 (dd, J1 = 12.4Hz, J2 = 20.8Hz, 2H), 1.234 (s, 1H). MS (m/z) 459.1 (M + H)⁺. |
| 60 | 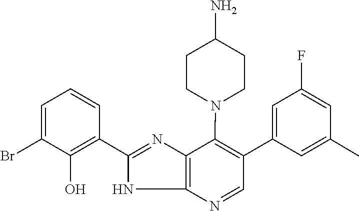 [SMILES: Cc1cc(F)cc(-c2cnc3[nH]c(-c4cccc(Br)c4O)nc3c2N2CCC(N)CC2)c1] | 1H NMR (400 MHz, DMSO-d6): δ ppm 14.133 (s, 1H), 8.145- 8.123 (d, J = 8.8Hz, 1H), 8.038 (s, 1H), 7.719-7.701 (d, J = 7.2Hz, 1H), 7.243 (s, 1H), 7.173-7.115 (m, 3H), 7.058-6.973 (m, 3H), 3.762-3.726 (d, J = 14.4Hz, 2H), 3.514 (s, 1H), 3.139-3.078 (t, J = 12Hz, 2H), 2.409 (s, 3H), 1.916-1.885 (d, J = 12.4Hz, 2H), 1.613-1.537 (m, 2H). MS (m/z) 496.1 (M + H)⁺. |
| 61 | 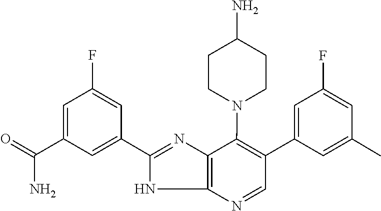 [SMILES: Cc1cc(F)cc(-c2cnc3[nH]c(-c4cc(F)cc(C(N)=O)c4)nc3c2N2CCC(N)CC2)c1] | 1H NMR (400 MHz, DMSO-d6): δ ppm 8.591-8.587 (t, J = 1.6Hz, 1H), 8.392 (s, 1H), 8.211 (s, 1H), 8.139 (s, 1H), 8.106 (m, 1H), 7.961 (s, 1H), 7.812-7.780 (m, 1H), 7.663 (s, 1H), 7.171 (s, 1H), 7.134-7.110 (d, J = 9.6Hz, 1H), 7.018-6.993 (d, J = 10Hz, 1H), 3.825-3.790 (d, J = 14Hz, 3H), 3.146-3.082 (t, J = 13.6Hz, 4H), 2.401 (s, 3H), 1.834-1.805 (d, J = 11.6Hz, 2H), 1.560-1.464 (m, 2H). MS (m/z) 463.2 (M + H)⁺. |
| 62 | 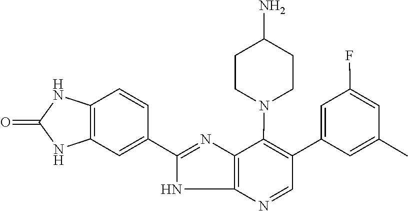 [SMILES: Cc1cc(F)cc(-c2cnc3[nH]c(-c4ccc5[nH]c(=O)[nH]c5c4)nc3c2N2CCC(N)CC2)c1] | 1H NMR (400 MHz, DMSO-d6):δ ppm 10.899 (s, 1H), 8.400 (s, 1H), 7.898-7.813 (m, 3H), 7.172-7.059 (m, 3H), 7.005- 6.981 (d, J = 9.6 Hz, 1H), 3.789-3.758 (d, J = 12.4 Hz, 2H), 3.137-3.180 (t, J = 12.0 Hz, 3H), 2.400 (s, 3H), 1.845-1.814 (d, J = 12.4 Hz, 2H), 1.536-1.516 (d, J = 8.0 Hz, 2H). MS (m/2) 458.3 (M + H)⁺. |
| 63 | 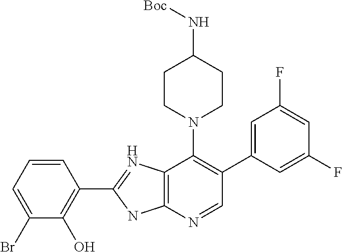 | 1H NMR (400 MHz, CDCl₃): δ ppm 8.12-8.11 (m, 1H), 7.65- 7.63 (m, 1H), 7.43-7.41 ( m, 1H ), 6.99-6.97 ( m, 2H ), 6.81- 6.79 (m, 2H), 4.52 (s, 1H), 3.81-3.77 (m, 2H), 3.75-3.74 (m, 1H), 3.26-3.22 (m, 2H), 2.02-2.00 (m, 2H), 1.52 (s, 9H), 1.32- 1.30 (m, 2H). MS (m/z) 600.2 (M + H)⁺. |
| 64 | 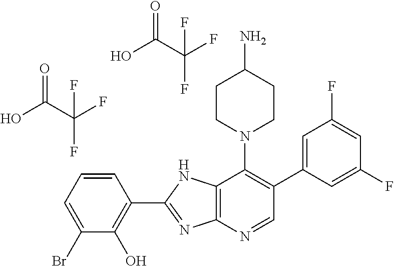 | 1H NMR (400 MHz, DMSO-d6): δ ppm 14.06 (s, 1H), 8.15- 8.12 ( dd, J1 = 1.2Hz, J₂ = 8.0Hz, 1H ), 8.08 (s, 1H), 7.92-7.86 ( m, 2H ), 7.73-7.70 (m, 1H), 7.27-7.24 (m, 3H), 7.01-6.98 (m, 1H), 3.91-3.86 (m, 2H), 3.31-3.30 (m, 1H), 3.15-3.09 (m, 2H), 1.94-1.91 (m, 2H), 1.62-1.58 (m, 2H). MS (m/z) 500.1 (M + H)⁺. |
| 65 | 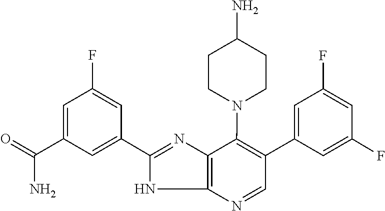 [SMILES: NC(=O)c1cc(F)cc(-c2nc3c(N4CCC(N)CC4)c(-c4cc(F)cc(F)c4)cnc3[nH]2)c1] | 1H NMR (400 MHz, DMSO-d6): δ ppm 8.606(s, 1H), 8.230(s, 1H), 8.145-8.120(d, J = 10Hz, 1H), 8.006(s, 1H), 7.818-7.794(d, J = 9.6Hz, 1H), 7.674(s, 1H), 7.255-7.192(dd, J1 = 13.6Hz, J2 = 18.8Hz, 4H), 3.830-3.800(d, J = 12Hz, 3H), 3.173-3.117(t, J = 10.8Hz, 4H), 1.864(s, 2H), 1.574(s, 2H). MS (m/z) 467.2 (M + H)⁺. |
| 66 | 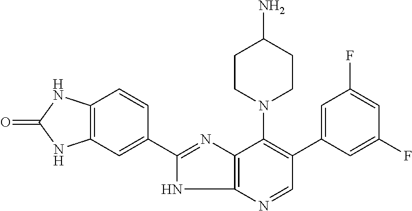 [SMILES: NC1CCN(c2c(-c3cc(F)cc(F)c3)cnc3[nH]c(-c4ccc5[nH]c(=O)[nH]c5c4)nc23)CC1] | 1H NMR (400 MHz, DMSO-d6): δ ppm 10.986 (s, 2H), 8.073- 7.999 (d, J = 29.6Hz, 4H), 7.903-7.882 (d, J = 8.4Hz, 1H), 7.825(s, 1H), 7.288-7.269 (d, J = 7.6Hz, 3H), 7.113-7.092 (d, J = 8.4Hz, 1H), 3.888-3.859 (d, J = 11.6Hz, 2H), 3.228-3.171 (m, 3H), 1.938-1.909 (d, J = 11.6Hz, 2H), 1.633-1.611 (m, 2H), MS (m/z) 466.2 (M + H)⁺. |

### Example 7

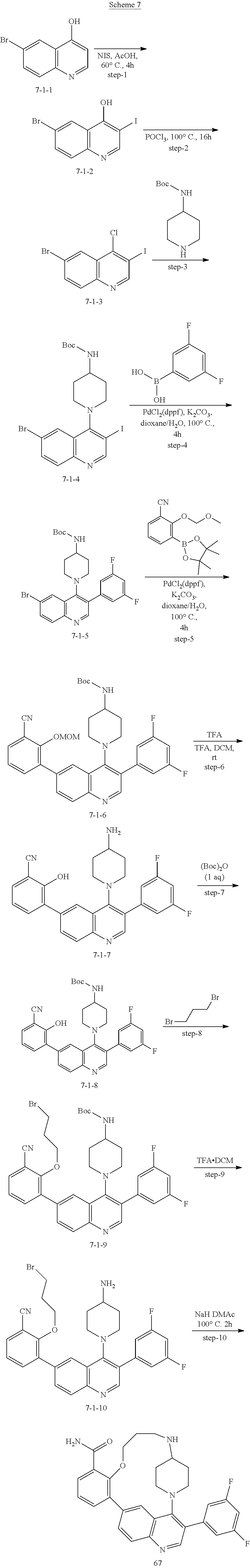

### Step 1: Synthesis of 6-bromo-3-iodoquinolin-4-ol (Compound 7-1-2)

To the solution of compound 7-1-1 (3000 mg, 13.389 mmol, 1 eq) in AcOH (50 mL) was added NIS (3012 mg, 13.389 mmol, 1 eq). The mixture was stirred at 60° C. for 3 h. The reaction was monitored by LCMS, which showed worked well. The solution was cooled to zero degree, and lots of solid was observed. After filter, 6-bromo-3-iodoquinolin-4-ol (compound 7-1-2, 4.4 g, 94% yield) was obtained as a white solid. MS (m/z) 349.9 (M+H)⁺.

### Step 2: Synthesis of 6-bromo-4-chloro-3-iodoquinoline (Compound 7-1-3)

The solution of compound 7-1-2 (2500 mg, 7.144 mmol, 1 eq) in POCl₃ (15 mL) the reaction was stirred at 100° C. for 3h. The reaction was complete detected by TLC and LCMS. LCMS show the reaction was worked complete. The solution was added NaHCO₃(aq) adjust pH to 7, then the reaction mixture was diluted with water (50 mL) and extracted with EA (80 mL×4). The combined organic extract and concentrated to give the crude material, then the crude material was purified by silica gel chromatography eluted with PE:EtOAc (from 100:1 to 5:1) to give product compound 7-1-3 (1.5 g) as light-yellow solid. MS (m/z) 367.8 (M+H)⁺.

### Step 3: Synthesis of tert-butyl (1-(6-bromo-3-iodoquinolin-4-yl) piperidin-4-yl) carbamate (Compound 7-14)

To a solution of compound 7-1-3 (1000 mg, 2.714 mmol, 1 eq) and tert-butyl piperidin-4-ylcarbamate (1082 mg, 5.429 mmol, 2 eq) in DMAc (15 mL) was added K₂CO₃ (1125 mg, 8.143 mmol, 3.0 eq) the reaction was stirred at 120° C. for 4 h. The reaction was complete detected by TLC and LCMS. LCMS show the reaction was worked complete. The reaction mixture was diluted with water (100 mL) and extracted with EA (100 mL×4). The combined organic extract and concentrated to give the crude material, then the crude product was purified by Flash (PE:EA=3:1) to afford compound 7-1-4 (570 mg, 40% yield) as white solid. MS (m/z) 531.9 (M+H)⁺.

### Step 4: Synthesis of tert-butyl (1-(6-bromo-3-(3,5-difluorophenyl)quinolin-4-yl)piperidin-4-yl)carbamate (Compound 7-1-5)

To a solution of compound 7-14 (570 mg, 1.07 mmol, 1 eq), (3,5-difluorophenyl)boronic acid (169 mg, 1.07 mmol, 1 eq) and K₂CO₃ (443 mg, 3.213 mmol, 3 eq) in the mixture of dioxane (10 mL) and H₂O (1 mL) was added PdCl₂(dppf) (78 mg, 0.107 mmol, 0.1 eq) under N₂. The suspension was degassed under vacuum and purged with N₂ several times. Then the reaction mixture was stirred at 90° C. for 4 h. The reaction was complete detected by TLC and LCMS. LCMS show the reaction was worked complete. The reaction mixture was diluted with water (30 mL) and extracted with EA (50 mL×4). The combined organic extract and concentrated to give the crude material, then the crude product was purified by Flash (PE:EA=1:1) to afford compound 7-1-5 (500 mg, 90% yield) as yellow solid. MS (m/z) 518.1 (M+H)⁺.

### Step 5: Synthesis of tert-butyl (1-(6-(3-cyano-2-(methoxymethoxy)phenyl)-3-(3,5-difluorophenyl)quinolin-4-yl)piperidin-4-yl)carbamate (Compound 7-1-6)

To a solution of compound 7-1-5 (500 mg, 0.965 mmol, 1 eq), 2-(methoxymethoxy)-3-(4,4,5,5-tetramethyl-1,3,2-dioxaborolan-2-yl)benzonitrile (307 mg, 1.061 mmol, 1.1 eq) and K₂CO₃ (399 mg, 2.894 mmol, 3 eq) in the mixture of dioxane (10 mL) and H₂O (1 mL) was added PdCl₂(dppf) (71 mg, 0.0% mmol, 0.1 eq) under N₂. The suspension was degassed under vacuum and purged with N₂ several times. Then the reaction mixture was stirred at 100° C. for 4 h. The reaction was complete detected by TLC and LCMS. LCMS show the reaction was worked complete. The reaction mixture was diluted with water (30 mL) and extracted with EA (50 mL×4). The combined organic extract and concentrated to give the crude material, then the crude product was purified by Flash (PE:EA=1:1) to afford compound 7-1-6 (370 mg, 63.9% yield) as yellow oil. MS (m/z) 601.3 (M+H)⁺.

### Step 6: Synthesis of 3-(4-(4-aminopiperidin-1-yl)-3-(3,5-difluorophenyl) quinolin-6-yl)-2-hydroxybenzonitrile (Compound 7-1-7)

To a solution of compound 7-1-6 (370 mg, 0.616 mmol, 1 eq) in the DCM (5 mL) was added TFA (1 mL). Then the reaction mixture was stirred at 25° C. for 1 h. The reaction was complete detected by TLC and LC-MS. TLC and LCMS showed the reaction was completed. The reaction mixture was concentrated to give the crude material compound 7-1-7 as TFA salt (300 mg). MS (m/z) 457.2 (M+H)⁺.

### Step 7: Synthesis of tert-butyl (1-(6-(3-cyano-2-hydroxyphenyl)-3-(3,5-difluorophenyl)quinolin-4-yl)piperidin-4-yl)carbamate (Compound 7-1-8)

To a solution of compound 7-1-7 (100 mg, 0.219 mmol, 1 eq) and DIPEA (85 mg, 0.657 mmol, 3 eq) in DCM (6 mL) was added (Boc)₂O (48 mg, 0.219 mmol, 1 eq). Then the reaction mixture was stirred at 25° C. for 1 h. The reaction was complete detected by TLC and LC-MS. TLC and LCMS showed the reaction was complete. The reaction mixture was diluted with water (20 mL) and extracted with EA (30 mL×3). The combined organic extract concentrated to give the crude material, the crude material was purified by silica gel chromatography eluted with PE:EtOAc (from 100:1 to 1:1) to give product compound 7-1-8 (70 mg, 57.4% yield) as yellow oil. MS (m/z) 557.3 (M+H)⁺.

LCMS: m/z: 557.3 (M+H⁺) at 1.68 min.

### Step 8: Synthesis of tert-butyl (1-(6-(2-(3-bromopropoxy)-3-cyanophenyl)-3-(3,5-difluorophenyl)quinolin-4-yl)piperidin-4-yl)carbamate (Compound 7-1-9)

To a solution of compound 7-1-8 (70 mg, 0.126 mmol, 1 eq) and 1,3-dibromopropane (38 mg, 0.189 mmol, 1.5 eq) in MeCN (5 mL) was added and K₂CO₃ (52 mg, 0.377 mmol, 3 eq). Then the reaction mixture was stirred at 90° C. for 1 h. The reaction was complete detected by TLC and LC-MS. TLC and LCMS showed the reaction was complete. The reaction mixture was diluted with water (20 mL) and extracted with EA (30 mL×3). The combined organic extract concentrated to give the crude material, the crude material was purified by silica gel chromatography eluted with PE:EtOAc (from 100:1 to 1:1) to give product compound 7-1-9 (40 mg, 47% yield) as yellow oil. MS (m/z) 677.3 (M+H)⁺.

### Step 9: Synthesis of 3-(4-(4-aminopiperidin-1-yl)-3-(3,5-difluorophenyl)quinolin-6-yl)-2-(3-bromopropoxy)benzonitrile (Compound 7-1-10)

To a solution of compound 7-1-9 (40 mg, 0.059 mmol, 1 eq) in DCM (3 mL) was added TFA (1 mL) under N2. Then the reaction mixture was stirred at 25° C. for 1 h. The reaction was complete detected by TLC and LC-MS. TLC and LCMS showed the reaction was complete. The reaction mixture was concentrated to give the compound 7-1-10 (50 mg crude) as yellow oil. MS (m/z) 577.1 (M+H)⁺.

The step of the intermediates of compound 68 and compound 69 were similar to compound 67, but the acid is HCl/EA.

### Step 10: Synthesis of 3-(4-(4-aminopiperidin-1-yl)-3-(3,5-difluorophenyl)quinolin-6-yl)-2-(3-bromopropoxy)benzonitrile (Compound 67)

To a solution of compound 7-1-10 (30 mg, 0.052 mmol, 1 eq) in DMAc (2 mL) was added NaH (6 mg, 0.156 mmol, 3 eq). The reaction mixture was stirred at 100° C. for 1 h. The reaction was complete detected by LC-MS. LCMS showed the reaction was completed. The reaction mixture was diluted with water (10 mL) and extracted with EA (10 mL×3). The combined organic extract was washed with brine (10 mL), dried over Na₂SO₄, filtered and concentrated to give the crude material, which was purified by Prep-HPLC, to provide compound 67 (5.7 mg) as yellow solid.

The syntheses of other compounds were similar to compound 67, but the base is K₂CO₃, the additive is KI, and the solvent is DMF.

Compounds 67-77 were synthesized according to the 10-step procedure described in Example 7. Analytical data for compounds 67-77 are provided below.

| Cpd. | | |
|---|---|---|
| No. | Structure | Analytical Data |
| 67 | 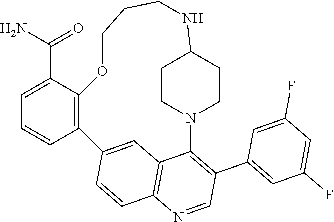 [SMILES: NC(=O)c1cccc2c1OCCCNC1CCN(CC1)c1c(-c3cc(F)cc(F)c3)cnc3ccc-2cc13] | 1H NMR (400 MHz, DMSO-d6): δ 8.585 (s, 1H), 8.327 (s, 1H), 8.267 (s, 1H), 8.118-8.096 (d, J = 8.8 Hz, 1H), 7.956-7.939 (m, 2H), 7.880-7.873 (m, 2H), 7.492-7.454 (t, J = 7.6 Hz, 1H), 7.193-7.174 (m, 2H), 3.826-3.793 (t, J = 6.4 Hz, 2 H), 3.410-3.326 (m, 4H), 2.697-2.670 (m, 3H). MS (m/z) 515.0 (M + H)⁺. |
| 68 | 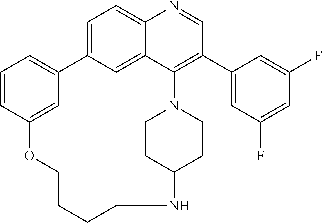 [SMILES: Fc1cc(F)cc(-c2cnc3ccc4cc3c2N2CCC(CC2)NCCCCOc2cccc-4c2)c1] | 1H NMR (400 MHz, DMSO-d6) δ 8.75-8.70 (m, 3H), 8.44 (s, 1H), 8.28 (d, J = 8.8 Hz, 1H), 8.10 (d, J = 8.6 Hz, 1H), 7.62 (s, 1H), 7.46-7.34 (m, 4H), 7.20 (d, J = 7.0 Hz, 2H), 7.03 (d, J = 6.3 Hz, 1H), 4.30-4.26 (m, 2H), 3.48-3.42 (m, 3H), 3.14-3.06 (m, 2H), 2.86 (t, J = 12.4 Hz, 2H), 2.22- 2.12 (m, 2H), 1.98-1.80 (s, 2H), 1.84-1.71 (m, 4H). MS (m/z) 486.35 (M + H)⁺. |
| 69 | 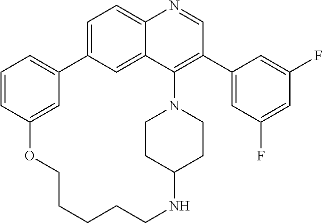 [SMILES: Fc1cc(F)cc(-c2cnc3ccc4cc3c2N2CCC(CC2)NCCCCCOc2cccc-4c2)c1] | ¹H NMR (400 MHz, DMSO-d₆) δ 8.68 (s, 1H), 8.59 (s, 2H), 8.40 (s, 1H), 8.27 (d, J = 8.8 Hz, 1H), 8.10 (d, J = 8.7 Hz, 1H), 7.65 (s, 1H), 7.47-7.42 (m, 2H), 7.37 (dd, J = 10.7, 8.1 Hz, 1H), 7.19 (d, J = 6.5 Hz, 2H), 7.02 (dd, J = 6.1, 3.5 Hz, 1H), 4.19 (t, J = 7.1 Hz, 2H), 3.04 (brs, 4H), 2.81 (t, J = 12.6 Hz, 2H), 2.35-2.30 (m, 1H), 2.17-2.05 (m, 2H), 2.04 1.83 (m, 2H), 1.82-1.74 (m, 2H), 1.73-1.65 (m, 2H), 1.60- 1.52 (m, 2H). MS (m/z) 500.20 (M + H)⁺. |
| 70 | 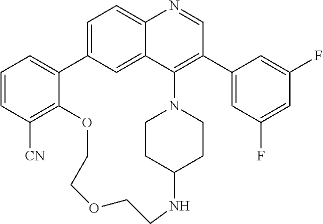 [SMILES: N#Cc1cccc2c1OCCOCCNC1CCN(CC1)c1c(-c3cc(F)cc(F)c3)cnc3ccc-2cc13] | ¹H NMR (399 MHz, CD3OD) δ 9.09 (s, 1H), 8.56 (s, 1H), 8.51 (s, 1H), 8,10 (d, J = 9.2 Hz, 1H), 8.04-7.99 (m, 2H), 7.72 (d. J = 6.4 Hz, 1H), 7.42 (t, J = 7.6 Hz, 1H), 7.06 (d, J = 7.6 Hz, 3H), 4.58 (s, 0H), 4.18 (s, 2H), 3.98 (s, 2H), 3.77 (s, 2H), 3.60 (d, J = 12.8 Hz, 2H), 3.16 (s, 3H), 2.79 (t, J = 11.6 Hz, 2H), 2.54 (d, J = 9.6 Hz, 2H), 1.72 (d, J = 10.8 Hz, 2H). MS (m/z) 527 (M + H)⁺. |
| 71 | 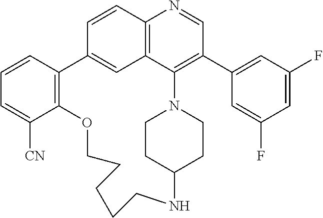 [SMILES: N#Cc1cccc2c1OCCCCCNC1CCN(CC1)c1c(-c3cc(F)cc(F)c3)cnc3ccc-2cc13] | ¹H NMR (399 MHz, cd3od) δ 8.97 (s, 1H), 8.60 (s, 1H), 8.52 (s, 1H), 8.12 (d, J = 8.8 Hz, 1H), 7.98 (d, J = 8.8 Hz, 1H), 7.91 (d. J = 7.6 Hz, 1H), 7.73 (d, J = 7.6 Hz, 1H), 7.42 (t, J = 7.6 Hz, 1H), 7.11-6.99 (m, 3H), 4.08 (s, 2H), 3.67 (s, 2H), 2.95 (s, 2H), 2.81 (t, J = 10.0Hz, 3H), 2.18 (s, 2H), 1.88 (s, 6H), 1.75 (s, 2H). MS (m/z) 525.3 (M + H)⁺. |
| 72 | 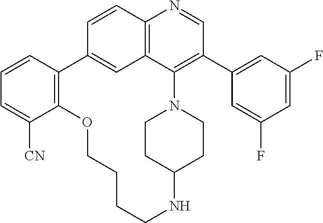 [SMILES: N#Cc1cccc2c1OCCCCNC1CCN(CC1)c1c(-c3cc(F)cc(F)c3)cnc3ccc-2cc13] | ¹H NMR (400 MHz, cd3od) δ 8.72 (s, 1H), 8.51 (s, 1H), 8.18 (q, J = 8.8 Hz, 2H), 7.75 (d, J = 7.6 Hz, 1H), 7.66 (d, J = 7.6 Hz, 1H), 7.18 (dd, J = 16.8, 6.8 Hz, 4H), 3.82 (d, J = 14.4 Hz, 2H), 3.65-3.35 (m, 3H), 3.07 (t, J = 12.4 Hz, 4H), 2.26 (d, J = 12.8 Hz, 2H), 1.98 (dd, J = 40.8, 23.6 Hz, 6H). MS (m/z) 511 (M + H)⁺. |
| 73 | 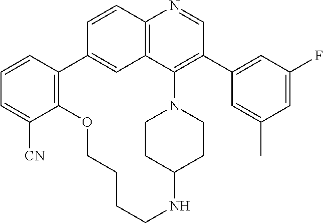 [SMILES: Cc1cc(F)cc(-c2cnc3ccc4cc3c2N2CCC(CC2)NCCCCOc2c(C#N)cccc2-4)c1] | 1H NMR (400 MHz, DMSO-d6): δ 9.19 (s, 1H), 8.70 (s, 1H), 8.29 (d, J = 8.2 Hz, 1H), 8.19 (d, J = 8.7 Hz, 1H), 8.04 (d, J = 8.4 Hz, 1H), 7.80 (d, J = 7.2 Hz, 1H), 7.51 (t, J = 8.7, 1H), 7.13 (s, 1H), 7.08 (t, J = 10,9 Hz, 2H), 4.05 (s, 2H), 3.94 (d, J = 13, 2H), 3.84 (s, 2H), 3.29 (m, 2H), 3.01 (t, J = 13, 2H), 2.56 (m, 5H), 2.25 (t, 2H), 2.05 (m, 4H). MS (m/z) 507 (M + H)⁺. |
| 74 | 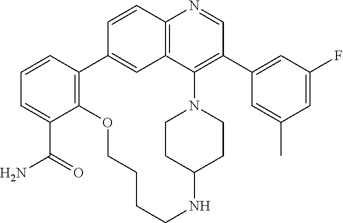 [SMILES: Cc1cc(F)cc(-c2cnc3ccc4cc3c2N2CCC(CC2)NCCCCOc2c(C(N)=O)cccc2-4)c1] | 1H NMR (400 MHz, DMSO-d6): δ 8.66 (s, 1H), 8.46 (s, 1H), 8.17 (s, 2H), 7.82 (dd, J = 25.8, 7.7 Hz, 2H), 7.45 (s, 1H), 7.11 (d, J = 8.1 Hz, 2H), 7.04 (d, J = 8.6 Hz, 1H), 3.83 (t, J = 6.1 Hz, 2H), 3.70 (d, J = 13.1 Hz, 2H), 3.40 (t, J = 6.2 Hz, 2H), 3.29 (d, J = 1.4 Hz, 1H), 3.03 (t, J = 12.1 Hz, 2H), 2.46 (s, 3H), 2.12-1.56 (m, 6H), 1.56-1.26 (m, 2H). MS (m/z) 525 (M + H)⁺. |
| 75 | 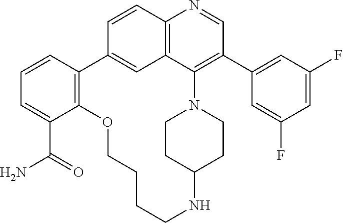 [SMILES: NC(=O)c1cccc2c1OCCCCNC1CCN(CC1)c1c(-c3cc(F)cc(F)c3)cnc3ccc-2cc13] | ¹H NMR (399 MHz, cd3od) δ 8.69 (s, 1H), 8.46 (s, 1H), 8.21-8.11 (m, 2H), 7.85 (d, J = 8.0 Hz, 1H), 7.79 (d, J = 8.0 Hz, 1H), 7.45 (t, J = 7.6 Hz, 1H), 7.15 (dd, J = 12.8, 8.0 Hz, 3H), 3.82 (t, J = 6.4 Hz, 2H), 3.69 (d, J = 12.8 Hz, 2H), 3.47- 3.31 (m, 3H), 3.04 (t, J = 12.4 Hz, 2H), 2.07 (d, J = 11.6 Hz, 2H), 1.83 (d, J = 12.0 Hz, 2H), 1.66-1.57 (m, 2H), 1.46 (dd, J = 14.0, 7.6 Hz, 2H). MS (m/z) 529 (M + H)⁺. |
| 76 | 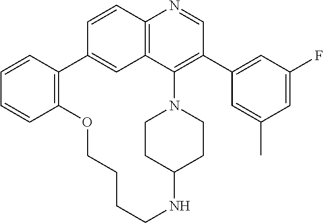 [SMILES: Cc1cc(F)cc(-c2cnc3ccc4cc3c2N2CCC(CC2)NCCCCOc2ccccc2-4)c1] | ¹H NMR (400 MHz, cd3od) δ 8.77 (s, 1H), 8.68 (s, 1H), 8.12 (q, J = 8.8 Hz, 2H), 7.51 (dd, J = 7.6, 1.6 Hz, 1H), 7.44 (t, J = 8.8 Hz, 1H), 7.27 (d, J = 8.8 Hz, 1H), 7.16 (t, J = 7.6 Hz, 1H), 7.10 (d, J = 10.0 Hz, 2H), 7.04 (d, J = 9.6 Hz, 1H), 4.25 (t, J = 4.0 Hz, 2H), 3.81 (dd, J = 17.2, 10.8 Hz, 3H), 3.26 (d, J = 6.4 Hz, 2H), 2.97 (t, J = 11.6 Hz, 2H), 2.48- 2.39 (m, 5H), 2.18-2.09 (m, 2H), 2.01 (dd, J = 12.8, 5.6 Hz, 4H). MS (m/z) 482.4 (M + H)⁺. |
| 77 | 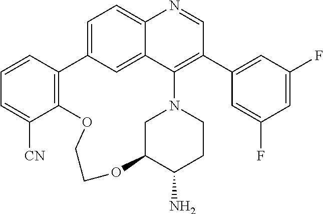 [SMILES: N#Cc1cccc2c1OCCO[C@H]1CN(CC[C@@H]1N)c1c(-c3cc(F)cc(F)c3)cnc3ccc-2cc13] | ¹H NMR (400 MHz, cd3od) δ 8.94-8.91 (m, 1H), 8.66- 8.61 (m, 1H), 8.27-8.23 (m, 1H), 8.17-8.12 (m, 1H), 8.09- 8.03 (m, 1H), 7.79-7.73 (m, 1H), 7.40-7.34 (m, 1H), 7.21-7.02 (m, 3H), 5.22 (dd, J = 14.8, 7.4 Hz, 1H), 4.77- 4.55 (m, 4H), 4.22 (dt, J = 18.8, 11.2 Hz, 2H), 3.13-3.06 (m, 2H), 2.77 (t, J = 10.8 Hz, 1H), 2.04 (d, J = 13.2 Hz, 1H), 1.88-1.68 (m, 1H). MS (m/z) 499 (M + H)⁺. |

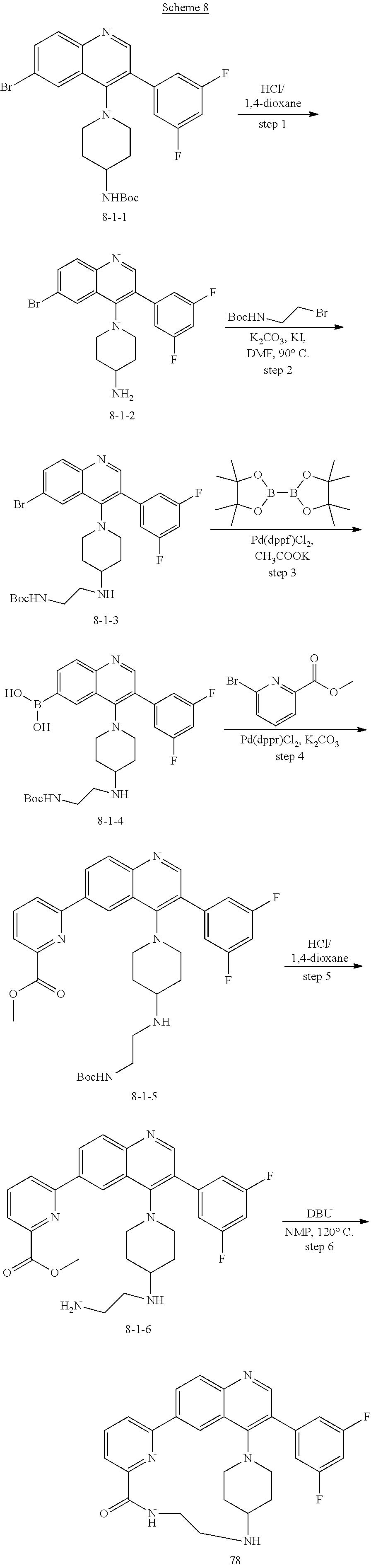

### Step 1: Synthesis of 1-(6-bromo-3-(3,5-difluorophenyl) quinolin-4-yl) piperidin-4-amine (Compound 8-1-2)

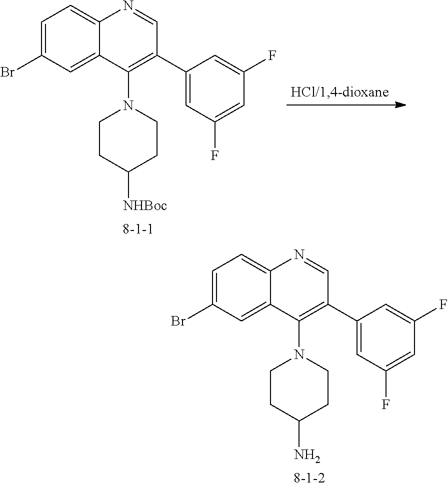 [SMILES: CC(C)(C)OC(=O)NC1CCN(c2c(-c3cc(F)cc(F)c3)cnc3ccc(Br)cc23)CC1 ; NC1CCN(c2c(-c3cc(F)cc(F)c3)cnc3ccc(Br)cc23)CC1]

Tert-butyl (1-(6-bromo-3-(3,5-difluorophenyl) quinolin-4-yl) piperidin-4-yl) carbamate (compound 8-1-1) (500 mg, 0.97 mmol, 1.0 equiv) was dissolved in the solution of HCl/1,4-dioxane (4M, 10 mL). The reaction mixture was stirred at room temperature for 2 h. The desired product was detected by LCMS. The reaction mixture was concentrated under vacuum to afford crude product 1-(6-bromo-3-(3,5-difluorophenyl) quinolin-4-yl) piperidin-4-amine (compound 8-1-2, 404 mg, 0.97 mmol, 100%) for next step. MS (m/z) 418.1 (M+H)⁺.

### Step 2: Synthesis of tert-butyl (2-((1-(6-bromo-3-(3,5-difluorophenyl)quinolin-4-yl)piperidin-4-yl)amino)ethyl)carbamate (Compound 8-1-3)

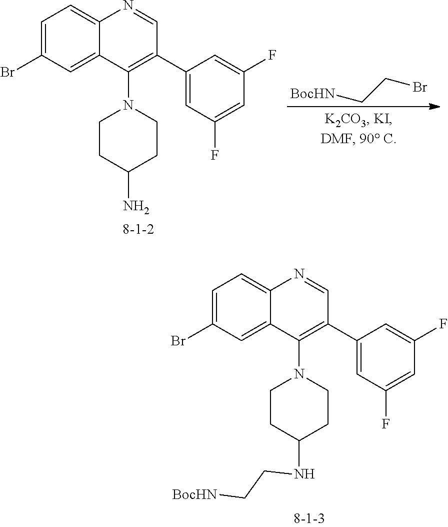 [SMILES: NC1CCN(c2c(-c3cc(F)cc(F)c3)cnc3ccc(Br)cc23)CC1 ; CC(C)(C)OC(=O)NCCBr ; CC(C)(C)OC(=O)NCCNC1CCN(c2c(-c3cc(F)cc(F)c3)cnc3ccc(Br)cc23)CC1]

1-(6-bromo-3-(3,5-difluorophenyl)quinolin-4-yl)piperidin-4-amine (4.7 g, 9.7 mmol, 1.0 equiv), methyl 6-bromopicolinate (compound 8-1-2,404 mg, 0.97 mmol, 1.0 equiv), K₂CO₃ (400 mg, 2.9 mmol, 3.0 equiv) and KI (249 mg, 1.5 mmol, 1.5 equiv) were dissolved in DMF (10 mL), then tert-butyl (2-bromoethyl)carbamate (434 mg, 1.94 mmol, 2.0 equiv) was added to the solution. The reaction mixture was stirred and heated to 90° C. for 3 h. The desired product was detected by LCMS. The reaction mixture was extracted with ethyl acetate (50 mL*3), washed with brine, dried over anhydrous Na₂SO₄. The filtrate was concentrated under vacuum and the residue was purified by FCC (PE:EA=3:1) to afford tert-butyl (2-((1-(6-bromo-3-(3,5-difluorophenyl)quinolin-4-yl)piperidin-4-yl)amino)ethyl)-carbamate (compound 8-1-3, 170 mg, 0.30 mmol, 31%) as yellow oil. MS (m/z) 561.2 (M+H)⁺.

### Step 3: Synthesis of (4-(4-((2-((tert-butoxycarbonyl)amino)ethyl)amino)piperidin-1-yl)-3-(3,5-difluorophenyl)quinolin-6-yl)boronic Acid (Compound 8-14)

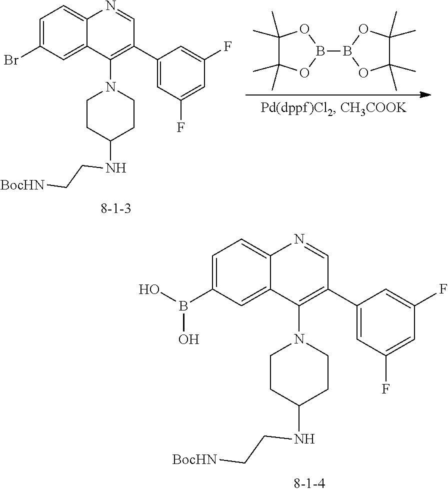

(4-(4-((2-((tert-butoxycarbonyl)amino)ethyl)amino)piperidin-1-yl)-3-(3,5-difluorophenyl)quinolin-6-yl)boronic acid (compound 8-1-3, 170 mg, 0.3 nmol, 1.0 equiv), 4,4,4′,4′,5,5,5′,5′-octamethyl-2,2′-bi(1,3,2-dioxaborolane) (120 mg, 0.47 mmol, 1.5 equiv) and CH₃COOK (61 mg, 0.62 mmol, 2.0 equiv) were dissolved in 1,4-dioxane (6 mL), then Pd(dppf)Cl₂ (46 mg, 0.062 mmol, 0.2 equiv) was added to the solution under nitrogen atmosphere. The reaction mixture was stirred for 3 h. The desired and heated to 90° C. for 2 h under nitrogen atmosphere. The desired product was detected by TLC. The reaction mixture was filtered through diatomite and the filtrate was concentrated under vacuum to afford crude product (4-(4-((2-((tert-butoxycarbonyl)amino)ethyl)amino)piperidin-1-yl)-3-(3,5-difluorophenyl)quinolin-6-yl)boronic acid (compound 8-1-4, 157 mg, 0.3 mmol, 100%) for next step.

### Step 4: Synthesis of methyl 6-((2E,4E)-4-(4-(4-((2-((tert-butoxycarbonyl-yl)amino)ethyl)amino)piperidin-1-yl)-5-(3,5-difluorophenyl) pyridin-2(3H)-ylid-ene)but-2-en-2-yl)picolinate (Compound 8-1-5)

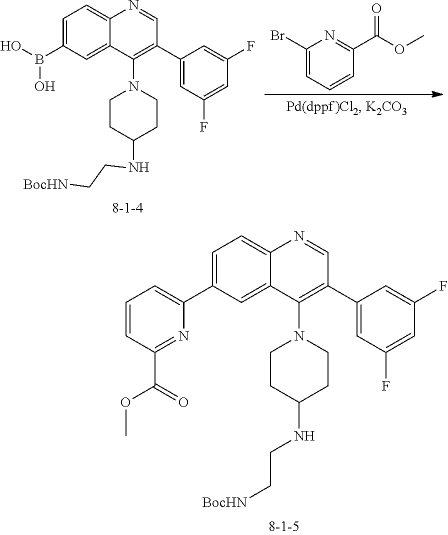

Methyl-6-((2E,4E)-4-(4-(4-((2-((tert-butoxycarbonyl)amino)ethyl)amino) piperidin-1-yl)-5-(3,5-difluorophenyl)pyridin-2(3H)-ylidene)but-2-en-2-yl)picolinate (compound 8-14, 157 mg, 0.3 mmol, 1.0 equiv), methyl 6-bromopicolinate (102 mg, 0.47 mmol, 1.5 equiv) and K₂CO₃ (86 mg, 0.62 mmol, 2.0 equiv) were dissolved in 1,4-dioxane (4 mL): H2O (1 mL)=4:1, then Pd(dppf)Cl₂ (46 mg, 0.06 mmol, 0.2 equiv) was added to the solution under nitrogen atmosphere. The reaction mixture was stirred and heated to 90° C. for 2 h under nitrogen atmosphere. The desired product was detected by LCMS. The reaction mixture was extracted with ethyl acetate (20 mL*3), washed with brine, dried over anhydrous Na₂SO₄. The filtrate was concentrated under vacuum and the residue was purified by Pre-TLC(PE:EA=1:1) to afford methyl 6-((2E,4E)-4-(4-(4-((2-((tert-butoxycarbonyl)amino)ethyl) amino)piperidin-1-yl)-5-(3,5-difluorophenyl)pyridin-2(3H)-ylidene)but-2-en-2-yl)picolinate (compound 8-1-5, 100 mg, 0.16 mmol, 53%) as gray solid. MS (m/z) 618.5 (M+H)⁺.

### Step 5: Synthesis of methyl 6-(4-(4-((2-aminoethyl)amino)piperidin-1-yl)-3-(3,5-difluorophenyl)quinolin-6-yl)picolinate (Compound 8-1-6)

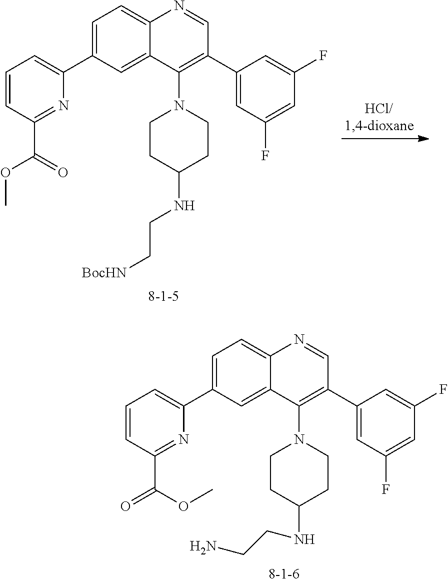 [SMILES: COC(=O)c1cccc(-c2ccc3ncc(-c4cc(F)cc(F)c4)c(N4CCC(NCCNC(=O)OC(C)(C)C)CC4)c3c2)n1 ; COC(=O)c1cccc(-c2ccc3ncc(-c4cc(F)cc(F)c4)c(N4CCC(NCCN)CC4)c3c2)n1]

Methyl-6-(4-(4-((2-((tert-butoxycarbonyl)amino)ethyl)amino)piperidin-1-yl)-3-(3,5-difluoro-phenyl)quinolin-6-yl)picolinate (compound 8-1-5, 100 mg, 0.16 mmol, 1.0 equiv) was dissolved in the solution of HCl/1,4-dioxane(4M, 4 mL). The reaction mixture was stirred at room temperature for 1 h. The desired product was detected by TLC. The reaction mixture was concentrated under vacuum to afford crude product methyl 6-(4-(4-((2-aminoethyl)amino)piperidin-1-yl)-3-(3,5-difluorophenyl)quinolin-6-yl)picolinate (compound 8-1-6, 83 mg, 0.16 mmol, 100%) as gray solid which was used for next step.

### Step 6: Synthesis of Compound 78

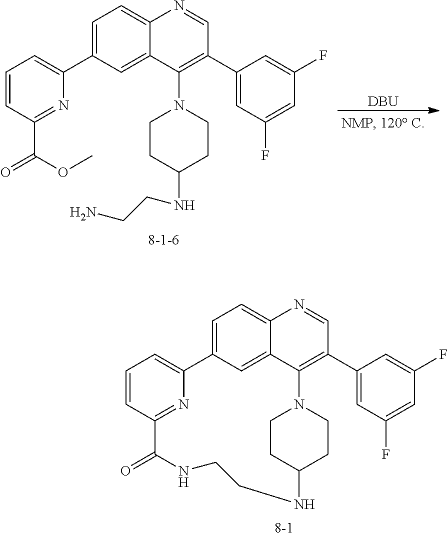

Methyl-6-(4-(4-((2-aminoethyl)amino)piperidin-1-yl)-3-(3,5-difluorophenyl) quinolin-6-yl)picolinate (compound 8-1-6, 83 mg, 0.16 mmol, 1.0 equiv) was dissolved in NMP (4 mL), then DBU(48 mg, 0.32 mmol, 2.0 equiv) was added to the solution. The reaction mixture was stirred and heated to 120° C. for 3 h. The desired product was detected by LCMS. The reaction mixture was extracted with ethyl acetate (20 mL*3), washed with brine, dried over anhydrous Na₂SO₄. The filtrate was concentrated under vacuum, and the residue was purified by pre-TLC (EA:PE=1:0, Rf=0.16) to afford crude product. The crude product was purified by pre-HPLC (0.1% TFA in water) to afford 23-(3,5-difluorophenyl)-4,7-diaza-2(6,4)-quinolina-1(2,6)-pyridina-3(1.4)-piperidina-cyclooctaphan-8-one (compound 78, 18.3 mg, 0.038 mmol, 24%) as yellow solid.

### Synthesis of Compound 79

The synthetic method of compound 8-2-5 was similar to compound 8-1-5.

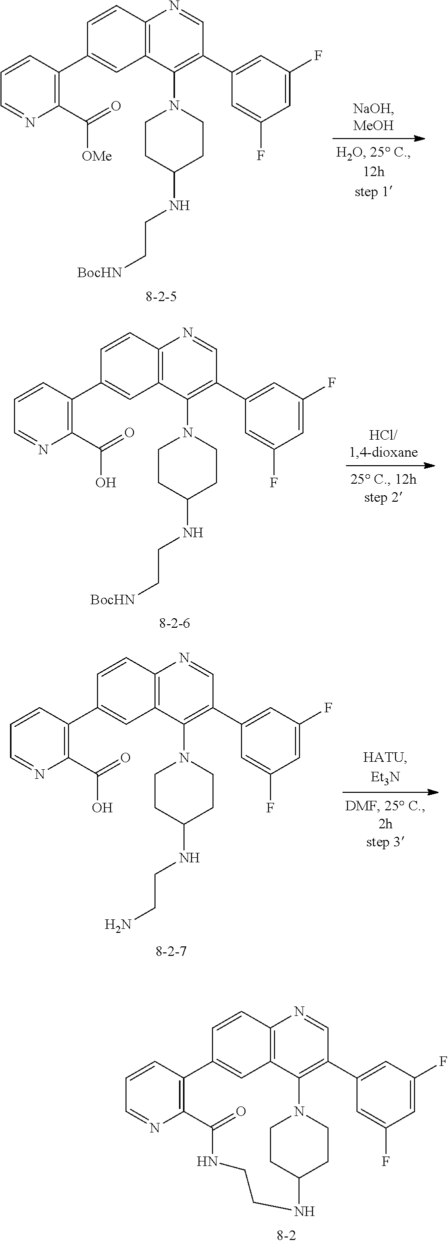 [SMILES: COC(=O)c1ncccc1-c1ccc2ncc(-c3cc(F)cc(F)c3)c(N3CCC(NCCNC(=O)OC(C)(C)C)CC3)c2c1 ; CC(C)(C)OC(=O)NCCNC1CCN(c2c(-c3cc(F)cc(F)c3)cnc3ccc(-c4cccnc4C(=O)O)cc23)CC1 ; NCCNC1CCN(c2c(-c3cc(F)cc(F)c3)cnc3ccc(-c4cccnc4C(=O)O)cc23)CC1 ; O=C1NCCNC2CCN(CC2)c2c(-c3cc(F)cc(F)c3)cnc3ccc(cc23)-c2cccnc21]

### Step 1′: Synthesis of 3-(4-(4-((2-((tert-butoxycarbonyl)amino)ethyl)amino) piperidin-1-yl)-3-(3,5-difluorophenyl)quinolin-6-yl)picolinic Acid (Compound 8-2-6)

To a solution of methyl 3-(4-(4-((2-((tert-butoxycarbonyl)amino)ethyl) amino)piperidin-1-yl)-3-(3,5-difluorophenyl)quinolin-6-yl)picolinate (compound 8-2-5, 210 mg, 0.34 mmol, 1 equiv.) in THF (4 ml), MeOH (1.5 ml), H₂O (1.5 ml) was added sodium hydroxide (136 mg, 3.4 mmol, 10 equiv.) and the solution was stirred at 25° C. for 12 h. The reaction was monitored by TLC, after completion, the reaction mixture was added with HCl (2M) and was filtered. The solid was concentrated under reduced pressure to afford 3-(4-(4-((2-((tert-butoxycarbonyl)amino) ethyl)amino)piperidin-1-yl)-3-(3,5-difluorophenyl)quinolin-6-yl)picolinic acid (compound 8-2-6, 140 mg, 0.23 mmol, 68.21%) as white solid. MS (m/z) 604 (M+H)⁺.

### Step 6: Synthesis of 3-(4-(4-((2-aminoethyl)amino)piperidin-1-yl)-3-(3,5-difluorophenyl) quinolin-6-yl)picolinic Acid (Compound 8-2-7)

To a solution of 3-(4-{4-[(2-{[(tert-butoxy)carbonyl]amino}ethyl)amino]piperidin-1-yl}-3-(3,5-difluorophenyl)quinolin-6-yl)pyridine-2-carboxylic acid (compound 8-2-6, 210 mg, 0.35 mmol, 1 equiv.) in EA (2 ml) was added HCl/1,4-dioxane (2 ml) and the solution was stirred at 25° C. for 12 h. The reaction was monitored by TLC, after completion, the reaction mixture was concentrated under reduced pressure to afford crude compound 8-2-7 as yellow oil. MS (m/z) 504 (M+H)⁺.

### Step 7: Synthesis of 2³-(3,5-difluorophenyl)-4,7-diaza-2(6,4)-quinolina-1(3,2)-pyridina-3(1,4)-piperidinacyclooctaphan-8-one (Compound 79)

To a solution of 3-(4-{4-[(2-aminoethyl)amino]piperidin-1-yl}-3-(3,5-difluoro phenyl)quinolin-6-yl)pyridine-2-carboxylic acid (compound 8-2-7, 135 mg, 0.27 mmol, 1 equiv.) and ethylbis(propan-2-yl) amine (70 mg, 0.54 mmol, 2 equiv.) in DMF (2.5 ml, 100.0%) was added [bis(dimethylamino)methylidene]((3H-[1,2,3]triazolo[4,5-b]pyridin-3-yl))oxidant-ium hexafluoro-λ⁵-phosphanuide (154 mg, 0.405 mmol, 1.5 equiv.) and the solution was stirred at 25° C. for 2 h. The reaction was monitored by LCMS and TLC, after completion, the reaction mixture was diluted with water (100 mL) and extracted with EA (200 mL×3). The combined organic extract and concentrated to give the crude material, the above mixture was purified by Prep-HPLC, to provide 2³-(3,5-difluorophenyl)-4,7-diaza-2(6,4)-quinolina-1(3,2)-pyridina-3(1,4)-piperidinacyclooctaphan-8-one (compound 79, 2 mg, 1.54%) as yellow solid.

The synthetic method of compound 80 was similar to compound 79.

Compounds 78-80 were synthesized according to the 6- or 7-step procedure described in Example 8. Analytical data for compounds 78-80 are provided below.

| Cpd. | | |
|---|---|---|
| No. | Structure | Analytical Data |
| 78 | 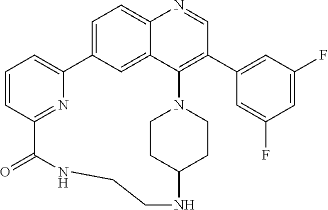 [SMILES: O=C1NCCNC2CCN(CC2)c2c(-c3cc(F)cc(F)c3)cnc3ccc(cc23)-c2cccc1n2] | ¹H NMR (400 MHz, CD₃OD) δ 9.93 (s, 1H), 8.74-8.67 (m, 2H), 8.39 (d, J = 7.6 Hz, 1H), 8.16 (dd, J = 14.4, 8.1 Hz, 2H), 7.99 (d, J = 8.0 Hz, 1H), 7.16 (s, 3H), 3.92-3.74 (m, 6H), 3.54 (s, 2H), 3.19 (t, J = 12.8 Hz, 3H), 2.66 (d, J = 13.6 Hz, 2H), 2.29 (d, J = 11,6 Hz, 2H). MS (m/z) 486.4 (M + H)⁺. |
| 79 | 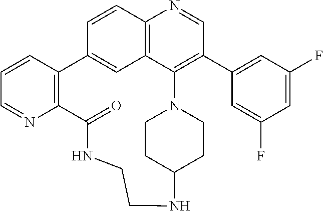 [SMILES: O=C1NCCNC2CCN(CC2)c2c(-c3cc(F)cc(F)c3)cnc3ccc(cc23)-c2cccnc21] | 1H NMR (400 MHz, DMSO-d6): δ ppm δ 8.68 (s, 1H), 8.67-8.63 (m, 1H), 8.36 (s, 1H), 8.11 (d, J = 8.7 Hz, 1H), 7.97-7.87 (m, 2H), 7.71 (dd, J = 7.9, 4.8 Hz, 1H), 7.15 (m, 3H), 3.84-3.64 (m, 2H), 3.60-3.38 (m, 3H), 3.26-3.14 (m, 2H), 2.94 (t, J = 12.2 Hz, 2H), 2.15 (m, 2H), 1.94 (m, 2H). MS (m/z) 487 (M + H)⁺. |
| 80 | 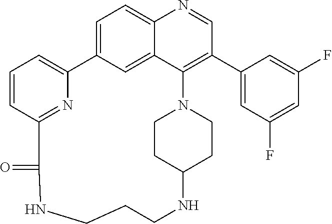 [SMILES: O=C1NCCCNC2CCN(CC2)c2c(-c3cc(F)cc(F)c3)cnc3ccc(cc23)-c2cccc1n2] | ¹H NMR (400 MHz, cd3od) δ 9.40 (s, 1H), 8.72 (s, 1H), 8.67 (d. J = 8.4 Hz, 1H), 8.37 (d, J = 7.6 Hz, 1H), 8.17 (dd, J = 17.6, 8.4 Hz, 2H), 8.04 (d, J = 7.6 Hz, 1H), 7.15 (d, J = 8.4 Hz, 3H), 3.73 (t, J = 16.8 Hz, 3H), 3.59 (d, J = 4.0 Hz, 2H), 3.42-3.35 (m, 2H), 3.03 (t, J = 11.2 Hz, 2H), 2.47- 2.34 (m, 2H), 2.08-1.99 (m, 4H). MS (m/z) 500 (M + H)⁺. |

### Example 9

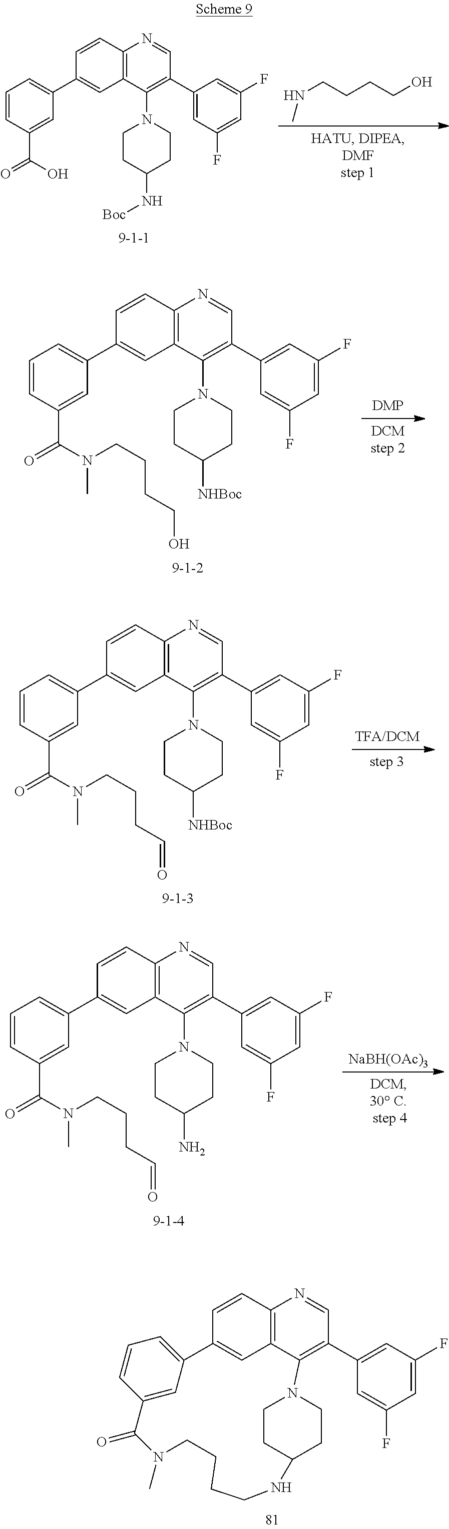

### Step 1: Synthesis of tert-butyl (1-(3-(3, 5-difluorophenyl)-6-(3-((4-hydroxybutyl) (methyl)carbamoyl)phenyl)quinolin-4-yl)piperidin-4-yl)carbamate (Compound 9-1-2)

To a solution of compound 9-1-1 (201.46 mg, 0.36 mmol, 1 equiv.) in DMF (5 mL, 100.0%) was added [[bis(dimethylamino)methylidene]({3H-[1,2,3]triazolo[4,5-b]pyridin-3-yl})oxidanium hexafluoro-λ⁵-phosphanuide (509.6 mg, 1.34 mmol, 3 equiv.) and DIPEA (139.58 mg, 1.08 mmol, 3 equiv.) and the solution was stirred at 20° C. for 10 min. The mixture was added 4-(methylamino)butan-1-ol (92.84 mg, 0.9 mmol, 2 equiv.). The solution was stirred at 20° C. for 3 h. The reaction was diluted with EA and water. The solution was extracted with EA (3×25 mL). The organic layer was separated, washed with further saturated NaCl solution, and concentrated in vacuo. The residue was purified using silica gel column chromatography eluting with ethyl acetate in petroleum ether to afford compound 9-1-2 (270 mg, 0.42 mmol, 93.33%). MS (m/z) 645 (M+H)⁺.

### Step 2: Synthesis of tert-butyl (1-(3-(3, 5-difluorophenyl)-6-(3-(methyl (4-oxobutyl) carbamoyl) phenyl) quinolin-4-yl) piperidin-4-yl)carbamate (Compound 9-1-3)

To a solution of compound 9-1-2 (150 mg, 0.23 mmol, 1 equiv.) in DCM (5 mL) was added Pyridinium chlorochromate (100.3 mg, 0.47 mmol, 2 equiv.) and the solution was stirred at 20° C. for 3 h. TLC (DCM:MeOH=10:1) showed the material was consumed and a main spot was detected. The reaction was diluted with DCM and water. The solution was extracted with EA (3×25 mL). The organic layer was separated, washed with further saturated NaCl solution, and concentrated in vacuo. The residue was purified using silica gel column chromatography eluting with ethyl acetate in petroleum ether to afford compound 9-1-3 (72 mg, 0.11 mmol, 48.15%).

### Step 3: Synthesis of 3-(4-(4-aminopiperidin-1-yl)-3-(3,5-difluorophenyl) quinolin-6-yl)-N-methyl-N-(4-oxobutyl) benzamide (Compound 9-1-4)

To a solution of compound 9-1-3 (32.2375 mg, 0.05 mmol, 1 equiv.) in [1,4-dioxane (3 mL)] was added (hydrogen chloride 4 mol/L.) and the solution was stirred at 20° C. for 3 h. TLC (DCM:MeOH=10:1) showed the material was consumed and a main spot was detected. After completion, the mixture was filtered and the filtrate was concentrated under reduced pressure to give compound 9-1-4 (10 mg, 0.02 mmol, 31.12%).

### Step 4: Synthesis of 23-(3, 5-difluorophenyl)-5-methyl-5, 10-diaza-2(4, 6)-quinolina-1(1, 4)-piperidina-3(1,3)-benzenacyclodecaphan-4-one (Compound 81)

To a solution of compound 5 (10 mg, 0.02 mmol, 1 equiv.) in DCM (5 mL) was added trimethyl sodio-λ⁴-boranetricarboxylate (8.44 mg, 0.04 mmol, 2 equiv.) and the solution was stirred at Temperature (30° C.) for Time (16h). The reaction was diluted with DCM and water. The solution was extracted with DCM (2-5 mL). The organic layer was separated, washed with further saturated NaCl solution, and concentrated in vacuo. The residue was purified by prep-HPLC to afford compound 81 (2 mg, 18.99%).

Analytical data for compound 81 is provided below.

| Cpd. | | |
|---|---|---|
| No. | Structure | Analytical Data |
| 81 | 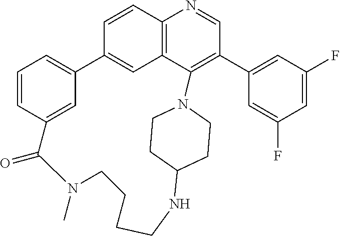 [SMILES: CN1CCCCNC2CCN(CC2)c2c(-c3cc(F)cc(F)c3)cnc3ccc(cc23)-c2cccc(c2)C1=O] | ¹H NMR (399 MHz, CD₃OD) δ8.69 (s, 1H), 8.43(s, 1H), 8.36- 8.20 (m, 2H), 7.95-7.80 (m, 2H), 7.70-7.50 (m, 2H), 7.25- 7.13 (m, 3H), 3.75-3.55 (m, 4H), 3.49-3.35 (m, 1H), 3.25- 3.21 (m, 2H), 3.11 (s, 3H), 3.10-2.95 (m, 2H), 2.30-2.95 (m, 8H). MS (m/z) 527.70 (M + H)⁺. |

### Example 10

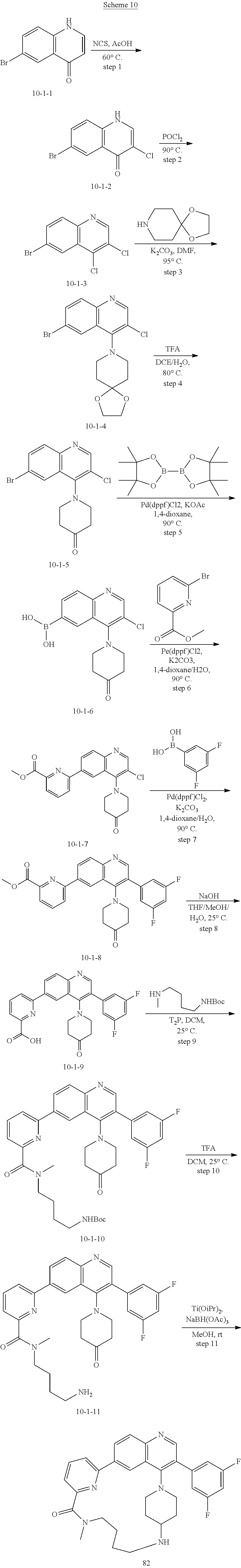

### Step 1: Synthesis of 6-bromo-3-chloro-1,4-dihydroquinolin-4-one (Compound 10-1-2)

To a stirred solution of 6-bromo-1,4-dihydroquinolin-4-one (compound 10-1-1, 30 g, 133.9 mmol) in AcOH (500 mL) was added 1-chloropyrrolidine-2,5-dione (17.88 g, 133.9 mmol) at 0° C. The reaction was stirred at 60° C. for 2 hours. TLC (PE:EtOAc=1:1) indicated the reaction was complete. The reaction mixture was filtered, and concentrated and evaporated to afford 6-bromo-3-chloro-1,4-dihydroquinolin-4-one (compound 10-1-2, 25 g, 96.71 mmol, 72.23%) as a white solid. MS (m/z) 257.9/259.9 (M+H)⁺.

### Step 2: Synthesis of 6-bromo-3,4-dichloroquinoline (Compound 10-1-3)

To a stirred solution of 6-bromo-3-chloro-1,4-dihydroquinolin-4-one (compound 10-1-2, 24.83 g, 96.05 mmol, 1 equiv.) in phosphoryl trichloride (250 ml, 1629.97 mmol, 16.97 equiv.) was added N,N-dimethylformamide (702.03 mg, 9.61 mmol, 0.1 equiv.) at 20° C. The reaction was stirred at 97° C. for 2 hours. TLC (PE:EtOAc=5:1) indicated the reaction was complete. The reaction mixture was concentrated under reduced pressure. The residue was diluted with EtOAc (500 mL), washed with sat. NaHCO₃ (500 mL), dried over Na₂SO₄ and evaporated to give 6-bromo-3,4-dichloroquinoline (compound 10-1-3, 26 g, 93.88 mmol, 97.74%) as a white solid. MS (m/z) 275.9/277.9 (M+H)⁺.

### Step 3: Synthesis of 8-(6-bromo-3-chloroquinolin-4-yl)-1,4-dioxa-8-azaspiro[4.5]decane (Compound 10-1-4)

To a stirred solution of 6-bromo-3,4-dichloroquinoline (compound 10-1-3, 15.00 g, 54.16 mmol, 1 equiv.) in DMF (150 ml) was added 1,4-dioxa-8-azaspiro[4.5]decane (11.6322 g, 81.24 mmol, 1.5 equiv.) and dipotassium carbonate (14.97 g, 108.32 mmol, 2 equiv.) at 20° C. The reaction was stirred at 100° C. for 16 hours. TLC (PE:EtOAc=5:1) indicated the reaction was complete. The reaction mixture was poured into H₂O (1 L), filtered, and concentrated to afford 8-(6-bromo-3-chloroquinolin-4-yl)-1,4-dioxa-8-azaspiro[4.5] decane (compound 10-1-4, 20 g, 52.13 mmol, 96.25%). MS (m/z) 383.1/385.1 (M+H)⁺.

### Step 4: Synthesis of 1-(6-bromo-3-chloroquinolin-4-yl)piperidin-4-one (Compound 10-1-5)

To a stirred solution of 8-(6-bromo-3-chloroquinolin-4-yl)-1,4-dioxa-8-azaspiro[4.5]decane (compound 10-14, 20.0006 g, 52.13 mmol, 1 equiv.) in H₂O (100 ml) was added trifluoroacetic acid (100 ml, 877 mmol, 16.82 equiv.) and 1,2-dichloroethane (100 ml, 1011 mmol, 19.39 equiv.) at 20° C. The reaction was stirred at 87° C. for 16 hours. TLC (PE:EtOAc=5:1) indicated the reaction was complete. The reaction mixture was concentrated under reduced pressure. The residue was diluted with EtOAc (500 mL), washed with sat.NaHCO₃ (500 mL), dried over Na₂SO₄ and evaporated to give 1-(6-bromo-3-chloroquinolin-4-yl)piperidin-4-one (compound 10-1-5, 15 g, 44.17 mmol, 84.73%) as a white solid. MS (m/z) 339/341 (M+H)⁺.

### Step 5: Synthesis of 1-[3-chloro-6-(4,4,5,5-tetramethyl-1,3,2-dioxaborolan-2-yl)quinolin-4-yl]piperidin-4-one (Compound 10-1-6)

To a stirred solution of 1-[3-chloro-6-(4,4,5,5-tetramethyl-1,3,2-dioxaborolan-2-yl)quinolin-4-yl]piperidin-4-one (compound 10-1-5, 2 g, 5.17 mmol, 87.83%) in 1,4-dioxane (70 ml) was added 4,4,5,5-tetramethyl-2-(4,4,5,5-tetramethyl-1,3,2-dioxaborolan-2-yl)-1,3,2-dioxaborolane (2243.22 mg, 8.83 mmol, 1.5 equiv.), potassium acetate (1155.91 mg, 11.78 mmol, 2 equiv.) and 1,1′-Bis(diphenylphosphino)ferrocene-palladium(II)dichloride dichloromethane complex (449.23 mg, 0.59 mmol, 0.1 equiv.) at 20° C. The reaction was stirred at 100° C. for 1 hours. TLC (PE:EtOAc=5:1) indicated the reaction was complete. The reaction mixture was concentrated under reduced pressure. The residue was diluted with EtOAc (100 mL), washed with sat.NaHCO₃ (500 mL), dried over Na₂SO₄ and evaporated to give 1-[3-chloro-6-(4,4,5,5-tetramethyl-1,3,2-dioxaborolan-2-yl)quinolin-4-yl]piperidin-4-one (compound 10-1-6, 2 g, 5.17 mmol, 87.83%) as a white solid. MS (m/z) 387.2 (M+H)⁺.

### Step 6: Synthesis of methyl 6-[3-chloro-4-(4-oxopiperidin-1-yl)quinolin-6-yl]pyridine-2-carboxylate (Compound 10-1-7)

To a stirred solution of 1-[3-chloro-6-(4,4,5,5-tetramethyl-1,3,2-dioxaborolan-2-yl)quinolin-4-yl]piperidin-4-one (compound 10-1-6, 2 g, 5.17 mmol, 1 equiv.) in 1,4-Dioxane (60 ml) was added water (15 ml), methyl 6-bromopyridine-2-carboxylate (1116.88 mg, 5.17 mmol, 1 equiv.), 1,1′-Bis(diphenylphosphino)ferrocene-palladium(II)dichloride dichloromethane complex (394.37 mg, 0.52 mmol, 0.1 equiv.) and dipotassium carbonate (1429.09 mg, 10.34 mmol, 2 equiv.) at 20° C. The reaction was stirred at 90° C. for 1 hours. TLC (PE:EtOAc=5:1) indicated the reaction was complete. The reaction mixture was concentrated under reduced pressure. The residue was diluted with EtOAc (100 mL), washed with sat.NaHCO₃ (50 mL), dried over Na₂SO₄ and evaporated to give the residue. The residue was purified by column chromatography on silica gel eluted with PE/EtOAc (9:1-1:1) to give methyl 6-[3-chloro-4-(4-oxopiperidin-1-yl)quinolin-6-yl]pyridine-2-carboxylate (compound 10-1-7, 1.9 g, 4.8 mmol, 92.84%) as a white solid. MS (m/z) 396.1 (M+H)⁺.

### Step 7: Synthesis of methyl 6-[3-(3,5-difluorophenyl)-4-(4-oxopiperidin-1-yl)quinolin-6-yl]pyridine-2-carboxylate (Compound 10-1-8)

To a stirred solution of methyl 6-[3-chloro-4-(4-oxopiperidin-1-yl)quinolin-6-yl]pyridine-2-carboxylate (compound 10-1-7, 1.9 g, 4.8 mmol, 1 equiv.) in 1,4-dioxane (60 ml) was added water (15 ml), (3,5-difluorophenyl)boronic acid (757.97 mg, 4.8 mmol, 1 equiv.), dipotassium carbonate (1326.82 mg, 9.6 mmol, 2 equiv.) and dichlorobis[di-tert-butyl(4-dimethylaminophenyl)phosphino]palladium(I) (339.87 mg, 0.48 mmol, 0.1 equiv.) at 20° C. The reaction was stirred at 100° C. for 1 hours. TLC (PE:EtOAc=1:1) indicated the reaction was complete. The reaction mixture was concentrated under reduced pressure. The residue was diluted with EtOAc (100 mL), washed with sat. NaHCO₃ (50 mL), dried over Na₂SO₄ and evaporated to give the residue. The residue was purified by column chromatography on silica gel eluted with PE/EtOAc (9:1˜1:1) to give methyl 6-[3-(3,5-difluorophenyl)-4-(4-oxopiperidin-1-yl)quinolin-6-yl]pyridine-2-carboxylate (compound 10-1-8, 1.6 g, 3.38 mmol, 70.42%) as a white solid. MS (m/z) 474.2 (M+H)⁺. LCMS: m/z: 474.2(M+H).

### Step 8: Synthesis of 6-[3-(3,5-difluorophenyl)-4-(4-oxopiperidin-1-yl)quinolin-6-yl]pyridine-2-carboxylic Acid (Compound 10-1-9)

To a stirred solution of methyl 6-[3-(3,5-difluorophenyl)-4-(4-oxopiperidin-1-yl)quinolin-6-yl]pyridine-2-carboxylate (compound 10-1-8, 1.4 g, 3.05 mmol, 90.17%) in THF (20 ml) was added sodium hydroxide (675.95 mg, 16.9 mmol, 5 equiv.) and water (5 ml) at 0° C. The reaction was stirred at 40° C. for 2 hours. TLC (PE:EtOAc=1:1) indicated the reaction was complete. The PH of the reaction mixture was adjusted to 34. The reaction mixture was filtered, and concentrated and evaporated to afford 6-[3-(3,5-difluorophenyl)-4-(4-oxopiperidin-1-yl)quinolin-6-yl]pyridine-2-carboxylic acid (compound 10-1-9, 1.4 g, 3.05 mmol, 90.24%) as a white solid. MS (m/z) 460.2 (M+H)⁺.

### Step 9: Synthesis of tert-butyl N-[4-(1-{3-[3-(3,5-difluorophenyl)-4-(4-oxopiperidin-1-yl)quinolin-6-yl]phenyl}-N-methylformamido)butyl]carbamate (Compound 10-1-10)

To a stirred solution of 6-[3-(3,5-difluorophenyl)-4-(4-oxopiperidin-1-yl)quinolin-6-yl]pyridine-2-carboxylic acid (compound 10-1-9, 750 mg, 1.63 mmol, 1 equiv.) in DMF (10 ml) was added tert-butyl N-[4-(methylamino)butyl]carbarnate (330.22 mg, 1.63 mmol, 1 equiv.), tripropyl-1,3,5,2λ⁵,4λ⁵,6λ⁵-trioxatriphosphinane-2,4,6-trione (1038.81 mg, 3.26 mmol, 2 equiv.) and ethylbis(propan-2-yl)amine (1054.87 mg, 8.16 mmol, 5 equiv.) at 20° C. The reaction was stirred at 20° C. for 1 hours. TLC (PE:EtOAc=1:1) indicated the reaction was complete. The reaction mixture was concentrated under reduced pressure. The residue was diluted with EtOAc (100 mL), washed with sat.NaHCO₃ (50 mL), dried over Na₂SO₄ and concentrated to dryness. The residue was purified by column chromatography on silica gel eluted with PE/EtOAc (9:1-1:1) to give tert-butyl N-[4-(1-{3-[3-(3,5-difluorophenyl)-4-(4-oxopiperidin-1-yl)quinolin-6-yl]phenyl}-N-methylformamido)butyl]carbamate (compound 10-1-10, 520 mg, 0.81 mmol, 49.62%) as a white solid. MS (m/z) 644.3 (M+H)⁺.

### Step 10: Synthesis of N-(4-aminobutyl)-6-(3-(3,5-difluorophenyl)-4-(4-oxopiperidin-1-yl)quinolin-6-yl)-N-methylpicolinamide (Compound 10-1-11)

To a stirred solution of tert-butyl (4-(6-(3-(3,5-difluorophenyl)-4-(4-oxopiperidin-1-yl)quinolin-6-yl)-N-methylpicolinamido)butyl)carbamate (compound 10-1-10, 520 mg, 0.81 mmol, 1 equiv.) in DCM (4 ml, 100.0%) was added trifluoroacetic acid (1 ml, 8.77 mmol, 10.84 equiv.) at 20° C. The reaction was stirred at 20° C. for 1 hours. TLC (DCM:MeOH=10:1) indicated the reaction was complete. The reaction mixture was concentrated under reduced pressure to give N-(4-aminobutyl)-6-(3-(3,5-difluorophenyl)-4-(4-oxopiperidin-1-yl)quinolin-6-yl)-N-methylpicolinamide (compound 10-1-11, 400 mg, 0.74 mmol, 91.11%) as a white solid. MS (m/z) 543.3 (M+H)⁺.

### Step 11: Synthesis of (Compound 82)

To a stirred solution of N-(4-aminobutyl)-6-(3-(3,5-difluorophenyl)-4-(4-oxopiperidin-1-yl)quinolin-6-yl)-N-methylpicolinamide (compound 10-1-11, 97.6714 mg, 0.18 mmol, 1 equiv.) in MeOH (5 ml, 100.0%) was added titanium(4+) tetrakis(propan-2-olate) (261.9 mg, 0.92 mmol, 5 equiv.) at 20° C. After stirring at 20° C. for 1 h, to the reaction was slowly added trimethyl sodio-λ⁴-boranetricarboxylate (388.73 mg, 1.84 mmol, 10 equiv.). The reaction was stirred at 20° C. for another 1 h. TLC (DCM:MeOH=10:1) indicated the reaction was complete. The reaction mixture was quenched with H₂O (5 mL) and extracted with DCM (30 mL×2). The combined organic phase was washed with brine (10 mL), dried over Na₂SO₄ and concentrated. The residue was purified by prep-TLC(DCM:MeOH=10:1) to give 24-(3,5-difluorophenyl)-11-methyl-2,6,11,22,27-pentaazapentacyclo[16.6.2.2²,⁵.1¹³,¹⁷.0²¹,²⁵]nonacosa-1(24),13(27),14,16,18(26),19,21(25),22-octaen-12-one (compound 82, 26 mg, 0.05 mmol, 27.13%) as a white solid.

Compounds 82-86 were synthesized according to the 11-step procedure described in Example 10. Analytical data for compounds 82-86 are provided below.

| Cpd. | | |
|---|---|---|
| No. | Structure | Analytical Data |
| 82 | 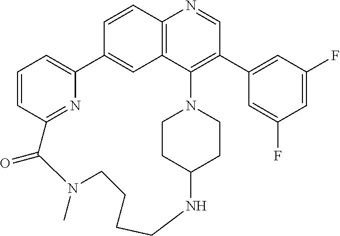 [SMILES: CN1CCCCNC2CCN(CC2)c2c(-c3cc(F)cc(F)c3)cnc3ccc(cc23)-c2cccc(n2)C1=O] | 1H NMR (400 MHz, CD3OD): 9.42 (s, 1H), 8.53 (s, 1H), 8.45 (d, J = 9.1 Hz, 1H), 8.24 (d, J = 7.9 Hz, 1H), 8.06 (dd, J = 17.9, 8.8 Hz, 2H), 7.46 (d, J = 7.3 Hz, 1H), 7.06 (d, J = 8.3 Hz, 3H), 3.45 (s, 2H), 3.28-3.16 (m, 3H), 3.02 (d, J = 16.8 Hz, 5H), 2.80 (s, 2H), 2.22 (s, 2H), 1.96-1.78 (m, 6H). MS (m/z) 528.3 (M + H)⁺. |
| 83 | 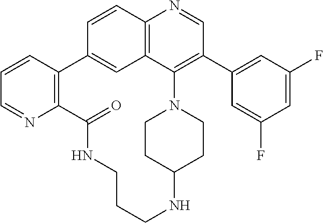 [SMILES: O=C1NCCCNC2CCN(CC2)c2c(-c3cc(F)cc(F)c3)cnc3ccc(cc23)-c2cccnc21] | ¹H NMR (400 MHz, cd3od) δ 8.74 (dd, J = 4.8, 1.2 Hz, 1H), 8.71 (s, 1H), 8.19-8.15 (m, 2H), 8.07 (dd, J = 8.0, 1.4 Hz, 1H), 8,00 (dd, J = 8.8, 1.6 Hz, 1H), 7.73 (dd, J = 8.0, 4.8 Hz, 1H), 7.19 (t, J = 9.2 Hz, 1H), 7.11 (d, J = 7.6 Hz, 2H), 3.61 (d, J = 13.2 Hz, 2H), 3.45 (d, J = 12.8 Hz, 2H), 3.25 (s, 2H), 3.14 (d, J = 8.8 Hz, 2H), 2.94 (t, J = 8.8 Hz, 2H), 2.22 (d, J = 13.6 Hz, 2H), 1.86 (dd, J = 11.2, 4.8Hz, 4H). MS (m/z) 500.8 (M + H)⁺. |
| 84 | 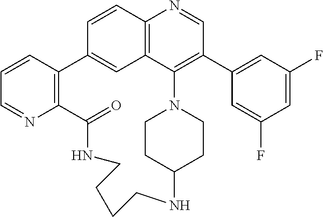 [SMILES: O=C1NCCCCNC2CCN(CC2)c2c(-c3cc(F)cc(F)c3)cnc3ccc(cc23)-c2cccnc21] | ¹H NMR (399 MHz, cd3od) δ 8.66 (d, J = 5.2 Hz, 2H), 8.18 (s, 0H), 8.11 (d, J = 8.8 Hz, 1H), 7.99 (d. J = 8.0 Hz, 0H), 7.94 (d. J = 8.8 Hz, 0H), 7.71-7.65 (m, 0H), 7.18 (t, J = 7.6 Hz, 0H), 7.11 (d, J = 5.6 Hz, 1H), 3.56 (d, J = 11.6 Hz, 1H), 3.32 (s, 0H), 3,22 (s, 1H), 3.09 (d, J = 7.5 Hz, 1H), 2.93 (s, 1H), 2.09 (s, 1H), 1.81 (s, 1H), 1.69 (s, 1H), 1.57 (s, 1H). MS (m/z) 514.7 (M + H)⁺. |
| 85 | 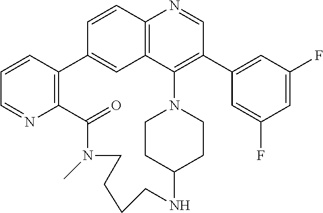 [SMILES: CN1CCCCNC2CCN(CC2)c2c(-c3cc(F)cc(F)c3)cnc3ccc(cc23)-c2cccnc2C1=O] | 1H NMR (400 MHz, CD3OD) δ 8.76 (s, 1H), 8.69 (d, J = 3.8 Hz, 1H), 8.35 (s, 1H), 8.23 (d, J = 8.7 Hz, 1H), 8.12 (d, J = 6.9 Hz, 2H), 7.74 (dd, J = 7.9, 4.9 Hz, 1H), 7.25 (s, 1H), 7.16 (d, J = 5.4 Hz, 2H), 3.70 (d, J = 12.7 Hz, 2H), 3.47 (s, 1H), 3.33 (s, 2H), 3.18 (s, 2H), 3.06 (t, J = 12.3 Hz, 2H), 2.50 (s, 3H), 2.21 (s, 2H), 1.91 (d, J = 9.8 Hz, 2H), 1.69 (s, 2H), 1.54 (s, 2H). MS (m/z) 528.3 (M + H)⁺. |
| 86 | 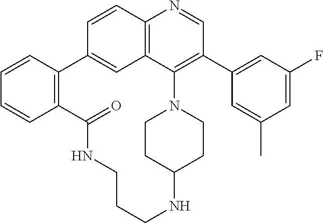 [SMILES: Cc1cc(F)cc(-c2cnc3ccc4cc3c2N2CCC(CC2)NCCCNC(=O)c2ccccc2-4)c1] | ¹H NMR (400 MHz, cd3od) δ 8.68 (s, 1H), 8.19 (d, J = 8.4 Hz, 1H), 8.12 (d, J = 8.8 Hz, 2H), 7.75 (d, J = 7.6 Hz, 1H), 7.66 (t, J = 7.2 Hz, 1H), 7.58 (dd, J = 14.4, 7.6 Hz, 2H), 7.17- 7.07 (m, 2H), 7.02 (d, J = 8.4 Hz, 1H), 3,63 (s, 2H), 3.38 (s, 1H), 3.18 (s, 2H), 3.11 (s, 2H), 2.98 (s, 2H), 2.49 (s, 3H), 2.31 (d, J = 15.2 Hz, 2H), 1.92 (d. J = 5.2 Hz, 2H), 1.83- 1.77 (m, 2H). MS (m/z) 495.6 (M + H)⁺. |

### Example 11

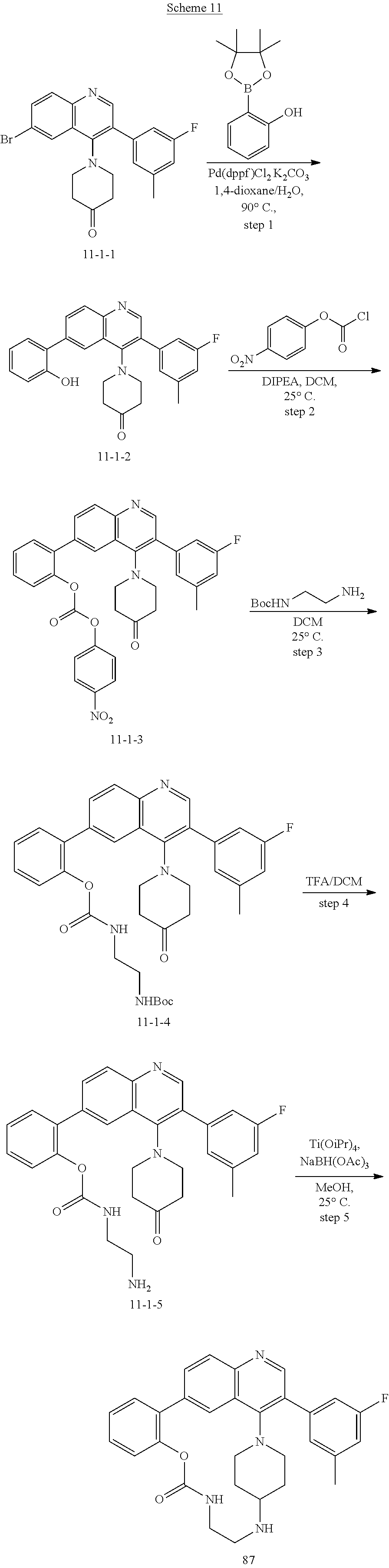

### Step 1: Synthesis of 1-(3-(3-fluoro-5-methylphenyl)-6-(2-hydroxyphenyl)quinolin-4-yl)piperidin-4-one (Compound 11-1-2)

1-(3-(3-fluoro-5-methylphenyl)-6-(2-hydroxyphenyl)quinolin-4-yl)piperidin-4-one (compound 11-1-1, 2 g, 4.84 mmol, 1.0 equiv), 2-(4,4,5,5-tetramethyl-1,3,2-dioxaborolan-2-yl)phenol (1 g, 7.26 mmol, 1.5 equiv) and K₂CO (1.33 g, 9.68 mmol, 2.0 equiv) were dissolved in 1,4-dioxane (16 mL):H₂O(4 mL)=4:1, then Pd(dppf)Cl₂ (353 mg, 0.484 mmol, 0.1 equiv) was added to the solution. The reaction mixture was stirred and heated to 90° C. for 2 h under nitrogen atmosphere. The desired product was detected by LCMS. The reaction mixture was extracted with ethyl acetate (50 mL*3), washed with brine, dried over anhydrous Na₂SO₄. The filtrate was concentrated under vacuum and the residue was purified by FCC (EA:PE=1:2) to afford 1-(3-(3-fluoro-5-methylphenyl)-6-(2-hydroxyphenyl)quinolin-4-yl)piperidin-4-one (compound 11-1-2, 800 mg, 1.87 mmol, 38.6%) as red solid. MS (m/z) 427.25 (M+H)⁻.

### Step 2: Synthesis of 2-(3-(3-fluoro-5-methylphenyl)-4-(4-oxopiperidin-1-yl)quinolin-6-yl)phenyl (4-nitrophenyl) carbonate (Compound 11-1-3)

1-(3-(3-fluoro-5-methylphenyl)-6-(2-hydroxyphenyl)quinolin-4-yl)piperidin-4-one (compound 11-1-2, 200 mg, 0.47 mmol, 1.0 equiv), 4-nitrophenyl carbonochloridate (113 mg, 0.564 mmol, 1.2 equiv) and DIPEA (151 mg, 1.175 mmol, 2.5 equiv) were dissolved in DCM (5 mL). The reaction mixture was stirred at 25° C. for 3 h under nitrogen atmosphere. The desired product was detected by LCMS. The reaction mixture was extracted with CH₂Cl₂ (20 mL*3), washed with brine, dried over anhydrous Na₂SO₄. The filtrate was concentrated under vacuum and the residue was purified by FCC (PE:EA=2:1) to afford 2-(3-(3-fluoro-5-methylphenyl)-4-(4-oxopiperidin-1-yl)quinolin-6-yl)phenyl (4-nitrophenyl) carbonate (compound 11-1-3, 200 mg, 0.338 mmol, 71.9%) as white oil. MS (n/z) 592.35 (M+H)⁺.

### Step 3: Synthesis of tert-butyl (2-(3-(3-fluoro-5-methylphenyl)-4-(4-oxopiperidin-1-yl)quinolin-6-yl)phenyl) ethane-1,2-diyldicarbamate (Compound 11-14)

2-(3-(3-fluoro-5-methylphenyl)-4-(4-oxopiperidin-1-yl)quinolin-6-yl)phenyl (4-nitrophenyl) carbonate (compound 11-1-3, 200 mg, 0.338 mmol, 1.0 equiv) was dissolved in DCM(4 mL), then tert-butyl (2-aminoethyl)carbamate (64 mg, 0.40 mmol, 1.2 equiv) was added to the solution. The reaction mixture was stirred at 25° C. for 1 h. The desired product was detected by LCMS. The reaction mixture was extracted with DCM (20 mL*3), washed with brine, dried over anhydrous Na₂SO₄. The filtrate was concentrated under vacuum and the residue was purified by FCC (EA:PE=2:1) to afford tert-butyl (2-(3-(3-fluoro-5-methylphenyl)-4-(4-oxopiperidin-1-yl)quinolin-6-yl)phenyl) ethane-1,2-diyldicarbamate (compound 11-14, 140 mg, 0.229 mmol, 67.7%) as yellow oil. MS (m/z) 613.5 (M+H)⁺.

### Step 4: Synthesis of 2-(3-(3-fluoro-5-methylphenyl)-4-(4-oxopiperidin-1-yl)quinolin-6-yl)phenyl (2-aminoethyl)carbamate (Compound 11-1-5)

Tert-butyl (2-(3-(3-fluoro-5-methylphenyl)-4-(4-oxopiperidin-1-yl)quinolin-6-yl) phenyl) ethane-1,2-diyldicarbamate (compound 11-14, 70 mg, 0.114 mmol, 1.0 equiv) was dissolved in the solution of TFA(1 mL):DCM(3 mL)=1:3. The reaction mixture was stirred at 25° C. for 1 h. The desired product was detected by LCMS. The reaction mixture was concentrated under vacuum to afford 2-(3-(3-fluoro-5-methylphenyl)-4-(4-oxopiperidin-1-yl)quinolin-6-yl)phenyl (2-aminoethyl)carbamate (compound 11-1-5, 58 mg, 0.114 mmol, 100%). MS (m/z) 513.35 (M+H)⁺.

### Step 5: Synthesis of Compound 87

2-(3-(3-fluoro-5-methylphenyl)-4-(4-oxopiperidin-1-yl)quinolin-6-yl)phenyl (2-aminoethyl) carbamate (compound 11-1-5, 58 mg, 0.114 mmol, 1.0 equiv) was dissolved in MeOH (4 mL). Then Ti(OiPr)₄ (321 mg, 1.14 mmol, 10 equiv) was added to the solution under nitrogen atmosphere. The reaction mixture was stirred at 25° C. for 30 min. Then NaBH(OAc)₃ (241 mg, 1.14 mmol, 10 equiv) was added to the solution. The reaction mixture was stirred at 25° C. for 12 h. The desired product was detected by LCMS. The reaction mixture was filtered through celite and the filtrate was concentrated under vacuum to afford crude product. The crude product was purified by pre-HPLC (0.1% TFA in water) to afford 23-(3-fluoro-5-methylphenyl)-4-oxa-6,9-diaza-2(4,6)-quinolina-1(1,4)-piperidina-3(1,2)-benzenacyclononaphan-5-one (compound 87, 5.0 mg, 0.0084 mmol, 7.4%) as yellow solid.

Analytical data for compound 87 is provided below.

| Cpd. | | |
|---|---|---|
| No. | Structure | Analytical Data |
| 87 | 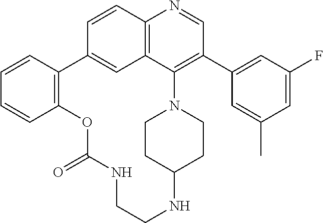 [SMILES: Cc1cc(F)cc(-c2cnc3ccc4cc3c2N2CCC(CC2)NCCNC(=O)Oc2ccccc2-4)c1] | ¹H NMR (400 MHz, cd3od) δ 8.74 (s, 1H), 8.47 (s, 1H), 8.18 (s, 2H), 7.78 (d, J = 8.0 Hz, 1H), 7.45 (dd, J = 13.6, 6.8 Hz, 3H), 7.12 (d, J = 8.4 Hz, 2H), 7.04 (d, J = 10.4 Hz, 1H), 3.65 (d, J = 5.2 Hz, 5H), 3.52 (d, J = 7.6 Hz, 2H), 2.88 (d, J = 5.2 Hz, 2H), 2.47 (s, 3H), 2.23 (d, J = 10.4 Hz, 2H), 1.94 (d, J = 6.4 Hz, 2H). MS (m/z) 497.35 (M + H)⁺. |

### Example 12

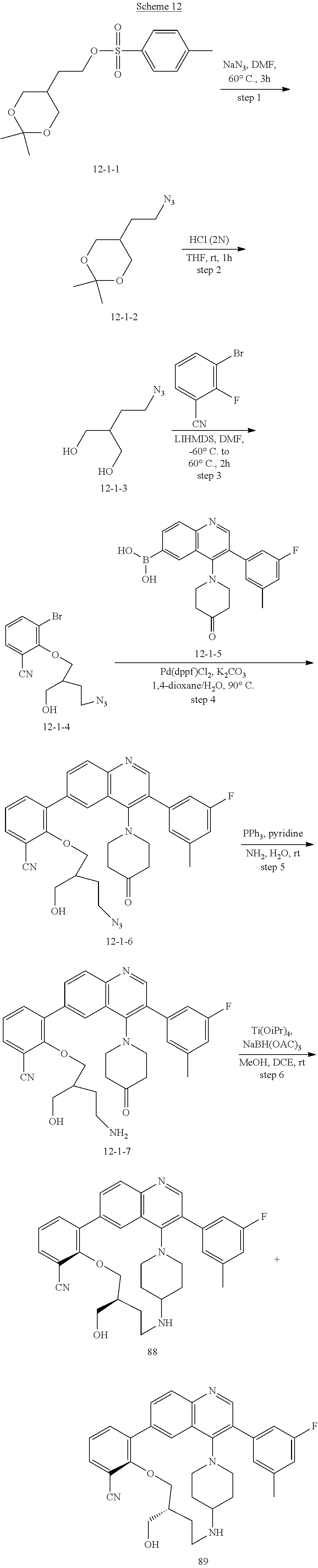

### Step 1: Synthesis of 5-(2-azidoethyl)-2,2-dimethyl-1,3-dioxane (Compound 12-1-2)

To a solution of 2-(2,2-dimethyl-1,3-dioxan-5-yl) ethyl 4-methylbenzenesulfonate (compound 12-1-1, 3.0 g, 9.55 mmol) in DMF (60 ml) was added sodium azide (1240.39 mg, 19.08 mmol, 2 equiv.) and the solution was stirred at 60° C. for 3 h. After completion, the reaction mixture was extracted with EA (500 mL×3). The combined organic phase was washed with brine, dried over anhydrous sodium sulfate and concentrated under reduced pressure to afford 5-(2-azidoethyl)-2,2-dimethyl-1,3-dioxane (compound 12-1-2, 1.74 g, 9.39 mmol) as colorless oil. MS (m/z) 186 (M+H)⁺.

### Step 2: Synthesis of 2-(2-azidoethyl)propane-1,3-diol (Compound 12-1-3)

To a solution of 5-(2-azidoethyl)-2,2-dimethyl-1,3-dioxane (compound 12-1-2, 1.74 g, 9.39 mmol, 1 equiv.) in THE (40 ml) was added HCl (2M) 8 ml and the solution was stirred at 25° C. for 1 h. After completion, the reaction mixture was concentrated under reduced pressure to afford the crude in water. The crude was freeze-dried to afford 2-(2-azidoethyl)propane-1,3-diol (compound 12-1-3, 1.61 g, 11.09 mmol) as yellow oil. MS (m/z) 146 (M+H)⁺.

### Step 3: Synthesis of 2-(4-azido-2-(hydroxymethyl)butoxy)-3-bromobenzonitrile (Compound 12-14)

To a solution of 2-(2-azidoethyl)propane-1,3-diol (compound 12-1-3, 1.61 g, 11.09 mmol) in DMF (12 ml) was added LiHMDS (11.7 ml, 11.7 mmol, 1.1 equiv.) at -60° C., the above mixture was stirred at −60° C. for 30 min, then 3-bromo-2-fluorobenzonitrile (1.70 g, 8.5 mmol) was added to the above mixture at 60° C. for 2 h. After completion, the reaction mixture was extracted with EA (500 mL×3). The combined organic phase was washed with brine, dried over anhydrous sodium sulfate and concentrated under reduced pressure, the crude product was purified by Flash (DCM:MeOH=12:1) to afford 2-(4-azido-2-(hydroxymethyl)butoxy)-3-bromobenzonitrile (compound 12-14, 1.29 g, 3.97 mmol) as white solid. MS (m/z) 326 (M+H)⁺.

### Step 4: Synthesis of 2-(4-azido-2-(hydroxymethyl)butoxy)-3-(3-(3-fluoro-5-methylphenyl)-4-(4-oxopiperidin-1-yl)quinolin-6-yl)benzonitrile (Compound 12-1-6)

To a solution of 2-(4-azido-2-(hydroxymethyl)butoxy)-3-bromobenzonitrile (compound 12-14, 1.29 g, 3.97 mmol) and (3-(3-fluoro-5-methylphenyl)-4-(4-oxopiperidin-1-yl)quinolin-6-yl)boronic acid (compound 12-1-5, 1.50 g, 3.97 mmol, 1 equiv.) in 1,4-Dioxane (30 ml) and H₂O (6 ml) was added dipotassium carbonate (1.09 g, 8 mmol, 2 equiv.) and [1,1′-bis(diphenylphosphino)ferrocene]palladium(II) chloride (290 mg, 0.397 mmol, 0.1 equiv.) and the solution was stirred at 100° C. for 1 h. The reaction was monitored by LCMS and TLC, after completion, the reaction mixture was diluted with water (50 mL) and extracted with EA (100 mL×2). The combined organic extract and concentrated to give the crude material, then the crude product was purified by Flash (DCM:MeOH=10:1) to afford 2-(4-azido-2-(hydroxymethyl)butoxy)-3-(3-(3-fluoro-5-methylphenyl)-4-(4-oxopiperidin-1-yl)quin-olin-6-yl)benzonitrile (compound 12-1-6, 800 mg, 1.38 mmol) as white solid. MS (m/z) 579 (M+H)⁺.

### Step 5: Synthesis of 2-(4-amino-2-(hydroxymethyl)butoxy)-3-(3-(3-fluoro-5-methylphenyl)-4-(4-oxopiperidin-1-yl)quinolin-6-yl)benzonitrile (Compound 12-1-7)

To a solution of 2-(4-azido-2-(hydroxymethyl)butoxy)-3-(3-(3-fluoro-5-methylphenyl)-4-(4-oxopiperidin-1-yl)quinolin-6-yl)benzonitrile (compound 12-1-6, 220 mg, 0.398 mmol, 1 equiv.) in NH₄OH (4 ml), pyridine (4 ml) was stirred at 25° C. for 1 h. The above mixture was added PPh₃ (100 mg, 0.398 mmol, 1 equiv) and stirred at 25° C. for 12 h. The reaction was monitored by TLC, after completion, the reaction mixture was diluted with water (50 mL) and extracted with EA (100 mL×2). The combined organic extract and concentrated to give the crude material, then the crude product was purified by Flash (DCM:MeOH=10:1) to afford 2-(4-amino-2-(hydroxymethyl)butoxy)-3-(3-(3-fluoro-5-methylphenyl)-4-(4-oxo-piperidin-1-yl)quinolin-6-yl)benzonitrile (compound 12-1-7, 140 mg, 0.253 mmol) as white solid. MS (m/z) 553 (M+H)⁺.

### Step 6: Synthesis of (S)-2³-(3-fluoro-5-methylphenyl)-6-(hydroxymethyl)-4-oxa-9-aza-2(4,6)-quinolina-1(1,4)-piperidina-3(1,2)-benzenacyclononaphane-3³-carbonitrile (Compound 88) and (R)-2³-(3-fluoro-5-methylphenyl)-6-(hydroxymethyl)-4-oxa-9-aza-2(4,6)-quinolina-1(1,4)-piperidina-3(1,2)-benzenacyclononaphane-3³-carbonitrile (Compound 89)

To a solution of 2-(4-amino-2-(hydroxymethyl)butoxy)-3-(3-(3-fluoro-5-methyl phenyl)-4-(4-oxopiperidin-1-yl)quinolin-6-yl)benzonitrile (compound 12-1-7, 120 mg, 0.223 mmol, 1 equiv.) and Ti(OiPr)₄ (308 mg, 1.115 mmol, 5 equiv.) in MeOH (1.8 ml) and DCE (1.8 ml) was stirred at 25° C. for 1 h. Then the NaBH(OAC)₃ (460 mg, 2.23 mmol, 10 equiv) was added to the above mixture and stirred at 25° C. for 12 h. The reaction was monitored by LCMS and TLC, after completion, the reaction mixture was diluted with water (100 mL) and extracted with EA (200 mL×3). The combined organic extract and concentrated to give the crude material, the above mixture was purified by Prep-HPLC, to provide 2³-(3-fluoro-5-methylphenyl)-6-(hydroxymethyl)-4-oxa-9-aza-2(4,6)-quinolina-1(1.4)-piperidina-3(1,2)-benacyclononaphane-33-carbonitrile (compound 88, 21.1 mg, 0.039 mmol) and 2³-(3-fluoro-5-methylphenyl)-6-(hydroxymethyl)-4-oxa-9-aza-2(4,6)-quinolina-1(1,4)-piperidina-3(1,2)-benacyclononaphane-33-carbonitrile (compound 89, 4.4 mg, 0.008 mmol) as yellow solid. The stereochemistry is arbitrarily defined.

Compounds 88-93 were synthesized according to the 6-step procedure described in Example 12. Analytical data for compounds 88-93 are provided below.

| Cpd. | | |
|---|---|---|
| No. | Structure | Analytical Data |
| 88* | 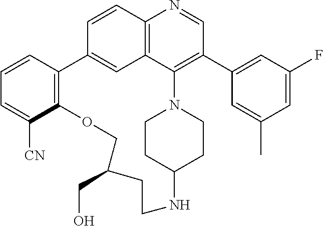 | 1H NMR (400 MHz, DMSO-d6): δ 8.70 (d, J = 6.3 Hz, 1H), 8.48 (d, J = 3.6 Hz, 1H), 8.22 (dd, J = 8.7, 3.0 Hz, 1H), 8.10 (d, J = 8.9 Hz, 1H), 7.84 (d, J = 7.8 Hz, 1H), 7.72 (d, J = 6.0 Hz, 1H), 7.50 (td, J = 7.8, 4.3 Hz, 1H), 7.09 (dd, J = 29.2, 12.7 Hz, 3H), 3.82 (d, J = 8.5 Hz, 2H), 3.62 (dd, J = 48.0, 38.4 Hz, 5H), 3.26-2.90 (m, 4H), 2.48 (d, J = 2.5 Hz, 3H), 2.01 (dd, J = 109.9, 48.3 Hz, 7H). MS (m/z) 538 (M + H)⁺. |
| 89* | 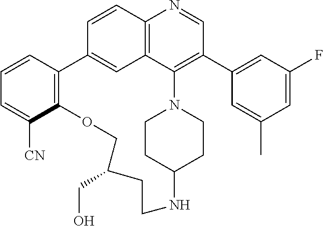 | 1H NMR (400 MHz, DMSO-d6): δ 8.65 (s, 1H), 8.32 (s, 1H), 8.16 (s, 1H), 8.07 (d, J = 9.6 Hz, 1H), 7.81 (s, 2H), 7.47 (t, J = 7.7 Hz, 1H), 7.07 (s, 2H), 6.99 (d, J = 8.8 Hz, 1H), 3.85-3.54 (m, 4H), 3.35 (s, 2H), 3.28-2.89 (m, 5H), 2.46 (s, 3H), 2.11 (s, 2H), 1.80 (s, 5H). MS (m/z) 538 (M + H)⁺. |
| 90 | 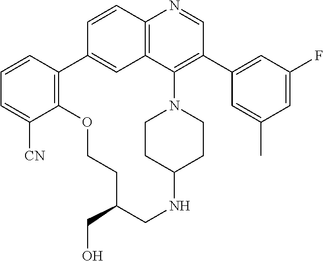 [SMILES: Cc1cc(F)cc(-c2cnc3ccc4cc3c2N2CCC(CC2)NC[C@@H](CO)CCOc2c(C#N)cccc2-4)c1] | 1H NMR (400 MHz, DMSO-d6): δ 8.60-8.48 (m, 2H), 8.17- 1.23 (m, 1H), 7.95-7.90 (m, 2H), 7.77 (d, J = 8.0 Hz, 1H), 7.48 (t, J = 8.0 Hz, 1H), 7.06-6.80 (m, 3H), 3.60-3.40 (m, 4H), 2.95-2.60 (m, 5H), 2.49-2.27 (m, 5H), 2.15-1.30 (m, 7H). MS (m/z) 538 (M + H)⁺. |
| 91* | 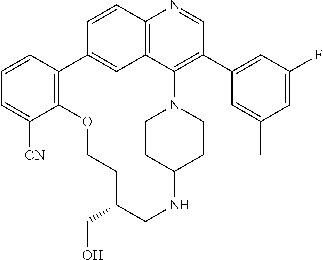 [SMILES: Cc1cc(F)cc(-c2cnc3ccc4cc3c2N2CCC(CC2)NC[C@H](CO)CCOc2c(C#N)cccc2-4)c1] | 1H NMR (400 MHz, DMSO-d6): δ 8.52 (s, 1H), 8.41 (s, 1H), 8.12 (d, J = 8.0 Hz, 1H), 7.83 (brs, 1H), 7.73 (brs, 1H), 7.63 (brs, 1H), 7.45 (t, J = 8.0 Hz, 1H), 7.05 (d, J = 8.0 Hz, 1H), 6.99 (s, 1H), 6.90 (d, J = 8.0 Hz, 1H), 3.82-3.40 (m, 5H), 3.04- 2.62 (m, 6H), 2.43 (s, 3H), 2.25-1.42 (m, 7H). MS (m/z) 538 (M + H)⁺. |
| 92 | 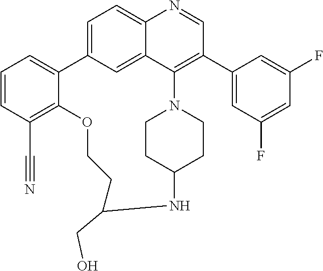 [SMILES: N#Cc1cccc2c1OCCC(CO)NC1CCN(CC1)c1c(-c3cc(F)cc(F)c3)cnc3ccc-2cc13] | ¹H NMR (400 MHz, cd3od) δ 8.72 (s, 1H), 8.55 (s, 1H), 8.24 (d, J = 8.8 Hz, 1H), 8.10 (d, J = 7.2 Hz, 1H), 7.88 (d, J = 5.6 Hz, 1H), 7.68 (s, 1H), 7.53 (t, J = 7.2 Hz, 1H), 7.16 (dd, J = 26.4, 8.8 Hz, 3H), 3.87 (t, J = 44.0 Hz, 8H), 2.85 (d, J = 71.2 Hz, 2H), 2.12 (t, J = 95.6 Hz, 6H). MS (m/z) 527 (M + H)⁺. |
| 93 | 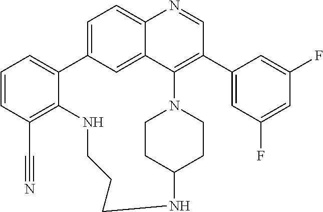 [SMILES: N#Cc1cccc2c1NCCCNC1CCN(CC1)c1c(-c3cc(F)cc(F)c3)cnc3ccc-2cc13] | ¹H NMR (399 MHz, cd₃od) δ 8.55 (s, 1H), 8.42 (s, 1H), 8.13- 8.07 (m, 2H), 7.74 (d, J = 8.0 Hz, 1H), 7.53 (d, J = 7.6 Hz, 1H), 7.41 (d, J = 7.2 Hz, 1H), 7.09 (t, J = 8.4 Hz, 1H), 7.04- 6.91 (m, 3H), 3,21 (s, 3H), 2.76 (dd, J = 36.8, 28.8 Hz, 6H), 1.76 (dt, J = 50.4, 27.6 Hz, 6H). MS (m/z) 496 (M + H)⁺. |

*The stereochemistry is arbitrarily assigned.

### Example 13

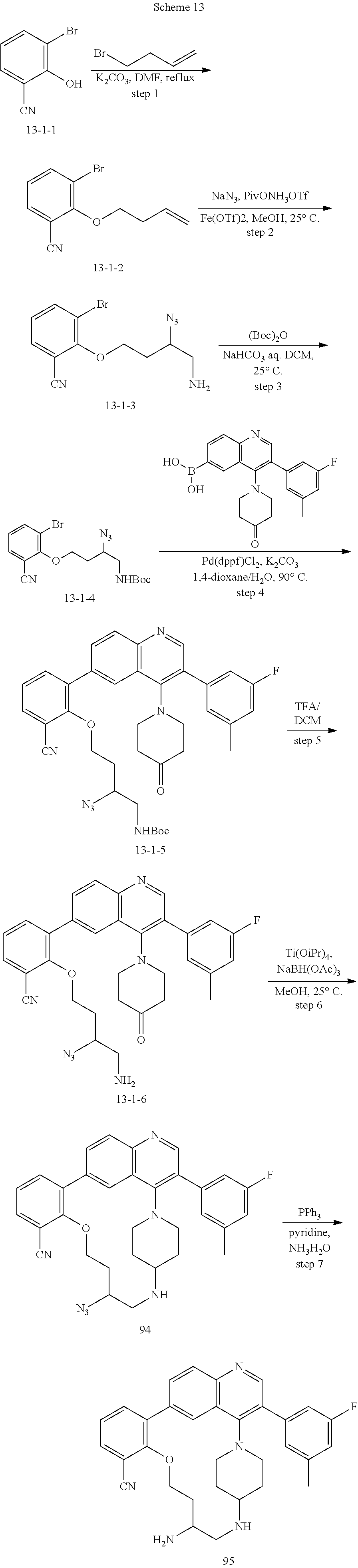

### Step 1: Synthesis of 3-bromo-2-(but-3-en-1-yloxy)benzonitrile (Compound 13-1-2)

3-bromo-2-hydroxybenzonitrile (compound 13-1-1, 5 g, 25.51 mmol, 1.0 equiv) and K₂CO₃ (7 g, 51 mmol, 2.0 equiv) were dissolved in MeCN (50 mL), then 4-bromobut-1-ene (6.8 g, 51 mmol, 2.0 equiv) was added to the solution. The reaction mixture was stirred and heated to 90° C. for 12 h under nitrogen atmosphere. The desired product was detected by LCMS. The reaction mixture was extracted with ethyl acetate (100 mL*3), washed with brine, dried over anhydrous Na₂SO₄. The filtrate was concentrated under vacuum and the residue was purified by FCC (EA:PE=1:2) to afford 3-bromo-2-(but-3-en-1-yloxy)benzonitrile (compound 13-1-2, 1.9 g, 757 mmol, 29.6%) as white oil. MS (m/z) 250 (M−1)⁻.

### Step 2: Synthesis of 2-(4-amino-3-azidobutoxy)-3-bromobenzonitrile (Compound 13-1-3)

3-bromo-2-(but-3-en-1-yloxy)benzonitrile (compound 13-1-2, 1 g, 4.0 mmol, 1.0 equiv) and Fe(OTf)₂ (107 mg, 0.4 mmol, 0.1 equiv) were dissolved in MeOH (10 mL), then PivONH₃OTf (2.67 g, 10 mmol, 2.5 equiv) and NaN₃ (312 mg, 4.8 mmol, 1.2 equiv) were added to the solution under nitrogen atmosphere. The reaction mixture was stirred at room temperature for 12 h under nitrogen atmosphere. The desired product was detected by LCMS. The reaction solution was concentrated under vacuum to afford crude product 2-(4-amino-3-azidobutoxy)-3-bromobenzonitrile (compound 13-1-3) which was for next step. MS (m/z) 310 (M+H)⁺.

### Step 3: Synthesis of tert-butyl (2-azido-4-(2-bromo-6-cyanophenoxy)butyl)carbamate (Compound 13-14)

2-(4-amino-3-azidobutoxy)-3-bromobenzonitrile (compound 13-1-3, 1 g, 4.0 mmol, 1.0 equiv) was dissolved in the solution of DCM (5 mL):NaHCO₃(aq) (5 mL)=1:1, then (Boc)₂O (1.7 g, 8 mmol, 2.0 equiv) was added to the solution. The reaction mixture was stirred at 25° C. for 12 h. The desired product was detected by LCMS. The reaction mixture was extracted with DCM (50 mL*3), washed with brine, dried over anhydrous Na₂SO₄. The filtrate was concentrated under vacuum to afford crude product tert-butyl (2-azido-4-(2-bromo-6-cyanophenoxy)butyl)carbamate (compound 13-14, 1.2 g 3.0 mmol, 75%). MS (m/z) 310/354/356 (M−100/M−56/M−56+2)⁺

### Step 4: Synthesis of tert-butyl (2-azido-4-(2-cyano-6-(3-(3-fluoro-5-methylphenyl)-4-(4-oxopiperidin-1-yl)quinolin-6-yl)phenoxy)butyl)carbamate (Compound 13-1-5)

Tert-butyl(2-azido-4-(2-bromo-6-cyanophenoxy)butyl)carbamate(compound 13-1-4, 400 mg, 1.0 mmol, 1.0 equiv). (3-(3-fluoro-5-methylphenyl)-4-(4-oxopiperidin-1-yl)quinolin-6-yl)boronic acid (378 mmol, 1.0 equiv) and K₂CO₃(196 mg, 1.5 mmol, 1.5 equiv) were dissolved in the solution of 1,4-dioxane(12 mL): H₂O(3 mL)=1:4, then Pd(dppf)Cl₂ (73.01 mg, 0.1 mmol, 0.1 equiv) was added to the solution. The reaction mixture was stirred heated to 90° C. for 2 h under nitrogen atmosphere. The desired product was detected by LCMS. The reaction solution was extracted with ethyl acetate (30 mL*3), washed with brine and dried over anhydrous Na₂SO₄. The filtrate was concentrated under vacuum and the residue was purified by FCC(PE:EA=2:1) to afford tert-butyl (2-azido-4-(2-cyano-6-(3-(3-fluoro-5-methylphenyl)-4-(4-oxopiperidin-1-yl)quinolin-6-yl)phenoxy)butyl)carbamate (compound 13-1-5, 230 mg, 0.347 mmol, 34.7%) as yellow solid. MS (m/z) 664.45 (M+H)⁺.

### Step 5: Synthesis of 2-(4-amino-3-azidobutoxy)-3-(3-(3-fluoro-5-methylphenyl)-4-(4-oxopiperidin-1-yl)quinolin-6-yl)benzonitrile (Compound 13-1-6)

Tert-butyl-(2-azido-4-(2-cyano-6-(3-(3-fluoro-5-methylphenyl)-4-(4-oxopiperidin-1-yl)quinolin-6-yl)phenoxy)butyl)carbamatee (compound 13-1-5.230 mg, 0.347 mmol, 1.0 equiv) was dissolved in the solution of TFA(1 mL):DCM(3 mL)=1:3. The reaction solution was stirred at 25° C. for 1 h. The desired product was detected by LCMS. The reaction solution was concentrated under vacuum to afford crude product 2-(4-amino-3-azidobutoxy)-3-(3-(3-fluoro-5-methylphenyl)-4-(4-oxopiperidin-1-yl)quinolin-6-yl)benzonitrile (compound 13-1-6, 195 mg, 0.347 mmol, 100%). MS (m/z) 564.4 (M+H)⁺.

### Step 6: Synthesis of Compound 94

2-(4-amino-3-azidobutoxy)-3-(3-(3-fluoro-5-methylphenyl)-4-(4-oxopiperidin-1-yl)quinolin-6-yl)benzonitrile (compound 13-1-6, 195 mg, 0.347 mmol, 1.0 equiv) was dissolved in MeOH (4 mL). Then Ti(OiPr)₄ (497 mg, 1.75 mmol, 5 equiv) was added to the solution under nitrogen atmosphere. The reaction mixture was stirred at 25° C. for 30 min. Then NaBH(OAc)₃(742 mg, 3.5 mmol, 10 equiv) was added to the solution. The reaction mixture was stirred at 25° C. for 12 h. The desired product was detected by LCMS. The reaction mixture was filtered through celite and the filtrate was concentrated under vacuum to afford crude product. The crude product was purified by pre-HPLC (0.1% TFA in water) to afford 7-azido-23-(3-fluoro-5-methylphenyl)-4-oxa-9-aza-2(4.6)-quinolina-1(1.4)-piperidina-3(1,2)-benzenacyclononaphane-33-carbonitrile (compound 94, 2.0 mg, 0.00366 mmol, 1.0%) as yellow solid.

### Step 7: Synthesis of Compound 95

7-azido-23-(3-fluoro-5-methylphenyl)-4-oxa-9-aza-2(4,6)-quinolina-1(1,4)-piperidina-3(1,2)-benzenacyclononaphane-3³-carbonitrile (compound 13-1, 10 mg, 0.0183 mmol, 1.0 equiv) and PPh3(7 mg, 0.022 mmol, 1.5 equiv) were dissolved in pyridine (2 mL). The reaction mixture was stirred at 25° C. for 30 mm under nitrogen atmosphere. Then NH₃H2O (2 mL) was added to the solution. The reaction mixture was stirred at 25° C. for 12 h under nitrogen atmosphere. The desired product was detected by LCMS. The reaction solution was concentrated under vacuum, and the residue was purified by Pre-HPL (0.1% TFA in water) to afford 7-amino-23-(3-fluoro-5-methylphenyl)-4-oxa-9-aza-2(4,6)-quinolina-1(1,4)-piperidina-3(1.2)-benzenacyclononaphane-33-carbonitrile (compound 95, 1.0 mg, 0.00192 mmol, 10%) as white solid.

Compounds 94-103 were synthesized according to the 6-step procedure described in Example 13. Analytical data for compounds 94-103 are provided below.

| Cpd. | | |
|---|---|---|
| No. | Structure | Analytical Data |
| 94 | 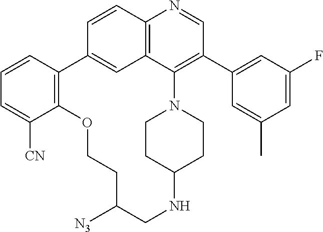 [SMILES: Cc1cc(F)cc(-c2cnc3ccc4cc3c2N2CCC(CC2)NCC(N=[N+]=[N-])CCOc2c(C#N)cccc2-4)c1] | ¹H NMR (400 MHz, cd3od) δ 8.69 (s, 1H), 8.56-8.50 (m, 1H), 8.23 (d, J = 8.4 Hz, 1H), 8.12-8.06 (m, 1H), 7.86 (dd, J = 6.0, 4.6 Hz, 1H), 7.59 (dd, J = 10.8, 8.8 Hz, 1H), 7.49 (d, J = 6.4 Hz, 1H), 7.15-7.02 (m, 3H), 4.22-4.11 (m, 1H), 4.03- 3.76 (m, 3H), 3.52 (dd, J = 29.2, 14.8 Hz, 3H), 3.24-3.07 (m, 2H), 3.00-2.85 (m, 1H), 2.48 (s, 3H), 2.24-1.89 (m, 6H). MS (m/z) 548.7 (M + H)⁺. |
| 95 | 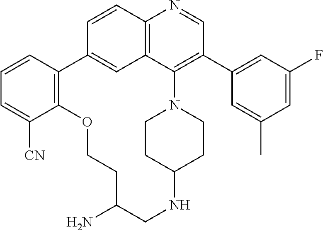 [SMILES: Cc1cc(F)cc(-c2cnc3ccc4cc3c2N2CCC(CC2)NCC(N)CCOc2c(C#N)cccc2-4)c1] | ¹H NMR (400 MHz, cd3od) δ 8.63 (d, J = 2.4 Hz, 1H), 8.51 (d, J = 15.6 Hz, 1H), 8.18 (d, J = 9.2 Hz, 1H), 8.06 (d, J = 8.4 Hz, 1H), 7.84 (d, J = 7.6 Hz, 1H), 7.75 (dd, J = 10.0, 5.2 Hz, 1H), 7.50 (dd, J = 15.2, 5.2 Hz, 1H), 7.12 (dd, J = 18.4, 10.0 Hz, 2H), 7.01 (d, J = 9.2 Hz, 1H), 3.98-3.38 (m, 7H), 3.16 2.81 (m, 3H), 2.47 (d, J = 4.0 Hz, 3H), 2.16-1.80 (m, 6H). MS (m/z) 522.65 (M + H)⁺. |
| 96* | 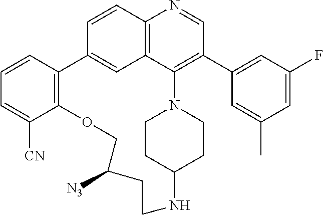 [SMILES: Cc1cc(F)cc(-c2cnc3ccc4cc3c2N2CCC(CC2)NCC[C@@H](N=[N+]=[N-])COc2c(C#N)cccc2-4)c1] | 1H NMR (400 MHz, DMSO-d6): δ 8.51-8.35 (m, 1H), 8.31- 8.15 (m, 1H), 8.05 (d, J = 8.8 Hz, 1H), 7.79 (dd, J = 13.7, 6.3 Hz, 3H), 7.38 (t, J = 7.7 Hz, 1H), 7.07 (d, J = 8.5 Hz, 1H), 6.99- 6.79 (m, 2H), 3.68 (s, 3H), 3.09-2.65 (m, 2H), 2.40-2.03 (m, 8H), 1.55-1.11 (m, 6H). MS (m/z) 549 (M + H)⁺. |
| 97* | 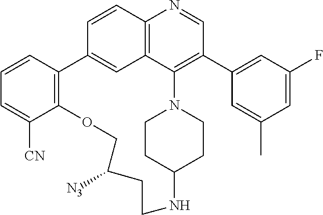 [SMILES: Cc1cc(F)cc(-c2cnc3ccc4cc3c2N2CCC(CC2)NCC[C@H](N=[N+]=[N-])COc2c(C#N)cccc2-4)c1] | 1H NMR (400 MHz, DMSO-d6): δ 8.46 (s, 1H), 8.29-8.23 (m, 1H), 8.05 (d, J = 8.8 Hz, 1H), 7.82 (dd, J = 13.7, 6.3 Hz, 3H), 7.40 (t, J = 7.7 Hz, 1H), 7.07 (d, J = 8.5 Hz, 1H), 6.88-6.75 (m, 2H), 3.76-3.70 (m, 3H), 3.23-3.04 (m, 2H), 2.40-2.23 (m, 8H), 1.76-1.21 (m, 6H). MS (m/z) 549 (M + H)⁺. |
| 98* | 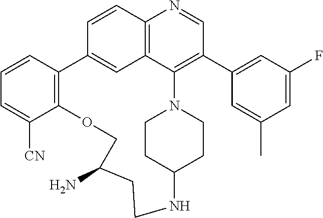 [SMILES: Cc1cc(F)cc(-c2cnc3ccc4cc3c2N2CCC(CC2)NCC[C@@H](N)COc2c(C#N)cccc2-4)c1] | 1H NMR (400 MHz, cd3od) δ 8.60 (s, 1H), 8.23 (d, J = 0.8 Hz, 1H), 8.12 (s, 1H), 8.04-7.99 (m, 1H), 7.78 (ddd, J = 19.2, 7.6, 1.2 Hz, 2H), 7.47 (t, J = 8.0 Hz, 1H), 7.14-7.00 (m, 2H), 6.92 (d, J = 10.0 Hz, 1H), 3.92 (s, 2H), 3.66 (d, J = 21.2 Hz, 2H), 3.42 (s, 1H), 2.97 (t, J = 24.8 Hz, 4H), 2.47 (s, 3H), 1.98 (dd, J = 95.6, 48.0 Hz, 6H). MS (m/z) 522.75 (M + H)⁺. |
| 99* | 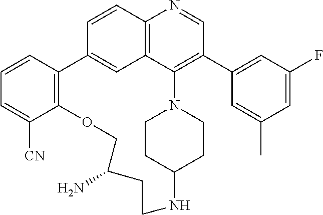 [SMILES: Cc1cc(F)cc(-c2cnc3ccc4cc3c2N2CCC(CC2)NCC[C@H](N)COc2c(C#N)cccc2-4)c1] | ¹H NMR (400 MHz, cd3od) δ 8.65 (s, 1H), 8.25 (s, 1H), 8.15 (d, J = 8.8 Hz, 1H), 8.04 (d, J = 8.4 Hz, 1H), 7.84-7.73 (m, 2H), 7.47 (t, J = 7.6 Hz, 1H), 7.10 (d, J = 9.6 Hz, 1H), 7.04 (s, 1H), 6.94 (d, J = 8.0 Hz, 1H), 3.90 (dd, J = 10.4, 5.2 Hz, 2H), 3.73-3.35 (m, 4H), 2.99 (dd, J = 30.4, 14.8 Hz, 4H), 2.47 (s, 3H), 1.98 (dd, J = 92.4, 60.4 Hz, 6H). MS (m/z) 522.65 (M + H)⁺. |
| 100* | 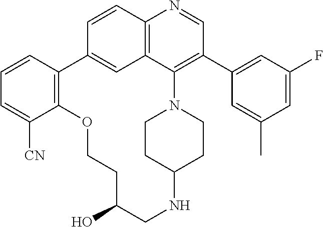 [SMILES: Cc1cc(F)cc(-c2cnc3ccc4cc3c2N2CCC(CC2)NC[C@@H](O)CCOc2c(C#N)cccc2-4)c1] | 1H NMR (400 MHz, DMSO-d6): δ 8.77 (s, 1H), 8.71 (s, 1H), 8.21 (t, J = 8.7 Hz, 2H), 7.97 (d, J = 7.8 Hz, 1H), 7.69 (d, J = 7.8 Hz, 1H), 7.51 (t, J = 7.8 Hz, 1H), 7.17-7.00 (m, 3H), 4.42 (s, 1H), 4.12 (d, J = 24.6 Hz, 2H), 3.81 (s, 1H), 3.44 (d, J = 12.7 Hz, 3H), 3.19 (d, J = 11.8 Hz, 2H), 2.74 (s, 1H), 2.49 (s, 3H), 2.32 (s, 1H), 2.16(d, J = 12.8 Hz, 2H), 1.92 (s, 2H), 1.68 (s, 1H). MS (m/z) 523 (M + H)⁺. |
| 101* | 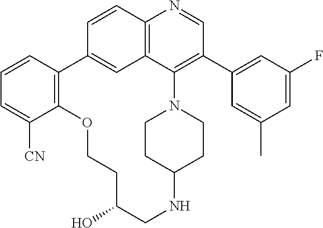 [SMILES: Cc1cc(F)cc(-c2cnc3ccc4cc3c2N2CCC(CC2)NC[C@H](O)CCOc2c(C#N)cccc2-4)c1] | 1H NMR (400 MHz, DMSO-d6): δ 8.79 (s, 1H), 8.72 (s, 1H), 8.21 (d, J = 3.0 Hz, 2H), 7.98 (d, J = 7.9 Hz, 1H), 7.69 (d, J = 7.7 Hz, 1H), 7.51 (t, J = 7.8 Hz, 1H), 7.18-6.99 (m, 3H), 4.43 (s, 1H), 4.25-4.04 (m, 2H), 3.80 (d, J = 10.7 Hz, 1H), 3.54- 3.38 (m, 3H), 3.22 (s, 2H), 2.75 (t, J = 12.6 Hz, 1H), 2.49 (s, 3H), 2.17 (dd, J = 15.1, 4.6 Hz, 2H), 1.93 (s, 2H). MS (m/z) 523 (M + H)⁺. |
| 102* | 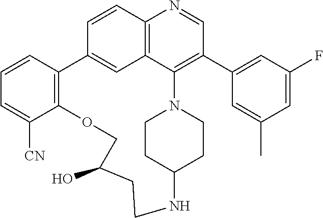 [SMILES: Cc1cc(F)cc(-c2cnc3ccc4cc3c2N2CCC(CC2)NCC[C@@H](O)COc2c(C#N)cccc2-4)c1] | 1H NMR (400 MHz, CD₃OD-d4): δ 8.71-8.61 (m, 2H), 8.20 (d, J = 8.8 Hz, 1H), 8.11 (d, J = 9.4 Hz, 1H), 7.78 (d, J = 7.8 Hz, 1H), 7.50 (t, J = 7.8 Hz, 1H), 7.12 (d, J = 17.3 Hz, 2H), 7.04 (d, J = 9.1 Hz, 1H), 3.75 (d, J = 48.1 Hz, 5H), 3.40 (s, 1H), 3.24 (s, 2H), 3,04 (dd, J = 27.8, 13.3 Hz, 2H), 2.48 (s, 3H), 2.13 (s, 4H), 1.94 (s, 2H). MS (m/z) 523 (M + H)⁺. |
| 103* | 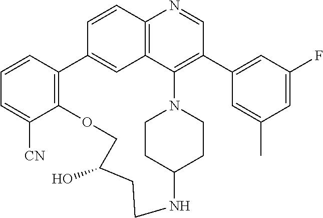 [SMILES: Cc1cc(F)cc(-c2cnc3ccc4cc3c2N2CCC(CC2)NCC[C@H](O)COc2c(C#N)cccc2-4)c1] | 1H NMR (400 MHz, CD₃OD-d4): δ 8.67 (s, 1H), 8.46 (d, J = 1.8 Hz, 1H), 8.18 (d, J = 8.7 Hz, 1H), 8.10 (d, J = 9.0 Hz, 1H), 7.82 (ddd, J = 13.3, 7.8, 1.7 Hz, 2H), 7.48 (t, J = 7.8 Hz, 1H), 7.17-7.05 (m, 2H), 7.02 (d, J = 9.0 Hz, 1H), 3.89-3.75 (m, 2H), 3.66 (t, J = 5.8 Hz, 3H), 3.37-3.27 (m, 1H), 3.19 (t, J = 7.4 Hz, 2H), 2.98 (t, J = 13.1 Hz, 2H), 2.47 (s, 3H), 2.16 (s, 2 H), 2.03 (s, 1H), 1.99-1.88 (m, 3H). MS (m/z) 523 (M + H)⁺. |

*The stereochemistry is arbitrarily assigned.

### Example 14

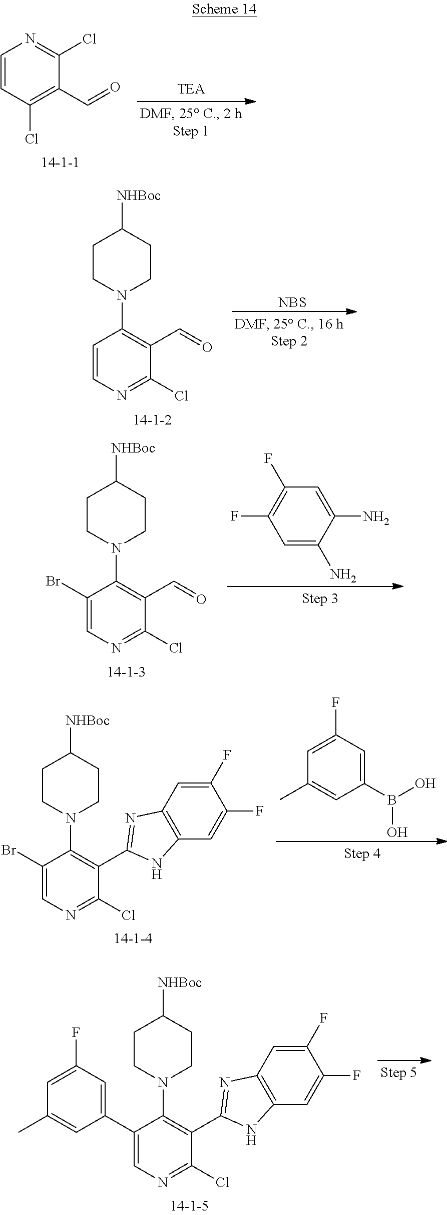

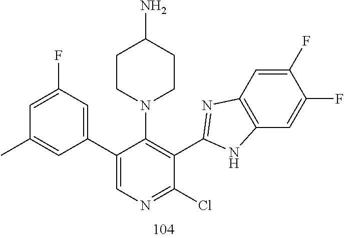 [SMILES: Cc1cc(F)cc(-c2cnc(Cl)c(-c3nc4cc(F)c(F)cc4[nH]3)c2N2CCC(N)CC2)c1]

### Step 1: Synthesis of (tert-butyl N-[1-(2-chloro-3-formyl-4-pyridyl)-4-piperidyl]carbamate (Compound 14-1-2)

To a stirred mixture of compound 14-1-1 (10 g, 56.82 mmol) and tert-butyl N-(4-piperidyl)carbamate (11.38 g, 56.82 mmol) in DMF (100 mL) was added DIPEA (36.65 g, 284.09 mmol) at 25° C. for 2 hr. The reaction was monitored by LCMS, which showed the reaction was completed. The mixture was diluted with water (60 mL), then lots of solid was precipitated out of the reaction solution. Filter to collect the filter cake, which was dissolved with ethyl acetate, followed by dryness over anhydrous sodium sulfate, filter and concentration in vacuo to get the compound 14-1-2 (17.50 g, 51.50 mmol, 90.64% yield) as a yellow solid. MS (m/z) 340 (M+H)⁻.

### Step 2: Synthesis of tert-butyl N-[1-(5-bromo-2-chloro-3-formyl-4-pyridyl)-4-piperidyl]carbamate (Compound 14-1-3)

A solution of compound 14-1-2 (17.5 g, 51.50 mmol) in DMF (100 mL) was added NBS (9.17 g, 51.50 mmol) at 25° C., then the resulting mixture was stirred at 25° C. for 16 hr. The reaction was monitored by LCMS, which showed the reaction was completed. The mixture was diluted with water (20 mL), washed with ethyl acetate (3*20 mL), the organic phase was separated, dried over anhydrous sodium sulfate, filtered and concentrated in vacuo to get the compound 14-1-3 (7.00 g, crude) as a yellow solid. MS (m/z) 420 (M+H)⁺.

### Step 3: Synthesis of tert-butyl N-[1-[5-bromo-2-chloro-3-(5,6-difluoro-1H-benzimidazol-2-yl)-4-pyridyl]-4-piperidyl]carbamate (Compound 14-14)

To a solution of compound 14-1-3 (7.0 g, 16.72 mmol) and 4,5-difluorobenzene-1,2-diamine (2.41 g, 16.72 mmol) in tetrahydrofuran (70 mL) was added acetic acid (3.01 g, 50.15 mmol), then the mixture was stirred at 60° C. for 2 hr, then FeCl_{b}(8.14 g, 50.15 mmol) was added at 25° C., the resulting mixture was then stirred at 60° C. for 2 hr. The reaction was monitored by LCMS and TLC, which showed the reaction was completed. The reaction mixture was diluted with water (50 mL). Then it was extracted with ethyl acetate (50 mL*2). Combined all organic phases and dried over anhydrous Na₂SO₄. The organic phase was concentrated to obtain compound 14-14 (6.70 g, crude) as a brown solid. MS (m/z) 544 (M+H)⁺.

### Step 4: Synthesis of tert-butyl N-[1-[2-chloro-3-(5,6-difluoro-1H-benzimidazol-2-yl)-5-(3-fluoro-5-methyl-phenyl)-4-pyridyl]-4-piperidyl]carbamate (Compound 14-1-5)

To a solution of compound 14-14 (6.7 g, 12.34 mmol) and (3-fluoro-5-methyl-phenyl)boronic acid (1.71 g, 11.11 mmol) in 1,4-dioxane (40 mL) and water (10 mL) was added NaHCO₃ (3.11 g, 37.03 mmol) and PdCl2(dppf) (905.65 mg, 1.23 mmol) at 25° C., the resulting mixture was then stirred at 90° C. for 3 hr in N₂. The reaction was monitored by LCMS, which showed the reaction was completed. The mixture was diluted with water (50 mL), and extracted with ethyl acetate (3*50 mL). The organic phase was separated, dried over anhydrous sodium sulfate, filtered and concentrated in vacuo. The crude product was purified by silica gel column chromatography (EA/PE=20%-50%) to afford compound 14-1-5 (2.05 g, 3.58 mmol, 28.96% yield) as a brown solid. MS (m/z) 572.2 (M+H)⁺.

### Step 5: Synthesis of 1-(2-chloro-3-(5,6-difluoro-1H-benzo[d]imidazol-2-yl)-5-(3-fluoro-5-methylphenyl)pyridin-4-yl)piperidin-4-amine (Compound 104)

To a stirred solution of compound 14-1-5 (100 mg, 174.82 μmol) in DCM (5 mL) was added TFA (199.33 mg, 1.75 mmol) at 25° C., the mixture was stirred at room temperature for 16 hr. The reaction was monitored by LCMS, which showed the reaction was completed. The mixture was concentrated in vacuo, the residue was dissolved in methanol (3 mL), treated with NaHCO₃(solid) to adjust to pH 8, then filtered. The filtrate was concentrated in vacuo. The soiled was purified by Prep-HPLC. Lyophilized to afford desired product compound 104 (11 mg, 23.31 μmol, 13.33% yield) as a white solid.

Analytical data for compound 104 is provided below

| Cpd. | | |
|---|---|---|
| No. | Structure | Analytical Data |
| 104 | 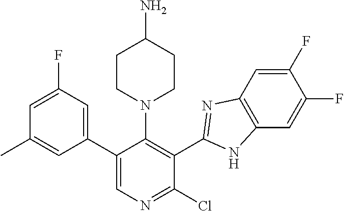 [SMILES: Cc1cc(F)cc(-c2cnc(Cl)c(-c3nc4cc(F)c(F)cc4[nH]3)c2N2CCC(N)CC2)c1] | 1H NMR (400 MHz, DMSO-d6) δ 8.12 (s, 1H), 7.71-7.66 (m, 2H), 7.13-7.05(m, 3H), 2.85 (d, J = 1.6 Hz, 2H), 2.35 (s, 3H), 2.29-2.25 (m, 1H), 2.16 (t, J = 1.2 Hz, 2H), 1.17 (d, J = 1.2 Hz, 2H), 0.70-062(m, 2H). MS (m/z) 472.40 (M + H)⁺. |

### Example 15

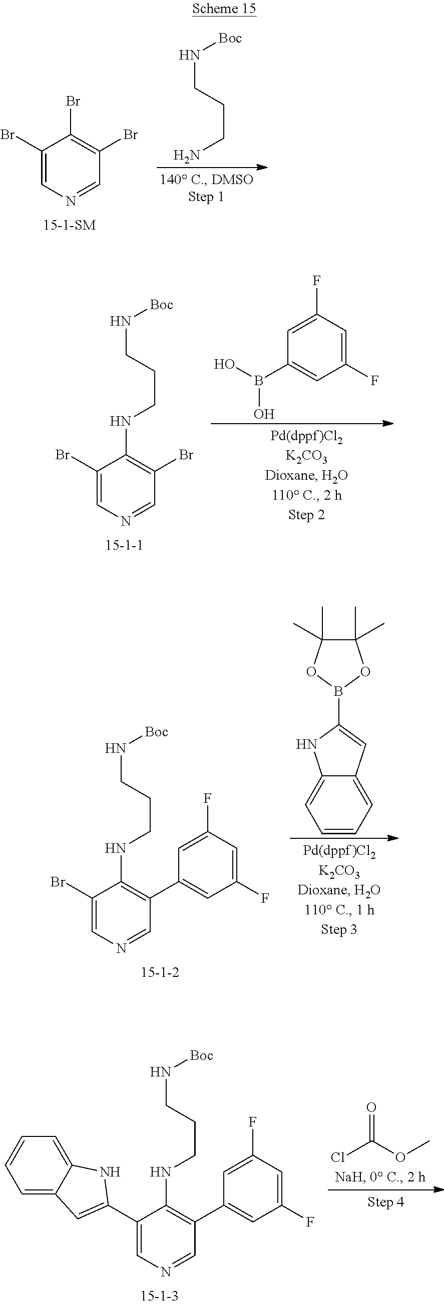

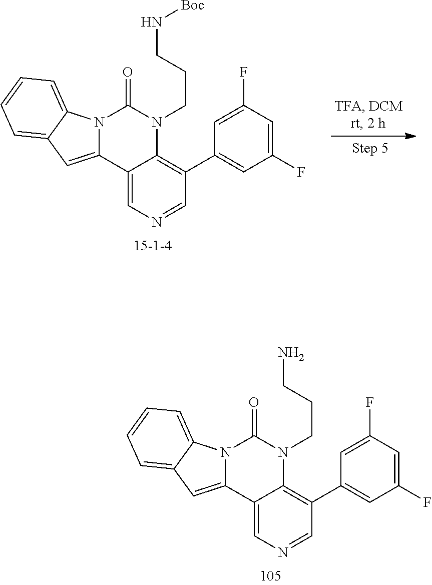 [SMILES: CC(C)(C)OC(=O)NCCCn1c(=O)n2c3ccccc3cc2c2cncc(-c3cc(F)cc(F)c3)c21 ; NCCCn1c(=O)n2c3ccccc3cc2c2cncc(-c3cc(F)cc(F)c3)c21]

### Step 1: Synthesis of tert-butyl (3-((3,5-dibromopyridin-4-yl)amino)propyl)carbamate (Compound 15-1-1)

A solution of 3,4,5-tribromopyridine (compound 15-1-SM, 350 mg, 1.11 mmol) and tert-butyl N-(3-aminopropyl)carbamate (772.47 mg, 4.43 mmol) in DMSO (0.05 mL) was stirred at 140° C. for 30 min. The progress of the reaction was monitored by LCMS. Cooled to room temperature and poured into ice-water (25 mL), and lots of solid was observed. Collect solid, which was then was washed with H₂O (10 mL*3), dried under vacuum to afford title compound (compound 15-1-1, 400.00 mg, 977.72 μmol, 88.21% yield) as a white solid. MS (m/z) 408.0 (M+H)⁻.

### Step 2: Synthesis of tert-butyl (3-((3-bromo-5-(1H-indol-2-yl)pyridin-4-yl)amino)propyl)carbamate (Compound 15-1-2)

To a solution of compound 15-1-1 (350 mg, 855.50 μmol) and (3,5-difluorophenyl)boronic acid (121.58 mg, 769.95 μmol) in mixed solvent of dioxane (6 mL) and water (0.6 mL) was added [1,1′-Bis(diphenylphosphino)ferrocene]dichlompalladium(II) (94.15 mg, 128.33 μmol) and K₂CO (181.35 mg, 1.71 mmol) at 25° C., the resulting mixture was degassed completely and stirred at 110° C. for 2 hr under N₂ condition. The progress of the reaction was monitored by LCMS. The reaction was cooled to 25° C. diluted with EtOAc and water. The mixture was separated then water phase was extracted with EtOAc twice. The combined organic phase was washed by brine, dried over Na₂SO₄, filtered and concentrated to give the residue, which was purified by silica gel chromatography eluted with DCM:MeOH=8:1 to give title compound (compound 15-1-2, 320.00 mg, 723.50 μmol, 84.57% yield) as yellow oil. MS (m/z) 442.2 (M+H)⁺.

### Step 3: Synthesis of tert-butyl (3-((3-(3,5-difluorophenyl)-5-(1H-indol-2-yl)pyridin-4-yl)amino)propyl)carbamate (Compound 15-1-3)

To a solution of compound 15-1-2(240 mg, 542.62 μmol), 2-(4,4,5,5-tetramethyl-1,3,2-dioxaborolan-2-yl)-1H-indole (240 mg, 651.15 pol) in mixed solvent of dioxane (3 mL) and water (0.3 mL) was added [1,1′-Bis(diphenylphosphino)ferrocene]dichloropalladium(II) (79.63 mg, 108.52 μmol) and K₂CO₃ (149.99 mg, 1.09 mmol), then the resulting mixture was stirred at 110° C. for 2 hr under N₂ condition. The progress of the reaction was monitored by LCMS. The reaction was cooled to 25° C., diluted with EtOAc and water. The mixture was separated then water phase was extracted with EtOAc twice. The combined organic phase was washed by brine, dried over Na₂SO₄, filtered and concentrated to give the residue, which was purified by silica gel chromatography eluted with PE:EtOAc=1:1 to give title compound (compound 15-1-3, 150.00 mg, 313.46 μmol, 57.77% yield) as yellow solid. MS (m/z) 479.1 (M+H)⁻.

### Step 4: Synthesis of tert-butyl (3-(4-(3,5-difluorophenyl)-6-oxopyrido[4′,3′:4,5]pyrimido[1,6-a]indol-5(6H)-yl)propyl)carbamate (Compound 15-1-4)

To a solution of compound 15-1-3 (75 mg, 145.77 μmol) in DMF (2 mL) was added sodium hydride (17.49 mg, 437.31 μmol, 60% purity) at 0° C., the resulting mixture was then stirred at 0° C. for 10 min, methyl carbonochloridate (42 mg, 437.31 μmol) was added. The reaction solution was stirred at 25° C. overnight. The reaction mixture was quenched with 3 mL of saturated aqueous NH₄Cl. The resulting solution was extracted with EtOAc (10 mL×2). The combined organic layers were washed with brine (10 ml), dried over Na₂SO₄, then filtered and concentrated under reduced pressure to give the residue, which was purified by silica gel chromatography eluted with PE:EtOAc=10:1 to give title compound 15-1-4 (30.00 mg, 55.50 μmol, 38.08% yield) as yellow solid. MS (m/z) 505.4 (M+H)⁺.

### Step 5: Synthesis of 5-(3-aminopropyl)-4-(3,5-difluorophenyl)pyrido[4′,3′:4,5]pyrimido [1,6-a]indol-6(5H)-one (Compound 105)

To a solution of compound 15-1-4 (30 mg, 59.46 μmol) in DCM (1.5 mL) was added TFA (1.25 g, 10.92 mmol, 0.5 mL) at 25° C. then the resulting mixture was stirred at 25° C. for 10 min. The progress of the reaction was monitored by LCMS. It worked well. Concentrated to remove solvent and TFA to dryness. Diluted with MeOH and adjusted pH=7 by using Na₂CO₃ (solid). The solution was purified by Prep-HPLC give title compound 105 (4.00 mg, 7.93 μmol, 13.33% yield) as white solid.

Analytical data for compound 105 is provided below.

| Cpd. No. | Structure | Analytical Data |
|---|---|---|
| 105 | 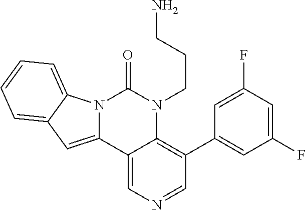 [SMILES: NCCCn1c(=O)n2c3ccccc3cc2c2cncc(-c3cc(F)cc(F)c3)c21] | 1H-NMR (400 MHz, DMSO-D6) δ 9.43-9.41 (m, 1H), 8.53 (d, J = 7.1 Hz, 1H), 8.34 (d, J = 8.0 Hz, 1H), 7.83-7.81 (m, 1H), 7.64 (d, J = 5.2 Hz, 1H), 7.45-7.38 (m, 5H), 3.79- 3.71 (m, 2H), 3.52-3.46 (m, 2H), 2.68-2.65 (m, 1H), 2.34- 2.32 (m, 1H), 2.07 (t, J = 6.9 Hz, 1H), 1.43-1.32 (m, 2H). MS (m/z) 405.2 (M + H)⁺. |

### Example 16

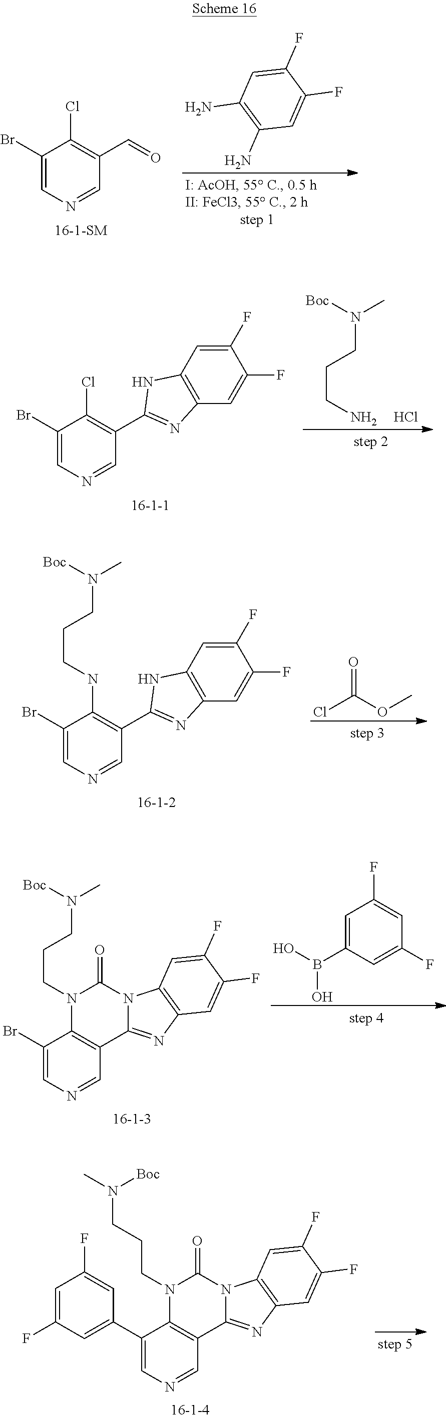

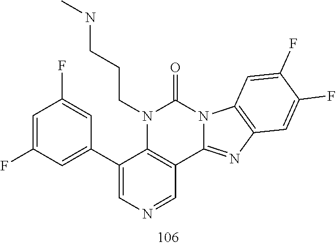 [SMILES: CNCCCn1c(=O)n2c3cc(F)c(F)cc3nc2c2cncc(-c3cc(F)cc(F)c3)c21]

### Step 1: Synthesis of 2-(5-bromo-4-chloropyridin-3-yl)-5,6-difluoro-1H-benzo[d]imidazole (Compound 16-1-1)

A solution of compound 16-1-SM (3 g, 13.61 mmol) and 4,5-difluorobenzene-1,2-diamine (1.96 g, 13.61 mmol) in tetrahydrofuran (10 mL) was added acetic acid (163.44 mg, 2.72 mmol), then stirred for 16 hr in N2 at 25° C. After FeCl₃ (331.10 mg, 2.04 mmol) was added at 55° C., the resulting mixture was stirred at 55° C. for 16 hr. The reaction was monitored by LCMS, which showed the reaction was completed. The resulting mixture was extracted with ethyl acetate (100 mL×3). The organic phases were combined, washed with brine and dried over sodium sulfate. The solvent was removed under reduced pressure and the crude product was purified by silica gel chromatography eluted with PE:EtOAc=1:1 to give the 2-(5-bromo-4-chloro-3-pyridyl)-5,6-difluoro-1H-benzimidazole (Compound 16-1-1, 2.50 g, 5.80 mmol, 42.66% yield, 80% purity) as yellow solid. MS (m/z) 344.0 (M+H)⁺.

### Step 2: Synthesis of tert-butyl (3-(3-bromo-5-(5,6-difluoro-1H-benzo[d]imidazol-2-yl)pyridin-4-yl)amino)propyl)(methyl)carbamate (Compound 16-1-2)

To the solution of compound 16-1-1 (813.37 mg, 2.12 mmol) and tert-butyl N-(3-aminopropyl)-N-methyl-carbamate (400 mg, 2.12 mmol) in N,N-dimethylformamide (3 mL) was added N,N-Diisopropylethylamine (823.77 mg, 6.37 mmol) at 25° C., the resulting mixture was stirred at 90° C. for 4 hr in N2. The reaction was monitored by LCMS, which showed the reaction was completed. The reaction mixture was diluted with ethyl acetate (100 mL). Then it was extracted with water (50 mL*2). The aqueous phase was stripped extracted with ethyl acetate (150 mL). Combined all organic phases and dried over anhydrous Na₂SO₄. Concentrated to move organic phase to obtain a crude product, which was purified by silica gel column (100-200 mesh) chromatography purification eluted with PE:EtOAc=1:1 to give the tert-butyl N-[3-[[3-bromo-5-(5,6-difluoro-1H-benzimidazol-2-yl)-4-pyridyl]amino]propyl]-N-methyl-carbamate (compound 16-1-2, 373.00 mg, 728.95 μmol, 34.31% yield, 97% purity) as yellow Solid. MS (m/z) 496.30 (M+H)⁻.

### Step 3: Synthesis of tert-butyl (3-(4-bromo-9,10-difluoro-6-oxobenzo[4,5]imidazo[1,2-c]pyrido[3,4-e]pyrimidin-5(6H)-yl)propyl)(methyl)carbamate (Compound 16-1-3)

To a solution of compound 16-1-2 (150 mg, 302.21 μmol) in N,N-dimethylformamide (3 mL) and then added sodium hydride (21.76 mg, 906.62 μmol), stirred at 0° C. for 0.5 hr and then added methyl carbonochloridate (57.12 mg, 604.42 μmol) at 0° C., the resulting mixture was stirred at 0° C. for 2 hr in N₂. The reaction was monitored by LCMS, which showed the reaction was completed. The reaction mixture was diluted with ethyl acetate (150 mL). Then it was extracted with water (50 mL*2). The aqueous phase was extracted with ethyl acetate (150 mL). Combined all organic phases and dried over anhydrous Na₂SO₄. Concentrated the organic phase to obtain the residue, the crude product was purified by silicagel column (100-200 mesh) chromatography purification eluted with DCM:MeOH=1:1 to give the tert-butyl (3-(4-bromo-9,10-difluoro-6-oxobenzo[4,5]imidazo[1,2-c]pyrido[3, 4-e]pyrimidin-5(6H)-yl)propyl)(methyl)carbamate (compound 16-1-3, 20.00 mg, 34.46 μmol, 11.40% yield, 90% purity) as yellow Oil. MS (m/z) 522.1 (M+H)⁺.

### Step 4: Synthesis of tert-butyl (3-(4-(3,5-difluorophenyl)-9,10-difluoro-6-oxobenzo[4,5]imidazo[1,2-c]pyrido[3,4-e]pyrimidin-5(6H)-yl)propyl)(methyl)carbamate (Compound 16-14)

A solution of compound 16-1-3 (20 mg, 38.29 μmol) and (3,5-difluoro phenyl)boronic acid (7.86 mg, 49.78 μmol) in 1,4-dioxane (2 mL) and H₂O (0.2 mL) was added [1,1′-Bis(diphenylphosphino)ferrocene]dichloropalladium(II) (5.62 mg, 7.66 μmol) and K₂CO₃ (15.88 mg, 114.87 μmol) at 25° C., the resulting mixture was stirred at 120° C. for 2 hr in N₂. The reaction was monitored by LCMS, which showed the reaction was completed. The reaction mixture was diluted with ethyl acetate (150 mL). Then it was extracted with water (50 mL*2). The aqueous phase was stripped extracted with ethyl acetate (150 mL). Combined all organic phases and dried over anhydrous Na₂SO₄. Concentrated the organic phase to obtain the residue, which was purified by silica gel column (100-200 mesh) chromatography purification eluted with PE:EtOAc=1:3 to give the tert-butyl (3-(4-(3,5-difluorophenyl)-9,10-difluoro-6-oxobenzo[4,5]imidazo[1,2-e]pyrido[3,4-c]pyrimidin-5(6H)-yl)propyl)(methyl)carbamate (compound 16-14, 15.00 mg, 24.30 μmol, 63.47% yield, 90% purity) as yellow oil. MS (m/z) 556.2 (M+H)⁺.

### Step 5: Synthesis of 4-(3,5-difluorophenyl)-9,10-difluoro-5-(3-(methylamino)propyl) benzo[4,5]imidazo[1,2-c]pyrido[3,4-e]pyrimidin-6(5H)-one (Compound 106)

A solution of compound 16-1-4 (15 mg, 27.00 μmol) in dichloromethane (1.5 mL) was added Trifluoroacetic acid (0.5 mL) at 25° C., the resulting mixture was stirred at 25° C. for 0.5 hr. The reaction solution was diluted with 20 mL dichloromethane, the organic phase was concentrated, and the above operation was repeated three times. The crude product was purified by Prep-HPLC (Prep-C18, 5 μM OBD, 19×250 mm, Column, Waters; gradient elution of 30% MeCN in water to 50% MeCN in water over a 10 min period, where both solvents contain 10 mmol/L NH₄HCO₃) to provide compound 106 (4.30 mg, 6.46 μmol, 23.91% yield, 98% purity) as a white solid.

Analytical data for compound 106 is provided below.

| Cpd. | | |
|---|---|---|
| No. | Structure | Analytical Data |
| 106 | 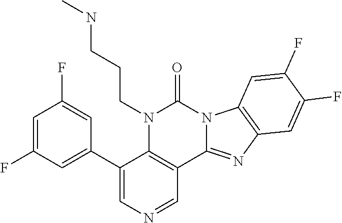 [SMILES: CNCCCn1c(=O)n2c3cc(F)c(F)cc3nc2c2cncc(-c3cc(F)cc(F)c3)c21] | 1H-NMR (400 MHz, DMSO-D6) δ 9.55 (s, 1H), 8.59 (s, 1H), 8.28 (dd, J = 9.9, 7.4 Hz, 1H), 8.11 (dd, J = 10.7, 7.4 Hz, 1H), 7.50-7.36 (m, 3H), 3.83 (t, J = 7.1 Hz, 2H), 3.52 (q, J = 14.9 Hz, 2H), 2.67-2.64 (m, 0H), 2.41 (t, J = 2.6 Hz, 3H), 2.32 (q, J = 1.8 Hz, 0H), 1.67-1.60 (m, 2H). MS (m/z) 456.2 (M + H)⁺. |

### Example 17

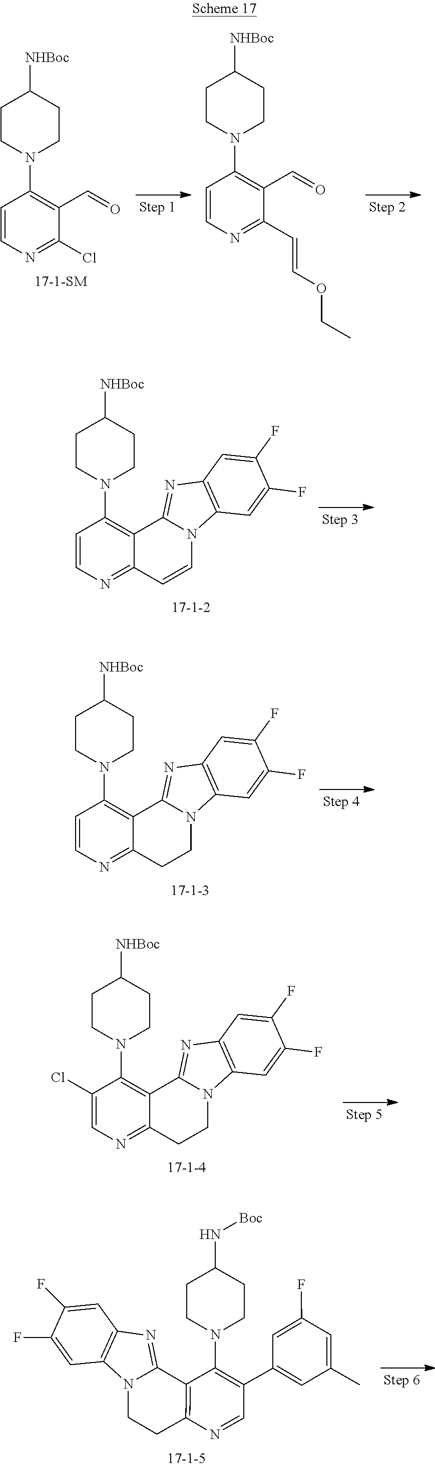 [SMILES: CC(C)(C)OC(=O)NC1CCN(c2ccnc(Cl)c2C=O)CC1 ; CCO/C=C/c1nccc(N2CCC(NC(=O)OC(C)(C)C)CC2)c1C=O ; CC(C)(C)OC(=O)NC1CCN(c2ccnc3ccn4c5cc(F)c(F)cc5nc4c23)CC1 ; CC(C)(C)OC(=O)NC1CCN(c2ccnc3c2-c2nc4cc(F)c(F)cc4n2CC3)CC1 ; CC(C)(C)OC(=O)NC1CCN(c2c(Cl)cnc3c2-c2nc4cc(F)c(F)cc4n2CC3)CC1 ; Cc1cc(F)cc(-c2cnc3c(c2N2CCC(NC(=O)OC(C)(C)C)CC2)-c2nc4cc(F)c(F)cc4n2CC3)c1]

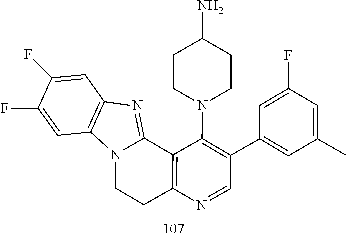 [SMILES: Cc1cc(F)cc(-c2cnc3c(c2N2CCC(N)CC2)-c2nc4cc(F)c(F)cc4n2CC3)c1]

### Step 1: Synthesis of (E)-tert-butyl (1-(2-(2-ethoxyvinyl)-3-formylpyridin-4-yl) piperidin-4-yl) carbamate (Compound 17-1-1)

A solution of compound 17-1-SM (2 g, 5.89 mmol) and 2-[(E)-2-ethoxyvinyl]-4,4,5,5-tetramethyl-1,3,2-dioxaborolane (1.40 g, 7.06 mmol) in 1,4-dioxane (10 mL) was added K₂CO—; (2.44 g, 17.66 mmol) and X-Phos-Pd-G2 (925.21 mg, 1.18 mmol) at 25° C., the resulting mixture was stirred at 100° C. for 5 hr in N₂. The reaction was monitored by LCMS and TLC. LCMS and TLC showed the raw material was disappeared. The reaction mixture was concentrated to dryness and diluted the concentrated reaction mixture with H₂O (100 mL). Then it was extracted with ethyl acetate (300 mL). The combined organic layer was washed with brine (100 mL), dried over Na₂SO₄, then filtered and dried under vacuum to afford (E)-tert-butyl (1-(2-(2-ethoxyvinyl)-3-formylpyridin-4-yl) piperidin-4-yl) carbamate (compound 17-1-1, 3.00 g, crude, 50% purity) as black solid. The crude product used to next step directly without further purification. MS (m/z) 376.2 (M+H)⁺.

### Step 2: Synthesis of tert-butyl (1-(9,10-difluorobenzo [4,5]imidazo[2,1-f][1,6]naphthyridin-1-yl)piperidin-4-yl)carbamate (Compound 17-1-2)

A solution of 4,5-difluorobenzene-1,2-diamine (621.84 mg, 4.31 mmol) and compound 17-1-1 (3 g, 3.60 mmol) in THF (6 mL) was added FeCl₃ (1.79 g, 10.79 mmol, 98% purity) at 25° C., the resulting mixture was stirred at 55° C. for 12 hr in N₂. The reaction was monitored by LCMS. LCMS showed the raw material was disappeared. The reaction mixture was concentrated to dryness and diluted the concentrated reaction mixture with H₂O (100 mL), then extracted with ethyl acetate (100 mL*3). The combined organic layers were washed with brine (100 mL), dried over Na₂SO₄, then filtered and concentrated under reduced pressure. The crude product was purified by silica gel chromatography eluted with DCM:MeOH (from 99/1 to 10/1) to give tert-butyl (1-(9,10-difluorobenzo[4,5]imidazo[2,1-f][1,6]naphthyridin-1-yl) piperidin-4-yl)carbamate (compound 17-1-2, 200.00 mg, 352.82 μmol, 50% purity) as yellow solid. MS (m/z) 454.4 (M+H)⁺.

### Step 3: Synthesis of tert-butyl(1-(9,10-difluorobenzo[4,5]imidazo[2,1-f][1,6]naphthayridin-1-yl) piperidin-4-yl) carbamate (Compound 17-1-3)

To a solution of compound 17-1-2 (200 mg, 352.82 μmol) in MeOH (20 mL) was added Ni (2.07 mg, 35.28 μmol) under N2. The suspension was degassed under vacuum and purged with H₂ several times. The mixture was stirred under H₂ balloon at 50° C. for 1 hr. The reaction was monitored by LCMS. LCMS showed the raw material was disappeared, and product was detected clearly. The reaction mixture was filtered and the precipitated solid was washed with ethyl acetate (10 mL*3) and vacuum dry filtrate to afford compound 17-1-3 (141.00 mg, crude, 80% purity) as a yellow solid. The crude product used to next step directly. MS (m/z) 456.1 (M+H)⁺.

### Step 4: Synthesis of tert-butyl (1-(9,10-difluoro-2-(3-fluoro-5-methylphenyl)-5,6-dihydrobenzo[4,5]imidazo[2,1-f][1,6]naphthyridin-1-yl)piperidin-4-yl)carbamate (Compound 17-14)

To the solution of compound 17-1-3 (72 mg, 158.07 μmol) in N,N-dimethylformamide (1 mL) was added N-chlorosuccinimide (42.21 mg, 316.14 μmol) at 25° C. the resulting mixture was stirred at 40° C. for 1 hr. The reaction was monitored by LCMS. LCMS showed the raw material was disappeared. The reaction mixture was diluted with water (20 mL), then extracted with ethyl acetate (20 mL*2). Combine all organic phases, dried over anhydrous Na₂SO₄, filtered and concentrated to give the residue, which was crude compound 17-14 (66.00 ng, crude, 50% purity) as a yellow solid. MS (m/z) 490.2 (M+H)⁺.

### Step 5: Synthesis of tert-butyl (1-(9,10-difluoro-2-(3-fluoro-5-methylphenyl)-5,6-dihydrobenzo[4,5]imidazo[2,1-f][1,6]naphthyridin-1-yl)piperidin-4-yl)carbamate (Compound 17-1-5)

A solution of compound 17-14 (66 mg, 67.35 μmol) and (3-fluoro-5-methyl-phenyl)boronic acid (12.44 mg, 80.83 μmol) in 1,4-dioxane (1 mL) and H₂O (0.25 mL) was added K₂CO₃ (27.93 mg, 202.06 μmol) and X-Phos-Pd-G2 (10.59 mg, 13.47 μmol) at 25° C., the resulting mixture was stirred at 110° C. for 1 hr in N₂. The reaction was monitored by LCMS. LCMS show the raw material was disappeared. The reaction was extracted with ethyl acetate (10 mL*3). The combined organic layer was washed with brine (10 mL), dried over Na₂SO₄, then filtered and concentrated under reduced pressure. The crude product was purified by silica gel chromatography eluted with PE:EtOAc=5:1 to give compound 17-1-5 (22.00 mg, 37.08 μmol, 55.06% yield, 95% purity) as yellow solid. MS (m/z) 564.3 (M+H)⁺.

### Step 6: Synthesis of 1-(9,10-difluoro-2-(3-fluoro-5-methylphenyl)-5,6-dihydrobenzo [4,5]imidazo[2,1-f][1,6]naphthyridin-1-yl)piperidin-4-amine (Compound 107)

To a solution of compound 17-1-5 (22 mg, 39.03 μmol) in dichloromethane (1 mL) was added trifluoroacetic acid (4.45 mg, 39.03 μmol) at 25° C., the resulting mixture was stirred at 25° C. for 2 hr. The reaction was monitored by TLC. TLC show the raw material was disappeared. The reaction solution was diluted with 20 mL dichloromethane, the organic phase was concentrated, and the above operation was repeated three times. The crude product was purified by Prep-HPLC (Prep-C18.5 μM OBD, 19×250 mm, Column, Waters; gradient elution of 40% MeCN in water to 50% MCCN in water over a 9 min period, where both solvents contain 10 mmol/L NH₄HCO₃) to provide compound 107 (5.00 mg, 9.71 μmol, 24.87% yield, 98.28% purity) as a write solid.

Analytical data for compound 107 is provided below.

| Cpd. | | |
|---|---|---|
| No. | Structure | Analytical Data |
| 107 | 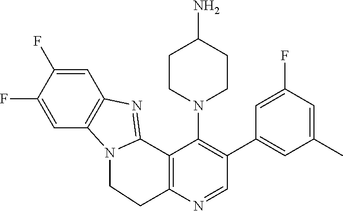 [SMILES: Cc1cc(F)cc(-c2cnc3c(c2N2CCC(N)CC2)-c2nc4cc(F)c(F)cc4n2CC3)c1] | 1H-NMR (400 MHz, DMSO-D6) δ −0.06 (s, 1H), −0.40 (dd, J = 10.3, 7.3 Hz, 1H), −0.52 (dd, J = 11.1, 7.3 Hz, 1H), −1.09 (s, 1H), −1.11 (s, 2H), −3.89 (t, J = 6.6 Hz, 2H), −4.38 (d, J = 13.2 Hz, 2H), −5.10 (t, J = 6.9 Hz, 2H), −5.20-−5.29 (m, 2H), −5.53 (d, J = 7.7 Hz, 1H), −5.81 (s, 3H), −5.86 (s, 1H), −6.37 (d. J = 7.7 Hz, 2H), −6.54 (s, 2H). MS (m/z) 464.3 (M + H)⁺. |

### Example 18

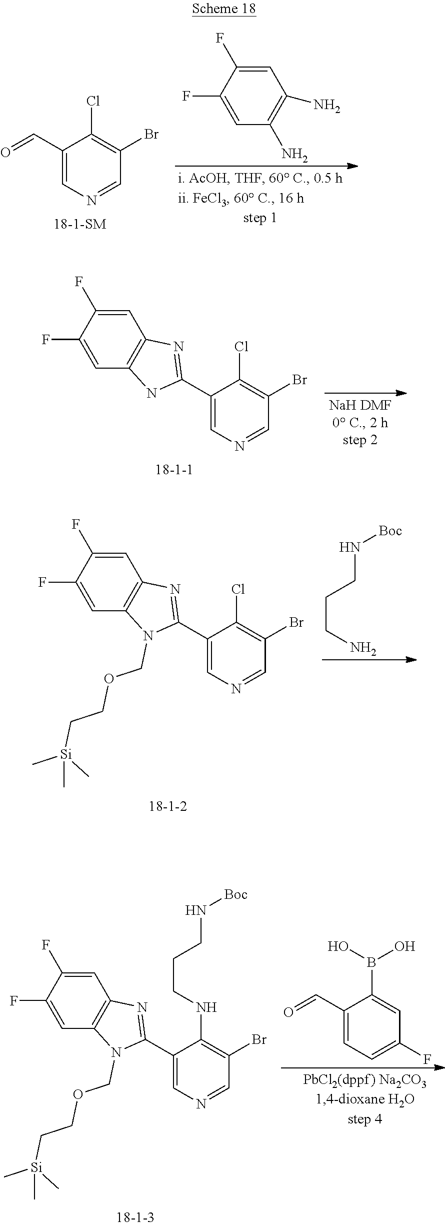

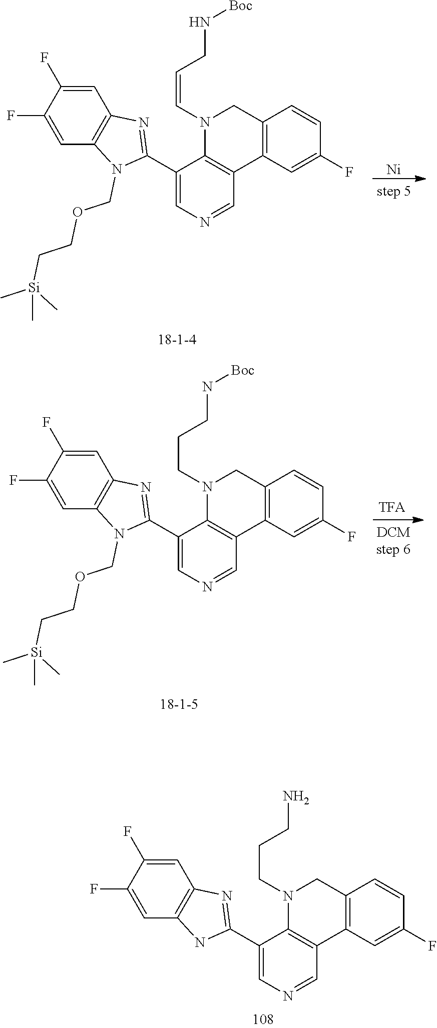

### Step 1: Synthesis of Compound 18-1-1

A solution of compound 18-1-SM (cas: 1060802-24-5, 3 g, 13.61 mmol) and 4,5-difluorobenzene-1,2-diamine (1.96 g, 13.61 mmol) in THF (10 mL) was added acetic acid (163.44 mg, 2.72 mmol) for 16 hr under N₂ at 25° C. After ferric;trichloride (331.10 mg, 2.04 mmol) was added at 55° C., the resulting mixture was stirred at 55° C. for 16 hr. The reaction mixture was diluted with ethyl acetate (150 mL). Then it was extracted with water (50 mL*2). The aqueous phase was stripped extracted with ethyl acetate (150 mL). Combined all organic phases and dried over anhydrous Na₂SO₄. Concentrated the organic phase to obtain a crude product. The crude product was purified by silica gel column (100-200 mesh) chromatography purification eluted with PE:EtOAc=1:1 to give the compound 18-1-1 (2.50 g, 5.80 mmol, 42.66% yield, 80% purity) as yellow solid. MS (m/z) 344.0 (M+H)⁺.

### Step 2: Synthesis of Compound 18-1-2

To a solution of compound 18-1-1 (1 g, 2.90 mmol) and SEM-C1 (629.06 mg, 3.77 mmol) in N,N-dimethylformamide (10 mL) was added sodium hydride (208.97 mg, 8.71 mmol) at 0° C., the resulting mixture was stirred at 0° C. for 2 hr in N₂. The reaction mixture was diluted with ethyl acetate (50 mL). Then it was extracted with water (50 mL*2). The aqueous phase was stripped extracted with ethyl acetate (150 mL). Combined all organic phases and dried over anhydrous Na₂SO₄. Concentrated the organic phase to obtain a crude product, which was purified by silicagel column (100-200 mesh) chromatography purification eluted with PE:EtOAc=3:1 to give the compound 18-1-2 (800.00 mg, 1.65 mmol, 56.89% yield, 98% purity) as yellow solid. MS (m/z) 474.20 (M+H)⁺.

### Step 3: Synthesis of Compound 18-1-3

A solution of compound 18-1-2 (800 mg, 1.68 mmol) and tert-butyl N-(3-aminopropyl)carbamate (440.37 mg, 2.53 mmol) in DMF (6 mL) was added DIPEA (653.29 mg, 5.05 mmol) at 25° C., the resulting mixture was stirred at 80° C. for 5 hr in N₂. The resulting solution extracted with EA (60 mL) and H₂O (20 mL) for three times. The combined organic layer was dried over Na₂SO₄ and concentrated. The crude product was purified by silicagel column (100-200 mesh) chromatography purification eluted with PE:EtOAc=1:1 to give compound 18-1-3 (780.00 mg, 1.25 mmol, 74.06% yield, 98% purity) as yellow oil. MS (m/z) 612.40 (M+H)⁺.

### Step 4: Synthesis of Compound 18-14

To the solution of compound 18-1-3 (300 mtg, 489.73 μmol) and (5-fluoro-2-formyl-phenyl)boronic acid (98.69 mg, 587.68 μmol) in 1,4-dioxane (5 mL) and H₂O (0.5 mL) was added Pd(dppf)Cl₂ (71.87 mg, 97.95 μmol) and K₂CO₃ (203.05 mg, 1.47 μmol) at 25° C., the resulting mixture was stirred at 120° C. for 2 hr under N₂ condition. The reaction mixture was diluted with ethyl acetate (150 mL). Then it was extracted with water (50 mL*2). The aqueous phase was stripped extracted with ethyl acetate (150 mL). Combined all organic phases and dried over anhydrous Na₂SO₄. Concentrated the organic phase to obtain a crude product. The crude product was purified by silicagel column (100-200 mesh) chromatography purification eluted with PE:EtOAc=1:4 to give the compound 18-1-4 (200.00 mg, 297.92 μmol, 60.83% yield, 95% purity) as yellow solid. MS (m/z) 638.3 (M+H)⁺.

### Step 5: Synthesis of Compound 18-1-5

A solution of compound 18-1-4 (100 mg, 156.80 μmol) in methanol (6 mL) was added Renay Ni (9.20 mg, 156.80 μmol) at 25° C., the resulting mixture was stirred at 50° C. for 14 hr under H₂. The reaction mixture was filtered to remove solid and this solid was washed with MeOH (25 mL*3). The filtrate was combined organic phase was dried under vacuum to afford compound 18-1-5 (65.00 mg, crude) as a yellow solid. MS (m/z) 640.2 (M+H)⁺.

### Step 6: Synthesis of Compound 108

A solution of compound 18-1-5 (75 mg, 117.60 μmol) in Trifluoroacetic acid (8 mL) was added dichloromethane (5 mL) at 25° C., the resulting mixture was stirred at 25° C. for 20 hr. The reaction solution was diluted with 20 mL dichloromethane, the organic phase was concentrated, and the above operation was repeated three times. The crude product was purified by Prep-HPLC (Prep-C18, 5 μM OBD, 19×250 mm, Column, Waters, gradient elution of 20% MeCN in water to 30% MCCN in water over a 10 min period, where both solvents contain 10 mmol/L FA) to provide target compound 108 (25.55 mg, 61.78 μmol, 52.54% yield, 99% purity) as a white solid.

Analytical data for compound 108 is provided below.

| Cpd. No. | Structure | Analytical Data |
|---|---|---|
| 108 | 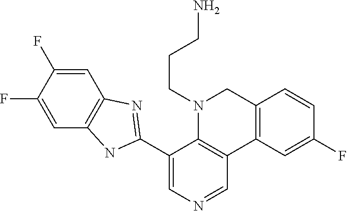 [SMILES: NCCCN1Cc2ccc(F)cc2-c2cncc(-c3nc4cc(F)c(F)cc4[nH]3)c21] | ¹H-NMR (400 MHz, MeOD-d4) δ 8.98 (s, 1H), 8.50 (s, 1H), 8.21 (s, 1H), 7.83 (dd, J = 10.6, 2.3 Hz, 1H), 7.60 (t, J = 8.9 Hz, 2H), 7.29-7.26 (m, 1H), 7.16-7.12 (m, 1H), 4.34 (s, 2H), 2.73 (d, J = 6.6 Hz, 2H), 2.19 (d, J = 29.7 Hz, 2H), 1.42 (s, 2H). MS (m/z) 410.2 (M + H)⁺. |

### Example 19

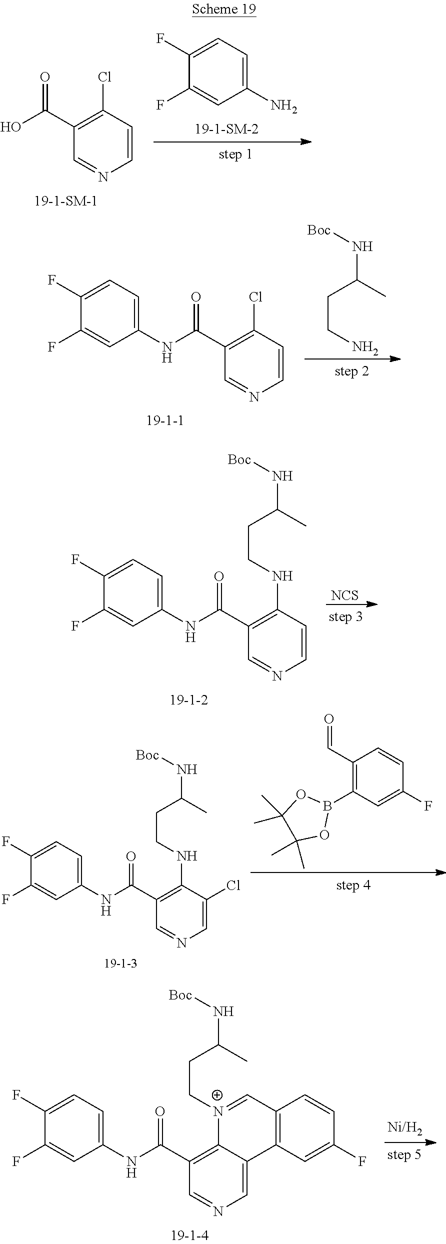

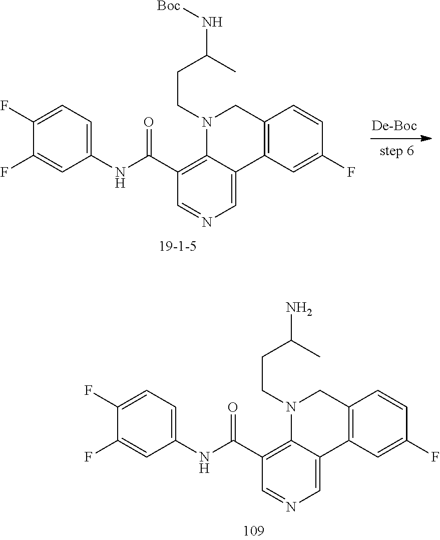 [SMILES: CC(CCN1Cc2ccc(F)cc2-c2cncc(C(=O)Nc3ccc(F)c(F)c3)c21)NC(=O)OC(C)(C)C ; CC(N)CCN1Cc2ccc(F)cc2-c2cncc(C(=O)Nc3ccc(F)c(F)c3)c21]

### Step 1: Synthesis of tert-butyl N-[(Z)-3-[4-[(3,4-difluorophenyl) carbamoyl]-9-fluoro-6H-benzo[c][1,6]naphthyridin-5-yl]-1-methyl-allyl]carbamate (Compound 19-1-1)

To the solution of compound 19-1-SM-2 (1 g, 7.75 mmol) and compound 19-1-SM-1 (1.46 g, 9.29 mmol) in DMF (8 mL) was added HATU (2.95 g, 7.75 mmol) and DIPEA (3.00 g, 23.24 mmol) at 25° C. The resulting mixture was then stirred at 25° C. for 2 hr. The reaction was monitored by LCMS. LCMS showed the main raw material was disappeared. Add water until lots of solid observed. The precipitated solid was collected and washed with H₂O (20 mL*3) and dried under vacuum to afford 4-chloro-N-(3,4-difluorophenyl)pyridine-3-carboxamide (compound 19-1-1, 1.83 g, 5.45 nmol, 70.36% yield, 80% purity) as a yellow solid. The crude product used to next step directly. MS (m/z) 269.1 (M+H)⁺.

### Step 2: Synthesis of tert-butyl N-[3-[[3-[(3,4-difluorophenyl)carbamoyl]-4-pyridyl]amino]-1-methyl-propyl]carbamate (Compound 19-1-2)

To a solution of compound 19-1-1 (356.74 mg, 1.06 mmol) and tert-butyl N-(3-amino-1-methyl-propyl)carbamate (200 mg, 1.06 mmol) in Dimethyl sulfoxide (1 mL) was added DIPEA (411.88 mg, 3.19 mmol) at 25° C., and the resulting mixture was then stirred at 120° C. for 2.5 hr. The reaction was complete detected by LC-MS, LCMS showed the reaction worked completely. The reaction mixture was diluted with H₂O (15 mL) and EtOAc (15 mL). The mixture was extracted with ethyl acetate (25 mL*2). The combined organic layer was washed with brine (15 mL), dried over Na₂SO₄, then filtered and concentrated under reduced pressure. The crude product was purified by silica gel chromatography eluted with PE:EtOAc (from 100/0 to 60/40) to give tert-butyl N-[3-1[3-[(3,4-difluorophenyl)carbamoyl]-4-pyridyl]amino]-1-methyl-propyl] carbamate (compound 19-1-2, 187.00 mg, 435.86 μmol, 41.03% yield, 98% purity) as yellow solid. MS (m/z) 421.2 (M+H)⁺.

### Step 3: Synthesis of tert-butyl N-[3-[[3-chloro-5-[(3,4-difluorophenyl)carbamoyl]-4-pyridyl]amino]-1-methyl-propyl]carbamate (Compound 19-1-3)

A solution of compound 19-1-2 (120 mg, 279.70 μmol) in N,N-dimethylformamide (0.5 mL) was added NCS (112.04 mg, 839.10 μmol) at 25° C., the resulting mixture was stirred at 30° C. for 8 hr. The reaction was monitored by LC-MS. LCMS showed the raw material was disappeared and product was detected clearly. The reaction mixture was diluted with H₂O (15 mL). Then it was extracted with ethyl acetate (15 mL*3). The combined organic layer was washed with brine (15 mL), dried over Na₂SO₄, then filtered and concentrated under reduced pressure. The crude product was purified by silica gel chromatography eluted with PE:EtOAc (from 100/0 to 64/36) to give tert-butyl N-[3-[[3-chloro-5-[(3,4-difluorophenyl)carbamoyl]-4-pyridyl]amino]-1-methyl-propyl] carbamate (compound 19-1-3, 115.00 mg, 247.75 μmol, 88.58% yield, 98% purity) as yellow solid. MS (m/z) 454.9 (M+H)⁺.

### Step 4: Synthesis of tert-butyl N-[(Z)-3-[4-[(3,4-difluorophenyl)carbamoyl]-9-fluoro-6H-benzo[c][1,6]naphthyridin-5-yl]-1-methyl-allyl]carbamate (Compound 19-1-4)

To a mixture of compound 19-1-3 (100 mg, 219.83 μmol). (5-fluoro-2-formyl-phenyl)boronic acid (44.30 mg, 263.80 μmol) in 1,4-dioxane (2 mL) and water (0.1 mL) was added XPhosPdG2 (17.27 mg, 21.98 μmol) and K₂CO₃ (91.01 mg, 659.49 μmol) at 25° C., the resulting mixture was degassed, then stirred at 110° C. for 3 hr under N₂. The reaction mixture was concentrated to dryness. The crude product was purified by silica gel chromatography eluted with PE:EtOAc (from 100/0 to 1/1) to give tert-butyl-N-[(Z)-3-[4-[(3,4-difluorophenyl)carbamoyl]-9-fluoro-6H-benzo[c][1,6]naphthyridin-5-yl]-1-methyl-allyl]carbamate (compound 19-14, 67.00 mg, 122.62 μmol, 55.78% yield, 96% purity) as yellow solid. MS (m/z) 525.2 (M+H)⁺.

### Step 5: Synthesis of tert-butyl-[3-[4-[(3,4-difluorophenyl)carbamoyl]-N9-fluoro-6H-benzo[c][1,6]naphthyridin-5-yl]-1-methyl-propyl]carbamate (Compound 19-1-5)

To a solution of compound 19-14 (50 mg, 95.32 μmol) in MeOH (10 mL) was added Raney Ni (100 mg) under N₂, then the suspension was degassed under H₂ completely. The mixture was stirred under H₂ balloon at 50° C. for 5 hr. The reaction was monitored by LC-MS. LCMS showed the raw material was disappeared. Worked completely. The reaction mixture was filtered and the precipitated solid was washed with MeOH (20 mL*3) and the filtrate was vacuum-dried to obtain tert-butyl-[3-[4-[(3,4-difluorophenyl)carbamoyl]-N9-fluoro-6H-benzo[c][1,6]naphthyridin-5-yl]-1-methyl-propyl]carbamate (compound 19-1-5, 33.00 mg, crude) as a white solid. The crude product used to next step directly. MS (m/z) 527.3 (M+H)⁺.

### Step 6: Synthesis of 5-(3-aminobutyl)-N-(3,4-difluorophenyl)-9-fluoro-6H-benzo [c][1,6]naphthyridine-4-carboxamide (Compound 109)

A solution of compound 19-1-5 (33 mg, 53.90 μmol) in dichloromethane (1 mL) and TFA (0.5 mL) was stirred at 25° C. for 2 hr. The reaction was monitored by LC-MS. LCMS showed the raw material was disappeared. Add excess dichloromethane vacuum rotary evaporation. The crude product was purified by Prep-HPLC (Prep-C18, 5 μM OBD, 19×250 mm, Column, Waters; gradient elution of 40% MCCN in water to 50% MCCN in water over a 9 min period, where both solvents contain 10 mmol/L NH₄HCO₃) to provide 5-(3-aminobutyl)-N-(3,4-difluorophenyl)-9-fluoro-6H-benzo[c][1.6]naphthyridine-4-carboxamide (compound 109, 15.00 mg, 34.12 μmol, 63.31% yield, 97% purity) as a write solid.

Analytical data for compound 109 is provided below.

| Cpd. | | |
|---|---|---|
| No. | Structure | Analytical Data |
| 109 | 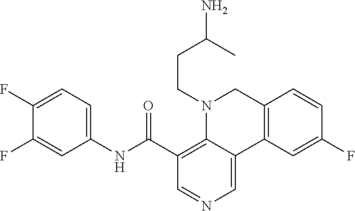 [SMILES: CC(N)CCN1Cc2ccc(F)cc2-c2cncc(C(=O)Nc3ccc(F)c(F)c3)c21] | 1H-NMR (400 MHz, DMSO-D6) δ 8.88 (d. J = 2.7 Hz, 1H), 8.21 (d, J = 6.0 Hz, 1H), 7.91 (q, J = 6.0 Hz, 1H), 7.79 (t, J = 7.4 Hz, 1H), 7.41 (dd, J = 19.2, 8.8 Hz, 2H), 7.32-7.25 (m, 1H), 7.16 (dd, J = 19.0, 10.2 Hz, 1H), 4.46 (d, J = 4.9 Hz, 2H), 3.36-3.49 (4H), 3.07-3.20 (2H), 1.58- 1.39 (m, 2H), 0.82-0.74 (m, 3H). MS (m/z) 426.9 (M + H)⁺. |

### Example 20

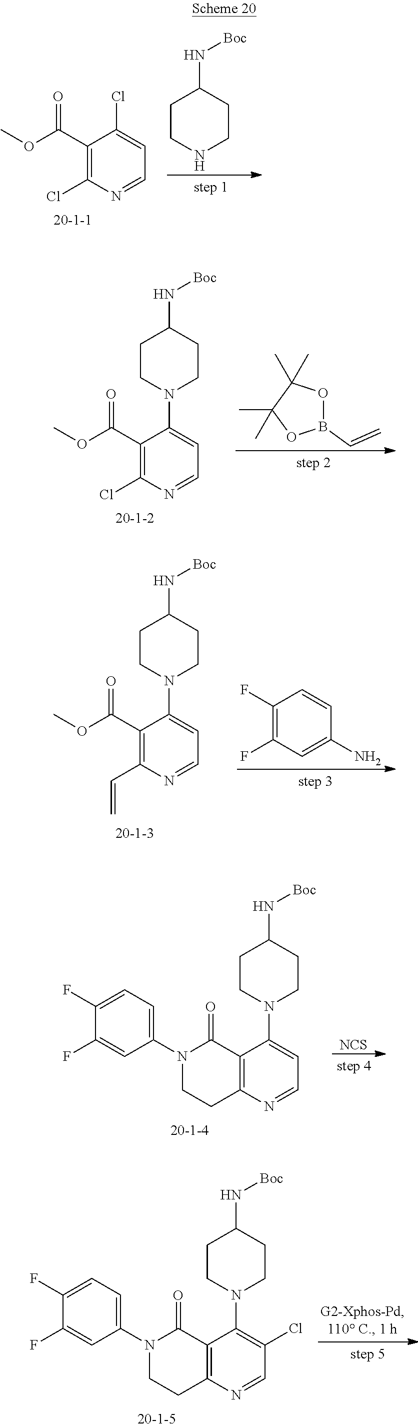

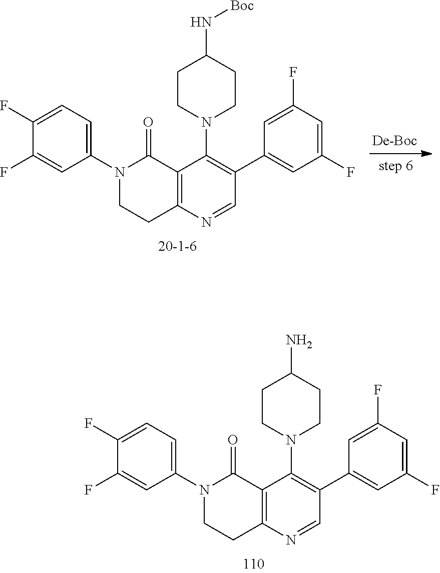 [SMILES: CC(C)(C)OC(=O)NC1CCN(c2c(-c3cc(F)cc(F)c3)cnc3c2C(=O)N(c2ccc(F)c(F)c2)CC3)CC1 ; NC1CCN(c2c(-c3cc(F)cc(F)c3)cnc3c2C(=O)N(c2ccc(F)c(F)c2)CC3)CC1]

### Step 1: Synthesis of Compound 20-1-2

To a solution of compound 20-1-1 (2 g, 9.71 mmol) in Dimethyl sulfoxide (8 mL) was added tert-butyl piperidin-4-ylcarbamate (2.92 g, 14.56 mmol) at 25° C., and the resulting mixture was stirred at 120° C. for 2 hr in N₂. The progress of the reaction was monitored by LCMS. The reaction mixture was diluted with H₂O (20 mL) and ethyl acetate (20 mL). The mixture was separated ad water phase was extracted with ethyl acetate twice. The combined organic layer was washed with brine, dried over Na₂SO₄, filtered and concentrated under reduced pressure to give the residue, which was purified by silica gel chromatography eluted with PE/EtOAc from 20/1 to 4/1 to give compound 20-1-2 (2.50 g, 6.08 mmol, 62.67% yield, 90% purity) as white solid. MS (m/z) 369.9 (M+H)⁺.

### Step 2: Synthesis of Compound 20-1-3

To a solution of compound 20-1-2 (1 g, 2.70 mmol) and 4,4,5,5-tetramethyl-2-vinyl-1,3,2-dioxaborolane (499.72 mg, 3.24 mmol) in 1,4-dioxane (2 mL) and H₂O (0.2 mL) was added potassium carbonate (934.25 mg, 6.76 mmol) and Xantphos Pd G2 (224.08 mg, 270.39 μmol) at 25° C., the resulting mixture was stirred at 110° C. for 2 hr in N2. The progress of the reaction was monitored by LCMS. Cooled to room temperature, the reaction mixture was quenched with H₂O (20 mL). Then it was extracted with ethyl acetate. The combined organic layers were washed with brine, dried over Na₂SO₄, then filtered and concentrated under reduced pressure. The crude product was purified by silica gel chromatography eluted with PE/EtOAc from 10/1 to 1/1 to give compound 20-1-3(500.00 mg, 1.38 mmol, 51.16% yield, 100% purity) as yellow solid. MS (m/z) 362.0 (M+H)⁺.

### Step 3: Synthesis of Compound 20-1-4

To a solution of compound 20-1-3 (300 mg, 830.03 μmol) and 3,4-difluoroaniline (535.81 mg, 4.15 mmol) in toluene (3 mL) and acetic acid (1 mL) at 25° C., the resulting mixture was stirred at 110° C. for 4 hr in N₂. The progress of the reaction was monitored by LCMS. LCMS show the raw material was disappeared. The reaction mixture was cooled to 25° C. and quenched with H₂O (20 mL). Then the mixture was separated, and water phase was washed with ethyl acetate twice. The combined organic phase was washed by brine, dried over Na₂SO₄, filtered, and concentrated to give the residue, which was purified by silica gel chromatography eluted with PE/EtOAc from 10/1 to 1/1 to give compound 20-14 (45.00 mg, 78.52 μmol, 9.46% yield, 80% purity) as yellow oil. MS (m/z) 458.9 (M+H)⁺.

### Step 4: Synthesis of Compound 20-1-5

To a solution of compound 20-14 (45 mg, 109.05 μmol) in DMF (1.5 mL) was added NCS (21.84 mg, 163.58 μmol) at 25° C. the resulting mixture was stirred at 30° C. for 2 hr. The progress of the reaction was monitored by LCMS. LCMS show the raw material was disappeared. The reaction mixture was cooled to room temperature and quenched with H₂O (20 mL). Then the mixture was separated, and water phase was separated by ethyl acetate twice. The combined organic phase was washed by brine, dried over Na₂SO₄, filtered, and concentrated to give the residue, which was purified by silica gel chromatography eluted with PE/EtOAc from 30/1 to 8/1 to give compound 20-1-5(40.00 mg, 81.14 μmol, 74.41% yield) as yellow solid. MS (m/z) 469.2 (M+H)⁺.

### Step 5: Synthesis of Compound 20-1-6

To a solution of compound 20-1-5 (40 mg, 81.14 μmol) and (3,5-difluorophenyl)boronic acid (19.22 mg, 121.72 μmol) in 1,4-dioxane (2 mL) and H₂O (0.2 mL) was added K₂CO₃ (28.04 mg, 202.86 μmol) and G2-Xphos-Pd (10.09 mg, 12.17 μmol) at 25° C., the resulting mixture was stirred at 110° C. for 2 hr in N₂. The progress of the reaction was monitored by LCMS. LCMS show the raw material was disappeared. The reaction mixture was cooled to 25° C. and quenched with H₂O (20 mL). Then the mixture was separated and water phase was separated by ethyl acetate twice. The combined organic phase was washed by brine, dried over Na₂SO₄, filtered and concentrated to give the residue, which was purified by silica gel chromatography eluted with PE/EtOAc from 5/1 to 3/1 to give compound 20-1-6 (20.00 mg, 35.05 μmol, 43.20% yield) as yellow oil. MS (m/z) 571.2 (M+H)⁺.

### Step 6: Synthesis of Target Compound 110

To a solution of compound 20-1-6 (20 mg, 35.05 μmol) in DCM (2.53 mL) was added TFA (707.63 mg, 6.21 mmol, 474.92 μL) at 25° C., the resulting mixture was stirred at 25° C. for 2 hr. The reaction solution was diluted with 20 mL dichloromethane, the organic phase was concentrated, and the above operation was repeated three times. The crude product was purified by Prep-HPLC (Prep-C18, 5 μM OBD, 19×250 mm, Column, Waters: gradient elution of 40% MeCN in water to 50% MeCN in water over a 9 min period, where both solvents contain 10 mmol/L NH₄HCO₃) to provide compound 110 (7.50 mg, 15.94 μmol, 45.48% yield, 100% purity) as a colorless oil.

Compounds 110-116 were synthesized according to the 6-step procedure described in Example 20. Analytical data for compounds 110-116 are provided below.

| Cpd. | | |
|---|---|---|
| No. | Structure | Analytical Data |
| 110 | 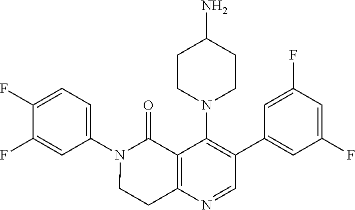 [SMILES: NC1CCN(c2c(-c3cc(F)cc(F)c3)cnc3c2C(=O)N(c2ccc(F)c(F)c2)CC3)CC1] | 1H-NMR (400 MHz, METHANOL-D4) δ 8.17 (s, 1H), 7.50 (ddd, J = 11.6, 7.2, 2.5 Hz, 1H), 7.41-7.34 (m, 1H), 7.30- 7.27 (m, 1H), 7.21-7.16 (m, 3H), 4.15 (t, J = 6.0 Hz, 2H), 3.50-3.42 (m, 4H), 3.31 (q, J = 1.6 Hz, 2H), 3.20 (t, J = 12.1 Hz, 1H), 1.85 (d, J = 9.9 Hz, 2H), 1.66 (dd, J = 12.0, 4.0 Hz, 2H). MS (m/z) 471.1 (M + H)⁺. |
| 111 | 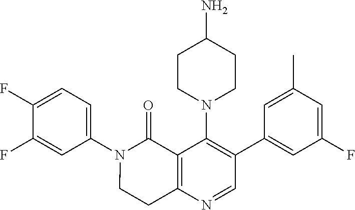 [SMILES: Cc1cc(F)cc(-c2cnc3c(c2N2CCC(N)CC2)C(=O)N(c2ccc(F)c(F)c2)CC3)c1] | 1H-NMR (400 MHz, DMSO- D6): δ8.19 (d, J = 4.7 Hz, 1H), 7.96 (s, 2H), 7.64 (ddd, J = 12.1, 7.4, 2.7 Hz, 1H), 7.53 (dd, J = 19.8, 9.1 Hz, 1H), 7.35- 7.33 (m, 1H), 7.18- 7.11 (m, 3H), 4.03 (t, J = 5.8 Hz, 2H), 3.29- 3.17 (m, 5H), 2.99 (t, J = 12.1 Hz, 2H), 2.42 (s, 3H), 1.68 (d J = 10.7 Hz, 2H), 1.47-1.39 (m, 2H). 19F-NMR (376 MHz, DMSO-D6): δ ppm −112.75 (s, 1 F), −137.46 (s, 1 F), −141.01 (s, 1 F). MS (m/z) 467.4 (M + H)⁺. |
| 112 | 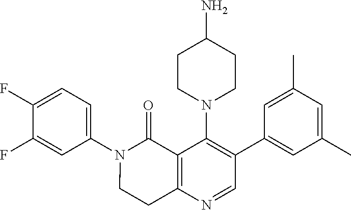 [SMILES: Cc1cc(C)cc(-c2cnc3c(c2N2CCC(N)CC2)C(=O)N(c2ccc(F)c(F)c2)CC3)c1] | 1H-NMR(400MHz, DMSO-d6): δ8.14 (s, 1H), 7.87 (s, 2H), 7.64 (ddd, J = 12.2, 7.4, 2.5 Hz, 1H), 7.53 (dd, J = 19.7, 9.2 Hz, 1H), 7.33 (t, J = 4.5 Hz, 1H), 7.09 (s, 1H), 7.04 (s, 2H), 4.02 (d, J = 4.9 Hz, 2H), 3.26 (s, 2H), 3.16 (s, 3H), 2.99-2.93 (m, 2H), 2.36- 2.33 (m, 6H), 1.64 (d, J = 11.0 Hz, 2H), 1.42 (d, J = 12.9 Hz, 2H). 19F-NMR (376 MHz, DMSO-D6): δ ppm −137.51 (s, 1F), −141.06 (s, 1F). MS (m/z) 463.4 (M + H)⁺. |
| 113 | 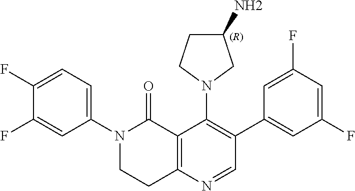 | 1H-NMR (400 MHz, DMSO-D6) δ 7.96 (s, 1H), 7.61 (ddd, J = 12.4, 7.4, 2.5 Hz, 1H), 7.53-7.46 (m, 1H), 7.31-7.21 (m, 2H), 7.04 (dd, J = 8.4, 2.3 Hz, 2H), 6.21 (s, 2H), 4.00 (t, J = 6.0 Hz, 2H), 3.17 (td, J = 11.2, 5.6 Hz, 2H), 3.09-3.01 (m, 4H), 2.76-2.67 (m, 1H), 1.84 (td, J = 12.6, 7.2 Hz, 1H), 1.67- 1.49 (m, 1H). MS (m/z) 456.9 (M + H)⁺. |
| 114 | 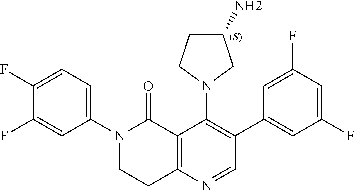 | 1H-NMR (400 MHz, DMSO-D6) δ 7.94 (s, 1H), 7.60 (ddd, J = 12.4, 7.4, 2.5 Hz, 1H), 7.52-7.45 (m, 1H), 7.32-7.20 (m, 2H), 7.06-7.02 (m, 2H), 4.00 (t, J = 6.0 Hz, 2H), 3.29 (t, J = 5.4 Hz, 4H), 3.17-3.00 (m, 4H), 2.68 (dd, J = 11.3, 4.7 Hz, 1H), 1.80 (td, J = 12.4, 7.2 Hz, 1H), 1.47 (td, J = 12.3, 6.0 Hz, 1H). MS (m/z) 456.8 (M + H)⁺. |
| 115 | 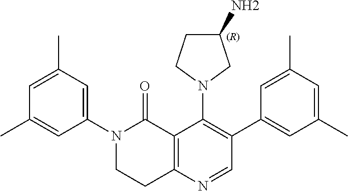 | 1H-NMR (400 MHz, DMSO-D6) δ 7.85 (s, 1H), 7.03 (s, 2H), 6.96 (s, 1H), 6.89 (d, J = 11.0 Hz, 3H), 3.96 (t, J = 6.9 Hz, 2H), 3.44 (t, J = 15.1 Hz, 2H), 3.23-3.28 (1H), 3.15- 2.96 (m, 5H), 2.72-2.65 (m, 1H), 2.30 (d, J = 7.7 Hz, 12H), 1.78 (td, J = 12.8, 7.0 Hz, 1H), 1.50-1.44 (m, 1H). MS (m/z) 441.0 (M + H)⁺. |
| 116 | 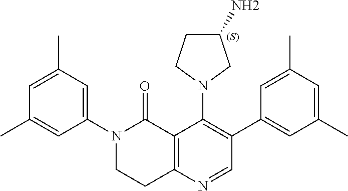 | 1H-NMR (400 MHz, DMSO-D6) δ 8.14 (s, 1H), 8.00 (s, 2H), 7.06 (s, 3H), 6.94 (d, J = 11.3 Hz, 3H), 4.09-3.98 (m, 2H), 3.74 (s, 1H), 3.45 (s, 1H), 3.24 (d, J = 5.5 Hz, 5H), 2.32 (d, J = 14.3 Hz, 12H), 2.14-2.06 (m, 1H), 1.89-1.85 (m, 1H). MS (m/z) 441.0 (M + H)⁺. |

### Example 21

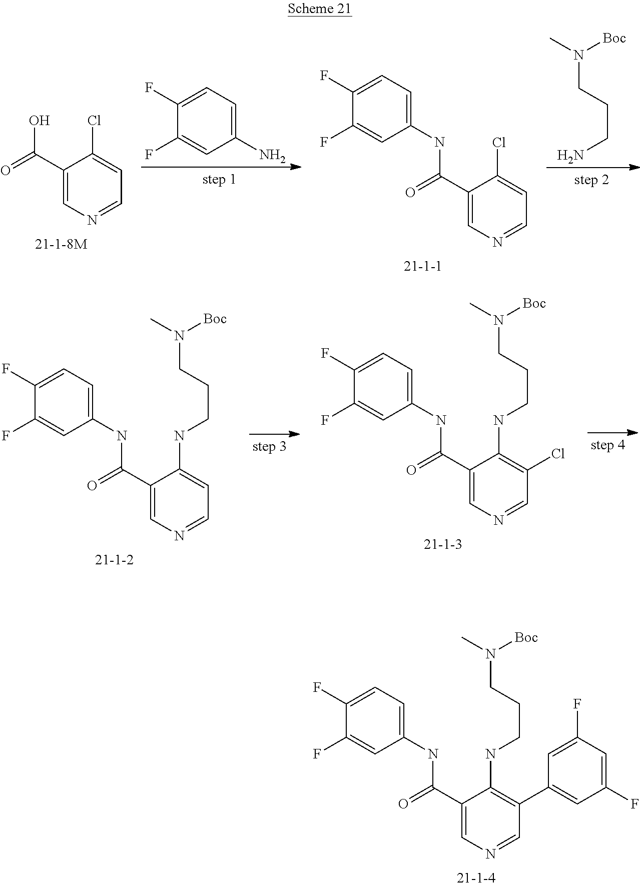

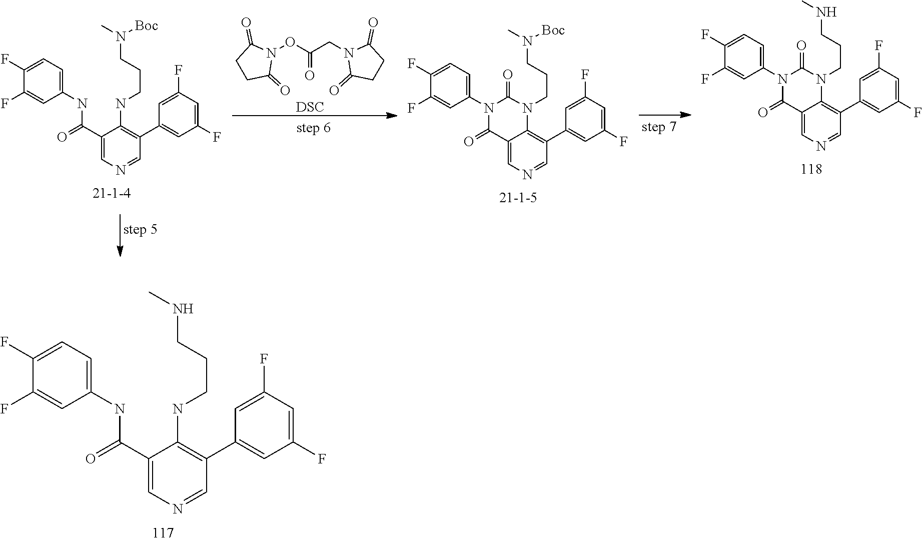

### Step 1: Synthesis of Compound 21-1-1

A solution of compound 21-1-SM (1 g, 6.35 mmol) and 3,4-difluoroaniline (901.39 mg, 6.98 mmol) in DMF (5 mL) was added HATU (2.43 g, 6.35 mmol) and DIPEA (2.46 g, 19.04 mmol) at 25° C., and the resulting mixture was stirred at 25° C. for 2 hr in N₂. The reaction was monitored by LCMS, which showed the reaction was completed. Add 20 mL water into the reaction solution, then filter to collect the solid, which was compound 21-1-1(1.46 g, 4.88 mmol, 76.93% yield, 89.84% purity) as white solid. MS (m/z) 269.0 (M+H)⁺.

### Step 2: Synthesis of Compound 21-1-2

A solution of compound 21-1-1 (800 mg, 2.98 mmol) and tert-butyl N-(3-aminopropyl)-N-methylcarbamate (2.80 g, 14.89 mmol) in DMSO (2 mL) was added DIPEA (1.92 g, 14.89 mmol) at 25° C., the resulting mixture was stirred at 120° C. for 2 hr. The reaction was monitored by LCMS, which showed the reaction was completed. The reaction mixture was diluted with ethyl acetate (100 ml) and water (50 mL). The aqueous phase was extracted with ethyl acetate (100 mL) twice. Combined all organic phases and dried over anhydrous Na₂SO₄. Concentrated the organic phase to obtain a crude product, which was purified by silica gel column (100-200 mesh) chromatography purification eluted with PE/EtOAc=4/1 to give compound 21-1-2 (1.03 g, 2.34 mmol, 78.49% yield, 95.14% purity) as yellow solid. MS (m/z) 421.2 (M+H)⁺.

### Step 3: Synthesis of Compound 21-1-3

A solution of compound 21-1-2 (600 mg, 1.43 mmol) and NCS (571.67 mg, 4.28 mmol) in DMF (5 mL) at 25° C., the resulting mixture was stirred at 30° C. for 12 hr. The reaction was monitored by LCMS, which showed the reaction was completed. The reaction mixture was diluted with ethyl acetate (100 mL) and water (50 ml). The aqueous phase was extracted with ethyl acetate (60 mL) twice. Combined all organic phases, dried over anhydrous Na₂SO₄, concentrated the organic phase to give a crude product, which was purified by silica gel column chromatography purification eluted with DCM/MeOH=1/1 to give the title compound (compound 21-1-3, 400 mg, 854.35 μmol, 59.87% yield, 97.16% purity) as brown oil. MS (m/z) 454.9 (M+H)⁺.

### Step 4: Synthesis of Compound 21-14

A solution of compound 21-1-3 (200 mg, 439.66 μmol) and (3,5-difluorophenyl)boronic acid (104.14 mg, 659.49 μmol) in 1,4-dioxane (10 mL) and water (1 mL) was added K₂CO₃ (182.29 mg, 1.32 mmol) and X-Phox-Pd-G2 (34.59 mg, 43.97 μmol) at 25° C., the resulting mixture was stirred at 110° C. for 1 hr in N₂ condition. The reaction was monitored by LCMS, which showed the reaction was completed. The reaction mixture was diluted with ethyl acetate (60 mL). Then it was extracted with water (20 mL*2). The aqueous phase was stripped extracted with ethyl acetate (50 mL). Combined all organic phases and dried over anhydrous Na₂SO₄. Concentrate the organic phase to obtain a crude product, which was purified by silica gel column (100-200 mesh) chromatography purification eluted with DCM:MeOH=10:1 to give the title compound (compound 21-14, 186.00 mg, 337.30 μmol, 76.72% yield, 96.57% purity) as yellow oil. MS (m/z) 533.2 (M+H)⁺.

### Step 5: Synthesis of Compound 117

A solution of compound 21-14 (50 mg, 93.89 μmol) in Methanolic hydrochloric acid solution (1 mL) and DCM (5 mL) was added and at 25° C., the resulting mixture was stirred at 40° C. for 1 hr. The reaction was monitored by LCMS, which showed the reaction was completed. Concentrated the organic phase to obtain a crude product. The crude product was washed 5 mL dichloromethane, and the above operation was repeated three times. Concentrated to provide compound 117 (22.77 mg, 49.85 μmol, 53.09% yield, 94.67% purity) as a gray solid.

### Step 6: Synthesis of Compound 21-1-5

A solution of compound 21-14 (100 mg, 187.78 μmol) and bis(2,5-dioxopyrrolidin-1-yl) carbonate (230.90 mg, 901.36 μmol) in DMF (2 mL) was added Sodium hydride (36.05 mg, 901.36 μmol) at 25° C. the resulting mixture was stirred at 60° C. for 6 hr in N₂. The reaction was monitored by LCMS, which showed the reaction was completed. The reaction mixture was diluted with ethyl acetate (30 mL). Then it was extracted with water (10 mL*2). The aqueous phase was stripped extracted with ethyl acetate (300 mL). Combined all organic phases and dried over anhydrous Na₂SO₄, filtered and concentrated the organic phase to obtain a crude product, which was purified by silica gel column chromatography purification eluted with DCM:MeOH=10:1 to give the title compound (compound 21-1-5, 12.00 mg, 20.85 μmol, 11.51% yield, 97.04% purity) as white solid. MS (m/z) 559.2 (M+H)⁺.

### Step 7: Synthesis of Compound 118

A solution of compound 21-1-5 (12 mg, 21.49 μmol) in methanolic hydrochloric acid solution (1 mL) and DCM (5 mL) was added and at 25° C., the resulting mixture was stirred at 40° C. for 2 hr. The reaction was monitored by LCMS, which showed the reaction was completed. The reaction was directly concentrated to obtain a crude product. The crude product was washed 5 mL dichloromethane, and the above operation was repeated three times. Concentrated to provide compound 118 (9.00 mg, 19.63 μmol, 91.38% yield, 100/purity) as a gray solid.

Analytical data for compounds 117 and 118 are provided below.

| Cpd. No. | Structure | Analytical Data |
|---|---|---|
| 117 | 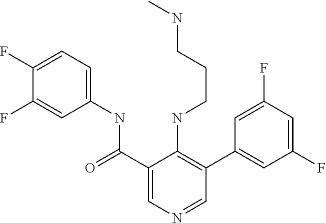 [SMILES: CNCCCNc1c(C(=O)Nc2ccc(F)c(F)c2)cncc1-c1cc(F)cc(F)c1] | 1H-NMR (DMSO-d6, 400 MHz): δ (ppm) 1.75- 1.87 (p, J = 7.1 Hz, 2H), 2.39-2.45 (t, J = 5.4 Hz, 3H), 2.61-2.75 (m, 2H), 3.02-3.16 (m, 2H),7.31-7.37 (m, 2H), 7.39-7.54 (m, 2H), 7.55-7.62 (m, 1H), 7.87-8.02 (m, 1H), 8.22-8.32 (s, 1H), 8.72-8.81 (s, 1H), 8.81-9.01 (s,2H), 11.43-11.62 (s, 1H). MS (m/z) 432.8 (M + H)⁺. |
| 118 | 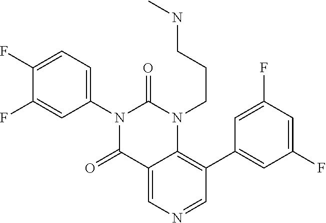 [SMILES: CNCCCn1c(=O)n(-c2ccc(F)c(F)c2)c(=O)c2cncc(-c3cc(F)cc(F)c3)c21] | 1H-NMR (400 MHz, DMSO-D6) δ 9.18 (s, 1H), 8.62 (s, 1H), 7.67-7.55 (m, 2H), 7.50-7.39 (m, 3H), 7.29 (t, J = 4.4 Hz, 1H), 3.69-3.65 (m, 2H), 2.42 (s, 3H), 2.12- 1.92 (m, 1H), 1.68-1.60 (m, 2H), 1.23 (s, 2H). MS (m/z) 458,9 (M + H)⁺. |

### Example 22

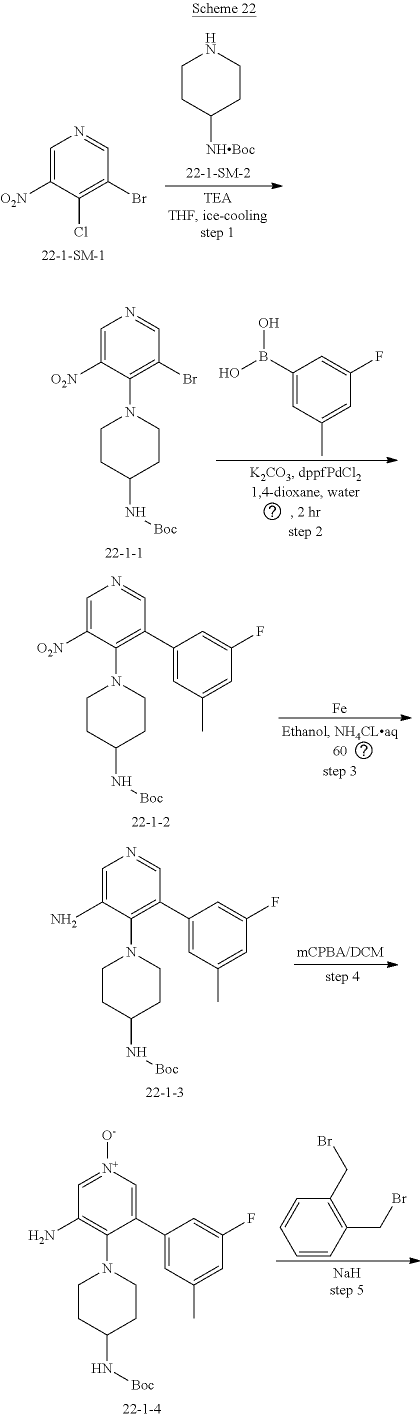

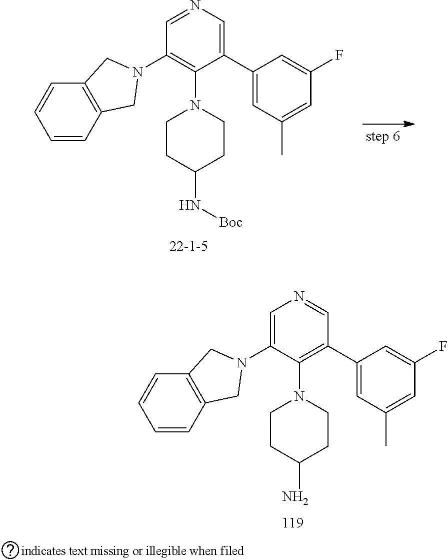 [SMILES: Cc1cc(F)cc(-c2cncc(N3Cc4ccccc4C3)c2N2CCC(NC(=O)OC(C)(C)C)CC2)c1 ; Cc1cc(F)cc(-c2cncc(N3Cc4ccccc4C3)c2N2CCC(N)CC2)c1]

A solution of compound 22-1-SM-1 (1 g, 4.21 mmol) and compound 22-1-SM-2 (1.01 g, 5.05 mmol) in THF (10 mL) was added TEA (1.28 g, 12.63 mmol) at 25° C. and the resulting mixture was stirred at 0° C. for 1 hr. The reaction mixture was added water (50 mL) and saturated salt solution (15 mL) then was extracted with EA (50 mL*3). The combined organic layer was dried over by Na₂SO₄ and was concentrated to give crude product compound 22-1-1 (1.82 g). MS (nm/z) 403.0) (M+H)⁺.

### Step 2: Synthesis of Compound 22-1-2

A solution of compound 22-1-1 (500 mg, 1.25 mmol) and (3-fluoro-5-methyl-phenyl)boronic acid (6-5 230.20 mg, 1.50 mmol) in 1,4-dioxane (2 mL) was added Pd(dppf)Cl₂ (91.43 mg, 124.61 μmol) and K₂CO₃ (516.65 mg, 3.74 mmol) at 25° C. and the resulting mixture was stirred at 120° C. for 2 hr in N₂. The crude product was purified by silica gel chromatography (100-200 mesh) eluted with PE:EtOAc (from 100:1 to 72:28) to give compound 22-1-2 (576.00 mg, 1.20 mmol, 96.64% yield, 90% purity) as yellow solid. MS (m/z) 431.3 (M+H)⁺.

### Step 3: Synthesis of Compound 22-1-3

To the solution of compound 22-1-2 (556 mg, 1.29 mmol) in EtOH (15 mL) and saturated NH₄Cl (69.09 mg, 1.29 mmol) was added Fe (721.36 mg, 12.92 mmol) at 25° C., and the resulting mixture was stirred at 80° C. for 2 hr. The reaction mixture was added water (50 mL) and saturated salt solution (15 mL) then was extracted with EA (50 mL*3). The combined organic layer was dried over by Na₂SO₄ and then was concentrated to give crude product compound 22-1-3 (409 mg). MS (m/z) 401.3 (M+H)⁺.

### Step 4: Synthesis of Compound 22-1-4

To the solution of compound 22-1-3 (80 mg, 199.76 μmol) in DCM (5 mL) was added 3-chlorobenzenecarboperoxoic acid (51.71 mg, 299.63 μmol) at 25° C., and the resulting mixture was stirred at 0° C. for 10 hr. The reaction mixture was diluted with ethyl acetate (150 mL). Then it was extracted with water (50 mL*2). The aqueous phase was stripped extracted with ethyl acetate (150 mL). Combined all organic phases and dried over anhydrous Na₂SO₄. Concentrated the organic phase to obtain a crude product. The crude product was purified by silicagel column (100-200 mesh) chromatography purification eluted with DCM:MeOH (15:1) to give compound 22-14 (41.00 mg, 96.67 μmol, 48.39% yield, 98.20% purity) as yellow solid. MS (m/z) 417.3 (M+H)⁺.

### Step 5: Synthesis of Compound 22-1-5

To the solution of compound 22-1-4 (41 mg, 98.44 μmol) and 1,3-bis(bromomethyl)benzene (38.98 mg, 147.66 μmol) in Tetrahydrofuran (2 mL) was added NaH (7.09 mg, 295.33 μmol) and at 25° C., and the resulting mixture was stirred at 40° C. for 16 hr. The reaction mixture was diluted with ethyl acetate (30 mL). Then it was extracted with water (10 mL*2). The aqueous phase was stripped extracted with ethyl acetate (30 mL). Combined all organic phases and dried over anhydrous Na₂SO₄. Concentrated the organic phase to obtain a crude product. The crude product was purified by silica gel column (100-200 mesh) chromatography purification eluted with DCM:MeOH (30:1) to give compound 22-1-5 (10.00 mg, 18.36 μmol, 18.65% yield, 92.28% purity) as yellow solid. MS (m/z) 503.3 (M+H)⁺.

### Step 6: Synthesis of Compound 119

To the solution of compound 22-1-5 (10 mg, 19.90 μmol) in DCM (5 mL) was added methanolic hydrochloric acid solution (1 mL) at 25° C., and the resulting mixture was stirred at 40° C. for 3 hr. The reaction solution was diluted with dichloromethane (20 mL), the organic phase was concentrated, and the above operation was repeated three times. The crude product was purified by tert-Butyl methyl ether to provide compound 119 (3.02 mg, 7.17 μmol, 36.06% yield, 95.62% purity) as brown solid.

Analytical data for compound 119 is provided below.

| Cpd. No. | Structure | Analytical Data |
|---|---|---|
| 119 | 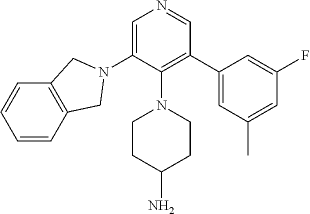 [SMILES: Cc1cc(F)cc(-c2cncc(N3Cc4ccccc4C3)c2N2CCC(N)CC2)c1] | 1H-NMR (400 MHz, D2O) δ ppm 8.23 (s, 1H), 7.91- 7.73 (m, 1H), 7.32-7.23 (m, 4H), 7.02- 6.87 (m, 3H), 4.45 (d, J = 13.7 Hz, 4H), 3.49 (d, J = 13.7 Hz, 2H), 3.15-3.09 (m, 1H), 2.78- 2.72 (m, 2H), 2.28 (s, 3H), 1.71-1.66 (m, 2H), 1.45- 1.35 (m, 2H). MS (m/z) 403.0 (M + H)⁺. |

### Example 23

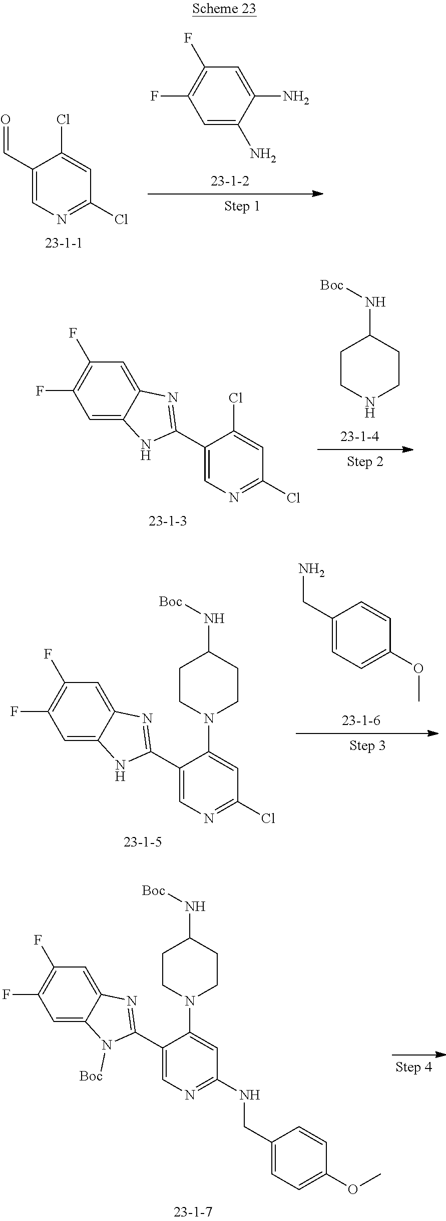

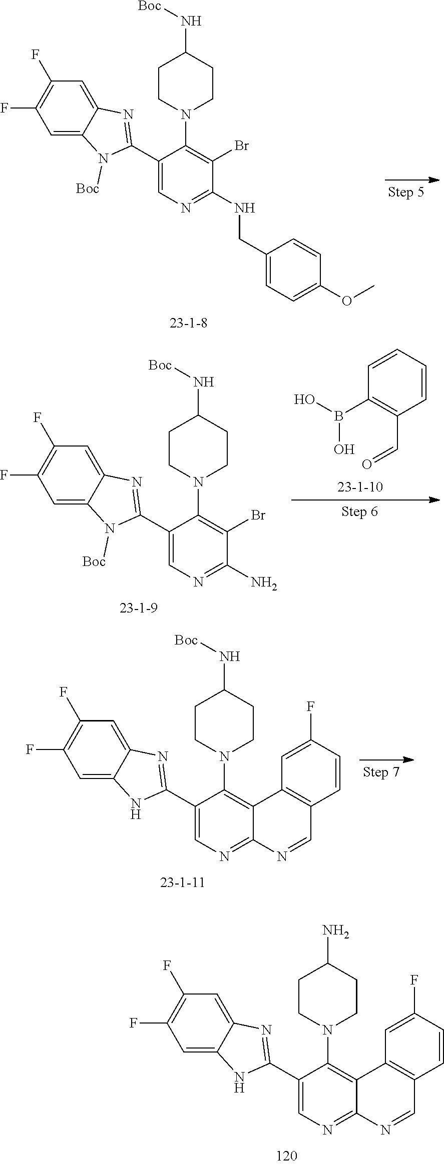 [SMILES: COc1ccc(CNc2ncc(-c3nc4cc(F)c(F)cc4n3C(=O)OC(C)(C)C)c(N3CCC(NC(=O)OC(C)(C)C)CC3)c2Br)cc1 ; CC(C)(C)OC(=O)NC1CCN(c2c(-c3nc4cc(F)c(F)cc4n3C(=O)OC(C)(C)C)cnc(N)c2Br)CC1 ; O=Cc1ccccc1B(O)O ; CC(C)(C)OC(=O)NC1CCN(c2c(-c3nc4cc(F)c(F)cc4[nH]3)cnc3ncc4ccc(F)cc4c23)CC1 ; NC1CCN(c2c(-c3nc4cc(F)c(F)cc4[nH]3)cnc3ncc4ccc(F)cc4c23)CC1]

### Step 1: Synthesis of Compound 23-1-3

A mixture of compound 23-1-1 (2.12 g, 12 mmol), compound 23-1-2 (1.75 g, 12.00 mmol) and AcOH (3.6 g, 60.00 mmol) in tetrahydrofuran (20 mL) was stirred at 60° C. for 2 h under N₂. Then FeCl₃ (7.78 g, 48.00 mmol) was added at 25 TC. The mixture was stirred at 60° C. for 2 h under N₂. Water (10 mL) was added, then the reaction mixture was extracted with EA (50 mL*2). The combined organic phase was dried by Na₂SO₄, filtered, concentrated and purified by silicagel column (100-200 mesh) chromatography purification eluted with PE:EA (from 100:0 to 3:1) to afford the compound 23-1-3 (2.3 g, 7.69 mmol, 64.10% yield) as yellow solid. MS (m/z) 300.1 (M+H)⁺.

### Step 2: Synthesis of Compound 23-1-5

A solution of compound 23-1-3 (2.30 g, 7.69 mmol), compound 23-1-4 (2.00 g, 9.99 mmol) and NN-Diisopropylethylamine (2.48 g, 19.23 mol) in DMO (20 mL) was stirred at 120° C. for 2 h. Water was added, then the mixture was filtered and washed by PE to give the crude compound 23-1-5 (2.8 g, 78.65% yield) as yellow solid. MS (m/z) 464.1 (M+H)⁺.

### Step 3: Synthesis of Compound 23-1-7

A mixture of compound 23-1-5 (2.8 g, 6.05 mmol), compound 23-1-6 (1.24 g, 9.07 mmol), Pd₂(dba)₃ (1.11 g, 1.21 mmol), ruphos (1.13 g, 2.42 mmol) and sodium; 2-methylpropan-2-olate (1.45 g, 15.13 mmol) in DMSO (20 mL) was stirred at 100° C. for 2 h under N₂. The mixture was cooled to 25° C., then (Boc)₂O (2.64 g, 12.1 mmol) was added, and the mixture was stirred at 25° C. for 1 h. Water (50 mL) was added, then the reaction mixture was extracted with EA (80 mL*2). The combined organic phase was dried by Na₂SO₄, filtered, concentrated and purified by silicagel column (100-200 mesh) chromatography purification eluted with PE:EA (from 100:0 to 1:1) to afford the compound 23-1-7 (800 mg, 1.21 mmol, 20.00% yield) as yellow solid. MS (m/z) 664.9 (M+H)⁺.

### Step 4: Synthesis of Compound 23-1-8

A mixture of compound 23-1-7 (800 mg, 1.21 mmol) and NBS (248.46 mg, 1.45 mmol) in DCM (12 mL) was stirred at 0° C. for 1 h. Water (15 mL) was added, then the reaction mixture was extracted with EA (20 mL*2). The combined organic phase was dried by Na₂SO₄, filtered, concentrated and purified by silicagel column (100-200 mesh) chromatography purification eluted with PE:EA (from 100:0 to 2:1) to afford the compound 23-1-8 (350 mg, 0.47 mmol, 38.84% yield) as yellow oil. MS (m/z) 743.3 (M+H)⁺.

### Step 5: Synthesis of Compound 23-1-9

A mixture of compound 23-1-8 (350 mg, 0.47 mmol) and DDQ (106.69 mg, 0.47 mmol) in DCM (12 mL) was stirred at 25° C. for 1 h. The reaction mixture was filtered and purified by silicagel column (100-200 mesh) chromatography purification eluted with PE:EA (from 100:0 to 2:1) to obtain compound 23-1-9 (150 mg, 0.24 mmol, 51.31% yield) as yellow oil. MS (m/z) 623.1 (M+H)⁺.

### Step 6: Synthesis of Compound 23-1-11

A mixture of compound 23-1-9 (150 mg, 0.24 mmol), compound 23-1-10 (80.64 mg, 0.48 mmol), Xphos-Pd-G4 (30.% mg, 0.036 mmol) and K₃PO₄ (127.20 mg, 0.60 mmol) in dioxane (10 mL) and water (2 mL) was stirred at 95° C. for 0.5 h under N₂. The reaction mixture was concentrated and purified by prep-TLC (DCM/MeOH=20/1) to obtain compound 23-1-11 (15 mg, 0.027 mmol, 11.41% yield) as yellow solid. MS (m/z) 549.2 (M+H)⁺.

### Step 7: Synthesis of Compound 120

A mixture of compound 23-1-11 (15 mg, 0.027 mmol) in DCM (2 mL) and TFA (2 mL) was stirred at 15° C. for 1 h. The reaction mixture was concentrated and purified by reserved phase column (MeOH/H2O=3/7) and prep-TLC (DCM/MeOH=5/1) to obtain compound 120 (4.06 mg, 0.009 mmol, 33.33% yield) as white solid.

Analytical data for compound 120 is provided below.

| Cpd. No. | Structure | Analytical Data |
|---|---|---|
| 120 | 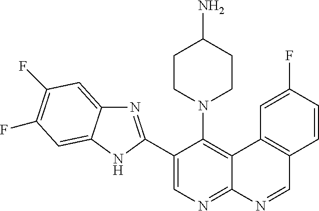 [SMILES: NC1CCN(c2c(-c3nc4cc(F)c(F)cc4[nH]3)cnc3ncc4ccc(F)cc4c23)CC1] | 1H NMR (400 MHz, Methanol-d4): δ 9.73 (s, 1H), 9.48 (s, 1H), 8.80 (s, 1H), 8.37 (dd, J = 8.7, 6.2 Hz, 1H), 7.70- 7.66 (s, 1H), 7.62-7.57 (m, 2H), 3.36 (d, J = 12.6 Hz, 2H), 3,01-2.81 (m, 2H), 2.01-1.91 (m, 3H), 1.26-1.25 (m, 2H). MS (m/z) 448.9 (M + H)⁺. |

### Example 24

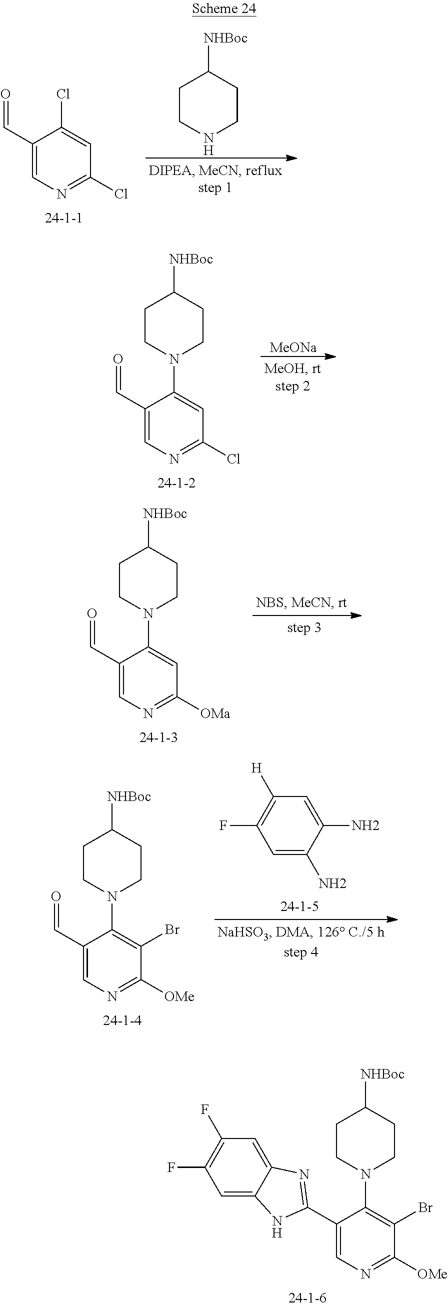

### Step 1: Synthesis of tert-butyl (1-(2-chloro-5-formylpyridin-4-yl) piperidin-4-yl) carbamate (Compound 24-1-2)

To a stirred solution of 4,6-dichloronicotinaldehyde (compound 24-1-1, 10 g, 56.8 mmol, 1 equiv.) in MeCN (150 ml) was added tert-butyl piperidin-4-ylcarbamate (11.36 g, 56.8 mmol, 1 equiv.), DIPEA (36.64 g, 284 mmol, 5 equiv.) at 20° C. The reaction was stirred at 20° C. for 16 hours. TLC (PE:EtOAc=1:1) indicated the reaction was complete. The reaction mixture was diluted with EtOAc (700 mL), washed with H₂O (500 mL×3), dried over Na₂SO₄ and evaporated to give the residue. The residue was purified by column chromatography on silica gel eluted with PE/EtOAc (100:1˜1:1) to give tert-butyl (1-(2-chloro-5-formylpyridin-4-yl) piperidin-4-yl) carbamate (compound 24-1-2, 8 g, 23.53 mmol, 42%) as a white solid. MS (m/z) 340.3 (M+H)⁺.

### Step 2: Tert-butyl (1-(5-formyl-2-methoxypyridin-4-yl) piperidin-4-yl) carbamate (Compound 24-1-3)

To a stirred solution of tert-butyl (1-(2-chloro-5-formylpyridin-4-yl) piperidin-4-yl) carbamate (compound 24-1-2, 8 g, 23.53 mmol, 1 equiv.) in MeOH (150 ml, 100.0%) was added MeONa (5.08 g, 94.12 mmol, 4 equiv.) at 0° C. The reaction was stirred at 60° C. for 3 hours. TLC (PE:EtOAc=1:1) indicated the reaction was complete. The reaction mixture was diluted with EtOAc (700 mL), washed with H₂O (500 mL×3), dried over Na₂SO₄ and evaporated to give tert-butyl (1-(5-formyl-2-methoxypyridin-4-yl)piperidin-4-yl)carbamate (7 g, 20.9 mmol, 88%) as a white solid. MS (m/z) 336.3 (M+H)⁺.

### Step 3: Synthesis of tert-butyl (1-(3-bromo-5-formyl-2-methoxypyridin-4-yl)piperidin-4-yl)carbamate (Compound 24-14)

To a stirred solution of tert-butyl (1-(5-formyl-2-methoxypyridin-4-yl)piperidin-4-yl)carbamate (compound 24-1-3, 7 g, 20.9 mmol, 1 equiv.) in MeCN (150 ml, 100.0%) was added NBS (3.72 g, 20.9 mmol, 1 equiv.) at 20° C. The reaction was stirred at 20° C. for 1 hours. TLC (PE:EtOAc=3:1) indicated the reaction was complete. The reaction mixture was diluted with EtOAc (700 mL), washed with H₂O (500 mL×3), dried over Na₂SO₄ and evaporated to give the residue. The residue was purified by column chromatography on silica gel eluted with PE/EtOAc (100:1-1:1) to give tert-butyl (1-(3-bromo-5-formyl-2-methoxypyridin-4-yl)piperidin-4-yl)carbamate (compound 24-14, 6 g, 14.5 mmol, 69%) as a white solid. MS (m/z) 414.1/416.1 (M+H)⁺.

### Step 4: Synthesis of tert-butyl (1-(3-bromo-5-(5,6-difluoro-1H-benzo[d]imidazol-2-yl)-2-methoxypyridin-4-yl)piperidin-4-yl)carbamate (Compound 24-1-6)

To a stirred solution of tert-butyl (1-(3-bromo-5-formyl-2-methoxypyridin-4-yl)piperidin-4-yl)carbamate (compound 24-14, 6 g, 14.5 mmol, 1 equiv.) in DMA (150 ml, 100.0%) was added 4,5-difluorobenzene-1,2-diamine (2.09 g, 14.5 mmol, 1 equiv.) and NaHSO₃ (6.03 g, 58 mmol, 4 equiv.) at 20° C. The reaction was stirred at 120° C. for 5 hours. TLC (PE:EtOAc=1:1) indicated the reaction was complete. The reaction mixture was diluted with EtOAc (700 mL), washed with H₂O (500 mL×3), dried over Na₂SO₄ and evaporated to give the residue. The residue was purified by column chromatography on silica gel eluted with PE/EtOAc (100:1-1:1) to give tert-butyl (1-(3-bromo-5-(5,6-difluoro-1H-benzo[d]imidazol-2-yl)-2-methoxypyridin-4-yl)piperidin-4-yl)carbamate (compound 24-1-6, 6.5 g, 12.1 mmol, 83%) as a white solid. MS (m/z) 538.2/540.2 (M+H)⁺.

### Step 5: tert-butyl (1-(5-(5,6-difluoro-1H-benzo[d]imidazol-2-yl)-3-(5-fluoro-2-formylphenyl)-2-methoxypyridin-4-yl)piperidin-4-yl)carbamate (Compound 24-1-8)

To a stirred solution of tert-butyl (1-(3-bromo-5-(5,6-difluoro-1H-benzo[d]imidazol-2-yl)-2-methoxypyridin-4-yl)piperidin-4-yl)carbarnate (compound 24-1-6, 6.5 g, 12.1 mmol, 1 equiv.) in toluene (60 ml), EtOH (40 ml), H₂O (20 ml) were added (5-fluoro-2-formylphenyl)boronic acid (2.03 g, 12.1 mmol, 1 equiv.) and K₂CO₃ (3.34 g, 24.2 mmol, 2 equiv.) at 20° C. The reaction was stirred at 90° C. for 5 hours. TLC (PE:EtOAc=1:1) indicated the reaction was complete. The reaction mixture was diluted with EtOAc(700 mL), washed with H₂O (500 mL×3), dried over Na₂SO₄ and evaporated to give the residue. The residue was purified by column chromatography on silica gel eluted with PE/EtOAc (100:1-1:1) to give tert-butyl (1-(5-(5,6-difluoro-1H-benzo[d]imidazol-2-yl)-3-(5-fluoro-2-formylphenyl)-2-methoxypyridin-4-yl)piperidin-4-yl)carbamate (compound 24-1-8, 4 g, 6.87 mmol, 57%) as a white solid. MS (m/z) 582.3 (M+H)⁺.

### Step 6:Tert-butyl (1-(5-(5,6-difluoro-1H-benzo[d]imidazol-2-yl)-3-(5-fluoro-2-(hydroxymethyl)phenyl)-2-methoxypyridin-4-yl)piperdine-4-yl)carbamate (Compound 24-1-9)

To a solution of tert-butyl (1-(5-(5,6-difluoro-1H-benzo[d]imidazol-2-yl)-3-(5-fluoro-2-formylphenyl)-2-methoxypyridin-4-yl)piperidin-4-yl)carbamate (compound 24-1-8, 4 g, 6.87 mmol) in MeOH (20 mL) at 25° C. was added NaBH₄ (522 mg, 13.74 mmol, 2 eq.) portion-wise. The solution was stirred at 25° C. for 2 hours. The reaction mixture was quenched by 1N HCl solution and extracted by EA (20 mL*3). The combined layers were combined and concentrated under reduced pressure to give tert-butyl (1-(5-(5,6-difluoro-1H-benzo[d]imidazol-2-yl)-3-(5-fluoro-2-(hydroxymethyl)phenyl)-2-methoxypyridin-4-yl)piperidin-4-yl)carbamate (compound 24-1-9, 3.5 g, crude) as colorless oil.

### Step 7: Tert-butyl (1-(5-(5,6-difluoro-1H-benzo[d]imidazol-2-yl)-3-(5-fluoro-2-(hydroxymethyl)phenyl)-2-hydroxypyridin-4-yl)piperidin-4-yl)carbamate (Compound 24-1-10)

A solution of tert-butyl (1-(5-(5,6-difluoro-1H-benzo[d]imidazol-2-yl)-3-(5-fluoro-2-(hydroxymethyl)phenyl)-2-methoxypyridin-4-yl)piperidin-4-yl)carbamate (compound 24-1-9, 3.5 g, crude) was dissolved in DCE (20 mL), followed by addition of BBr₃ (10 mL) and the solution was heated at 80° C. for 4 hours. The reaction was quenched by saturated NaHCO₃ solution until PH was about 8-9. The mixture was extracted by DCM (10 mL*3) and the combined layers were concentrated under reduced pressure to deliver tert-butyl (1-(5-(5,6-difluoro-1H-benzo[d]imidazol-2-yl)-3-(5-fluoro-2-(hydroxymethyl)phenyl)-2-hydroxypyridin-4-yl)piperidin-4-yl)carbamate (compound 24-1-10, 250 mg, 0.44 mmol, 10% yield) as white solid.

### Step 8: tert-butyl (1-(2-(5,6-difluoro-1H-benzo[d]imidazol-2-yl)-9-fluoro-6H-isochromeno[3,4-b]pyridin-1-yl)piperidin-4-yl)carbamate (Compound 24-1-11)

To a solution of tert-butyl (1-(5-(5,6-difluoro-1H-benzo[d]imidazol-2-yl)-3-(5-fluoro-2-(hydroxymethyl)phenyl)-2-hydroxypyridin-4-yl)piperidin-4-yl)carbamate (compound 24-1-10, 250 mg, 0.44 mmol) in THE (10 mL) at 0° C. was added DIAD (111 mg, 0.55 mmol, 1.1 eq.) and PPh_{b}(144 mg, 0.55 mmol, 1.1 eq.) respectively. Then the solution was stirred at 25° C. overnight. The reaction mixture was extracted by EA (10 mL*3) and the combined layers were concentrated under reduced pressure to give tert-butyl (1-(2-(5,6-difluoro-1H-benzo[d]imidazol-2-yl)-9-fluoro-6H-isochromeno[3,4-b]pyridin-1-yl)piperidin-4-yl)carbamate (compound 24-1-11, 100 mg, white solid).

### Step 9: 1-(2-(5,6-difluoro-1H-benzo[d]imidazol-2-yl)-9-fluoro-6H-isochromeno[3,4-b]pyridin-1-yl)piperidin-4-amine (Compound 121)

A solution of tert-butyl (1-(2-(5,6-difluoro-1H-benzo[d]imidazol-2-yl)-9-fluoro-6H-isochromeno[3,4-b]pyridin-1-yl)piperidin-4-yl)carbamate (compound 24-1-11, 100 mg, 0.18 mmol) in DCM (5 mL) was added TFA (5 mL). The reaction mixture was concentrated under reduced pressure to give the residue which was purified by prep-HPLC (0.5% FA as additive) to give 1-(2-(5,6-difluoro-1H-benzo[d]imidazol-2-yl)-9-fluoro-6H-isochromeno[3,4-b]pyridin-1-yl)piperidin-4-amine (compound 121, 13.8 mg, white solid) as white solid.

Analytical data for compound 121 is provided below.

| Cpd. No. | Structure | Analytical Data |
|---|---|---|
| 121 | 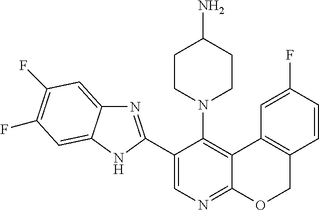 [SMILES: NC1CCN(c2c(-c3nc4cc(F)c(F)cc4[nH]3)cnc3c2-c2cc(F)ccc2CO3)CC1] | ¹H NMR (400 MHz, CD₃OD) δ 8.50 (s, 1H), 8.03 (s, 1H), 7.62-7.50 (m, 2H), 7.47-7.38 (m, 1H), 7.20-7.02 (m, 1H), 5.14 (s, 2H), 3.29-3.21 (m, 2H), 3.13-3.08 (m, 1H), 3.01-2.92 (m, 2H), 1.78-1.52 (m, 4H). MS (m/z) 452.25 (M + H)⁺. |

### Example 125

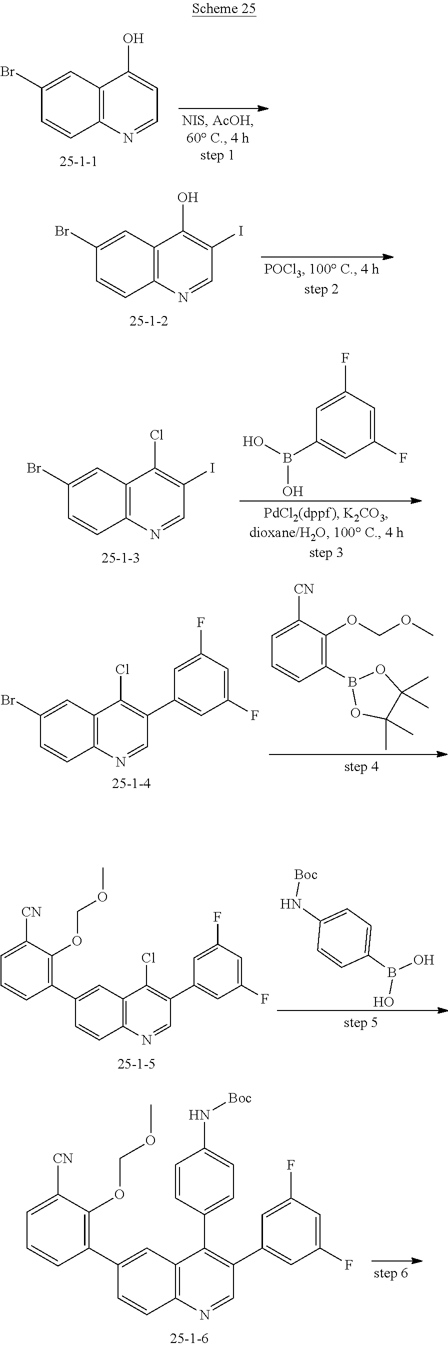

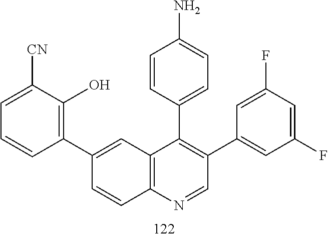 [SMILES: N#Cc1cccc(-c2ccc3ncc(-c4cc(F)cc(F)c4)c(-c4ccc(N)cc4)c3c2)c1O]

### Step 1: Synthesis of 6-bromo-3-iodoquinolin-4-ol (Compound 25-1-2)

The mixture of compound 25-1-1 (3000 mg, 13.389 mmol, 1 eq) was dissolved in AcOH (50 mL) was added NIS (3012 mg, 13.389 mmol, 1 eq). The mixture was stirred at 60° C. for 3 h. The mixture was cooled to zero degree, and lots of solid was observed. LCMS showed solid was title compound with good purity. After filtering, 6-bromo-3-iodoquinolin-4-ol (compound 25-1-2, 4.4 g, yield 94%) was obtained as a white solid. MS (m/z) 349.9 (M+H)⁺.

### Step 2: Synthesis of 6-bromo-4-chloro-3-iodoquinoline (Compound 25-1-3)

A solution of compound 25-1-2 (4.4 g, crude) in POCl₃ (45 mL) was stirred at 100° C. for 16 h. The mixture was cooled to zero degree, then poured into ice-NaHCO₃ (aq), continue to add NaHCO₃ solid until pH=7. The solution was extracted with EA (50 mL×4). The combined organic extract and concentrated to give the crude material, the crude material was purified by silica gel chromatography eluted with PE:EtOAc (from 100:1 to 5:1) to give product 6-bromo-4-chloro-3-iodoquinoline (compound 25-1-3, 4.4 g) as white solid. MS (m/z) 367.8 (M+H)⁺.

### Step 3: Synthesis of 6-bromo-4-chloro-3-(3,5-difluorophenyl)quinoline (Compound 25-1-4)

To a solution of compound 25-1-3 (4.4 g, crude), (3,5-difluorophenyl)boronic acid (1.16 g, 7.36 mmol, 1.1 eq) and K₂CO₃ (3.05 g, 22.08 mmol, 3 eq) in the mixture of dioxane (40 mL) was added PdCl₂(dppf) (600 mg, 0.74 mmol, 0.1 eq) under N₂. The suspension was degassed under vacuum and purged with N₂ several times. Then the reaction mixture was stirred at 90° C. for 4 h. The reaction mixture was filtered and washed with EA (200 mL×3). The combined organic extract and concentrated to give the crude material, the crude material was purified by silica gel chromatography eluted with PE:EtOAc (from 100:1 to 5:1) to give product 6-bromo-4-chloro-3-iodoquinoline (compound 25-14, 2.3 g) as white solid. MS (m/z) 354.0 (M+H)⁺.

### Step 4: Synthesis of 3-(4-chloro-3-(3,5-difluorophenyl)quinolin-6-yl)-2-(methoxy methoxy)benzonitrile (Compound 25-1-5)

The mixture of compound 25-1-4 (300 mg, 0.85 mmol, 1 eq) and 2-(methoxymethoxy)-3-(4,4,5,5-tetramethyl-1,3,2-dioxaborolan-2-yl) benzonitrile (400 mg, 0.85 mmol, 1 eq) was dissolved in dioxane (10 mL) was added PdCl₂(dppf) (73 mg, 0.056 mmol, 0.1 eq) and K₂CO₃ (352 mg, 2.25 mmol, 3 eq). The mixture was stirred at 90° C. for 2 h. The reaction mixture was diluted with H₂O (20 mL) and extracted with EtOAc (20 mL×3). The combined organic phase was washed with brine, dried with Na₂SO₄, filtered. The organic layer was concentrated under the reduced pressure to give the crude product and purified by the silica gel column chromatography (PE:EA from 10/1 to 1/l to give the compound 25-1-5 as yellow solid (200 mg, 53.95% yield). MS (m/z) 437.1 (M+H)⁺.

### Step 5: Synthesis of tert-butyl(4-(6-(3-cyano-2-(methoxymethoxy)phenyl)-3-(3,5-difluoro phenyl)quinolin-4-yl)phenyl)carbamate (Compound 25-1-6)

The mixture of compound 25-1-5 (100 mg, 0.23 mmol, 1 eq) and (4-((tert-butoxycarbonyl)amino)phenyl)boronic acid (112 mg, 0.47 mmol, 2 eq) was dissolved in THF (6 mL) and H₂O (0.5 mL) was added Pd(PPh₃)₄ (58 mg, 0.05 mmol, 0.2 eq) and K₂CO₃ (96 mg, 0.69 mmol, 3 eq). The mixture was stirred at 70° C. for 4 h. The reaction was monitored by LCMS, which showed it worked well. The reaction mixture was diluted with H₂O (20 mL) and extracted with EtOAc (20 mL×3). The combined organic phase was washed with brine, dried with Na₂SO₄, filtered. The organic layer was concentrated under the reduced pressure to give the crude product and purified by the silica gel column chromatography (PE:EA from 10/1 to 1/1 to give the compound 25-1-6 as yellow solid (70 mg, 51.31% yield). MS (m/z) 594.2 (M+H)⁺.

### Step 6: Synthesis of 3-(4-(4-aminophenyl)-3-(3,5-difluorophenyl)quinolin-6-yl)-2-hydroxy benzonitrile (Compound 122)

The mixture of compound 25-1-6 (70 mg, 0.12 mmol, 1 eq) dissolved in DCM (2 mL) was added TFA (2 mL). The mixture was stirred at 25° C. for 1 h. The reaction mixture was concentrated and purified by Prep-HPLC to give compound 122 (11.4 mg, 21.15% yield) as yellow solid.

Compounds 122-125 were synthesized according to the 6-step procedure described in Example 25. Analytical data for compounds 122-125 is provided below.

| Cpd. No. | Structure | Analytical Data |
|---|---|---|
| 122 | 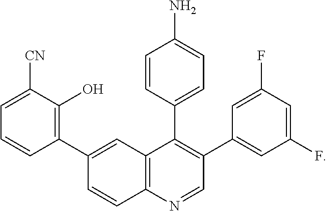 [SMILES: N#Cc1cccc(-c2ccc3ncc(-c4cc(F)cc(F)c4)c(-c4ccc(N)cc4)c3c2)c1O] | 1H NMR (400 MHz, DMSO-d6): δ ppm 8.91 (s, 1H), 8.13- 8.11 (d, J = 8.8 Hz, 1H), 7.97-7.94 (d, J = 9.2 Hz, 1H), 7.89- 7.88 (d, J = 1.6 Hz, 1H), 7.56-7.46 (m, 2H), 7.17-7.13 (m, 1H), 6.96-6.90 (m, 5H), 6.59-6.57 (d, J = 8 Hz, 2H), 5.33 (s, 2H). MS (m/z) 450.1 (M + H)⁺. |
| 123 | 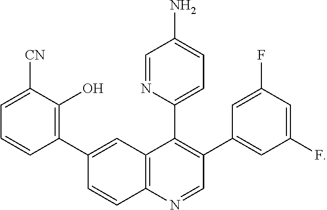 [SMILES: N#Cc1cccc(-c2ccc3ncc(-c4cc(F)cc(F)c4)c(-c4ccc(N)cn4)c3c2)c1O] | 1H NMR (400 MHz, DMSO-d6): δ ppm 8.97 (s, 1H), 8.15- 8.13 ( d, J = 8.4 Hz, 1H), 8.02-7.96 (m, 2H), 7.81-7.80 (d, J = 1.2 Hz, 1H), 7.56-7.54 (d, J = 7.2 Hz, 1H), 7.46-7.44 (d, J = 7.6 Hz, 1H), 7.20-7.15 (m, 1H), 6.97-6.91 (m, 5H), 5.58 (s, 2H). MS (m/z) 451.1 (M + H)⁺. |
| 124 | 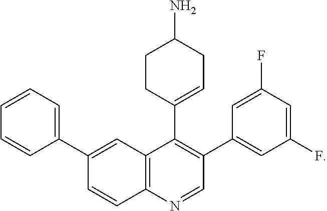 [SMILES: NC1CC=C(c2c(-c3cc(F)cc(F)c3)cnc3ccc(-c4ccccc4)cc23)CC1] | 1H NMR (400 MHz, DMSO-d6): δ ppm δ 8.86 (d, J = 7.8 Hz, 1H), 8.31 (d, J = 66.0 Hz, 1H), 8.17 (t, J = 6.8 Hz, 2H), 7.81 (d, J = 7.7 Hz, 2H), 7.55 (t, J = 7.6 Hz, 2H), 7.45 (t, J = 7.4 Hz, 1H), 7.40-7.24 (m, 3H), 5.80 (s, 1H), 2.95-2.86 (m, 1H), 2.56 (s, 1H), 2.44-2.36 (m, 1H), 1.90 (s, 2H), 1.86-1.69 (m, 1H), 1.24 (s, 1H). MS (m/z) 413.2 (M + H)⁺. |
| 125 | 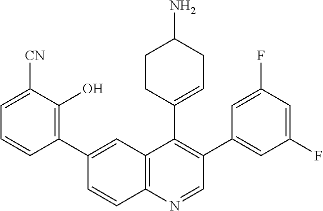 [SMILES: N#Cc1cccc(-c2ccc3ncc(-c4cc(F)cc(F)c4)c(C4=CCC(N)CC4)c3c2)c1O] | 1H NMR (400 MHz, DMSO-d6): δ ppm 8.800-8.754 (d, J = 18.4 Hz, 1.74H), 8.469 (s, 0.51H), 8.053 (s, 0.47H), 8.063- 7.991 (m, 2H), 7.526-7.231 (m, 5H), 6.468-6.359 (m, 1H), 5.697 (s, 1H). MS (m/z) 454.1 (M + H)⁺. |

### Example 26

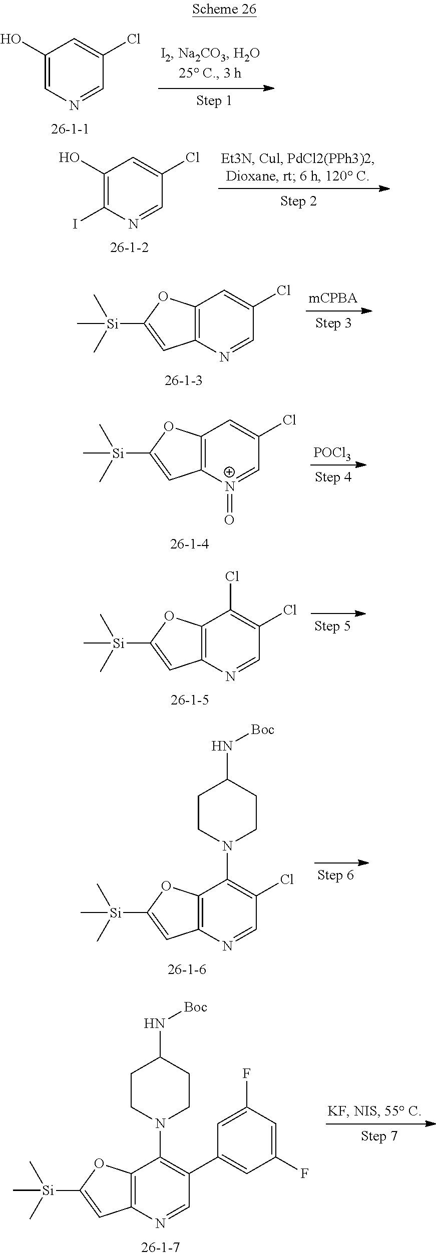

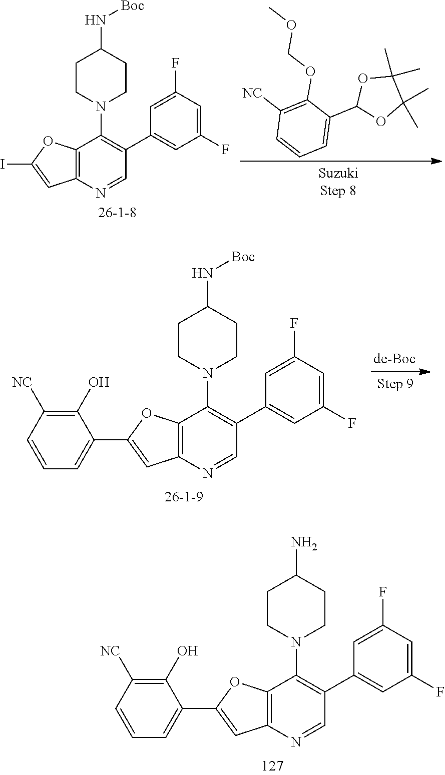 [SMILES: CC(C)(C)OC(=O)NC1CCN(c2c(-c3cc(F)cc(F)c3)cnc3cc(I)oc23)CC1 ; COCOc1c(C#N)cccc1C1OC(C)(C)C(C)(C)O1 ; CC(C)(C)OC(=O)NC1CCN(c2c(-c3cc(F)cc(F)c3)cnc3cc(-c4cccc(C#N)c4O)oc23)CC1 ; N#Cc1cccc(-c2cc3ncc(-c4cc(F)cc(F)c4)c(N4CCC(N)CC4)c3o2)c1O]

### Step 1: Synthesis of 5-chloro-2-iodopyridin-3-ol (Compound 26-1-2)

To a solution of sodium carbonate (4.8 g, 46.41 mmol) in water (45 mL) was added 5-chloropyridin-3-ol (compound 26-1-1, 3.0 g, 23.16 mmol) and I₂ (6.0 g, 23.16 mmol) at 25° C. The resulting mixture was stirred for 5 h at 25° C. Treated with a hydrochloric acid solution (90 mL, 2 M), the precipitate that formed was collected by filtration and re-dissolved in ethyl acetate (200 mL). The mixture was washed with brine (100 mL×2) and the organic phase was dried over sodium sulfate. The solvent was removed under reduced pressure to give 5-chloro-2-iodopyridin-3-ol as yellow solid (compound 26-1-2, 5.7 g, 96%). LCMS: m/z: 255.9 (M+H⁺) at 1.49 min.

### Step 2: Synthesis of 6-chloro-2-(trimethylsilyl)furo[3,2-b]pyridine (Compound 26-1-3)

To a solution of compound 26-1-2 (5.7 g, 22.3 mmol) in dioxane (91.2 mL, 1.076 mol) was added ethynyltrimethylsilane (4.75 g, 48.38 mmol), Pd(PPh₃)₂Cl₂ (1.695 g, 2.42 mmol), CuI (57 mg, 0.285 mmol) and triethylamine (12.1 g, 119.7 mmol) at 25° C. The resulting mixture was stirred for 6 h at 120° C. The mixture was cooled to 25° C., and treated with water (350 mL). The resulting mixture was extracted with ethyl acetate (350 mL×3). The organic phases were combined, washed with brine and dried over sodium sulfate. The solvent was removed under reduced pressure and the residue was purified by flash chromatography eluting with ethyl acetate in hexane (0% to 5% gradient) to give 6-chloro-2-(trimethylsilyl) furo[3,2-b]pyridine (compound 26-1-3, 1.5 g, 70%) as brown solid. MS (m/z) 226.0 (M+H)⁺.

### Step 3: Synthesis of 6-chloro-2-(trimethylsilyl)furo[3,2-b]pyridine-4oxide (Compound 26-1-4)

To a solution of compound 26-1-3 (1.5 g, 6.64 mmol, 1.00 equiv) in dichloromethane (50 mL) was added m-CPBA (1.71 g, 9.96 mmol) in portions at 0° C. The resulting solution was stirred for 30 min at 0° C. then warmed up to room temperature and stirred for another 16 h at 25° C. The reaction mixture was treated with water (100 mL) and adjusted pH=8 by using a saturated solution of sodium bicarbonate. The resulting mixture was then extracted with dichloromethane (100 mL×2) and the organic phases were combined, washed with brine and dried over sodium sulfate. The solvent was removed under reduced pressure to give compound 26-14 (1.9 g, crude). MS (m/z) 242.1 (M+H)⁺.

### Step 4: Synthesis of 6,7-dichloro-2-(trimethylsilyl)furo[3,2-b]pyridine (Compound 26-1-5)

To a solution of compound 26-14 (1.9 g, crude) in toluene (50 mL) was added POCl₃ (2 g, 13.07 mmol) at 25° C. The resulting solution was then stirred for 3 h at 95° C. After cooling to 25° C., the reaction mixture was concentrated under reduced pressure and the resulting residue was poured into ice-water (70 mL). The pH value of the mixture was adjusted to 9 with a saturated solution of sodium carbonate. The resulting mixture was then extracted with dichloromethane (70 mL×3). The solvent was removed under reduced pressure and the residue was purified by flash chromatography eluting with ethyl acetate in hexane (0% to 2% gradient) to give compound 26-1-5 (800 mg, 47% over 2 steps). MS (m/z) 260.0 (M+H)⁺.

### Step 5: Synthesis of tert-butyl (1-(6-chloro-2-(trimethylsilyl)furo[3,2-b]pyridin-7-yl) piperidin-4-yl)carbamate (Compound 26-1-6)

To a solution of compound 26-1-5 (300 mg, 1.2 mmol, 1 eq) in DMSO (0.05 mL) was added tert-butyl piperidin-4-ylcarbamate (2317 mg, 11.6 mmol, 10 eq). The reaction mixture was stirred at 140° C. for 2 h. The reaction mixture was diluted with water (40 mL) and extracted with EA (100 mL×3). The solvent was removed under reduced pressure and the residue was purified by flash chromatography eluting with ethyl acetate in hexane (0% to 2% gradient) to give compound 26-1-6 as yellow solid (400 mg, 81.7%). MS (m/z) 424.4 (M+H)⁺.

### Step 6: Synthesis of tert-butyl (1-(6-(3,5-difluorophenyl)-2-(trimethylsilyl)furo[3,2-b]pyridin-7-yl)piperidin-4-yl)carbamate (Compound 26-1-7)

To a solution of compound 26-1-6 (400 mg, 0.243 mmol, 1 eq) and (3,5-difluorophenyl)boronic acid (46 mg, 0.84 mmol, 1.2 eq) in the mixture of dioxane:H₂O=10:1 (6 mL) was added K₂CO₃ (67 mg, 0.487 mmol, 2 eq) and Pd-G2 (19 mg, 0.024 mmol, 0.1 eq). The reaction mixture was stirred at 110° C. for 2 h. The reaction mixture was cooled and diluted with water (30 mL) and extracted with EA (50 mL×3). The combined organic extract was concentrated to give the crude material, which was purified by chromatography through a Redi-Sep pre-packed silica gel column (12 g), eluting with a gradient of 0% to 10% MeOH in DCM, to give compound 26-1-7 (300 mg) as grey solid. MS (m/z) 502.1 (M+H)⁺.

### Step 7: Synthesis of tert-butyl(1-(6-(3,5-difluorophenyl)-2-iodofuro[3,2-b]pyridin-7-yl) piperidin-4-yl)carbamate (Compound 26-1-8)

To a solution of compound 26-1-7 (300 mg, 0.92 mmol, 1 eq) in CH₃CN (10 mL) were added potassium fluoride (161 mg, 2.7 mmol) and NIS (621 mg, 3 eq) at 25° C. The resulting solution was stirred for 3 h at 55° C. The reaction mixture was cooled to 25° C. and treated with NaHSO₃ solution (100 mL, 4 M). The resulting mixture was extracted with ethyl acetate (50 mL×3). The organic phases were combined, washed with brine and dried over sodium sulfate. The solvent was removed under reduced pressure to give compound 26-1-8 (190 mg) as yellow solid. MS (m/z) 556.0 (M+H)⁺.

### Step 8: Synthesis of tert-butyl (1-(2-(3-cyano-2-hydroxyphenyl)-6-(3,5-difluorophenyl) furo[3,2-b]pyridin-7-yl)piperidin-4-yl)carbamate (Compound 26-1-9)

To a solution of compound 26-1-8 (190 mg, 0.342 mmol, 1 eq), 2-(3-bromopropoxy)-3-(4,4,5,5-tetramethyl-1,3-dioxolan-2-yl)benzonitrile (81 mg, 0.513 mmol, 1.5 eq) and K₂CO; (142 mg, 1.027 mmol, 3 eq) in the mixed solvent of dioxane (8 mL) and H₂O (0.8 mL) was added Pd(dppf)Cl₂ (25 mg, 0.034 mmol, 0.1 eq). The reaction mixture was stirred at 110° C. for 2 h under N₂ condition. The reaction mixture was diluted with water (30 mL) and extracted with EA (50 mL×3). The combined organic extract and concentrated to give the crude material, which was purified by chromatography through a Redi-Sep pre-packed silica gel column (12 g), eluting with a gradient of 0% to 30% EA in PE, to provide compound 26-1-9 (45 mg) as yellow oil. MS (m/z) 547.1 (M+H)⁺.

### Step 9: Synthesis of 3-(7-(4-aminopiperidin-1-yl)-6-(3,5-difluorophenyl)furo[3,2-b]pyridine-2-yl)-2-hydroxybenzonitrile (Compound 127)

To a solution of compound 26-1-9 (45 mg, 0.076 mmol, 1 eq) in DCM (2 mL) was added TFA (1 mL). The reaction mixture was stirred at 25° C. for 2 h. The reaction was complete detected by LC-MS. Then concentrated to give the crude material, the crude product was purified by Prep-HPLC to give compound 127 (13.3 mg).

Compounds 127-131 were synthesized according to the 9-step procedure described in Example 26. Analytical data for compounds 127-131 are provided below.

| Cpd. No. | Structure | Analytical Data |
|---|---|---|
| 127 | 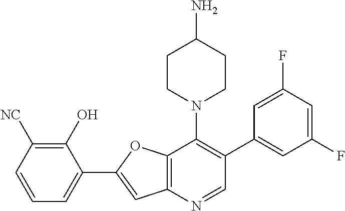 [SMILES: N#Cc1cccc(-c2cc3ncc(-c4cc(F)cc(F)c4)c(N4CCC(N)CC4)c3o2)c1O] | 1H NMR (400 MHz, DMSO-d6): δ ppm 8.000 (s, 1H), 7.927 (s, 1H), 7.786-7.762 (dd, J₁ = 8.8 Hz, J₂ = 2.0 Hz, 1H), 7.691 (s, 1H), 7.310-7.206 (m, 4H), 6.214 (s, 1H), 3.534-3.504 (d, J = 1 2 Hz, 2H), 3.247-3.199 (m, 1H), 3.161-3.102 (t, J = 12.0 Hz, 2 H), 1.916-1.889 (d, J = 10.8 Hz, 2H), 1.610-1.573 (m, 1H). MS (m/z) 447.2 (M + H)⁺. |
| 128 | 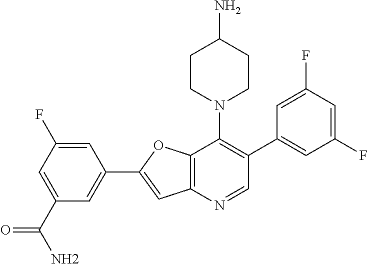 [SMILES: NC(=O)c1cc(F)cc(-c2cc3ncc(-c4cc(F)cc(F)c4)c(N4CCC(N)CC4)c3o2)c1] | 1H NMR (400 MHz, DMSO-d6): δ ppm 8.311 (s, 1H), 8.213- 8.219 (m, 2H), 7.982 (d, J = 13.2 Hz, 1H), 7.742-7.780 (m, 2H), 7.708 (s 1H ), 7.244-7.388 (m, 3H), 3.543 (d, J = 17.6 Hz, 2H), 3.064-3.203 (m, 3H), 1.846-1.881 (m, 2H), 1.446-1.519 (m, 2H). MS (m/z) 467.1 (M + H)⁺. |
| 129 | 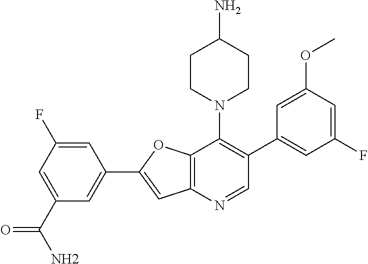 | 1H NMR (400 MHz, DMSO-d6): δ ppm 8.299 (s, 1H), 8.210 (s, 1H), 8.180 (s, 1H), 7.958 (d, J = 10.8 Hz, 1H), 7.744-7.111 (m, 3H), 6.968-6.988 (m, 2H), 6.838 (d, J = 13.2 Hz, 1H), 3.841 (s, 3H), 3.546 (d, J = 11.2 Hz, 2H), 3.107 (t, J = 10 Hz, 2H), 2.849- 2.899 (m, 1H), 1.740-1.814 (m, 2H), 1.323-1.374 (m, 2H). MS (m/z) 476.3 (M + H)⁺. |
| 130 | 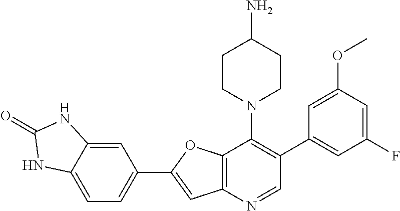 [SMILES: COc1cc(F)cc(-c2cnc3cc(-c4ccc5[nH]c(=O)[nH]c5c4)oc3c2N2CCC(N)CC2)c1] | 1H NMR (400 MHz, DMSO-d6): δ ppm 10.967 (s, 1H), 8.334 (s, 1H), 8.132 (s, 1H), 7.622-7.599 (m, 1H), 7.496 (s, 1H), 7.408 (s, 1H), 7.085-7.065 (d, J = 8.0 Hz, 1H), 6.986- 6.959 (m, 2H), 6.847-6.819 (m, 1H), 3.841 (s, 3H), 3.572-3.544 (d, J = 11.2 Hz, 4H), 3.162-3.102 (t, J = 12.0 Hz, 3H), 1.884-1.856 (d, J = 11.2 Hz, 2H), 1.522-1.496 (d, J = 10.4 Hz, 2H). MS (m/z) 474.2 (M + H)⁺. |
| 131 | 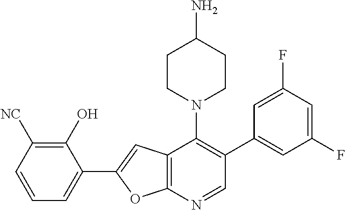 [SMILES: N#Cc1cccc(-c2cc3c(N4CCC(N)CC4)c(-c4cc(F)cc(F)c4)cnc3o2)c1O] | 1H NMR (400 MHz, DMSO-d6): δ ppm 8.029 (s, 3H), 7.847 (s, 1H), 7.737-7.718 (d, J = 7.6 Hz, 1H), 7.295-7.201 (m, 4H), 6.242 (s, 1H), 3.475-3.445 (d, J = 12.0 Hz, 2H), 3.209 (s, 1H), 3.084-3.026 (t, J = 11.2 Hz, 2H), 1,897-1.870 (d, J = 10.8 Hz, 2H), 1.544-1.461 (m, 2H). MS (m/z) 447.1 (M + H)⁺. |

### Example 27

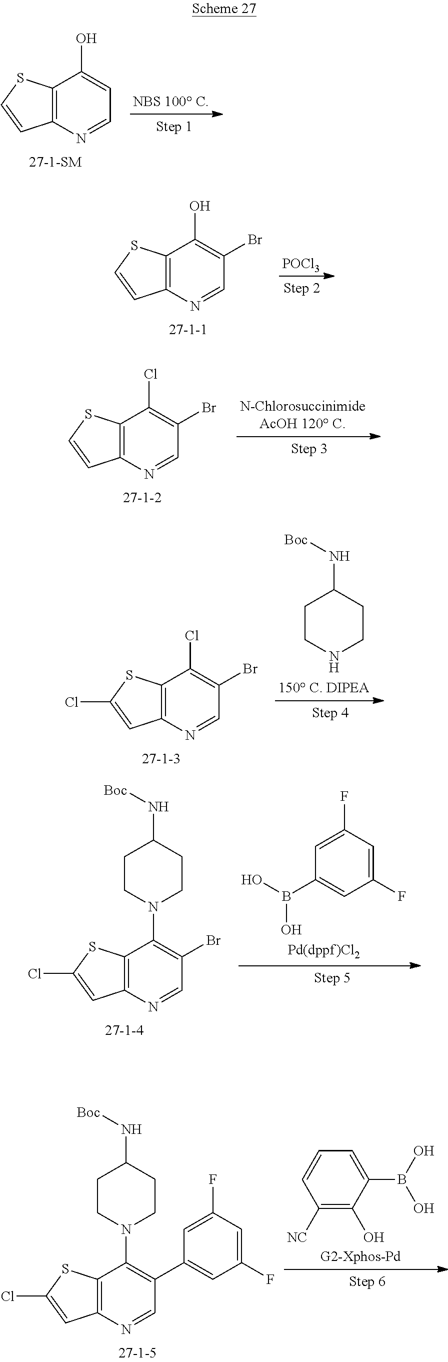

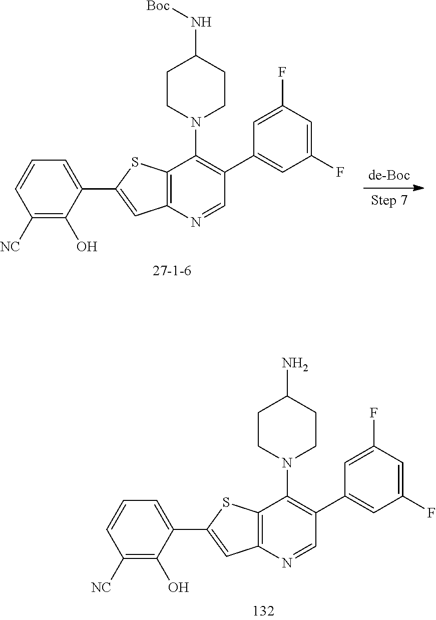 [SMILES: CC(C)(C)OC(=O)NC1CCN(c2c(-c3cc(F)cc(F)c3)cnc3cc(-c4cccc(C#N)c4O)sc23)CC1 ; N#Cc1cccc(-c2cc3ncc(-c4cc(F)cc(F)c4)c(N4CCC(N)CC4)c3s2)c1O]

### Step 1: Synthesis of Compound 27-1-1

A solution of compound 27-1-SM (5 g, 33.07 mmol) in acetonitrile (100 mL) was added NBS (7.65 g, 42.99 mmol), the resulting mixture was stirred at 100° C. for 3 hr. The reaction mixture was concentrated to dryness and used for the next step without further compound 27-1-1 (12.30 g, crude) as yellow solid. MS (m/z) 229.9 (M+H)⁺.

### Step 2: Synthesis of Compound 27-1-2

A solution of compound 27-1-1 (12.3 g, 53.46 mmol) in POCl₃ (164.50 g, 1.07 mol, 100 mL), the resulting mixture was stirred at 110° C. for 2 hr. The reaction mixture was cooled to 25° C., then concentrated to remove POCl₃, then diluted with DCM. Repeat to concentrate again. The residue was diluted with DCM (100 mL), then poured into ice-water slowly. The resulting mixture was extracted with CH2Cl2 (200 mL×2). The combined organic layer was dried over Na₂SO₄ and concentrated. The crude product was purified by silica gel chromatography eluted with PE:EtOAc=10:1 to give compound 27-1-2 (7.28 g, 24.90 mmol, 46.58% yield, 85% purity) as light-red solid. MS (m/z) 248.9 (M+H)⁺.

### Step 3: Synthesis of Compound 27-1-3

A solution of compound 27-1-2 (1.00 g, 3.82 mmol) in acetic acid (10 mL) was added NCS (1.53 g, 11.47 mmol) at 25° C., the resulting mixture was stirred at 120° C. for 12 hr in N₂. The reaction mixture was diluted with ethyl acetate (20 mL). Then it was extracted with water (20 mL*2). The aqueous phase was stripped extracted with ethyl acetate (20 mL). Combined all organic phases and dried over anhydrous Na₂SO₄. Concentrate the organic phase to obtain a crude product. The crude product was purified by silica gel column chromatography purification eluted with PE:EtOAc from 98:2 to 95:5 to give the compound 27-1-3 (470.00 mg, 1.25 mmol, 32.75% yield, 75.38% purity) as white solid. MS (m/z) 282.00 (M+H)⁺.

### Step 4: Synthesis of Compound 27-1-

A solution of compound 27-1-3 (350 mg, 932.35 μmol) and tert-butyl piperidin-4-ylcarbamate (186.73 mg, 932.35 μmol) in dimethyl sulfoxide (4 mL) was added DIPEA (361.50 mg, 2.80 mmol) at 25° C., and the resulting mixture was stirred at 150° C. for 4 hr. The reaction mixture was diluted with ethyl acetate (20 mL). Then it was extracted with water (20 mL*2). The aqueous phase was stripped extracted with ethyl acetate (20 mL). Combined all organic phases and dried over anhydrous Na₂SO₄. Concentrate the organic phase to obtain a crude product. The crude product was purified by silica gel column chromatography purification eluted with PE:EtOAc (V/V: from 90/10 to 75/25) to give compound 27-14 (200.00 mg, 348.62 μmol, 37.39% yield, 77.88% purity) as yellow solid. MS (m/z) 446.0 (M+H)⁺.

### Step 5: Synthesis of Compound 27-1-5

A solution of compound 27-14 (80 mg, 179.06 μmol) and (3,5-difluorophenyl)boronic acid (33.93 mg, 214.87 μmol) in 1,4-dioxane (2 mL) and H₂O (0.2 mL) was added K₂CO₃ (49.49 mg, 358.11 μmol) and Pd(dppf)Cl₂ (26.28 mg, 35.81 μmol) at 25° C. and the resulting mixture was stirred at 100° C. for 16 hr in N₂. The resulting mixture was then extracted with dichloromethane (100 mL 2) and the organic phases were combined, washed with brine and dried over sodium sulfate. The crude product was purified by silica gel chromatography eluted with PE/EtOAc from 15/1 to 5/1 to give compound 27-1-5 (65.00 mg, 135.43 μmol, 75.63% yield) as yellow oil. MS (m/z) 480.2 (M+H)⁺.

### Step 6: Synthesis of Compound 27-1-6

A solution of compound 27-1-5 (65 mg, 135.43 μmol) and (3-cyano-2-hydroxyphenyl)boronic acid (32.08 mg, 203.14 μmol) in 1,4-dioxane (2 mL) and H₂O (0.2 mL) was added K₂CO₃ (37.43 mg, 270.85 μmol) and G2-XPhos-Pd (15.96 mg, 20.31 μmol) at 25° C. and the resulting mixture was stirred at 100° C. for 16 hr in N₂. The reaction solution was directly concentrated to give the residue, which was purified by silica gel chromatography eluted with PE/EtOAc from 15/1 to 1/1 to give compound 27-1-6 (50.00 mg, 89.67 μmol, 66.21% yield) as yellow solid. MS (m/z) 558.2 (M+H)⁺.

### Step 7: Synthesis of Compound 132

To a solution of compound 27-1-6 (50 mg, 73.81 μmol) in dichloromethane (2 mL) was added TFA (1.49 g, 13.07 mmol, 1 mL) at 25° C. the resulting mixture was stirred at 25° C. for 2 hr. The reaction solution was concentrated to dryness, then diluted with dichlormethane (20 mL). Repeat concentration three times. The crude product was dissolved in MeOH, then added NaHCO₃ solid until pH=6. The methanol liquid was directly purified by Prep-HPLC (Prep-C18, 5 μM OBD, 19-250 mm. Column, Waters; gradient elution of 40% MeCN in water to 50% MeCN in water over a 9 min period, where both solvents contain 10 mmol/L NH₄HCO₃) to provide compound 132(5.00 mg, 10.93 μmol, 14.81% yield, 100% purity) as a white solid.

Compounds 132-134 were synthesized according to the 7-step procedure described in Example 27. Analytical data for compounds 132-134 are provided below.

| Cpd. No. | Structure | Analytical Data |
|---|---|---|
| 132 | 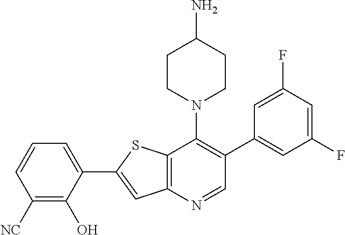 [SMILES: N#Cc1cccc(-c2cc3ncc(-c4cc(F)cc(F)c4)c(N4CCC(N)CC4)c3s2)c1O] | 1H-NMR (400 MHz, DMSO-D6) δ 9.01 (s, 1H), 8.43- 8.38 (m, 2H), 7.29-7.24 (m, 2H), 7.18-7.17 (d, J = 6.3 Hz, 2H), 6.41-6.38 (t, J = 7.6 Hz, 1H), 2.95-2.89 (m, 2H), 2.83- 2.77 (t, J = 11.7 Hz, 3H), 1.83-1.80 (d, J = 11.2 Hz, 2H), 1.58-1.49 (dd, J1 = 24.5 Hz, J2 = 10.2 Hz, 2H), MS (m/z) 463.2 (M + H)⁺. |
| 133 | 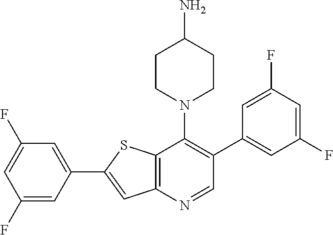 [SMILES: NC1CCN(c2c(-c3cc(F)cc(F)c3)cnc3cc(-c4cc(F)cc(F)c4)sc23)CC1] | 1H-NMR (400 MHz, DMSO-D6) δ 8.59 (s, 1H), 8.45 (s, 1 H), 7.96-7.94 (m, 2H), 7.30-7.21 (m, 4H), 3.38-3.40 (2H), 2.89 (q, J = 10.4 Hz, 2H), 2.70-2.65 (m, 1H), 1.75-1.67 (m, 2 H), 1.44-1.19 (m, 2H). MS (m/z) 458.2 (M + H)⁺. |
| 134 | 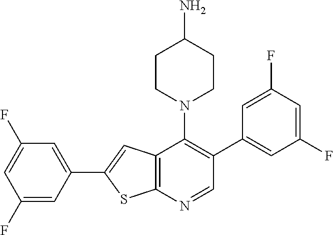 [SMILES: NC1CCN(c2c(-c3cc(F)cc(F)c3)cnc3sc(-c4cc(F)cc(F)c4)cc23)CC1] | 1H-NMR (400 MHz, DMSO-D6): δ 8.22 (s, 1H), 7.96 (s, 1H), 7.65 (d, J = 6.6 Hz, 2H), 7.35-7.23 (m, 4H), 3.43-3.31 (m, 2H), 3.08 (d, J = 11.8 Hz, 3H), 1.89 (d, J = 14.3 Hz, 2H), 1.58-1.47 (m, 2H). 19F-NMR (376 MHz, DMSO-D6): δ −73.35 (s, 0.13F), −108.69-−108.74 (t, J = 9.2 Hz, 2F), −109.21-−109.26 (t, J = 9.2 Hz, 2F). MS (m/z) 458.2 (M + H)⁺. |

### Example 28

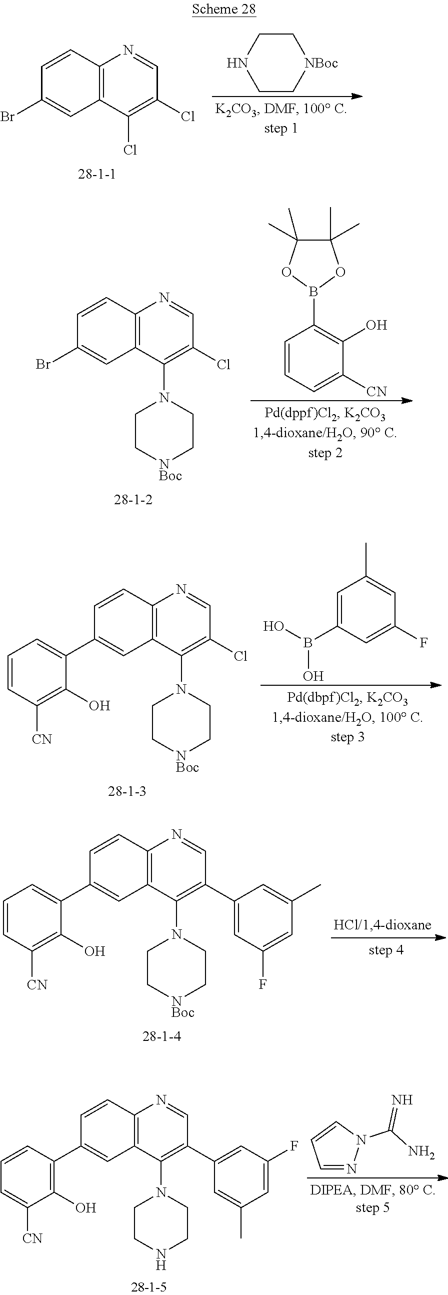

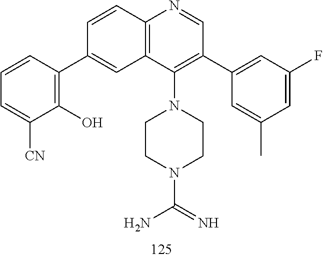 [SMILES: Cc1cc(F)cc(-c2cnc3ccc(-c4cccc(C#N)c4O)cc3c2N2CCN(C(=N)N)CC2)c1]

### Step 1: Synthesis of tert-butyl 4-(6-bromo-3-chloroquinolin-4-yl) piperazine-1-carboxylate (Compound 28-1-2)

To a stirred solution of 6-bromo-3,4-dichloroquinoline (compound 28-1-1, 2 g, 7.22 mmol, 1 equiv.) in DMF (20 ml, 100.0%) was added tert-butyl piperazine-1-carboxy late (1345.06 mg, 7.22 mmol, 1 equiv.), dipotassium carbonate (2 g, 14.44 mmol, 2 equiv.) at 20° C. The reaction was stirred at 100° C. for 16 hours. TLC (PE:EtOAc=5:1) indicated the reaction was complete. The reaction mixture was poured into H₂O (100 mL), filtered, and concentrated to afford tert-butyl 4-(6-bromo-3-chloroquinolin-4-yl) piperazine-1-carboxylate (compound 28-1-2, 2.3 g, 5.39 mmol, 74.63%). MS (m/z) 426.1/428.1 (M+H)⁺.

### Step 2: tert-butyl 4-[3-chloro-6-(3-cyano-2-hydroxyphenyl)quinolin-4-yl]piperazine-1-carboxylate (Compound 28-1-3)

To a stirred solution of tert-butyl 4-(6-bromo-3-chloroquinolin-4-yl) piperazine-1-carboxylate (compound 28-1-2, 900 mg, 2.11 mmol, 1 equiv.) in 1,4-dioxane (12 ml, 100.0%) was added water (3 ml, 166.53 mmol, 78.96 equiv.), 2-hydroxy-3-(4,4,5,5-tetramethyl-1,3,2-dioxaborolan-2-yl) benzonitrile (516.88 mg, 2.11 mmol, 1 equiv.), dipotassium carbonate (582.97 mg, 4.22 mmol, 2 equiv.) and 1,1′-Bis (diphenylphosphino) ferrocene-palladium (II) dichloride dichloromethane complex (160.88 mg, 0.21 mmol, 0.1 equiv.) at 20° C. The reaction was stirred at 90° C. for 1 hours. The reaction mixture was concentrated under reduced pressure. The residue was diluted with EtOAc (300 mL), washed with sat. NaHCO₃ (50 mL), dried over Na₂SO₄ and evaporated to give the residue. The residue was purified by column chromatography on silica gel eluted with PE/EtOAc (9:1˜1:1) to give tert-butyl 4-[3-chloro-6-(3-cyano-2-hydroxyphenyl) quinolin-4-yl]piperazine-1-carboxylate (compound 28-1-3, 500 mg, 1.08 mmol, 50.99%) as a white solid. MS (m/z) 465.2 (M+H)⁺.

### Step 3: Synthesis of tert-butyl 4-[6-(3-cyano-2-hydroxyphenyl)-3-(3-fluoro-5-methyl phenyl) quinolin-4-yl]piperazine-1-carboxylate (Compound 28-1-4)

To a stirred solution of tert-butyl 4-[3-chloro-6-(3-cyano-2-hydroxyphenyl)quinolin-4-yl]piperazine-1-carboxylate (compound 28-1-3, 500 mg, 1.08 mmol, 1 equiv.) in 1,4-dioxane (10 ml, 100.0%) was added water (2.5 ml, 138.78 mmol, 129.04 equiv.), (3-fluoro-5-methylphenyl)boronic acid (165.56 mg, 1.08 mmol, 1 equiv.), dipotassium carbonate (297.26 mg, 2.15 mmol, 2 equiv.) and dichlorobis[di-tert-butyl(4-dimethylaminophenyl)phosphino]palladium(II)(76.15 mg, 0.11 mmol, 0.1 equiv.) at 20° C. The reaction was stirred at 100° C. for 2 hours. The reaction mixture was concentrated under reduced pressure. The residue was diluted with EtOAc (300 mL), washed with sat.NaHCO₃ (50 mL), dried over Na₂SO₄ and evaporated to give the residue. The residue was purified by column chromatography on silica gel eluted with PE/EtOAc (9:1-1:1) to give tert-butyl 4-[6-(3-cyano-2-hydroxyphenyl)-3-(3-fluoro-5-methylphenyl)quinolin-4-yl]piperazine-1-carboxylate (compound 28-1-4, 400 mg, 0.74 mmol, 69.06%) as a white solid. MS (m/z) 539.3 (M+H)⁺.

### Step 4: Synthesis of 3-[3-(3-fluoro-5-methylphenyl)-4-(piperazin-1-yl)quinolin-6-yl]-2-hydroxybenzonitrile (Compound 28-1-5)

To a stirred solution of tert-butyl 4-[6-(3-cyano-2-hydroxyphenyl)-3-(3-fluoro-5-methylphenyl)quinolin-4-yl]piperazine-1-carboxylate (compound 28-1-4, 100 mg, 0.19 mmol, 1 equiv.) in DCM (3 ml) was added trifluoroacetic acid (1 ml, 8.77 mmol, 47.24 equiv.) at 20° C. The reaction was stirred at 20° C. for 2 hours. The reaction mixture was concentrated and evaporated to give 3-[3-(3-fluoro-5-methylphenyl)-4-(piperazin-1-yl)quinolin-6-yl]-2-hydroxybenzonitrile (compound 28-1-5, 80 mg, 0.18 mmol, 98.26%) as a white solid. MS (m/z) 439.3 (M+H)⁺.

### Step 5: Synthesis of Compound 135

To a stirred solution of 3-[3-(3-fluoro-5-methylphenyl)-4-(piperazin-1-yl)quinolin-6-yl]-2-hydroxybenzonitrile (compound 28-1-5, 80 mg, 0.18 mmol, 1 equiv.) in DMF (2 ml, 0%) was added pyrazole-1-carboximidamide (40.18 mg, 0.36 mmol, 2 equiv.) and ethylbis(propan-2-yl)amine (235.79 mg, 1.82 mmol, 10 equiv.) at 20° C. The reaction was stirred at 80° C. for 16 hours. The reaction mixture was concentrated under reduced pressure. The residue was diluted with EtOAc (50 mL), washed with H₂O (50 mL×3), dried over Na₂SO₄, and evaporated to give the residue. The residue was purified by HPLC to give 4-[6-(3-cyano-2-hydroxyphenyl)-3-(3-fluoro-5-methylphenyl)quinolin-4-yl]piperazine-1-carboximidamide (compound 135, 3.8 mg, 0.01 mmol, 4.33%) as a white solid.

Compounds 135 and 136 were synthesized according to the 5-step procedure described in Example 28. Analytical data for compounds 135 and 136 are provided below.

| Cpd. No. | Structure | Analytical Data |
|---|---|---|
| 135 | 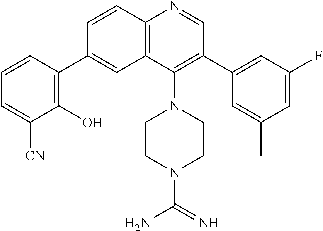 [SMILES: Cc1cc(F)cc(-c2cnc3ccc(-c4cccc(C#N)c4O)cc3c2N2CCN(C(=N)N)CC2)c1] | 1H NMR (400 MHz, CD3OD) δ 8.58 (s, 1H), 8,53 (s, 1H), 8.48 (s, 1H), 8.08 (d, J = 8.7 Hz, 1H), 7.92 (d, J = 8.8 Hz, 1H), 7.64 (d, J = 7.6 Hz, 1H), 7.51 (d, J = 6.1 Hz, 1H), 7.07 (s, 2H), 6.99 (t, J = 7.6 Hz, 2H), 3.61 (s, 4H), 3.14 (s, 4H), 2.45 (s, 3H). MS (m/z) 481.3 (M + H)⁺. |
| 136 | 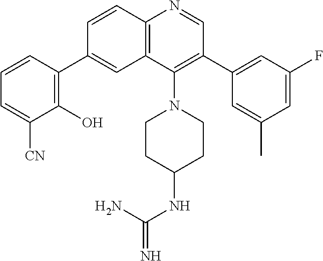 [SMILES: Cc1cc(F)cc(-c2cnc3ccc(-c4cccc(C#N)c4O)cc3c2N2CCC(NC(=N)N)CC2)c1] | 1H NMR (400 MHz, CD3OD) δ 8.51 (s, 1H), 8.48 (d, J = 1.8 Hz, 2H), 8.07 (d, J = 8.7 Hz, 1H), 7.90 (dd, J = 8.7, 1.9 Hz, 1H), 7.66 (dd, J = 7.6, 1.7 Hz, 1H), 7.56 (dd, J = 7.7, 1.7 Hz, 1H), 7.10-7.02 (m, 3H), 6.95 (d, J = 9.2 Hz, 1H), 3.48 (s, 1H), 3.38 (d, J = 13.1 Hz, 2H), 2.85 (t, J = 11.5 Hz, 2H), 2.45 (s, 3H), 1.95 (d, J = 9.6 Hz, 2H), 1.76 (d, J = 8.7 Hz, 2H). MS (m/z) 495.3 (M + H)⁺. |

### II. BIOLOGICAL EXAMPLES

### Example 29

### Biochemical Assay for Activation of Somatostatin Receptor 2 (SSTR2)

Agonistic activation of SSTRs leads to an increased production of intracellular cAMP levels. Cisbio's homogeneous time-resolved fluorescence (HTRF) cAMP assay was used to measure cAMP concentration in an high-throughput format. Increase in intracellular cAMP concentrations was measuring using a stably-transfected cell line expressing SSTR2. Test compounds were dissolved in DMSO. Cells were incubated with compounds for 1-2h, and subsequently competitive binding of cAMP produced due to the activation of SSTR2 by the test compounds was measured using the Cisbio cAMP Gs dynamic assay system (Perkin Elmer, Bedford, MA).

The average pEC₅₀ was determined for the test compounds. The data is provided in Table 2 below.

TABLE 2

| Cpd. | | |
|---|---|---|
| No. | Structure | pEC₅₀ |
| 1 | 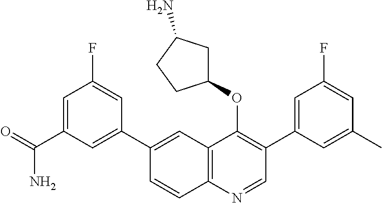 [SMILES: Cc1cc(F)cc(-c2cnc3ccc(-c4cc(F)cc(C(N)=O)c4)cc3c2O[C@H]2CC[C@H](N)C2)c1] | 9.5 |
| 2 | 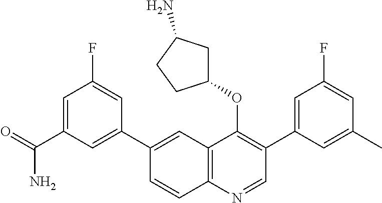 [SMILES: Cc1cc(F)cc(-c2cnc3ccc(-c4cc(F)cc(C(N)=O)c4)cc3c2O[C@@H]2CC[C@H](N)C2)c1] | 9 |
| 3 | 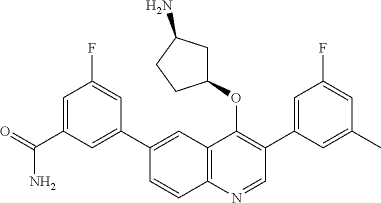 [SMILES: Cc1cc(F)cc(-c2cnc3ccc(-c4cc(F)cc(C(N)=O)c4)cc3c2O[C@H]2CC[C@@H](N)C2)c1] | 8.1 |
| 4 | 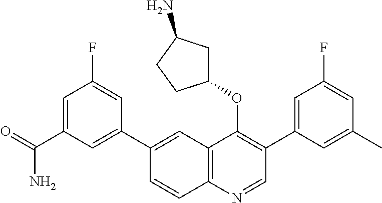 [SMILES: Cc1cc(F)cc(-c2cnc3ccc(-c4cc(F)cc(C(N)=O)c4)cc3c2O[C@@H]2CC[C@@H](N)C2)c1] | 9.4 |
| 5 | 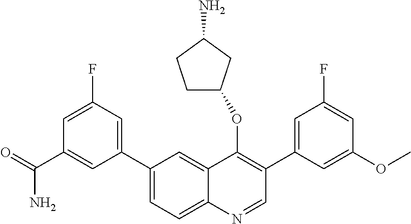 [SMILES: COc1cc(F)cc(-c2cnc3ccc(-c4cc(F)cc(C(N)=O)c4)cc3c2O[C@@H]2CC[C@H](N)C2)c1] | 8.5 |
| 6 | 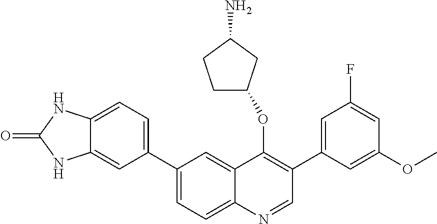 [SMILES: COc1cc(F)cc(-c2cnc3ccc(-c4ccc5[nH]c(=O)[nH]c5c4)cc3c2O[C@@H]2CC[C@H](N)C2)c1] | 7.2 |
| 7 | 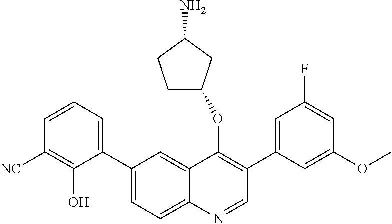 [SMILES: COc1cc(F)cc(-c2cnc3ccc(-c4cccc(C#N)c4O)cc3c2O[C@@H]2CC[C@H](N)C2)c1] | 7.7 |
| 8 | 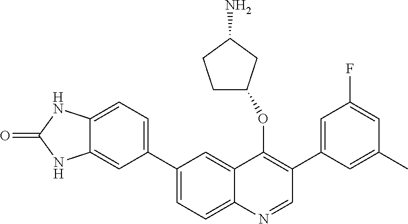 [SMILES: Cc1cc(F)cc(-c2cnc3ccc(-c4ccc5[nH]c(=O)[nH]c5c4)cc3c2O[C@@H]2CC[C@H](N)C2)c1] | 8.2 |
| 9 | 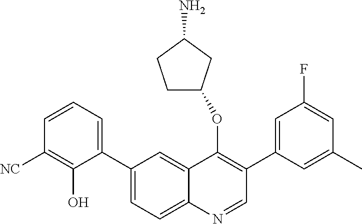 [SMILES: Cc1cc(F)cc(-c2cnc3ccc(-c4cccc(C#N)c4O)cc3c2O[C@@H]2CC[C@H](N)C2)c1] | 8.5 |
| 10 | 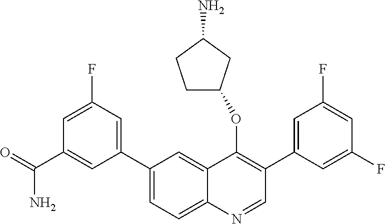 [SMILES: NC(=O)c1cc(F)cc(-c2ccc3ncc(-c4cc(F)cc(F)c4)c(O[C@@H]4CC[C@H](N)C4)c3c2)c1] | 8 |
| 11 | 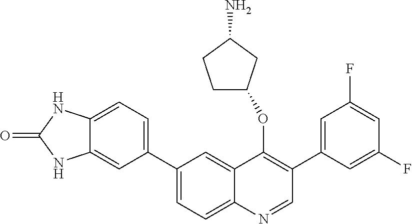 [SMILES: N[C@H]1CC[C@@H](Oc2c(-c3cc(F)cc(F)c3)cnc3ccc(-c4ccc5[nH]c(=O)[nH]c5c4)cc23)C1] | 6.4 |
| 12 | 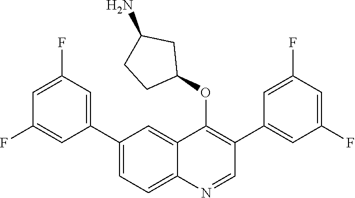 [SMILES: N[C@@H]1CC[C@H](Oc2c(-c3cc(F)cc(F)c3)cnc3ccc(-c4cc(F)cc(F)c4)cc23)C1] | 7.8 |
| 13 | 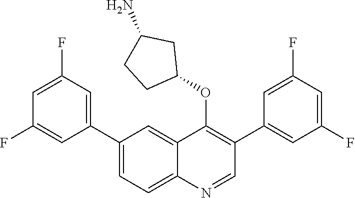 [SMILES: N[C@H]1CC[C@@H](Oc2c(-c3cc(F)cc(F)c3)cnc3ccc(-c4cc(F)cc(F)c4)cc23)C1] | 7 |
| 14 | 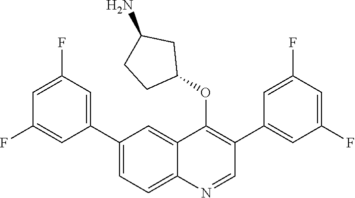 [SMILES: N[C@@H]1CC[C@@H](Oc2c(-c3cc(F)cc(F)c3)cnc3ccc(-c4cc(F)cc(F)c4)cc23)C1] | 7.9 |
| 15 | 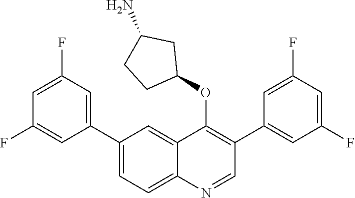 [SMILES: N[C@H]1CC[C@H](Oc2c(-c3cc(F)cc(F)c3)cnc3ccc(-c4cc(F)cc(F)c4)cc23)C1] | 7.9 |
| 16 | 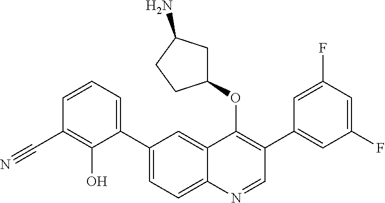 [SMILES: N#Cc1cccc(-c2ccc3ncc(-c4cc(F)cc(F)c4)c(O[C@H]4CC[C@@H](N)C4)c3c2)c1O] | 6.7 |
| 17 | 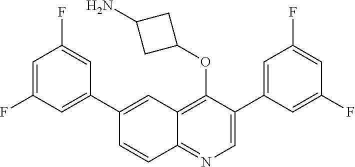 [SMILES: NC1CC(Oc2c(-c3cc(F)cc(F)c3)cnc3ccc(-c4cc(F)cc(F)c4)cc23)C1] | 7.9 |
| 18 | 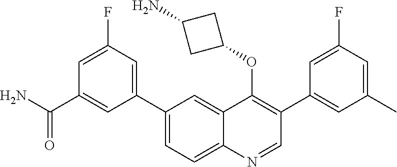 [SMILES: Cc1cc(F)cc(-c2cnc3ccc(-c4cc(F)cc(C(N)=O)c4)cc3c2O[C@H]2C[C@@H](N)C2)c1] | 7.9 |
| 19 | 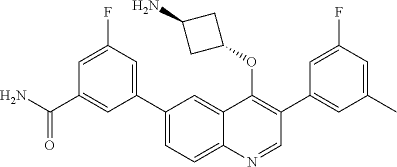 [SMILES: Cc1cc(F)cc(-c2cnc3ccc(-c4cc(F)cc(C(N)=O)c4)cc3c2O[C@H]2C[C@H](N)C2)c1] | 9 |
| 20 | 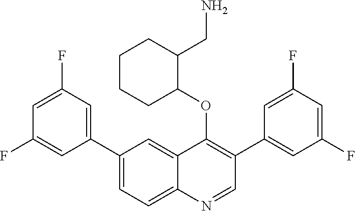 [SMILES: NCC1CCCCC1Oc1c(-c2cc(F)cc(F)c2)cnc2ccc(-c3cc(F)cc(F)c3)cc12] | 5.4 |
| 21 | 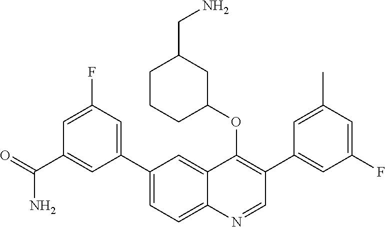 [SMILES: Cc1cc(F)cc(-c2cnc3ccc(-c4cc(F)cc(C(N)=O)c4)cc3c2OC2CCCC(CN)C2)c1] | 7.7 |
| 22 | 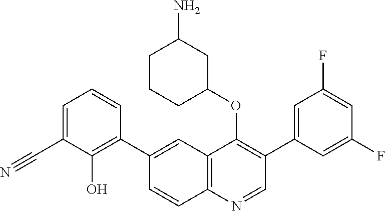 [SMILES: N#Cc1cccc(-c2ccc3ncc(-c4cc(F)cc(F)c4)c(OC4CCCC(N)C4)c3c2)c1O] | <5.0 |
| 23 | 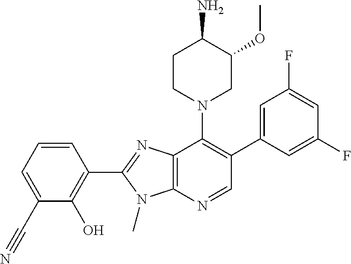 [SMILES: CO[C@@H]1CN(c2c(-c3cc(F)cc(F)c3)cnc3c2nc(-c2cccc(C#N)c2O)n3C)CC[C@H]1N] | <5.0 |
| 24 | 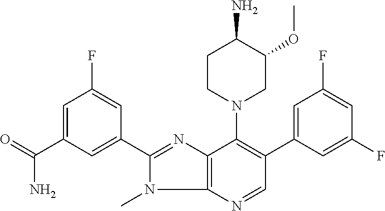 [SMILES: CO[C@@H]1CN(c2c(-c3cc(F)cc(F)c3)cnc3c2nc(-c2cc(F)cc(C(N)=O)c2)n3C)CC[C@H]1N] | <5.3 |
| 25 | 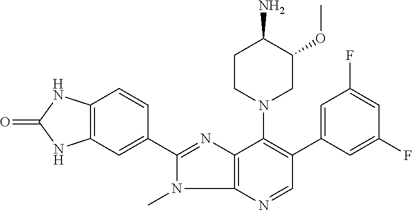 [SMILES: CO[C@@H]1CN(c2c(-c3cc(F)cc(F)c3)cnc3c2nc(-c2ccc4[nH]c(=O)[nH]c4c2)n3C)CC[C@H]1N] | <5.0 |
| 26 | 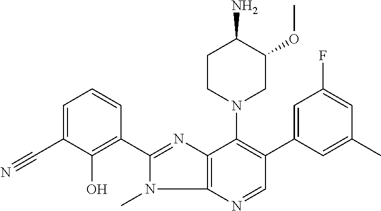 [SMILES: CO[C@@H]1CN(c2c(-c3cc(C)cc(F)c3)cnc3c2nc(-c2cccc(C#N)c2O)n3C)CC[C@H]1N] | 5 |
| 27 | 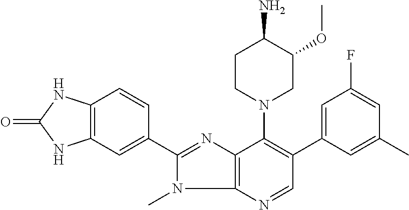 [SMILES: CO[C@@H]1CN(c2c(-c3cc(C)cc(F)c3)cnc3c2nc(-c2ccc4[nH]c(=O)[nH]c4c2)n3C)CC[C@H]1N] | <5.0 |
| 28 | 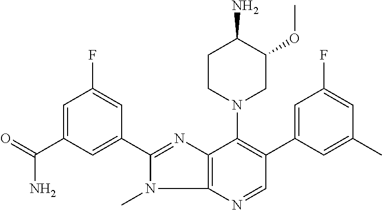 [SMILES: CO[C@@H]1CN(c2c(-c3cc(C)cc(F)c3)cnc3c2nc(-c2cc(F)cc(C(N)=O)c2)n3C)CC[C@H]1N] | 6.1 |
| 29 | 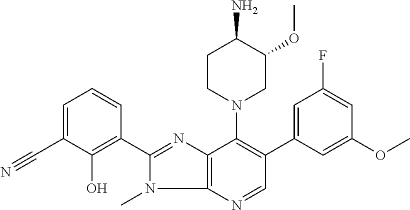 [SMILES: COc1cc(F)cc(-c2cnc3c(nc(-c4cccc(C#N)c4O)n3C)c2N2CC[C@@H](N)[C@H](OC)C2)c1] | <5.0 |
| 30 | 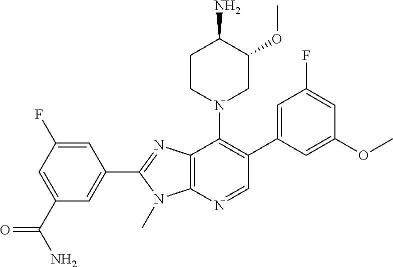 [SMILES: COc1cc(F)cc(-c2cnc3c(nc(-c4cc(F)cc(C(N)=O)c4)n3C)c2N2CC[C@@H](N)[C@H](OC)C2)c1] | 6.0 |
| 31 | 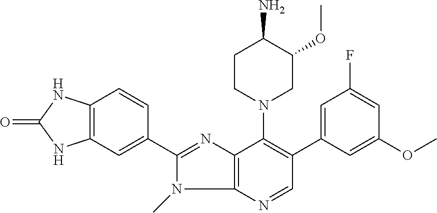 [SMILES: COc1cc(F)cc(-c2cnc3c(nc(-c4ccc5[nH]c(=O)[nH]c5c4)n3C)c2N2CC[C@@H](N)[C@H](OC)C2)c1] | <5.0 |
| 32 | 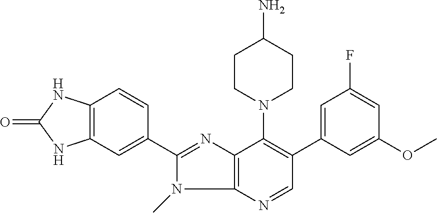 [SMILES: COc1cc(F)cc(-c2cnc3c(nc(-c4ccc5[nH]c(=O)[nH]c5c4)n3C)c2N2CCC(N)CC2)c1] | <5.0 |
| 33 | 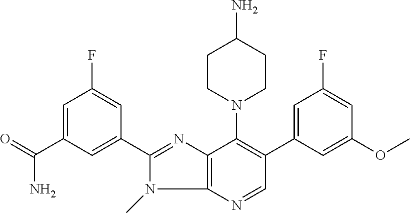 [SMILES: COc1cc(F)cc(-c2cnc3c(nc(-c4cc(F)cc(C(N)=O)c4)n3C)c2N2CCC(N)CC2)c1] | 5.8 |
| 34 | 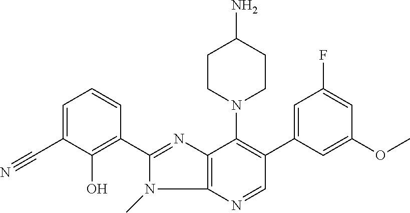 [SMILES: COc1cc(F)cc(-c2cnc3c(nc(-c4cccc(C#N)c4O)n3C)c2N2CCC(N)CC2)c1] | 6.3 |
| 35 | 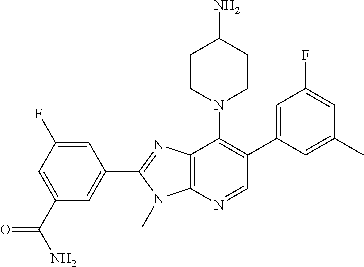 [SMILES: Cc1cc(F)cc(-c2cnc3c(nc(-c4cc(F)cc(C(N)=O)c4)n3C)c2N2CCC(N)CC2)c1] | 6.8 |
| 36 | 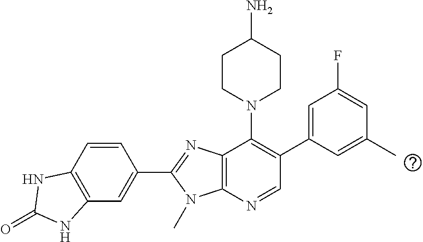 [SMILES: Cc1cc(F)cc(-c2cnc3c(nc(-c4ccc5[nH]c(=O)[nH]c5c4)n3C)c2N2CCC(N)CC2)c1] | <5.0 |
| 37 | 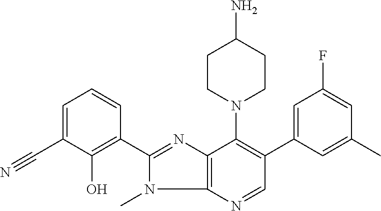 [SMILES: Cc1cc(F)cc(-c2cnc3c(nc(-c4cccc(C#N)c4O)n3C)c2N2CCC(N)CC2)c1] | 6.5 |
| 38 | 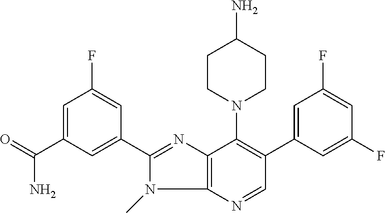 [SMILES: Cn1c(-c2cc(F)cc(C(N)=O)c2)nc2c(N3CCC(N)CC3)c(-c3cc(F)cc(F)c3)cnc21] | 5.7 |
| 39 | 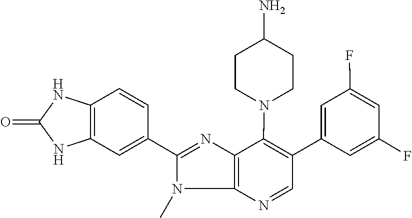 [SMILES: Cn1c(-c2ccc3[nH]c(=O)[nH]c3c2)nc2c(N3CCC(N)CC3)c(-c3cc(F)cc(F)c3)cnc21] | <5.0 |
| 40 | 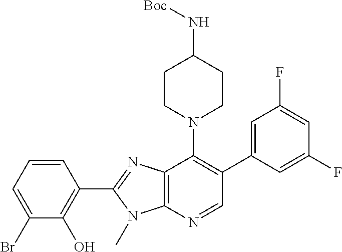 [SMILES: Cn1c(-c2cccc(Br)c2O)nc2c(N3CCC(NC(=O)OC(C)(C)C)CC3)c(-c3cc(F)cc(F)c3)cnc21] | <5.0 |
| 41 | 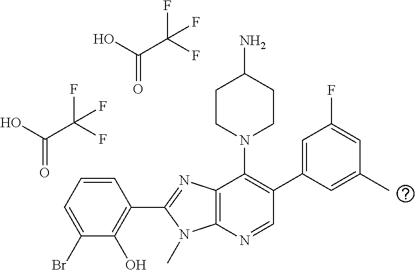 | 6.8 |
| 42 | 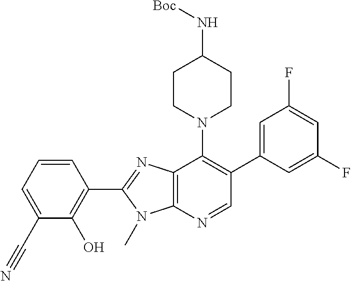 [SMILES: Cn1c(-c2cccc(C#N)c2O)nc2c(N3CCC(NC(=O)OC(C)(C)C)CC3)c(-c3cc(F)cc(F)c3)cnc21] | <5.0 |
| 43 | 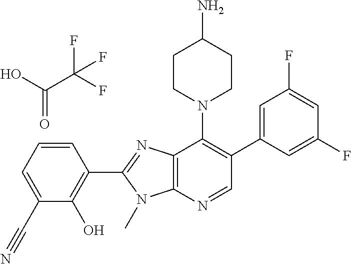 | 5.2 |
| 44 | 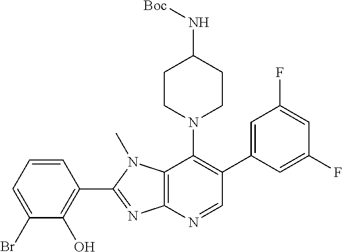 [SMILES: Cn1c(-c2cccc(Br)c2O)nc2ncc(-c3cc(F)cc(F)c3)c(N3CCC(NC(=O)OC(C)(C)C)CC3)c21] | <5.0 |
| 45 | 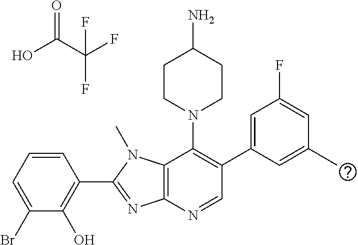 [SMILES: Cc1cc(F)cc(-c2cnc3nc(-c4cccc(Br)c4O)n(C)c3c2N2CCC(N)CC2)c1 ; O=C(O)C(F)(F)F] | 6.4 |
| 46 | 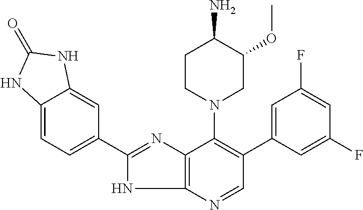 [SMILES: CO[C@@H]1CN(c2c(-c3cc(F)cc(F)c3)cnc3[nH]c(-c4ccc5[nH]c(=O)[nH]c5c4)nc23)CC[C@H]1N] | <5.0 |
| 47 | 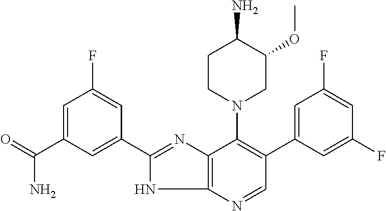 [SMILES: CO[C@@H]1CN(c2c(-c3cc(F)cc(F)c3)cnc3[nH]c(-c4cc(F)cc(C(N)=O)c4)nc23)CC[C@H]1N] | 6.0 |
| 48 | 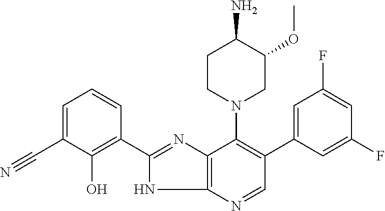 [SMILES: CO[C@@H]1CN(c2c(-c3cc(F)cc(F)c3)cnc3[nH]c(-c4cccc(C#N)c4O)nc23)CC[C@H]1N] | 6.3 |
| 49 | 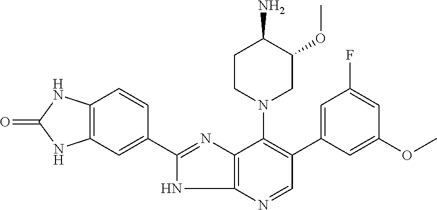 [SMILES: COc1cc(F)cc(-c2cnc3[nH]c(-c4ccc5[nH]c(=O)[nH]c5c4)nc3c2N2CC[C@@H](N)[C@H](OC)C2)c1] | <5.0 |
| 50 | 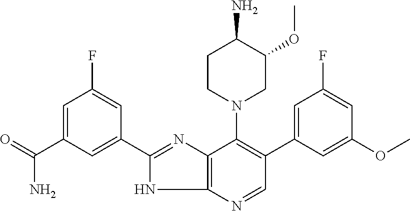 [SMILES: COc1cc(F)cc(-c2cnc3[nH]c(-c4cc(F)cc(C(N)=O)c4)nc3c2N2CC[C@@H](N)[C@H](OC)C2)c1] | <5.0 |
| 51 | 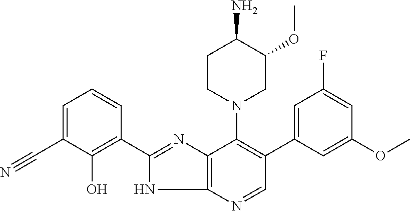 [SMILES: COc1cc(F)cc(-c2cnc3[nH]c(-c4cccc(C#N)c4O)nc3c2N2CC[C@@H](N)[C@H](OC)C2)c1] | <5.0 |
| 52 | 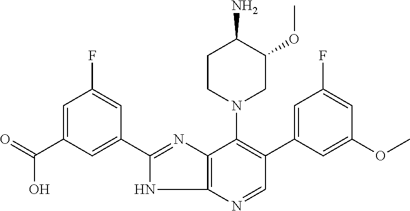 [SMILES: COc1cc(F)cc(-c2cnc3[nH]c(-c4cc(F)cc(C(=O)O)c4)nc3c2N2CC[C@@H](N)[C@H](OC)C2)c1] | <5.0 |
| 53 | 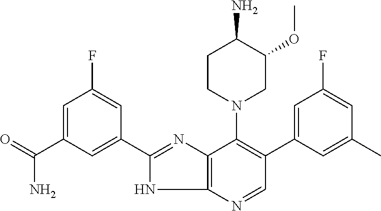 [SMILES: CO[C@@H]1CN(c2c(-c3cc(C)cc(F)c3)cnc3[nH]c(-c4cc(F)cc(C(N)=O)c4)nc23)CC[C@H]1N] | 6.0 |
| 54 | 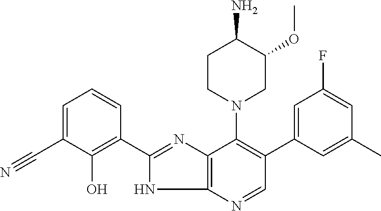 [SMILES: CO[C@@H]1CN(c2c(-c3cc(C)cc(F)c3)cnc3[nH]c(-c4cccc(C#N)c4O)nc23)CC[C@H]1N] | 5.6 |
| 55 | 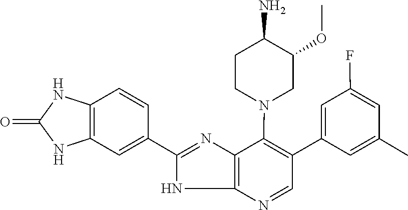 [SMILES: CO[C@@H]1CN(c2c(-c3cc(C)cc(F)c3)cnc3[nH]c(-c4ccc5[nH]c(=O)[nH]c5c4)nc23)CC[C@H]1N] | <5.0 |
| 56 | 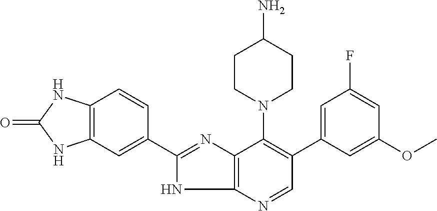 [SMILES: COc1cc(F)cc(-c2cnc3[nH]c(-c4ccc5[nH]c(=O)[nH]c5c4)nc3c2N2CCC(N)CC2)c1] | 5.8 |
| 57 | 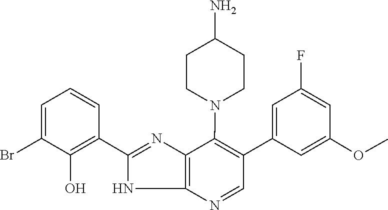 [SMILES: COc1cc(F)cc(-c2cnc3[nH]c(-c4cccc(Br)c4O)nc3c2N2CCC(N)CC2)c1] | 8.0 |
| 58 | 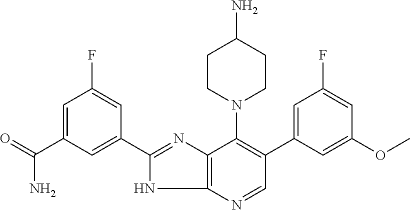 [SMILES: COc1cc(F)cc(-c2cnc3[nH]c(-c4cc(F)cc(C(N)=O)c4)nc3c2N2CCC(N)CC2)c1] | 6.3 |
| 59 | 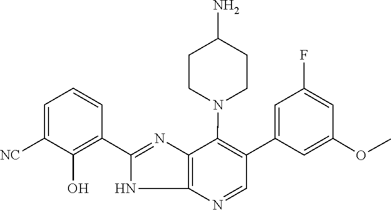 [SMILES: COc1cc(F)cc(-c2cnc3[nH]c(-c4cccc(C#N)c4O)nc3c2N2CCC(N)CC2)c1] | 6.2 |
| 60 | 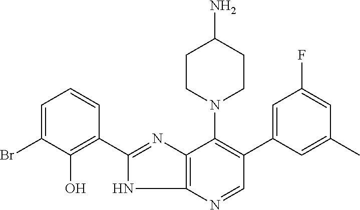 [SMILES: Cc1cc(F)cc(-c2cnc3[nH]c(-c4cccc(Br)c4O)nc3c2N2CCC(N)CC2)c1] | 8.5 |
| 61 | 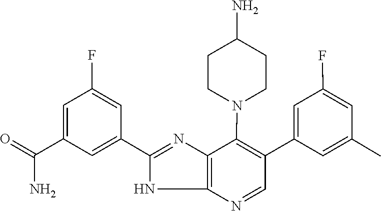 [SMILES: Cc1cc(F)cc(-c2cnc3[nH]c(-c4cc(F)cc(C(N)=O)c4)nc3c2N2CCC(N)CC2)c1] | 7.3 |
| 62 | 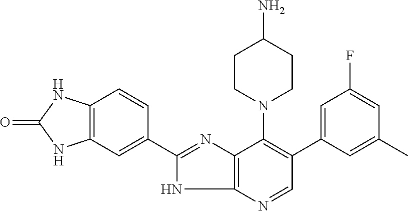 [SMILES: Cc1cc(F)cc(-c2cnc3[nH]c(-c4ccc5[nH]c(=O)[nH]c5c4)nc3c2N2CCC(N)CC2)c1] | 6.2 |
| 63 | 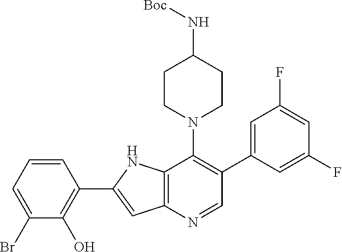 [SMILES: CC(C)(C)OC(=O)NC1CCN(c2c(-c3cc(F)cc(F)c3)cnc3cc(-c4cccc(Br)c4O)[nH]c23)CC1] | <5.0 |
| 64 | 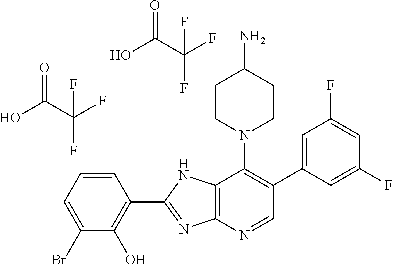 | 7.5 |
| 65 | 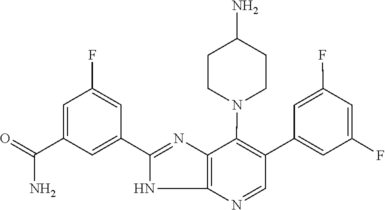 [SMILES: NC(=O)c1cc(F)cc(-c2nc3c(N4CCC(N)CC4)c(-c4cc(F)cc(F)c4)cnc3[nH]2)c1] | 5.5 |
| 66 | 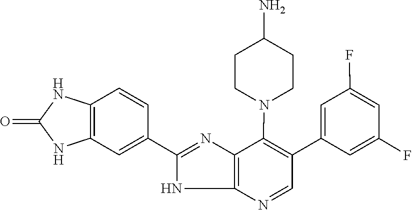 [SMILES: NC1CCN(c2c(-c3cc(F)cc(F)c3)cnc3[nH]c(-c4ccc5[nH]c(=O)[nH]c5c4)nc23)CC1] | <5.0 |
| 67 | 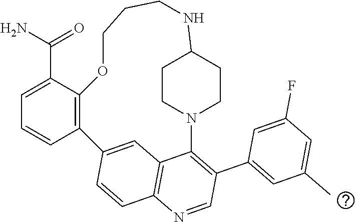 [SMILES: Cc1cc(F)cc(-c2cnc3ccc4cc3c2N2CCC(CC2)NCCCOc2c(C(N)=O)cccc2-4)c1] | 9.1 |
| 68 | 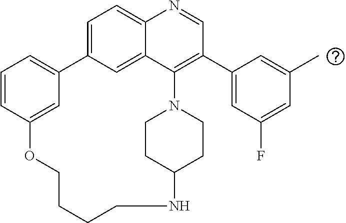 [SMILES: Cc1cc(F)cc(-c2cnc3ccc4cc3c2N2CCC(CC2)NCCCCOc2cccc-4c2)c1] | 8.1 |
| 69 | 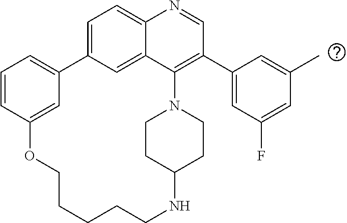 [SMILES: Cc1cc(F)cc(-c2cnc3ccc4cc3c2N2CCC(CC2)NCCCCCOc2cccc-4c2)c1] | 8.9 |
| 70 | 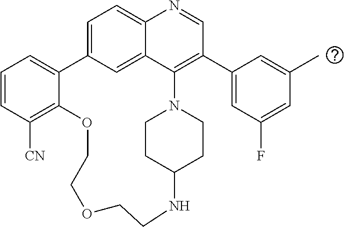 [SMILES: Cc1cc(F)cc(-c2cnc3ccc4cc3c2N2CCC(CC2)NCCOCCOc2c(C#N)cccc2-4)c1] | 9.1 |
| 71 | 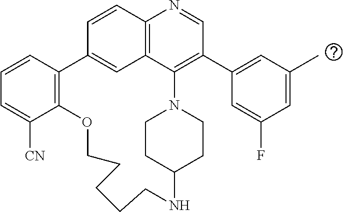 [SMILES: Cc1cc(F)cc(-c2cnc3ccc4cc3c2N2CCC(CC2)NCCCCCOc2c(C#N)cccc2-4)c1] | 9.8 |
| 72 | 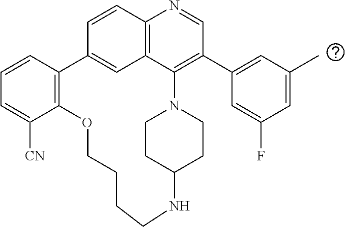 [SMILES: Cc1cc(F)cc(-c2cnc3ccc4cc3c2N2CCC(CC2)NCCCCOc2c(C#N)cccc2-4)c1] | 0 |
| 73 | 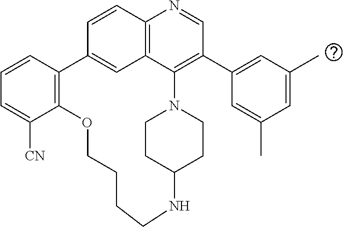 [SMILES: Cc1cc(C)cc(-c2cnc3ccc4cc3c2N2CCC(CC2)NCCCCOc2c(C#N)cccc2-4)c1] | 10.6 |
| 74 | 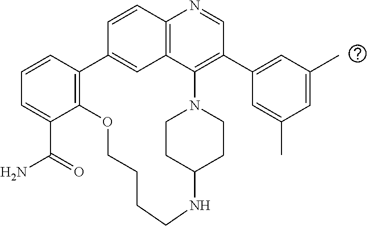 [SMILES: Cc1cc(C)cc(-c2cnc3ccc4cc3c2N2CCC(CC2)NCCCCOc2c(C(N)=O)cccc2-4)c1] | 9.6 |
| 75 | 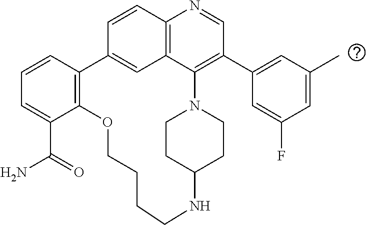 [SMILES: Cc1cc(F)cc(-c2cnc3ccc4cc3c2N2CCC(CC2)NCCCCOc2c(C(N)=O)cccc2-4)c1] | 8.7 |
| 76 | 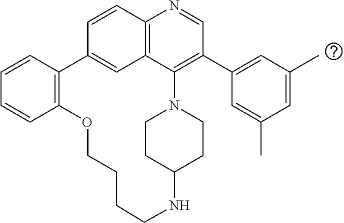 [SMILES: Cc1cc(C)cc(-c2cnc3ccc4cc3c2N2CCC(CC2)NCCCCOc2ccccc2-4)c1] | 10.1 |
| 77 | 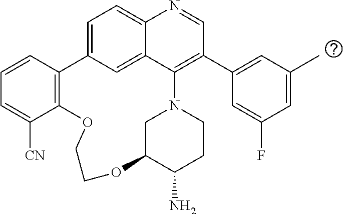 [SMILES: Cc1cc(F)cc(-c2cnc3ccc4cc3c2N2CC[C@H](N)[C@H](C2)OCCOc2c(C#N)cccc2-4)c1] | 8.2 |
| 78 | 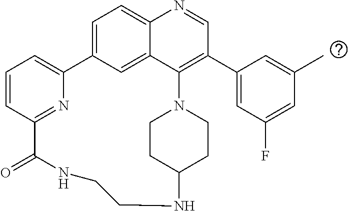 [SMILES: Cc1cc(F)cc(-c2cnc3ccc4cc3c2N2CCC(CC2)NCCNC(=O)c2cccc-4n2)c1] | 5.6 |
| 79 | 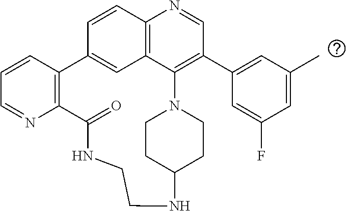 [SMILES: Cc1cc(F)cc(-c2cnc3ccc4cc3c2N2CCC(CC2)NCCNC(=O)c2ncccc2-4)c1] | <5.0 |
| 80 | 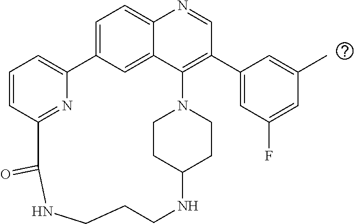 [SMILES: Cc1cc(F)cc(-c2cnc3ccc4cc3c2N2CCC(CC2)NCCCNC(=O)c2cccc-4n2)c1] | 8.2 |
| 81 | 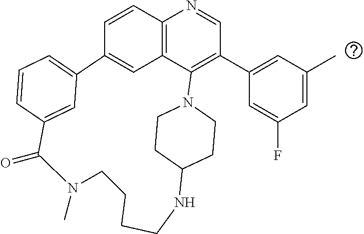 [SMILES: Cc1cc(F)cc(-c2cnc3ccc4cc3c2N2CCC(CC2)NCCCCN(C)C(=O)c2cccc-4c2)c1] | 5.7 |
| 82 | 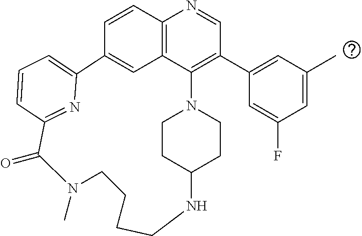 [SMILES: Cc1cc(F)cc(-c2cnc3ccc4cc3c2N2CCC(CC2)NCCCCN(C)C(=O)c2cccc-4n2)c1] | 7.6 |
| 83 | 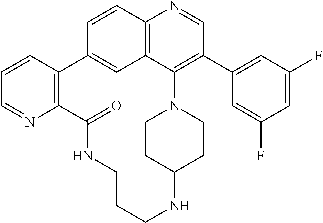 [SMILES: O=C1NCCCNC2CCN(CC2)c2c(-c3cc(F)cc(F)c3)cnc3ccc(cc23)-c2cccnc21] | 5.0 |
| 84 | 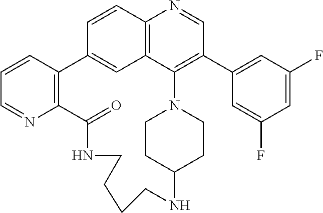 [SMILES: O=C1NCCCCNC2CCN(CC2)c2c(-c3cc(F)cc(F)c3)cnc3ccc(cc23)-c2cccnc21] | <5.0 |
| 85 | 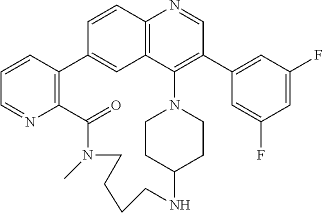 [SMILES: CN1CCCCNC2CCN(CC2)c2c(-c3cc(F)cc(F)c3)cnc3ccc(cc23)-c2cccnc2C1=O] | 5.2 |
| 86 | 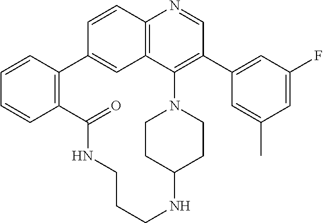 [SMILES: Cc1cc(F)cc(-c2cnc3ccc4cc3c2N2CCC(CC2)NCCCNC(=O)c2ccccc2-4)c1] | 5.2 |
| 87 | 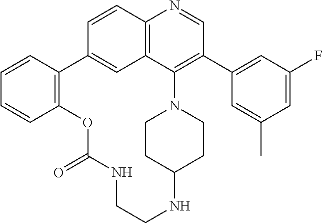 [SMILES: Cc1cc(F)cc(-c2cnc3ccc4cc3c2N2CCC(CC2)NCCNC(=O)Oc2ccccc2-4)c1] | 9.6 |
| 88* | 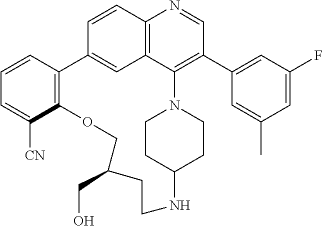 | 7.1 |
| 89* | 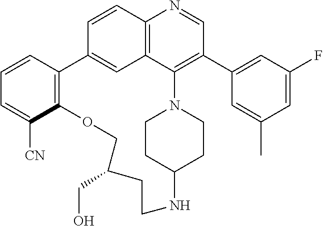 | 6.1 |
| 90* | 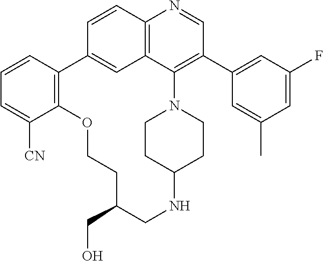 [SMILES: Cc1cc(F)cc(-c2cnc3ccc4cc3c2N2CCC(CC2)NC[C@@H](CO)CCOc2c(C#N)cccc2-4)c1] | 7.8 |
| 91* | 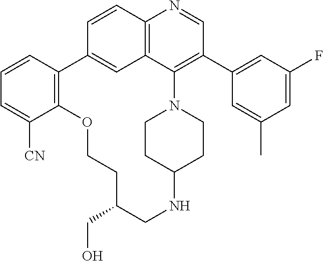 [SMILES: Cc1cc(F)cc(-c2cnc3ccc4cc3c2N2CCC(CC2)NC[C@H](CO)CCOc2c(C#N)cccc2-4)c1] | 8.2 |
| 92 | 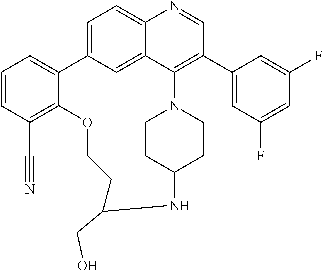 [SMILES: N#Cc1cccc2c1OCCC(CO)NC1CCN(CC1)c1c(-c3cc(F)cc(F)c3)cnc3ccc-2cc13] | <5.0 |
| 93 | 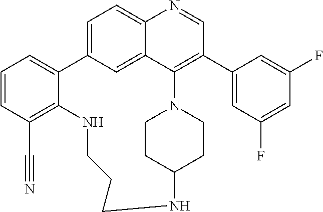 [SMILES: N#Cc1cccc2c1NCCCNC1CCN(CC1)c1c(-c3cc(F)cc(F)c3)cnc3ccc-2cc13] | 6.8 |
| 94 | 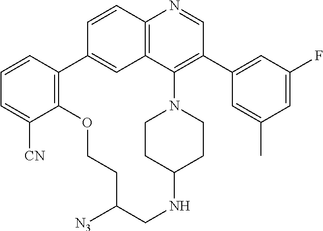 [SMILES: Cc1cc(F)cc(-c2cnc3ccc4cc3c2N2CCC(CC2)NCC(N=[N+]=[N-])CCOc2c(C#N)cccc2-4)c1] | 6.8 |
| 95 | 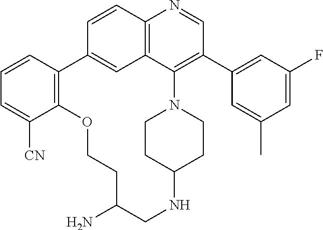 [SMILES: Cc1cc(F)cc(-c2cnc3ccc4cc3c2N2CCC(CC2)NCC(N)CCOc2c(C#N)cccc2-4)c1] | 8.1 |
| 96* | 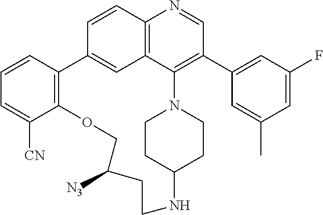 [SMILES: Cc1cc(F)cc(-c2cnc3ccc4cc3c2N2CCC(CC2)NCC[C@@H](N=[N+]=[N-])COc2c(C#N)cccc2-4)c1] | 9.1 |
| 97* | 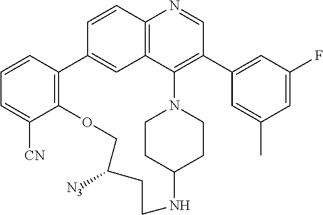 [SMILES: Cc1cc(F)cc(-c2cnc3ccc4cc3c2N2CCC(CC2)NCC[C@H](N=[N+]=[N-])COc2c(C#N)cccc2-4)c1] | 8.6 |
| 98* | 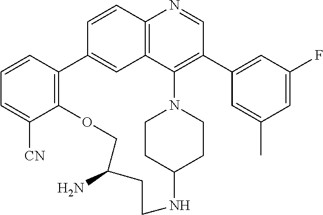 [SMILES: Cc1cc(F)cc(-c2cnc3ccc4cc3c2N2CCC(CC2)NCC[C@@H](N)COc2c(C#N)cccc2-4)c1] | 6.5 |
| 99* | 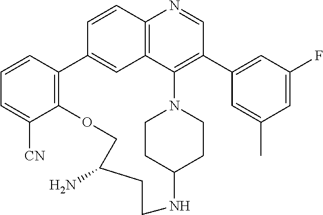 [SMILES: Cc1cc(F)cc(-c2cnc3ccc4cc3c2N2CCC(CC2)NCC[C@H](N)COc2c(C#N)cccc2-4)c1] | 8.1 |
| 100* | 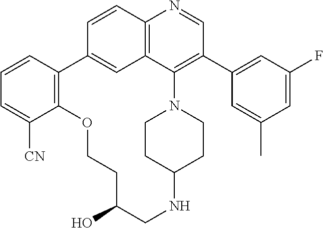 [SMILES: Cc1cc(F)cc(-c2cnc3ccc4cc3c2N2CCC(CC2)NC[C@@H](O)CCOc2c(C#N)cccc2-4)c1] | 5.6 |
| 101* | 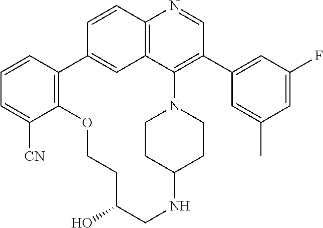 [SMILES: Cc1cc(F)cc(-c2cnc3ccc4cc3c2N2CCC(CC2)NC[C@H](O)CCOc2c(C#N)cccc2-4)c1] | 5.8 |
| 102* | 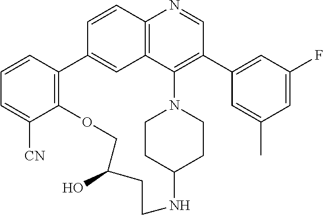 [SMILES: Cc1cc(F)cc(-c2cnc3ccc4cc3c2N2CCC(CC2)NCC[C@@H](O)COc2c(C#N)cccc2-4)c1] | 6.4 |
| 103* | 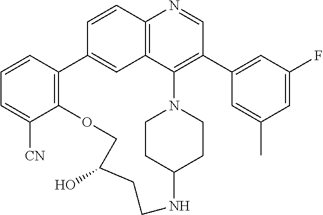 [SMILES: Cc1cc(F)cc(-c2cnc3ccc4cc3c2N2CCC(CC2)NCC[C@H](O)COc2c(C#N)cccc2-4)c1] | 5.4 |
| 104 | 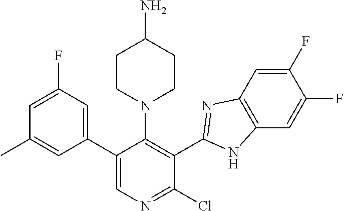 [SMILES: Cc1cc(F)cc(-c2cnc(Cl)c(-c3nc4cc(F)c(F)cc4[nH]3)c2N2CCC(N)CC2)c1] | 10.1 |
| 105 | 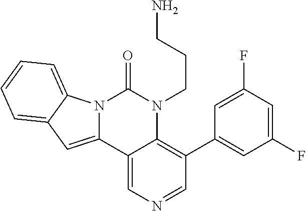 [SMILES: NCCCn1c(=O)n2c3ccccc3cc2c2cncc(-c3cc(F)cc(F)c3)c21] | <5.0 |
| 106 | 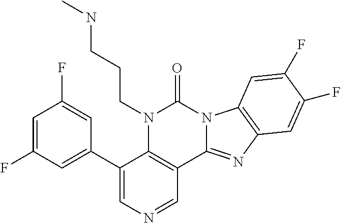 [SMILES: CNCCCn1c(=O)n2c3cc(F)c(F)cc3nc2c2cncc(-c3cc(F)cc(F)c3)c21] | 5.7 |
| 107 | 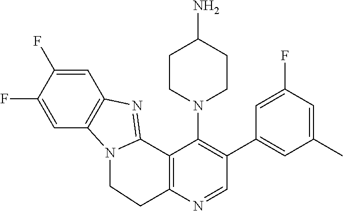 [SMILES: Cc1cc(F)cc(-c2cnc3c(c2N2CCC(N)CC2)-c2nc4cc(F)c(F)cc4n2CC3)c1] | <7.1 |
| 108 | 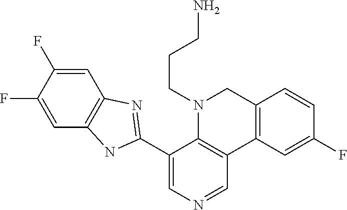 [SMILES: NCCCN1Cc2ccc(F)cc2-c2cncc(-c3nc4cc(F)c(F)cc4[nH]3)c21] | 6.9 |
| 109 | 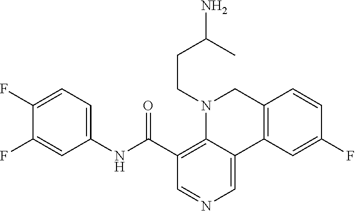 [SMILES: CC(N)CCN1Cc2ccc(F)cc2-c2cncc(C(=O)Nc3ccc(F)c(F)c3)c21] | 6.7 |
| 110 | 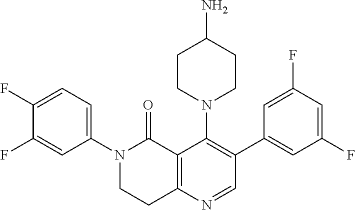 [SMILES: NC1CCN(c2c(-c3cc(F)cc(F)c3)cnc3c2C(=O)N(c2ccc(F)c(F)c2)CC3)CC1] | 6.8 |
| 111 | 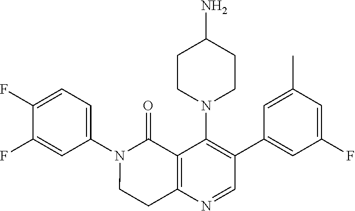 [SMILES: Cc1cc(F)cc(-c2cnc3c(c2N2CCC(N)CC2)C(=O)N(c2ccc(F)c(F)c2)CC3)c1] | 7.8 |
| 112 | 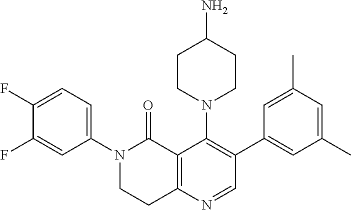 [SMILES: Cc1cc(C)cc(-c2cnc3c(c2N2CCC(N)CC2)C(=O)N(c2ccc(F)c(F)c2)CC3)c1] | 7.6 |
| 113 | 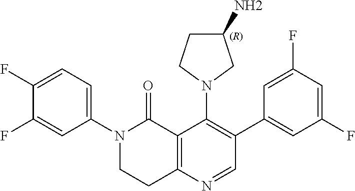 | <5.0 |
| 114 | 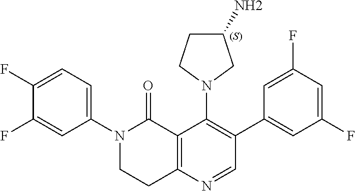 | <5.0 |
| 115 | 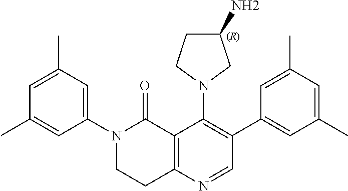 | 6.0 |
| 116 | 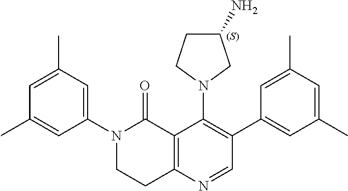 | 5.9 |
| 117 | 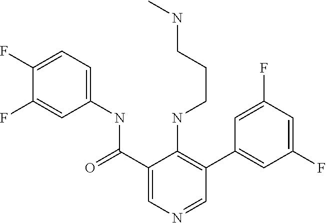 [SMILES: CNCCCNc1c(C(=O)Nc2ccc(F)c(F)c2)cncc1-c1cc(F)cc(F)c1] | 5.7 |
| 118 | 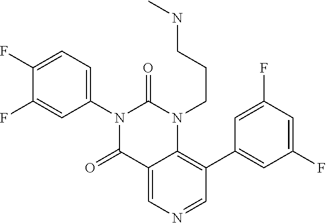 [SMILES: CNCCCn1c(=O)n(-c2ccc(F)c(F)c2)c(=O)c2cncc(-c3cc(F)cc(F)c3)c21] | <5.0 |
| 119 | 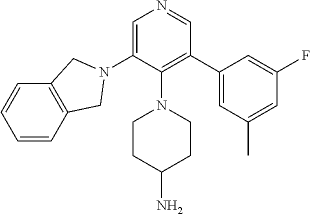 [SMILES: Cc1cc(F)cc(-c2cncc(N3Cc4ccccc4C3)c2N2CCC(N)CC2)c1] | 10.3 |
| 120 | 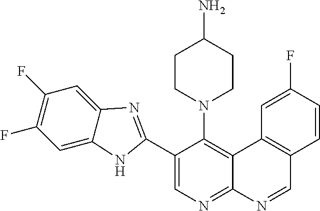 [SMILES: NC1CCN(c2c(-c3nc4cc(F)c(F)cc4[nH]3)cnc3ncc4ccc(F)cc4c23)CC1] | 5.2 |
| 121 | 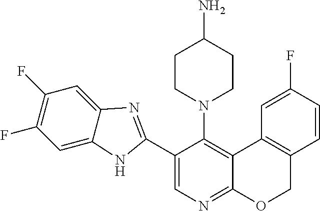 [SMILES: NC1CCN(c2c(-c3nc4cc(F)c(F)cc4[nH]3)cnc3c2-c2cc(F)ccc2CO3)CC1] | 6.2 |
| 122 | 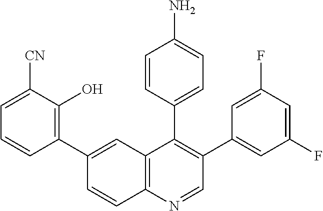 [SMILES: N#Cc1cccc(-c2ccc3ncc(-c4cc(F)cc(F)c4)c(-c4ccc(N)cc4)c3c2)c1O] | <5.0 |
| 123 | 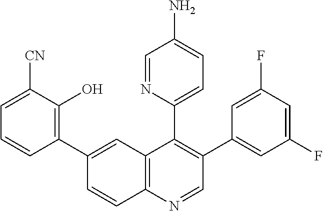 [SMILES: N#Cc1cccc(-c2ccc3ncc(-c4cc(F)cc(F)c4)c(-c4ccc(N)cn4)c3c2)c1O] | <5.0 |
| 124 | 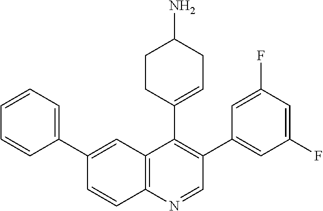 [SMILES: NC1CC=C(c2c(-c3cc(F)cc(F)c3)cnc3ccc(-c4ccccc4)cc23)CC1] | 7.1 |
| 125 | 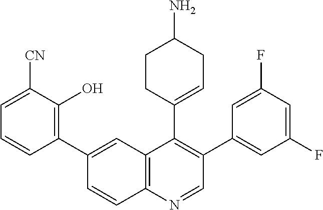 [SMILES: N#Cc1cccc(-c2ccc3ncc(-c4cc(F)cc(F)c4)c(C4=CCC(N)CC4)c3c2)c1O] | 8.3 |
| 127 | 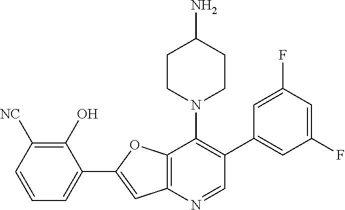 [SMILES: N#Cc1cccc(-c2cc3ncc(-c4cc(F)cc(F)c4)c(N4CCC(N)CC4)c3o2)c1O] | 6.6 |
| 128 | 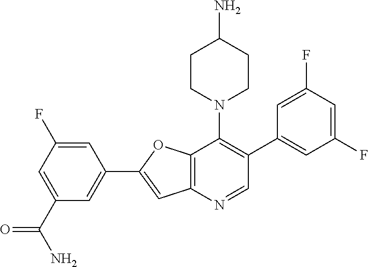 [SMILES: NC(=O)c1cc(F)cc(-c2cc3ncc(-c4cc(F)cc(F)c4)c(N4CCC(N)CC4)c3o2)c1] | 7.0 |
| 129 | 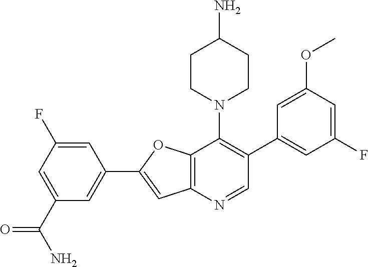 [SMILES: COc1cc(F)cc(-c2cnc3cc(-c4cc(F)cc(C(N)=O)c4)oc3c2N2CCC(N)CC2)c1] | 8.1 |
| 130 | 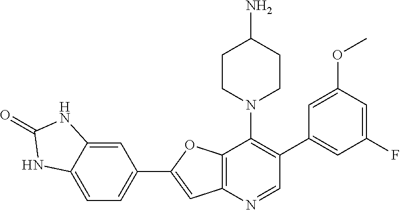 [SMILES: COc1cc(F)cc(-c2cnc3cc(-c4ccc5[nH]c(=O)[nH]c5c4)oc3c2N2CCC(N)CC2)c1] | 7.0 |
| 131 | 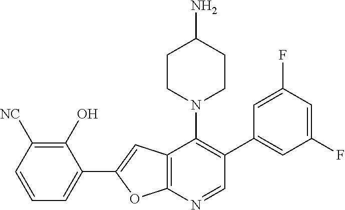 [SMILES: N#Cc1cccc(-c2cc3c(N4CCC(N)CC4)c(-c4cc(F)cc(F)c4)cnc3o2)c1O] | <5.0 |
| 132 | 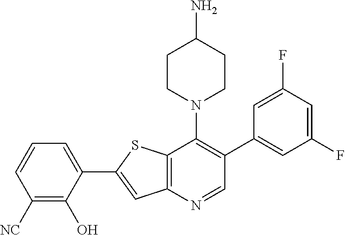 [SMILES: N#Cc1cccc(-c2cc3ncc(-c4cc(F)cc(F)c4)c(N4CCC(N)CC4)c3s2)c1O] | <5.0 |
| 133 | 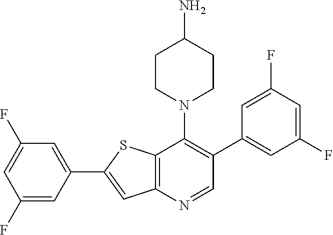 [SMILES: NC1CCN(c2c(-c3cc(F)cc(F)c3)cnc3cc(-c4cc(F)cc(F)c4)sc23)CC1] | 5.8 |
| 134 | 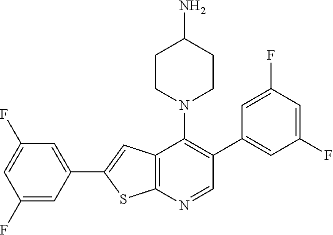 [SMILES: NC1CCN(c2c(-c3cc(F)cc(F)c3)cnc3sc(-c4cc(F)cc(F)c4)cc23)CC1] | 6.2 |
| 135 | 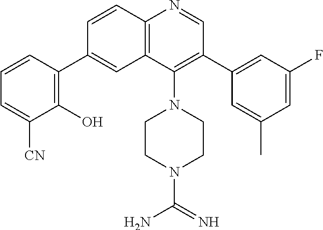 [SMILES: Cc1cc(F)cc(-c2cnc3ccc(-c4cccc(C#N)c4O)cc3c2N2CCN(C(=N)N)CC2)c1] | 6.1 |
| 136 | 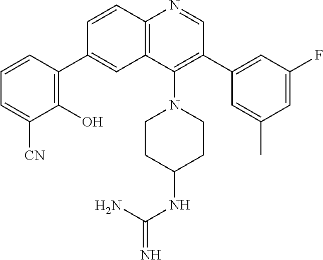 [SMILES: Cc1cc(F)cc(-c2cnc3ccc(-c4cccc(C#N)c4O)cc3c2N2CCC(NC(=N)N)CC2)c1] | 7.7 |
| 137 | 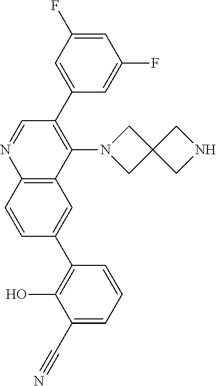 [SMILES: N#Cc1cccc(-c2ccc3ncc(-c4cc(F)cc(F)c4)c(N4CC5(CNC5)C4)c3c2)c1O] | <5.0 |
| 138 | 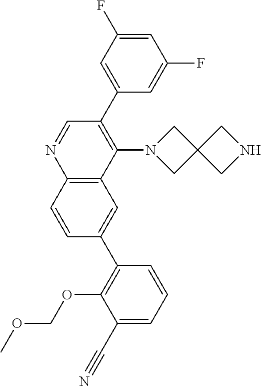 [SMILES: COCOc1c(C#N)cccc1-c1ccc2ncc(-c3cc(F)cc(F)c3)c(N3CC4(CNC4)C3)c2c1] | 5.2 |
| 139 | 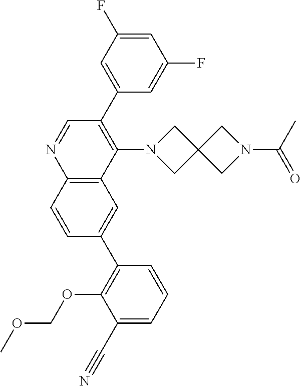 [SMILES: COCOc1c(C#N)cccc1-c1ccc2ncc(-c3cc(F)cc(F)c3)c(N3CC4(CN(C(C)=O)C4)C3)c2c1] | <5.3 |
| 140 | 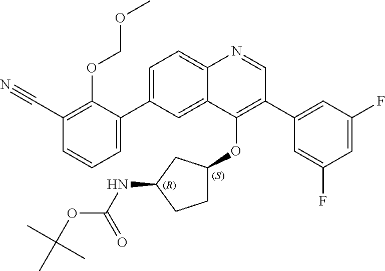 | <5.0 |
| 141 | 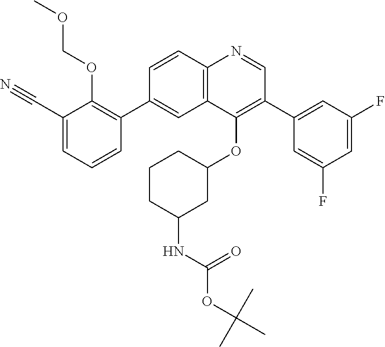 [SMILES: COCOc1c(C#N)cccc1-c1ccc2ncc(-c3cc(F)cc(F)c3)c(OC3CCCC(NC(=O)OC(C)(C)C)C3)c2c1] | <5.0 |
| 142 | 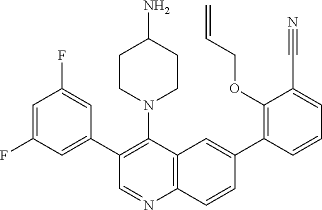 [SMILES: C=CCOc1c(C#N)cccc1-c1ccc2ncc(-c3cc(F)cc(F)c3)c(N3CCC(N)CC3)c2c1] | 9.3 |
| 143 | 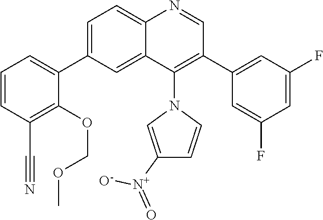 [SMILES: COCOc1c(C#N)cccc1-c1ccc2ncc(-c3cc(F)cc(F)c3)c(-n3ccc([N+](=O)[O-])c3)c2c1] | <5.0 |
| 144 | 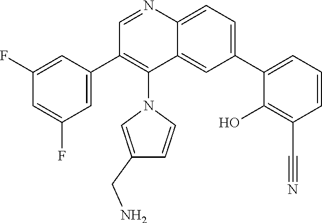 [SMILES: N#Cc1cccc(-c2ccc3ncc(-c4cc(F)cc(F)c4)c(-n4ccc(CN)c4)c3c2)c1O] | 5.9 |
| 145 | 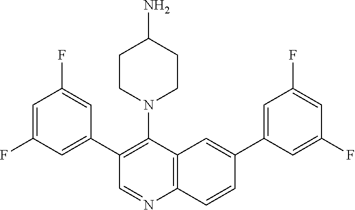 [SMILES: NC1CCN(c2c(-c3cc(F)cc(F)c3)cnc3ccc(-c4cc(F)cc(F)c4)cc23)CC1] | 10.5 |
| 146 | 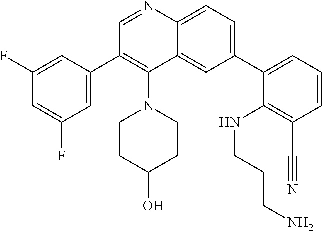 [SMILES: N#Cc1cccc(-c2ccc3ncc(-c4cc(F)cc(F)c4)c(N4CCC(O)CC4)c3c2)c1NCCCN] | 6.4 |
| 147 | 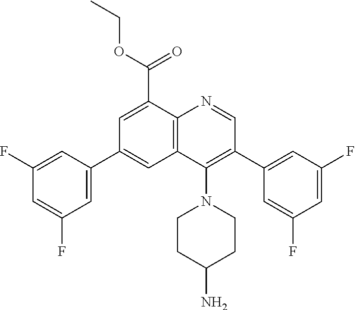 [SMILES: CCOC(=O)c1cc(-c2cc(F)cc(F)c2)cc2c(N3CCC(N)CC3)c(-c3cc(F)cc(F)c3)cnc12] | 10.4 |
| 148 | 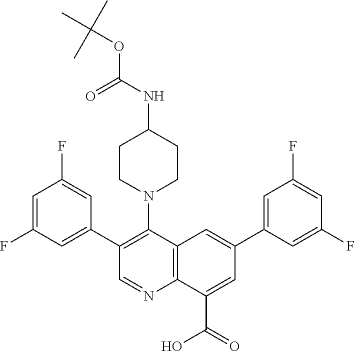 [SMILES: CC(C)(C)OC(=O)NC1CCN(c2c(-c3cc(F)cc(F)c3)cnc3c(C(=O)O)cc(-c4cc(F)cc(F)c4)cc23)CC1] | <6.1 |
| 149 | 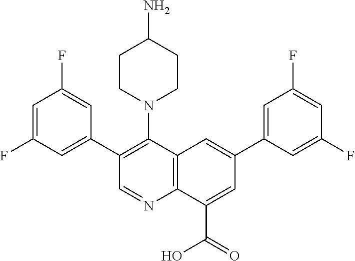 [SMILES: NC1CCN(c2c(-c3cc(F)cc(F)c3)cnc3c(C(=O)O)cc(-c4cc(F)cc(F)c4)cc23)CC1] | 9.4 |
| 150 | 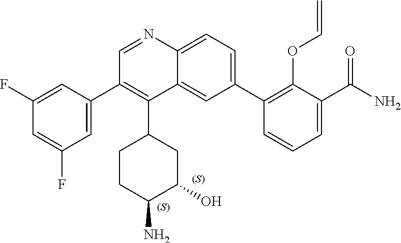 | 7 |
| 151 | 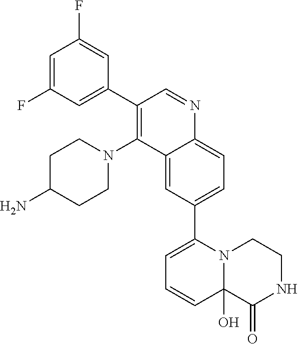 [SMILES: NC1CCN(c2c(-c3cc(F)cc(F)c3)cnc3ccc(C4=CC=CC5(O)C(=O)NCCN45)cc23)CC1] | 8.8 |
| 152 | 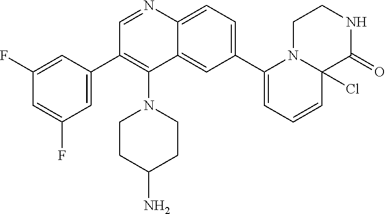 [SMILES: NC1CCN(c2c(-c3cc(F)cc(F)c3)cnc3ccc(C4=CC=CC5(Cl)C(=O)NCCN45)cc23)CC1] | 8.1 |
| 153 | 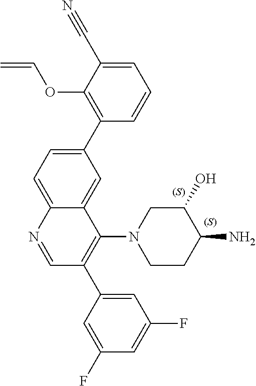 | 8.5 |
| 154 | 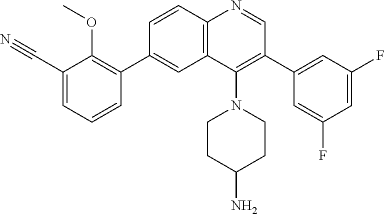 [SMILES: COc1c(C#N)cccc1-c1ccc2ncc(-c3cc(F)cc(F)c3)c(N3CCC(N)CC3)c2c1] | 9.5 |
| 155 | 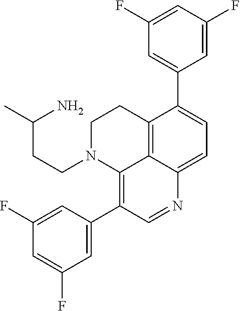 [SMILES: CC(N)CCN1CCc2c(-c3cc(F)cc(F)c3)ccc3ncc(-c4cc(F)cc(F)c4)c1c23] | 6.2 |
| 156 | 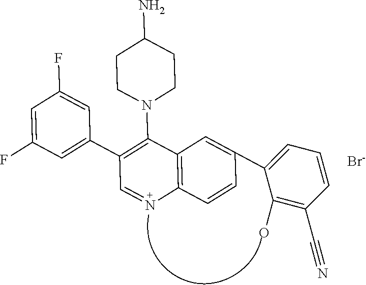 [SMILES: N#Cc1cccc(-c2ccc3ncc(-c4cc(F)cc(F)c4)c(N4CCC(N)CC4)c3c2)c1O ; [Br-]] | 9.6 |
| 157 | 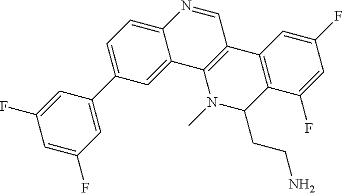 [SMILES: CN1c2c(cnc3ccc(-c4cc(F)cc(F)c4)cc23)-c2cc(F)cc(F)c2C1CCN] | 8 |
| 158 | 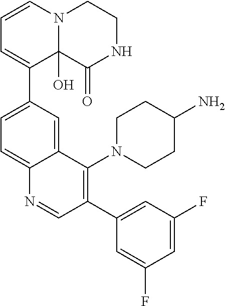 [SMILES: NC1CCN(c2c(-c3cc(F)cc(F)c3)cnc3ccc(C4=CC=CN5CCNC(=O)C45O)cc23)CC1] | 6.3 |
| 159 | 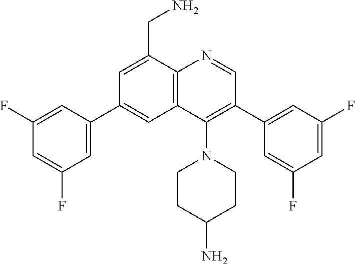 [SMILES: NCc1cc(-c2cc(F)cc(F)c2)cc2c(N3CCC(N)CC3)c(-c3cc(F)cc(F)c3)cnc12] | 9.4 |
| 160 | 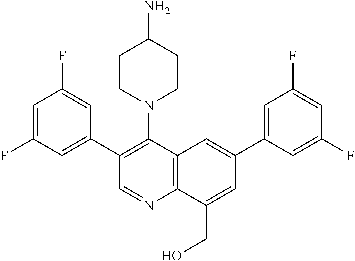 [SMILES: NC1CCN(c2c(-c3cc(F)cc(F)c3)cnc3c(CO)cc(-c4cc(F)cc(F)c4)cc23)CC1] | 10.6 |
| 161 | 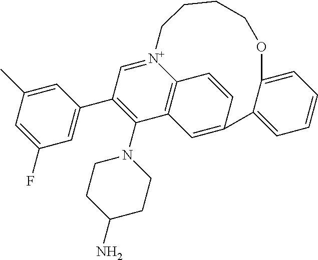 [SMILES: Cc1cc(F)cc(-c2c[n+]3c4ccc(cc4c2N2CCC(N)CC2)-c2ccccc2OCCCC3)c1] | 8 |
| 162 | 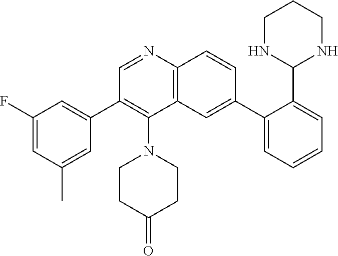 [SMILES: Cc1cc(F)cc(-c2cnc3ccc(-c4ccccc4C4NCCCN4)cc3c2N2CCC(=O)CC2)c1] | 6 |
| 163 | 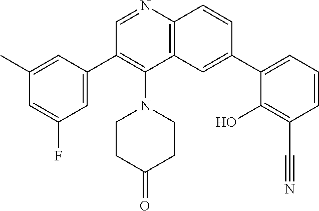 [SMILES: Cc1cc(F)cc(-c2cnc3ccc(-c4cccc(C#N)c4O)cc3c2N2CCC(=O)CC2)c1] | <5.0 |
| 164 | 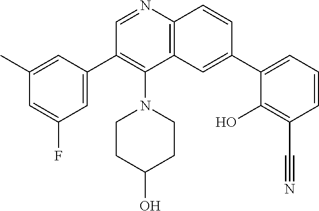 [SMILES: Cc1cc(F)cc(-c2cnc3ccc(-c4cccc(C#N)c4O)cc3c2N2CCC(O)CC2)c1] | <5.0 |
| 165 | 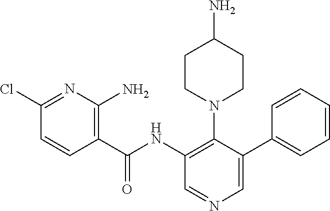 [SMILES: Nc1nc(Cl)ccc1C(=O)Nc1cncc(-c2ccccc2)c1N1CCC(N)CC1] | 7.9 |

*indicates that the stereochemistry is arbritrarily assigned.

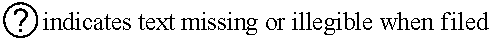

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A compound of Formula I: 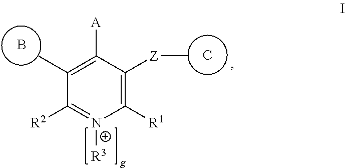
or a pharmaceutically acceptable salt thereof, wherein:
g is 1 or 0;
Z is absent, —N(H)—C(═O)— or —C(═O)—NH—;
Ring B is phenyl or pyridinyl, each of which is mono-substituted with R^{B1} or di-substituted with one R^{B1} and one R^{B2}, wherein:
R^{B1} and R^{B2} are independently hydrogen, cyano, C₁-C₆ alkyl, halogen, halo-C₁-C₆ alkyl, hydroxy-C₁-C₆ alkyl, hydroxy, C₁-C₆ alkoxy, —O—C₂-C₆ alkenyl, —NH—(CH₂)₁₋₅—NH₂, —O—(CH₂)_{q2}—O—(CH₂)ᵣ₂—CH₃, or —C(O)—NR^{Na}R^{Nb},
each R^{Na} and R^{Nb} is independently hydrogen or C₁-C₆ alkyl, and
q2 and r2 are independently an integer from 1 to 4;
Ring C is 4- to 10-membered monocyclic or bicyclic fused heterocyclyl, 6- to 10-membered aryl, or 5- to 10-membered heteroaryl, each of which is substituted with R^{C1}, R^{C2}, and R^{C3}, wherein:
said heterocyclyl or heteroaryl contains 1, 2, 3, or 4 ring heteroatoms selected from N, O, and S,
R^{C1} is hydrogen or is bonded to R¹, R³, or R⁶,
R^{C2} and R^{C3} are independently hydrogen, halogen, cyano, C₁-C₆ alkyl, hydroxy, —C(═O)NH₂, —O—C₂-C₆ alkenyl, —C₁-C₆ alkoxy, —NH—(CH₂)₁₋₅—NH₂, —O-piperidinyl, or —O—(CH₂)_{qC}—O—(CH₂)_{rC}—CH₃, and
qC and rC are independently an integer from 1 to 4;
R¹ is hydrogen or halogen, or
R¹ and Z together with the ring to which each is attached form a fused bicyclic ring, optionally substituted with one or two R⁸, or
R¹ and R^{C1} together with the ring to which each is attached form a fused tricyclic ring;
R² is hydrogen or halogen, or
R² and R^{B1} together with the ring to which each is attached form a fused tricyclic ring;
R³, if present, is a group covalently bound to R^{C1};
A is:
i. —NR⁴R⁵, wherein:
R⁴ is optionally substituted C₁-C₆ alkyl, —NH—C₁-C₆ alkyl-NHR^{4a}, or —C₁-C₆ alkyl-NHR^{4a}, wherein the optional substituents are selected from the group consisting of halogen and hydroxy,
R⁴ is hydrogen or methyl, and
R⁵ is hydrogen or optionally substituted C₁-C₆ alkyl,
wherein the optional substituents are selected from the group consisting of halogen and hydroxy, or
R⁵ and R^{B1} together with the N to which R⁵ is attached and Ring B to which R^{B1} is attached form a fused heterocyclyl, or
R⁵ and R^{C1} together with the N to which R⁵ is attached and Ring C to which R^{C1} is attached form a fused heterocyclyl, or
R⁵ and R⁸ together with the N to which R is attached and the ring to which R⁸ is attached form a fused heterocyclyl;
ii. spirocyclic, substituted with —NH—R⁶ or R^{S}, wherein R^{S} is hydrogen or —C(═O)—C₁-C₆ alkyl; or
iii. —O—(C₃-C₈ cycloalkyl), 5- to 11-membered heterocyclyl, or 5- to 6-membered heteroaryl, each of which is mono-substituted with —NH—R⁶ or di-substituted with one R^{F} and one R^{G}, wherein:
R^{F} is hydroxy, hydroxy-C₁-C₆ alkyl, nitro, —C(═O)H,
—C₁-C₆ alkoxy, —C(═NH)—NH₂, —NH—C(═NH)—NH₂,
—C(═O)—C₁-C₆ alkyl, —(C═O)—O—C₁-C₆ alkyl,
or —(C₁-C₆ alkyl)ₓ-NR^{F1}R^{F2},
x is 0 or 1,
R^{F1} and R^{F2} are independently H, —(C═O)—O—C₁-C₆-alkyl, or C₁-C₆ alkyl, and
R^{G} is hydrogen or —C₁-C₆ alkoxy; or
R^{G} together with R^{C1} form a —O—(CH₂)ₖ—O—, wherein k is an integer from 1 to 5; or
R^{F} and R^{G} together form carboxy; and
R⁶ and R^{C1} form: 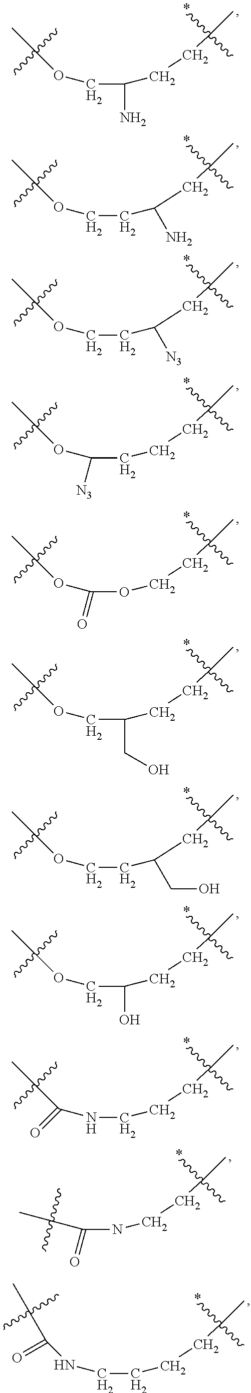 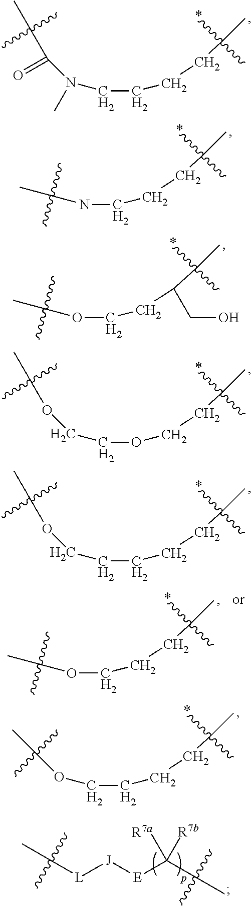
wherein 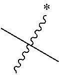
represents the attachment point of R⁶;
provided that the compound is not one of:

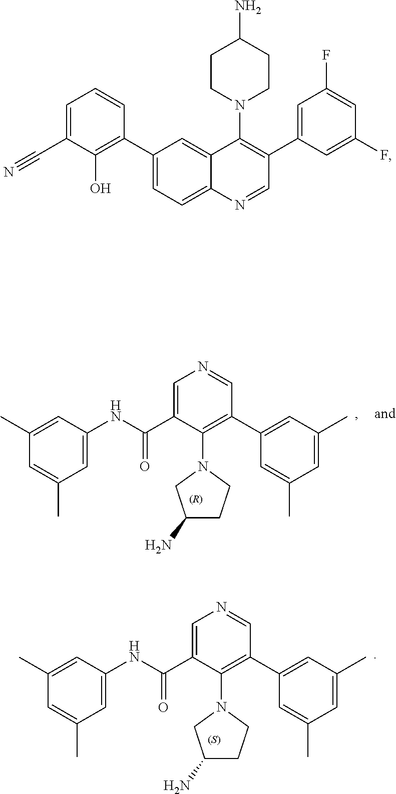

2. (canceled)

3. A compound having a structure of Formula Ia or Ib: 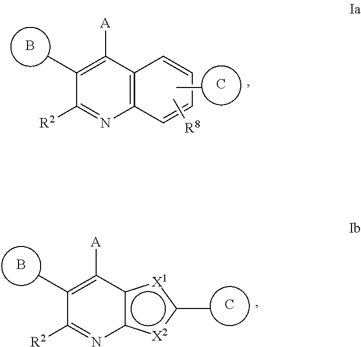
wherein:
Ring B is phenyl or pyridinyl, each of which is mono-substituted with R^{B1} or di-substituted with one R^{B1} and one R^{B2}, wherein:
R^{B1} and R^{B2} are independently hydrogen, cyano, C₁-C₆ alkyl, halogen, halo-C₁-C₆ alkyl, hydroxy-C₁-C₆ alkyl, hydroxy, C₁-C₆ alkoxy,
—O—C₂-C₆ alkenyl, —NH—(CH₂)₁₋₅—NH₂, —O—(CH₂)_{q2}—O—(CH₂)ᵣ₂—CH₃, or —C(O)—NR^{Na}R^{Nb},
each R^{Na} and R^{Nb} is independently hydrogen or C₁-C₆ alkyl, and
q2 and r2 are independently an integer from 1 to 4;
Ring C is 4- to 10-membered monocyclic or bicyclic fused heterocyclyl, 6- to 10-membered aryl, or 5- to 10-membered heteroaryl, each of which is substituted with R^{C1}, R^{C2}, and R^{C3}, wherein:
said heterocyclyl or heteroaryl contains 1, 2, 3, or 4 ring heteroatoms selected from N, O, and S,
R^{C1} is hydrogen, is bonded to R⁶, or is as further defined,
R^{C2} and R^{C3} are independently hydrogen, halogen, cyano, C₁-C₆ alkyl, hydroxy, —C(═O)NH₂, —O—C₂-C₆ alkenyl, —C₁-C₆ alkoxy, —NH—(CH₂)₁₋₅—NH₂, —O-piperidinyl, or —O—(CH₂)_{qC}—O—(CH₂)_{rC}—CH₃, and
qC and rC are independently an integer from 1 to 4;
R² is hydrogen or halogen, or
R² and R^{B1} together with the ring to which each is attached form a fused tricyclic ring;
A is:
i. —NR⁴R⁵, wherein:
R⁴ is optionally substituted C₁-C₆ alkyl, —NH—C₁-C₆ alkyl-NHR^{4a},
or —C₁-C₆ alkyl-NHR^{4a}, wherein the optional substituents are selected from the group consisting of halogen and hydroxy,
R^{4a} is hydrogen or methyl, and
R⁵ is hydrogen or optionally substituted C₁-C₆ alkyl, wherein the optional substituents are selected from the group consisting of halogen and hydroxy, or
R⁵ and R^{B1} together with the N to which R⁵ is attached and Ring B to which R^{B1} is attached form a fused heterocyclyl, or
R⁵ and R^{C1} together with the N to which R⁵ is attached and Ring C to which R^{C1} is attached form a fused heterocyclyl, or
R⁵ and R⁸ together with the N to which R is attached and the ring to which R⁸ is attached form a fused heterocyclyl:
ii. spirocylic, substituted with —NH—R⁶ or R^{S}, wherein R^{S} is hydrogen or —C(═O)—C₁-C₆ alkyl; or
iii. —O—(C₃-C₈ cycloalkyl), 5- to 11-membered heterocyclyl, or 5- to 6-membered heteroaryl, each of which is mono-substituted with —NH—R⁶ or di-substituted with one R^{F} and one R^{G}, wherein:
R^{F} is hydroxy, hydroxy-C₁-C₆ alkyl, nitro, —C(═O)H,
—C₁-C₆alkoxy, —C(═NH)—NH₂, —NH—C(═NH)—NH₂,
—C(═O)—C₁-C₆ alkyl, —(C═O)—O—C₁-C₆ alkyl, or —(C₁-C₆ alkyl)ₓ-NR^{F1}R^{F2},
x is 0 or 1,
R^{F1} and R^{F2} are independently H, —(C═O)—O—C₁-C₆-alkyl, or C₁-C₆ alkyl, and
R^{G} is hydrogen or —C₁-C₆ alkoxy; or
R^{G} together with R^{C1} form a —O—(CH₂)ₖ—O—, wherein k is an integer from 1 to 5; or
R^{F} and R^{G} together form carboxy; and
R⁶ and R^{C1} form: 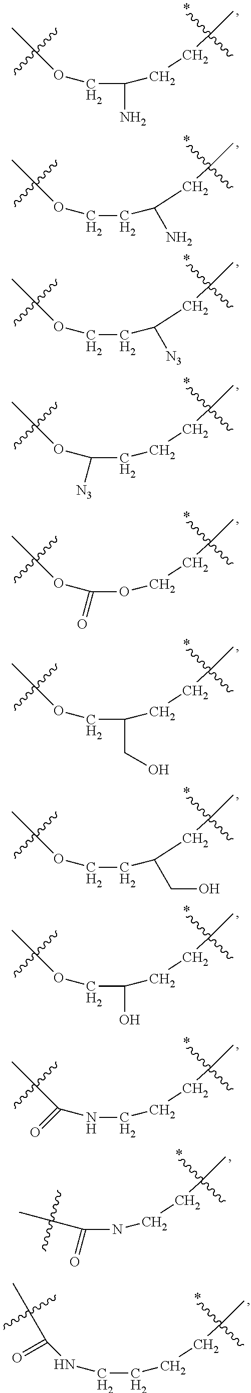 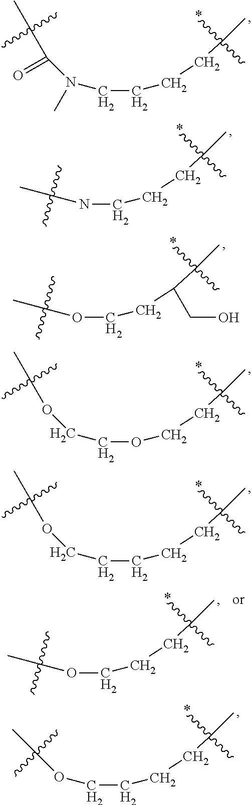
wherein 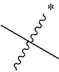
represents the attachment point of R⁶;
X¹ and X² are independently O, N, N—R⁸, S, or C—R⁸;
R⁸ is as defined in (i), or
R⁸ is hydrogen, optionally substituted C₁-C₆ alkyl, or —C(═O)OR^{8a}, wherein the optional substituents are selected from the group consisting of halogen, —NH₂, and hydroxy; and
R^{8a} is hydrogen or C₁-C₆ alkyl;
provided that the compound is not:

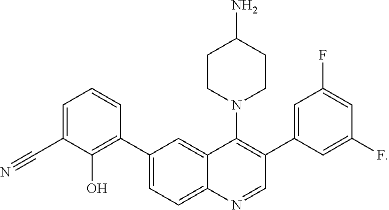 [SMILES: N#Cc1cccc(-c2ccc3ncc(-c4cc(F)cc(F)c4)c(N4CCC(N)CC4)c3c2)c1O]

4. The compound of claim 3, having a structure of Formula Ia-1, Ia-2, Ia-3, or Ib-1:

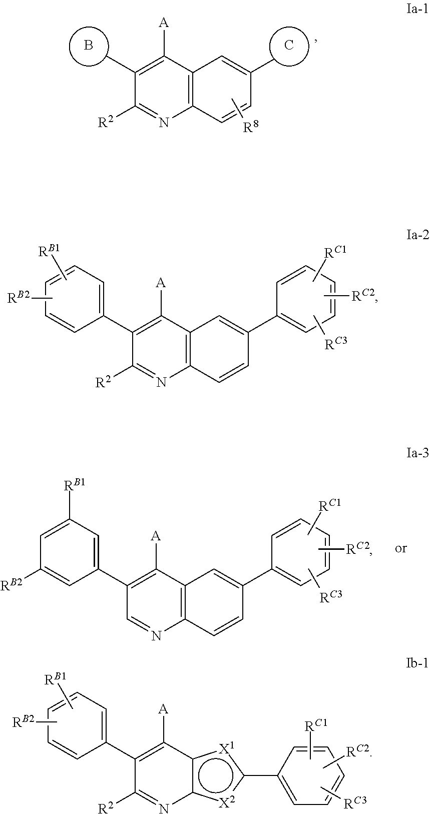

5-9. (canceled)

10. The compound of claim 3, wherein R^{B1} and R^{B2} are independently C₁-C₆ alkyl, halogen, or C₁-C₆ alkoxy.

11-22. (canceled)

23. The compound of claim 3, wherein
Ring B is phenyl di-substituted with R^{B1} and R^{B2}; and
Ring C is 4- to 10-membered monocyclic or bicyclic heterocyclyl.

24-25. (canceled)

26. The compound of claim 1, having a structure of Formula Ic: 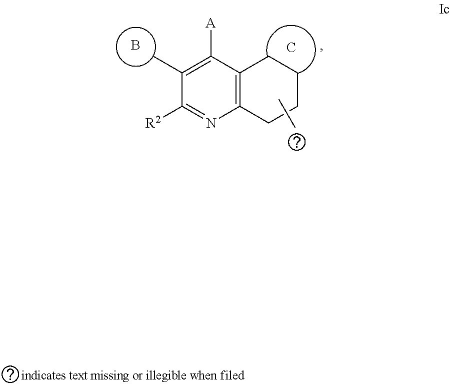
wherein R⁸ is hydrogen.

27. The compound of claim 26, wherein Ring C is 8- to 10-membered heteroaryl substituted with R^{C2} and R^{C3}, and Ring B is phenyl di-substituted with R^{B1} and R^{B2}.

28-34. (canceled)

35. The compound of claim 1, having a structure of Formula Id: 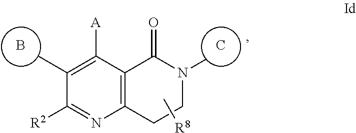
wherein R⁸ is hydrogen.

36. The compound of claim 35, wherein Ring C is phenyl substituted with R^{C1}, R^{C2}, and R^{C3}, and Ring B is phenyl di-substituted with R^{B1} and R^{B2}.

37-44. (canceled)

45. The compound of claim 1, having a structure of Formula Ie: 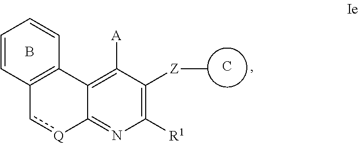
wherein:
Q is O or CH₂ and [symbol] is a single bond, or
Q is N and [symbol] is a double bond;
R¹ is hydrogen or halogen; and
Ring B is phenyl substituted with R^{B2}.

46. The compound of claim 45, wherein R¹ is hydrogen.

47. The compound of claim 45, wherein Ring C is 8- to 10-membered heteroaryl substituted with R^{C1}, R^{C2}, and R^{C3}.

48-50. (canceled)

51. The compound of claim 45, wherein Q is N and [symbol] is a double bond; or wherein Q is O and [symbol] is a single bond.

52. (canceled)

53. The compound of claim 1, wherein A is 5- to 11-membered heterocyclyl or 5- to 6-membered heteroaryl, each of which is mono-substituted with —NH—R⁶ or di-substituted with one R^{F} and one R^{G}.

54. The compound of claim 53, wherein A has the structure: 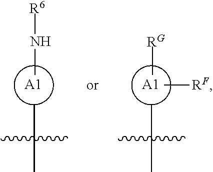
wherein Ring A1 is 5- to 6-membered heteroaryl or 5- to 6-membered heterocyclyl.

55-58. (canceled)

59. The compound of claim 1, wherein A is —O—(C₃-C₈ cycloalkyl) which is mono-substituted with —NH—R⁶ or di-substituted with one R^{F} and one R^{G}.

60. The compound of claim 59, wherein A has the structure: 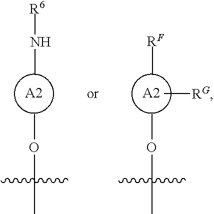
wherein Ring A2 is C₄-C₆ cycloalkyl.

61-62. (canceled)

63. The compound of claim 53, wherein R⁶ and R^{C1} form: 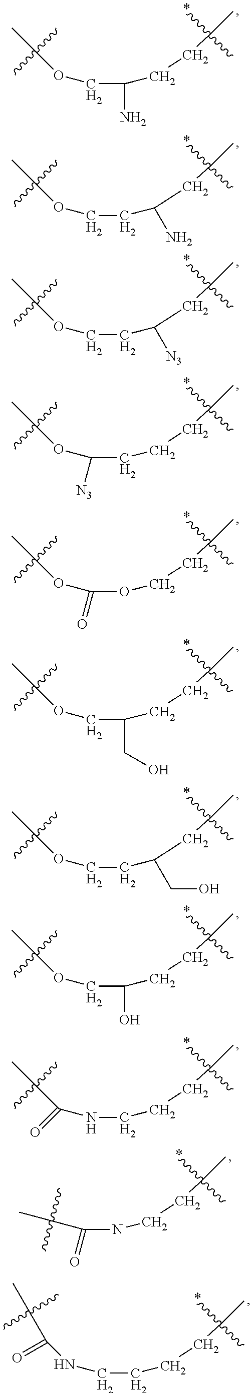 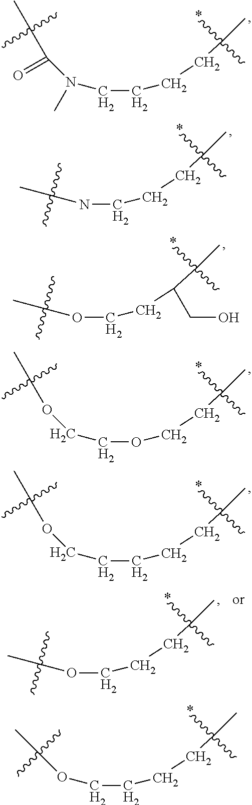
wherein 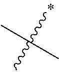
represents the attachment point of R⁶.

64. The compound of claim 63, having a structure of Formula Ia-4 or Ia-5: 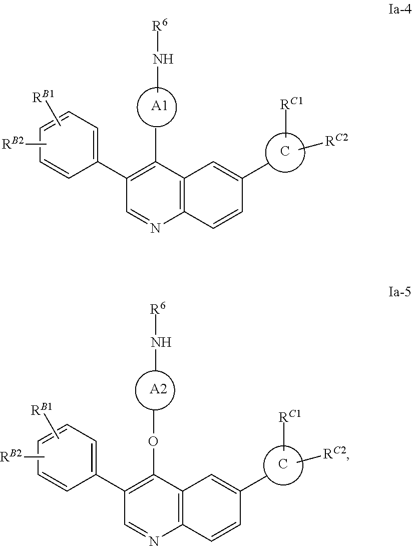
wherein Ring A1 is 5- to 6-membered heteroaryl or 5- to 6-membered heterocyclyl; and Ring A2 is C₄-C₆ cycloalkyl.

65. (canceled)

66. The compound of claim 64, wherein Ring C to which R^{C1} is attached has the structure:

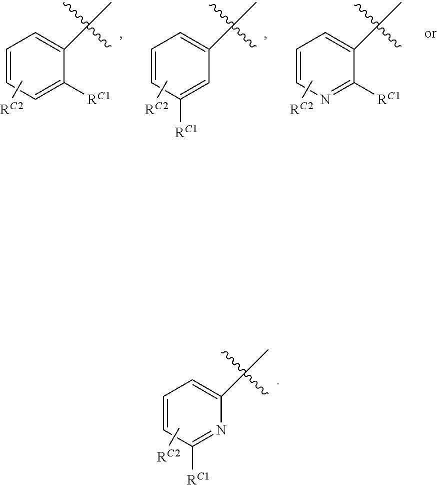

67-100. (canceled)

101. The compound of claim 1, having a structure of Formula I-2: 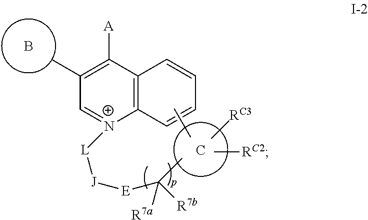
wherein -L-J-E-(CR^{7a}R^{7b})ₚ— form:

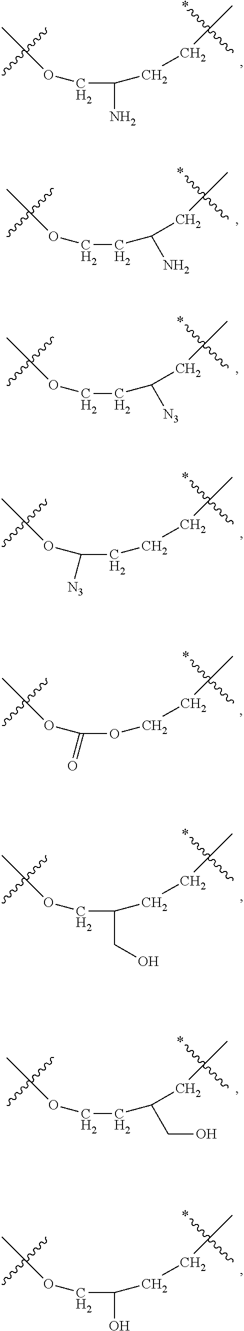

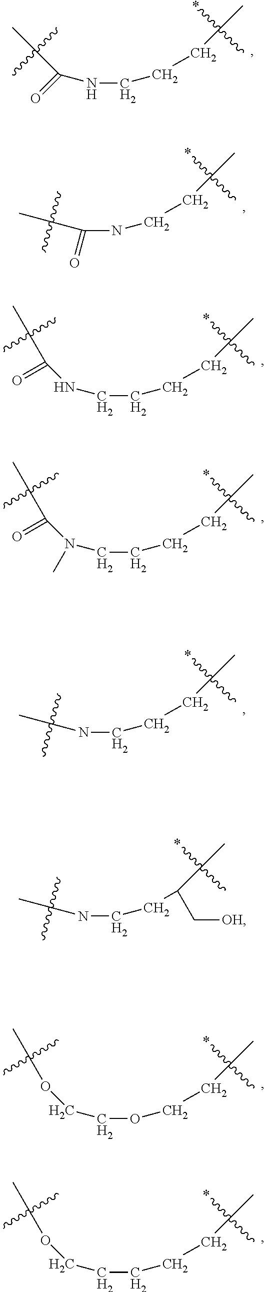

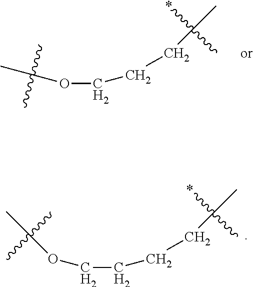

102. (canceled)

103. The compound of claim 11, wherein Ring B is phenyl di-substituted with R^{B1} which is halogen and R^{B2} which is halogen or C₁-C₆ alkyl.

104-112. (canceled)

113. A compound as shown

| Cpd. No. | Structure | Cpd. No. | Structure |
|---|---|---|---|
| 1 | 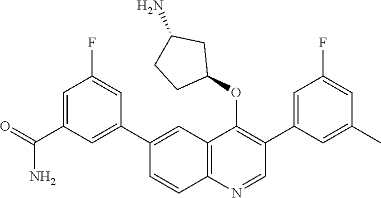 [SMILES: Cc1cc(F)cc(-c2cnc3ccc(-c4cc(F)cc(C(N)=O)c4)cc3c2O[C@H]2CC[C@H](N)C2)c1] | 83 | 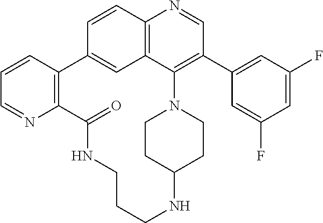 [SMILES: O=C1NCCCNC2CCN(CC2)c2c(-c3cc(F)cc(F)c3)cnc3ccc(cc23)-c2cccnc21] |
| 2 | 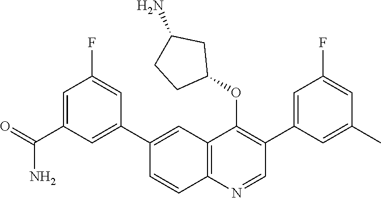 [SMILES: Cc1cc(F)cc(-c2cnc3ccc(-c4cc(F)cc(C(N)=O)c4)cc3c2O[C@@H]2CC[C@H](N)C2)c1] | 84 | 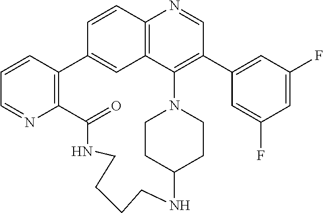 [SMILES: O=C1NCCCCNC2CCN(CC2)c2c(-c3cc(F)cc(F)c3)cnc3ccc(cc23)-c2cccnc21] |
| 3 | 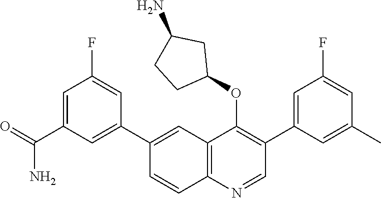 [SMILES: Cc1cc(F)cc(-c2cnc3ccc(-c4cc(F)cc(C(N)=O)c4)cc3c2O[C@H]2CC[C@@H](N)C2)c1] | 85 | 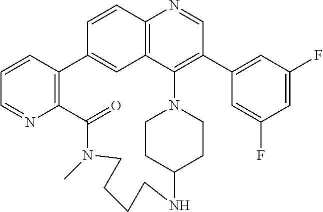 [SMILES: CN1CCCCNC2CCN(CC2)c2c(-c3cc(F)cc(F)c3)cnc3ccc(cc23)-c2cccnc2C1=O] |
| 4 | 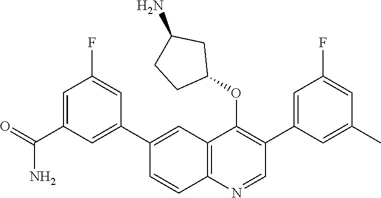 [SMILES: Cc1cc(F)cc(-c2cnc3ccc(-c4cc(F)cc(C(N)=O)c4)cc3c2O[C@@H]2CC[C@@H](N)C2)c1] | 86 | 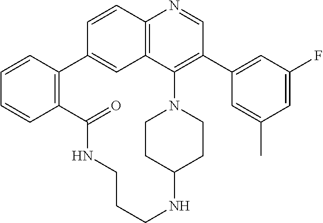 [SMILES: Cc1cc(F)cc(-c2cnc3ccc4cc3c2N2CCC(CC2)NCCCNC(=O)c2ccccc2-4)c1] |
| 5 | 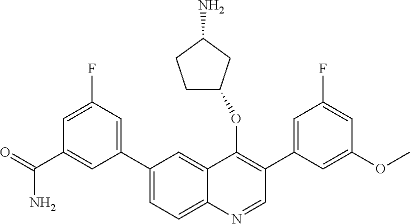 [SMILES: COc1cc(F)cc(-c2cnc3ccc(-c4cc(F)cc(C(N)=O)c4)cc3c2O[C@@H]2CC[C@H](N)C2)c1] | 87 | 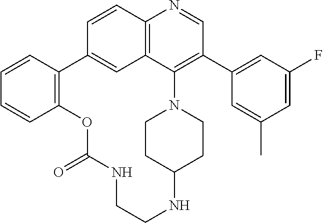 [SMILES: Cc1cc(F)cc(-c2cnc3ccc4cc3c2N2CCC(CC2)NCCNC(=O)Oc2ccccc2-4)c1] |
| 6 | 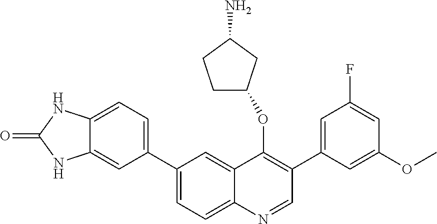 [SMILES: COc1cc(F)cc(-c2cnc3ccc(-c4ccc5[nH]c(=O)[nH]c5c4)cc3c2O[C@@H]2CC[C@H](N)C2)c1] | 88* | 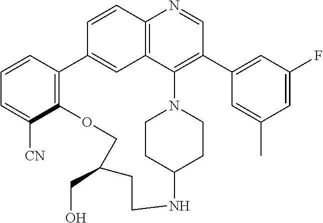 |
| 7 | 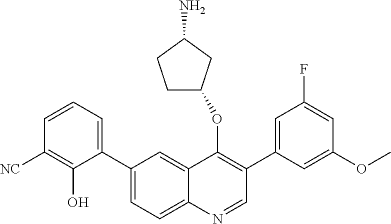 [SMILES: COc1cc(F)cc(-c2cnc3ccc(-c4cccc(C#N)c4O)cc3c2O[C@@H]2CC[C@H](N)C2)c1] | 89* | 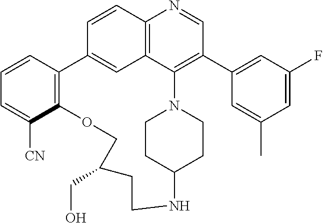 |
| 8 | 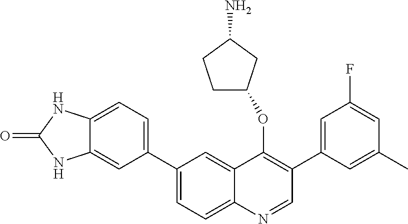 [SMILES: Cc1cc(F)cc(-c2cnc3ccc(-c4ccc5[nH]c(=O)[nH]c5c4)cc3c2O[C@@H]2CC[C@H](N)C2)c1] | 90* | 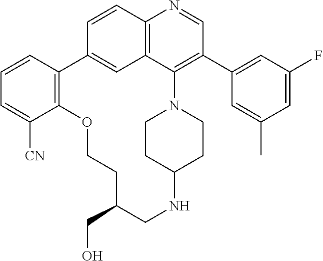 [SMILES: Cc1cc(F)cc(-c2cnc3ccc4cc3c2N2CCC(CC2)NC[C@@H](CO)CCOc2c(C#N)cccc2-4)c1] |
| 9 | 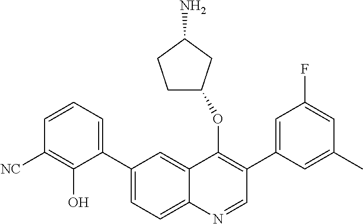 [SMILES: Cc1cc(F)cc(-c2cnc3ccc(-c4cccc(C#N)c4O)cc3c2O[C@@H]2CC[C@H](N)C2)c1] | 91* | 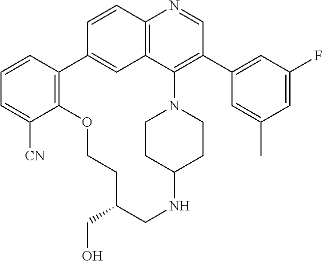 [SMILES: Cc1cc(F)cc(-c2cnc3ccc4cc3c2N2CCC(CC2)NC[C@H](CO)CCOc2c(C#N)cccc2-4)c1] |
| 10 | 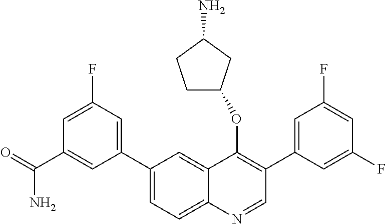 [SMILES: NC(=O)c1cc(F)cc(-c2ccc3ncc(-c4cc(F)cc(F)c4)c(O[C@@H]4CC[C@H](N)C4)c3c2)c1] | 92 | 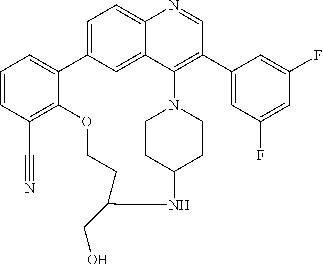 [SMILES: N#Cc1cccc2c1OCCC(CO)NC1CCN(CC1)c1c(-c3cc(F)cc(F)c3)cnc3ccc-2cc13] |
| 11 | 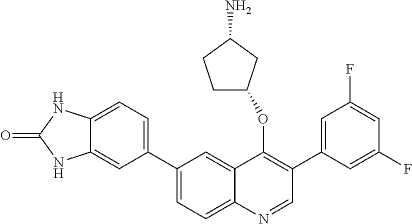 [SMILES: N[C@H]1CC[C@@H](Oc2c(-c3cc(F)cc(F)c3)cnc3ccc(-c4ccc5[nH]c(=O)[nH]c5c4)cc23)C1] | 93 | 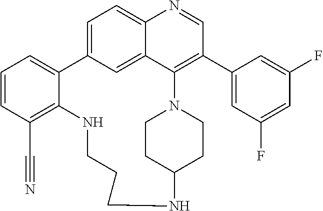 [SMILES: N#Cc1cccc2c1NCCCNC1CCN(CC1)c1c(-c3cc(F)cc(F)c3)cnc3ccc-2cc13] |
| 12 | 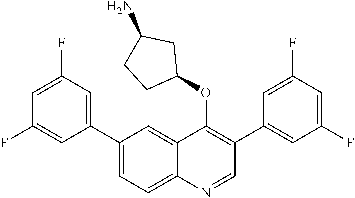 [SMILES: N[C@@H]1CC[C@H](Oc2c(-c3cc(F)cc(F)c3)cnc3ccc(-c4cc(F)cc(F)c4)cc23)C1] | 94 | 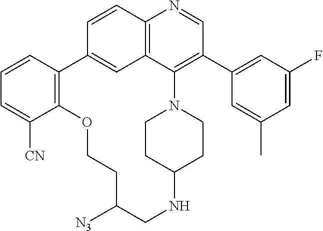 [SMILES: Cc1cc(F)cc(-c2cnc3ccc4cc3c2N2CCC(CC2)NCC(N=[N+]=[N-])CCOc2c(C#N)cccc2-4)c1] |
| 13 | 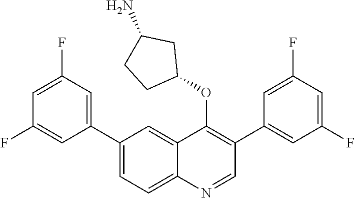 [SMILES: N[C@H]1CC[C@@H](Oc2c(-c3cc(F)cc(F)c3)cnc3ccc(-c4cc(F)cc(F)c4)cc23)C1] | 95 | 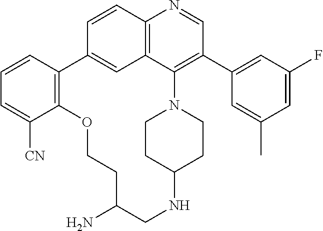 [SMILES: Cc1cc(F)cc(-c2cnc3ccc4cc3c2N2CCC(CC2)NCC(N)CCOc2c(C#N)cccc2-4)c1] |
| 14 | 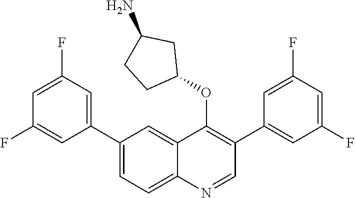 [SMILES: N[C@@H]1CC[C@@H](Oc2c(-c3cc(F)cc(F)c3)cnc3ccc(-c4cc(F)cc(F)c4)cc23)C1] | 96* | 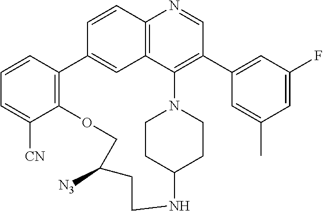 [SMILES: Cc1cc(F)cc(-c2cnc3ccc4cc3c2N2CCC(CC2)NCC[C@@H](N=[N+]=[N-])COc2c(C#N)cccc2-4)c1] |
| 15 | 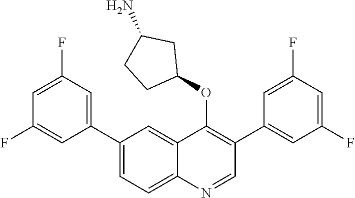 [SMILES: N[C@H]1CC[C@H](Oc2c(-c3cc(F)cc(F)c3)cnc3ccc(-c4cc(F)cc(F)c4)cc23)C1] | 97* | 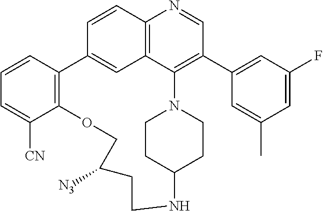 [SMILES: Cc1cc(F)cc(-c2cnc3ccc4cc3c2N2CCC(CC2)NCC[C@H](N=[N+]=[N-])COc2c(C#N)cccc2-4)c1] |
| 16 | 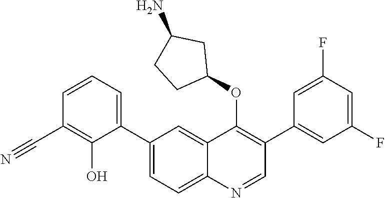 [SMILES: N#Cc1cccc(-c2ccc3ncc(-c4cc(F)cc(F)c4)c(O[C@H]4CC[C@@H](N)C4)c3c2)c1O] | 98* | 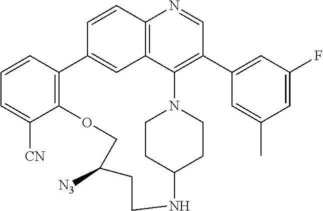 [SMILES: Cc1cc(F)cc(-c2cnc3ccc4cc3c2N2CCC(CC2)NCC[C@@H](N=[N+]=[N-])COc2c(C#N)cccc2-4)c1] |
| 17 | 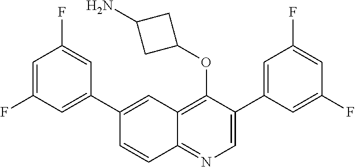 [SMILES: NC1CC(Oc2c(-c3cc(F)cc(F)c3)cnc3ccc(-c4cc(F)cc(F)c4)cc23)C1] | 99* | 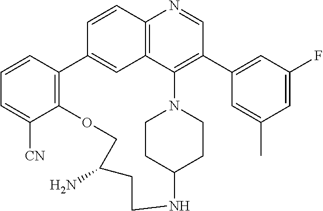 [SMILES: Cc1cc(F)cc(-c2cnc3ccc4cc3c2N2CCC(CC2)NCC[C@H](N)COc2c(C#N)cccc2-4)c1] |
| 18 | 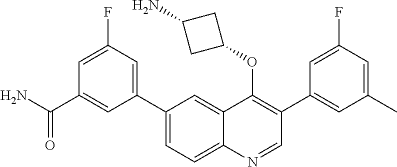 [SMILES: Cc1cc(F)cc(-c2cnc3ccc(-c4cc(F)cc(C(N)=O)c4)cc3c2O[C@H]2C[C@@H](N)C2)c1] | 100* | 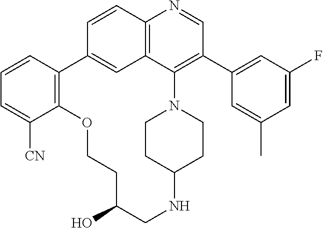 [SMILES: Cc1cc(F)cc(-c2cnc3ccc4cc3c2N2CCC(CC2)NC[C@@H](O)CCOc2c(C#N)cccc2-4)c1] |
| 19 | 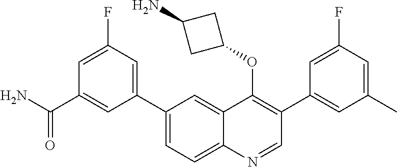 [SMILES: Cc1cc(F)cc(-c2cnc3ccc(-c4cc(F)cc(C(N)=O)c4)cc3c2O[C@H]2C[C@H](N)C2)c1] | 101* | 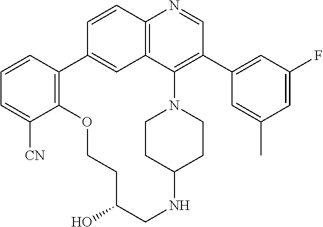 [SMILES: Cc1cc(F)cc(-c2cnc3ccc4cc3c2N2CCC(CC2)NC[C@H](O)CCOc2c(C#N)cccc2-4)c1] |
| 20 | 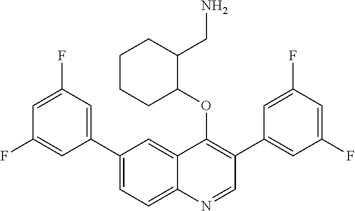 [SMILES: NCC1CCCCC1Oc1c(-c2cc(F)cc(F)c2)cnc2ccc(-c3cc(F)cc(F)c3)cc12] | 102* | 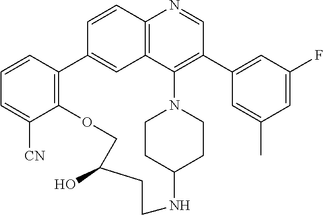 [SMILES: Cc1cc(F)cc(-c2cnc3ccc4cc3c2N2CCC(CC2)NCC[C@@H](O)COc2c(C#N)cccc2-4)c1] |
| 21 | 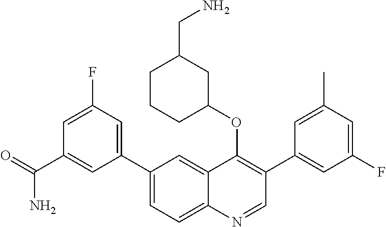 [SMILES: Cc1cc(F)cc(-c2cnc3ccc(-c4cc(F)cc(C(N)=O)c4)cc3c2OC2CCCC(CN)C2)c1] | 103* | 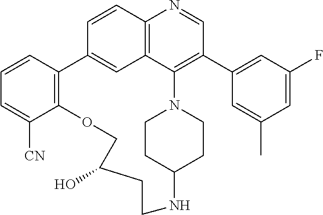 [SMILES: Cc1cc(F)cc(-c2cnc3ccc4cc3c2N2CCC(CC2)NCC[C@H](O)COc2c(C#N)cccc2-4)c1] |
| 22 | 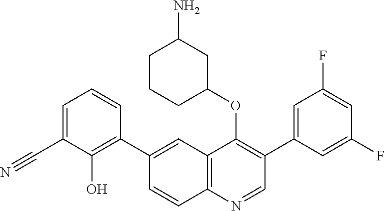 [SMILES: N#Cc1cccc(-c2ccc3ncc(-c4cc(F)cc(F)c4)c(OC4CCCC(N)C4)c3c2)c1O] | 104 | 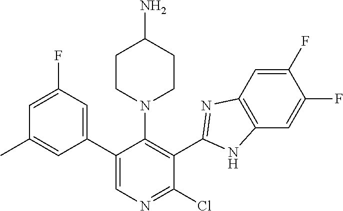 [SMILES: Cc1cc(F)cc(-c2cnc(Cl)c(-c3nc4cc(F)c(F)cc4[nH]3)c2N2CCC(N)CC2)c1] |
| 23 | 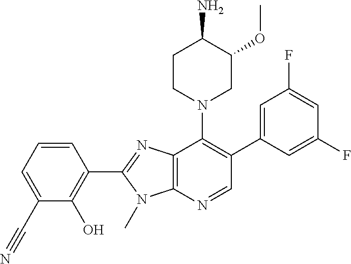 [SMILES: CO[C@@H]1CN(c2c(-c3cc(F)cc(F)c3)cnc3c2nc(-c2cccc(C#N)c2O)n3C)CC[C@H]1N] | 105 | 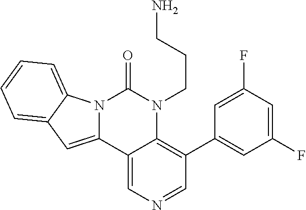 [SMILES: NCCCn1c(=O)n2c3ccccc3cc2c2cncc(-c3cc(F)cc(F)c3)c21] |
| 24 | 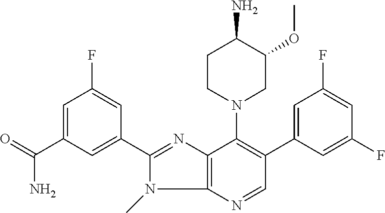 [SMILES: CO[C@@H]1CN(c2c(-c3cc(F)cc(F)c3)cnc3c2nc(-c2cc(F)cc(C(N)=O)c2)n3C)CC[C@H]1N] | 106 | 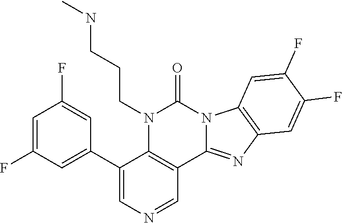 [SMILES: CNCCCn1c(=O)n2c3cc(F)c(F)cc3nc2c2cncc(-c3cc(F)cc(F)c3)c21] |
| 25 | 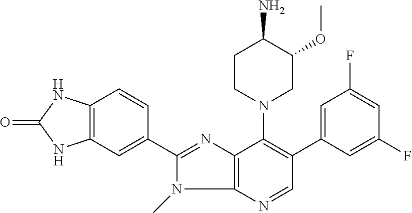 [SMILES: CO[C@@H]1CN(c2c(-c3cc(F)cc(F)c3)cnc3c2nc(-c2ccc4[nH]c(=O)[nH]c4c2)n3C)CC[C@H]1N] | 107 | 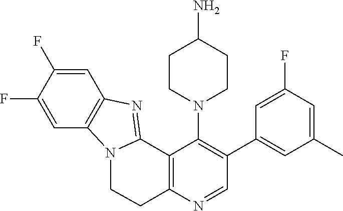 [SMILES: Cc1cc(F)cc(-c2cnc3c(c2N2CCC(N)CC2)-c2nc4cc(F)c(F)cc4n2CC3)c1] |
| 26 | 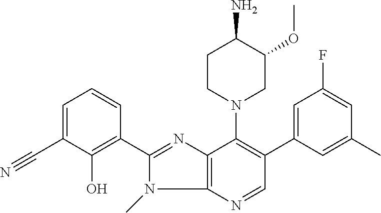 [SMILES: CO[C@@H]1CN(c2c(-c3cc(C)cc(F)c3)cnc3c2nc(-c2cccc(C#N)c2O)n3C)CC[C@H]1N] | 108 | 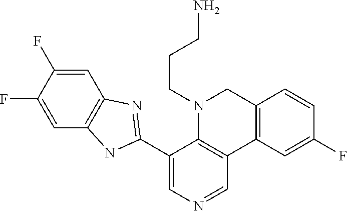 [SMILES: NCCCN1Cc2ccc(F)cc2-c2cncc(-c3nc4cc(F)c(F)cc4[nH]3)c21] |
| 27 | 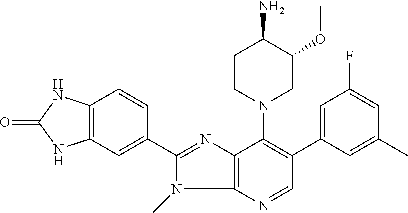 [SMILES: CO[C@@H]1CN(c2c(-c3cc(C)cc(F)c3)cnc3c2nc(-c2ccc4[nH]c(=O)[nH]c4c2)n3C)CC[C@H]1N] | 109 | 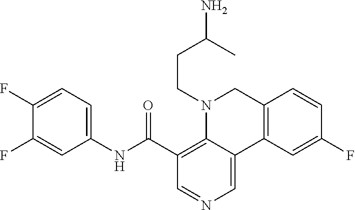 [SMILES: CC(N)CCN1Cc2ccc(F)cc2-c2cncc(C(=O)Nc3ccc(F)c(F)c3)c21] |
| 28 | 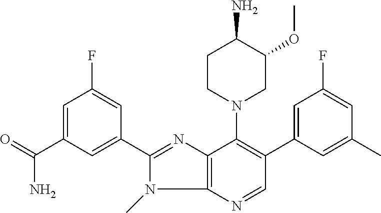 [SMILES: CO[C@@H]1CN(c2c(-c3cc(C)cc(F)c3)cnc3c2nc(-c2cc(F)cc(C(N)=O)c2)n3C)CC[C@H]1N] | 110 | 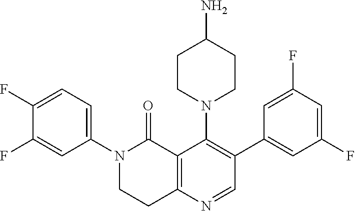 [SMILES: NC1CCN(c2c(-c3cc(F)cc(F)c3)cnc3c2C(=O)N(c2ccc(F)c(F)c2)CC3)CC1] |
| 29 | 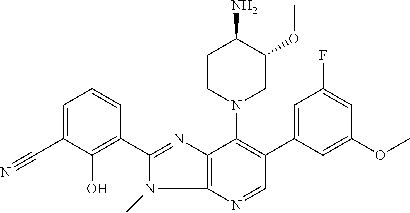 [SMILES: COc1cc(F)cc(-c2cnc3c(nc(-c4cccc(C#N)c4O)n3C)c2N2CC[C@@H](N)[C@H](OC)C2)c1] | 111 | 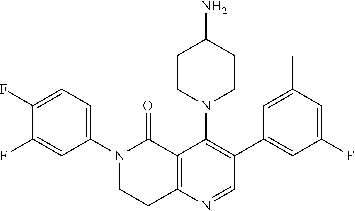 [SMILES: Cc1cc(F)cc(-c2cnc3c(c2N2CCC(N)CC2)C(=O)N(c2ccc(F)c(F)c2)CC3)c1] |
| 30 | 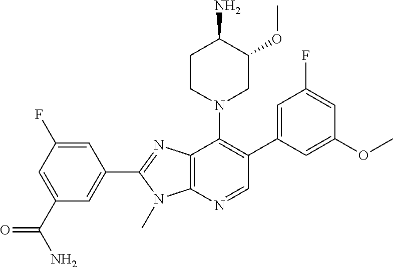 [SMILES: COc1cc(F)cc(-c2cnc3c(nc(-c4cc(F)cc(C(N)=O)c4)n3C)c2N2CC[C@@H](N)[C@H](OC)C2)c1] | 112 | 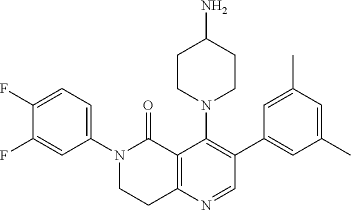 [SMILES: Cc1cc(C)cc(-c2cnc3c(c2N2CCC(N)CC2)C(=O)N(c2ccc(F)c(F)c2)CC3)c1] |
| 31 | 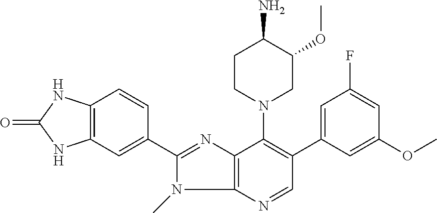 [SMILES: COc1cc(F)cc(-c2cnc3c(nc(-c4ccc5[nH]c(=O)[nH]c5c4)n3C)c2N2CC[C@@H](N)[C@H](OC)C2)c1] | 113 | 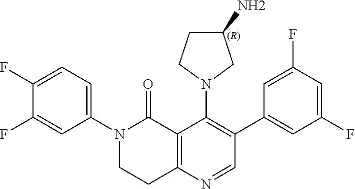 |
| 32 | 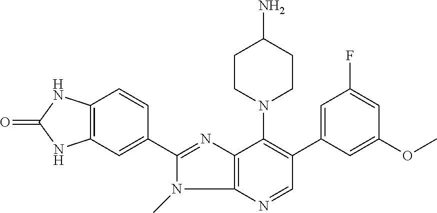 [SMILES: COc1cc(F)cc(-c2cnc3c(nc(-c4ccc5[nH]c(=O)[nH]c5c4)n3C)c2N2CCC(N)CC2)c1] | 114 | 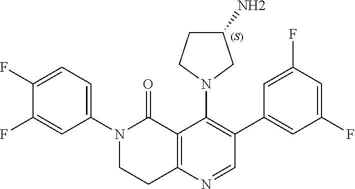 |
| 33 | 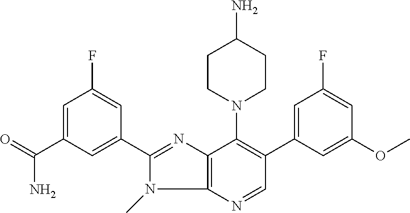 [SMILES: COc1cc(F)cc(-c2cnc3c(nc(-c4cc(F)cc(C(N)=O)c4)n3C)c2N2CCC(N)CC2)c1] | 115 | 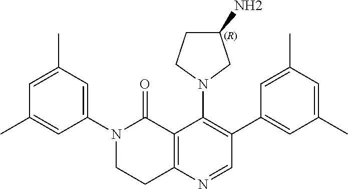 |
| 34 | 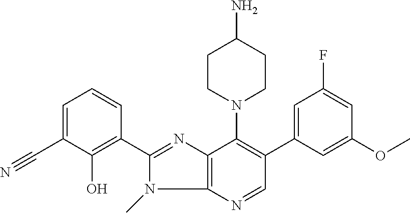 [SMILES: COc1cc(F)cc(-c2cnc3c(nc(-c4cccc(C#N)c4O)n3C)c2N2CCC(N)CC2)c1] | 116 | 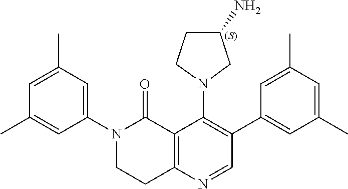 |
| 35 | 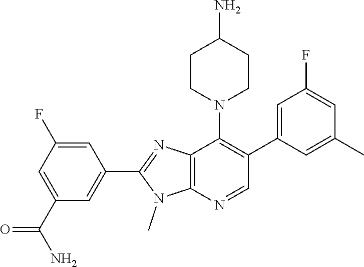 [SMILES: Cc1cc(F)cc(-c2cnc3c(nc(-c4cc(F)cc(C(N)=O)c4)n3C)c2N2CCC(N)CC2)c1] | 117 | 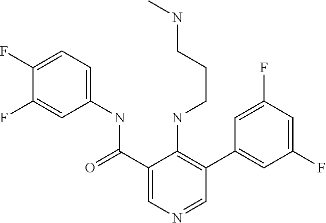 [SMILES: CNCCCNc1c(C(=O)Nc2ccc(F)c(F)c2)cncc1-c1cc(F)cc(F)c1] |
| 36 | 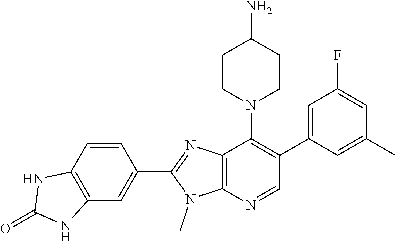 [SMILES: Cc1cc(F)cc(-c2cnc3c(nc(-c4ccc5[nH]c(=O)[nH]c5c4)n3C)c2N2CCC(N)CC2)c1] | 118 | 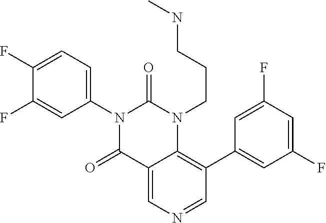 [SMILES: CNCCCn1c(=O)n(-c2ccc(F)c(F)c2)c(=O)c2cncc(-c3cc(F)cc(F)c3)c21] |
| 37 | 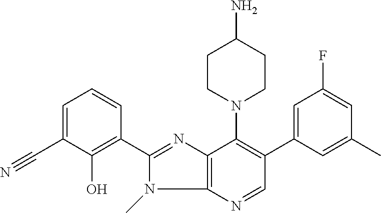 [SMILES: Cc1cc(F)cc(-c2cnc3c(nc(-c4cccc(C#N)c4O)n3C)c2N2CCC(N)CC2)c1] | 119 | 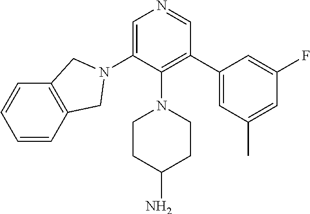 [SMILES: Cc1cc(F)cc(-c2cncc(N3Cc4ccccc4C3)c2N2CCC(N)CC2)c1] |
| 38 | 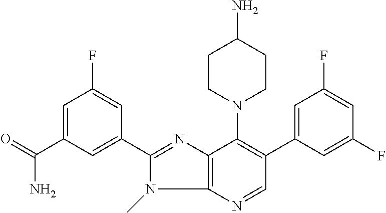 [SMILES: Cn1c(-c2cc(F)cc(C(N)=O)c2)nc2c(N3CCC(N)CC3)c(-c3cc(F)cc(F)c3)cnc21] | 120 | 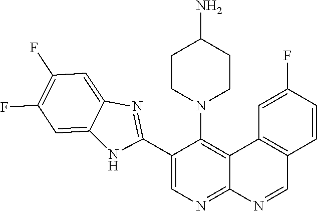 [SMILES: NC1CCN(c2c(-c3nc4cc(F)c(F)cc4[nH]3)cnc3ncc4ccc(F)cc4c23)CC1] |
| 39 | 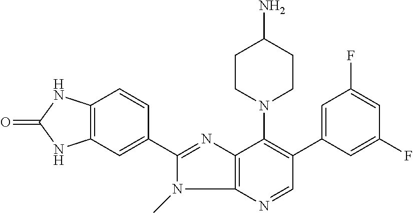 [SMILES: Cn1c(-c2ccc3[nH]c(=O)[nH]c3c2)nc2c(N3CCC(N)CC3)c(-c3cc(F)cc(F)c3)cnc21] | 121 | 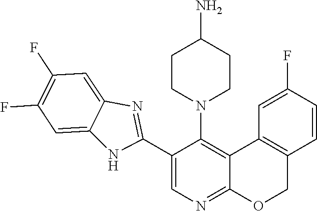 [SMILES: NC1CCN(c2c(-c3nc4cc(F)c(F)cc4[nH]3)cnc3c2-c2cc(F)ccc2CO3)CC1] |
| 40 | 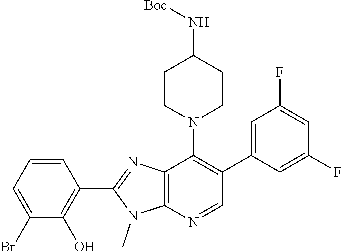 [SMILES: Cn1c(-c2cccc(Br)c2O)nc2c(N3CCC(NC(=O)OC(C)(C)C)CC3)c(-c3cc(F)cc(F)c3)cnc21] | 122 | 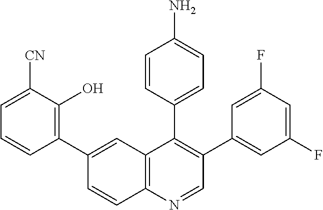 [SMILES: N#Cc1cccc(-c2ccc3ncc(-c4cc(F)cc(F)c4)c(-c4ccc(N)cc4)c3c2)c1O] |
| 41 | 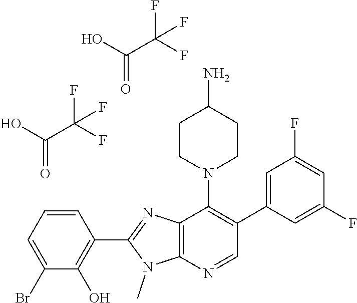 | 123 | 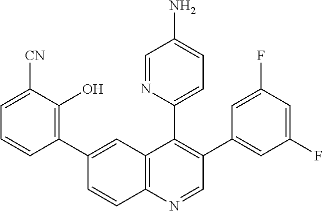 [SMILES: N#Cc1cccc(-c2ccc3ncc(-c4cc(F)cc(F)c4)c(-c4ccc(N)cn4)c3c2)c1O] |
| 42 | 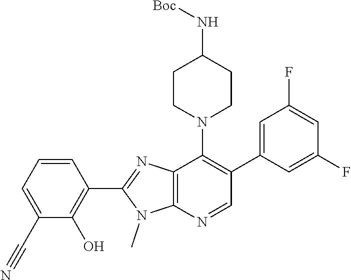 [SMILES: Cn1c(-c2cccc(C#N)c2O)nc2c(N3CCC(NC(=O)OC(C)(C)C)CC3)c(-c3cc(F)cc(F)c3)cnc21] | 124 | 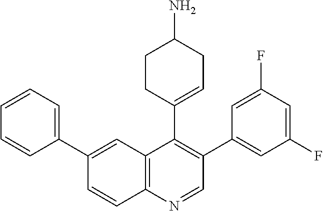 [SMILES: NC1CC=C(c2c(-c3cc(F)cc(F)c3)cnc3ccc(-c4ccccc4)cc23)CC1] |
| 43 | 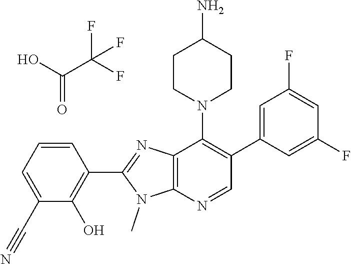 | 125 | 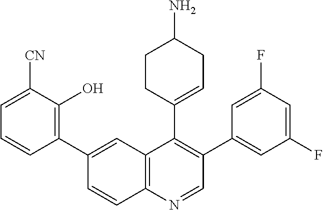 [SMILES: N#Cc1cccc(-c2ccc3ncc(-c4cc(F)cc(F)c4)c(C4=CCC(N)CC4)c3c2)c1O] |
| 44 | 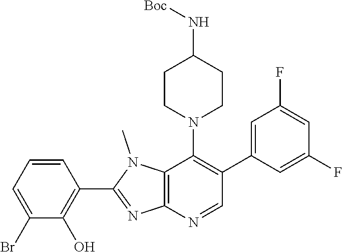 [SMILES: Cn1c(-c2cccc(Br)c2O)nc2ncc(-c3cc(F)cc(F)c3)c(N3CCC(NC(=O)OC(C)(C)C)CC3)c21] | 127 | 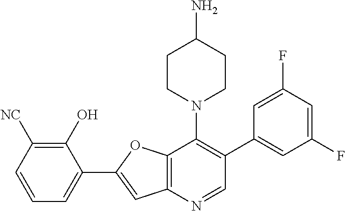 [SMILES: N#Cc1cccc(-c2cc3ncc(-c4cc(F)cc(F)c4)c(N4CCC(N)CC4)c3o2)c1O] |
| 45 | 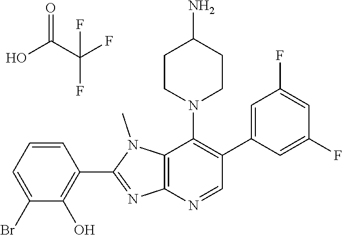 [SMILES: Cn1c(-c2cccc(Br)c2O)nc2ncc(-c3cc(F)cc(F)c3)c(N3CCC(N)CC3)c21 ; O=C(O)C(F)(F)F] | 128 | 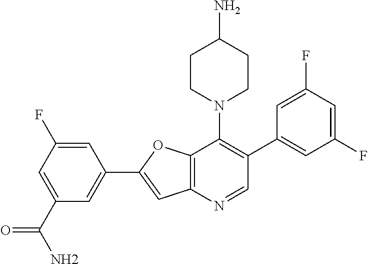 [SMILES: NC(=O)c1cc(F)cc(-c2cc3ncc(-c4cc(F)cc(F)c4)c(N4CCC(N)CC4)c3o2)c1] |
| 46 | 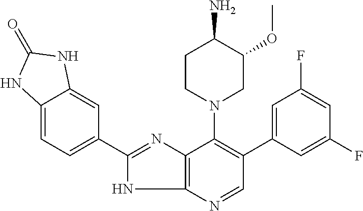 [SMILES: CO[C@@H]1CN(c2c(-c3cc(F)cc(F)c3)cnc3[nH]c(-c4ccc5[nH]c(=O)[nH]c5c4)nc23)CC[C@H]1N] | 129 | 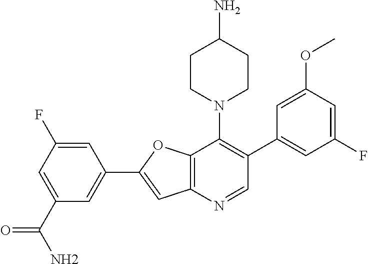 [SMILES: COc1cc(F)cc(-c2cnc3cc(-c4cc(F)cc(C(N)=O)c4)oc3c2N2CCC(N)CC2)c1] |
| 47 | 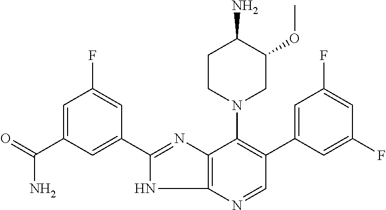 [SMILES: CO[C@@H]1CN(c2c(-c3cc(F)cc(F)c3)cnc3[nH]c(-c4cc(F)cc(C(N)=O)c4)nc23)CC[C@H]1N] | 130 | 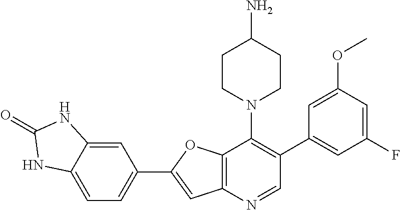 [SMILES: COc1cc(F)cc(-c2cnc3cc(-c4ccc5[nH]c(=O)[nH]c5c4)oc3c2N2CCC(N)CC2)c1] |
| 48 | 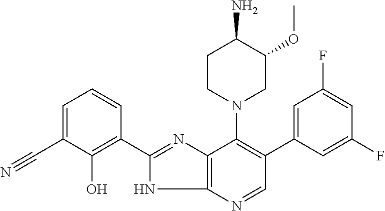 [SMILES: CO[C@@H]1CN(c2c(-c3cc(F)cc(F)c3)cnc3[nH]c(-c4cccc(C#N)c4O)nc23)CC[C@H]1N] | 131 | 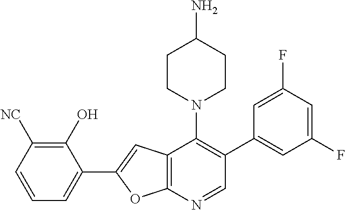 [SMILES: N#Cc1cccc(-c2cc3c(N4CCC(N)CC4)c(-c4cc(F)cc(F)c4)cnc3o2)c1O] |
| 49 | 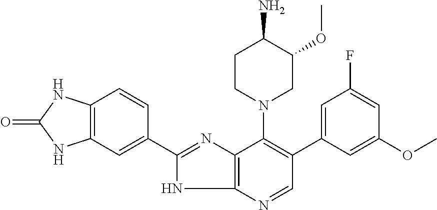 [SMILES: COc1cc(F)cc(-c2cnc3[nH]c(-c4ccc5[nH]c(=O)[nH]c5c4)nc3c2N2CC[C@@H](N)[C@H](OC)C2)c1] | 132 | 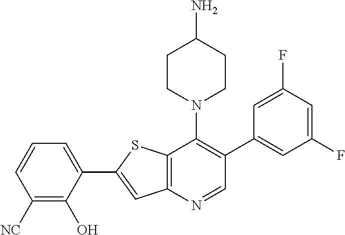 [SMILES: N#Cc1cccc(-c2cc3ncc(-c4cc(F)cc(F)c4)c(N4CCC(N)CC4)c3s2)c1O] |
| 50 | 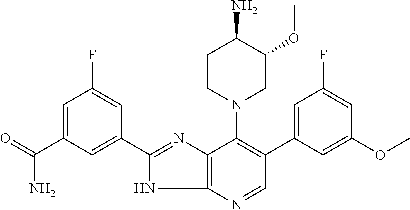 [SMILES: COc1cc(F)cc(-c2cnc3[nH]c(-c4cc(F)cc(C(N)=O)c4)nc3c2N2CC[C@@H](N)[C@H](OC)C2)c1] | 133 | 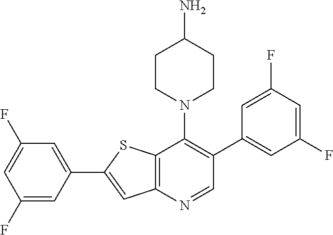 [SMILES: NC1CCN(c2c(-c3cc(F)cc(F)c3)cnc3cc(-c4cc(F)cc(F)c4)sc23)CC1] |
| 51 | 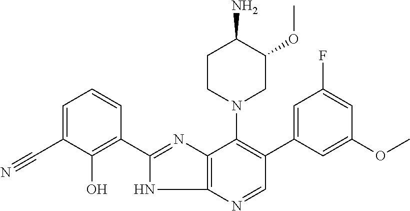 [SMILES: COc1cc(F)cc(-c2cnc3[nH]c(-c4cccc(C#N)c4O)nc3c2N2CC[C@@H](N)[C@H](OC)C2)c1] | 134 | 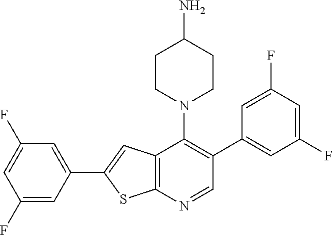 [SMILES: NC1CCN(c2c(-c3cc(F)cc(F)c3)cnc3sc(-c4cc(F)cc(F)c4)cc23)CC1] |
| 52 | 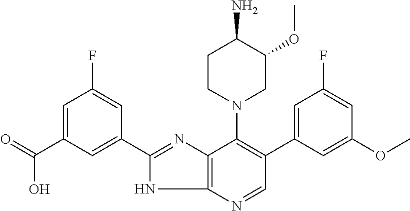 [SMILES: COc1cc(F)cc(-c2cnc3[nH]c(-c4cc(F)cc(C(=O)O)c4)nc3c2N2CC[C@@H](N)[C@H](OC)C2)c1] | 135 | 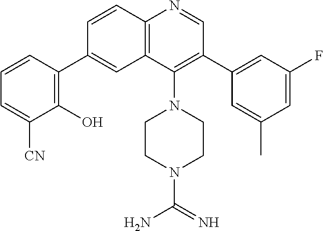 [SMILES: Cc1cc(F)cc(-c2cnc3ccc(-c4cccc(C#N)c4O)cc3c2N2CCN(C(=N)N)CC2)c1] |
| 53 | 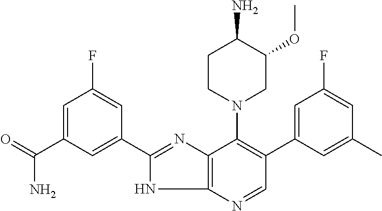 [SMILES: CO[C@@H]1CN(c2c(-c3cc(C)cc(F)c3)cnc3[nH]c(-c4cc(F)cc(C(N)=O)c4)nc23)CC[C@H]1N] | 136 | 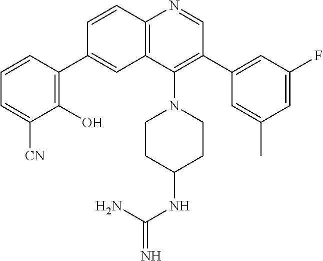 [SMILES: Cc1cc(F)cc(-c2cnc3ccc(-c4cccc(C#N)c4O)cc3c2N2CCC(NC(=N)N)CC2)c1] |
| 54 | 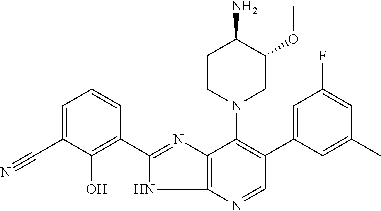 [SMILES: CO[C@@H]1CN(c2c(-c3cc(C)cc(F)c3)cnc3[nH]c(-c4cccc(C#N)c4O)nc23)CC[C@H]1N] | 137 | 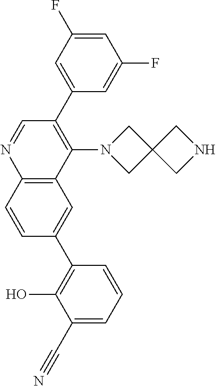 [SMILES: N#Cc1cccc(-c2ccc3ncc(-c4cc(F)cc(F)c4)c(N4CC5(CNC5)C4)c3c2)c1O] |
| 55 | 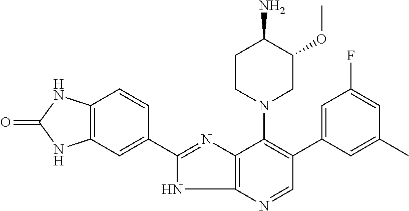 [SMILES: CO[C@@H]1CN(c2c(-c3cc(C)cc(F)c3)cnc3[nH]c(-c4ccc5[nH]c(=O)[nH]c5c4)nc23)CC[C@H]1N] | 138 | 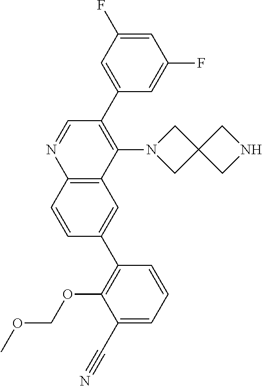 [SMILES: COCOc1c(C#N)cccc1-c1ccc2ncc(-c3cc(F)cc(F)c3)c(N3CC4(CNC4)C3)c2c1] |
| 56 | 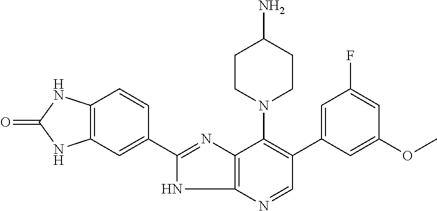 [SMILES: COc1cc(F)cc(-c2cnc3[nH]c(-c4ccc5[nH]c(=O)[nH]c5c4)nc3c2N2CCC(N)CC2)c1] | 139 | 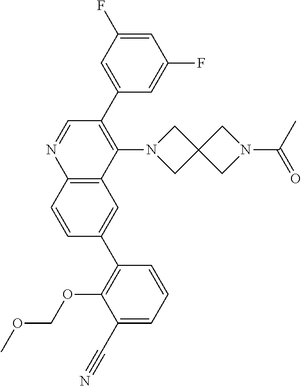 [SMILES: COCOc1c(C#N)cccc1-c1ccc2ncc(-c3cc(F)cc(F)c3)c(N3CC4(CN(C(C)=O)C4)C3)c2c1] |
| 57 | 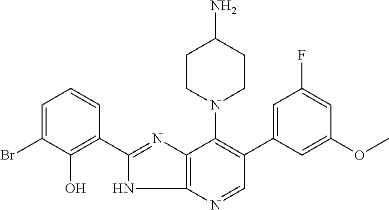 [SMILES: COc1cc(F)cc(-c2cnc3[nH]c(-c4cccc(Br)c4O)nc3c2N2CCC(N)CC2)c1] | 140 | 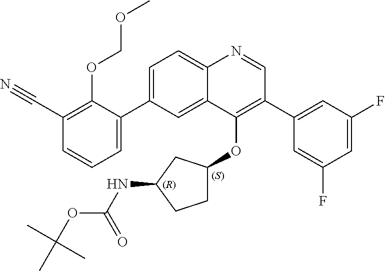 |
| 58 | 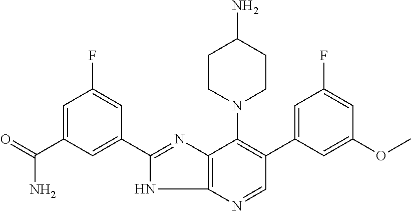 [SMILES: COc1cc(F)cc(-c2cnc3[nH]c(-c4cc(F)cc(C(N)=O)c4)nc3c2N2CCC(N)CC2)c1] | 141 | 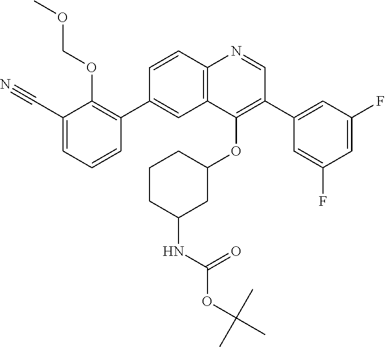 [SMILES: COCOc1c(C#N)cccc1-c1ccc2ncc(-c3cc(F)cc(F)c3)c(OC3CCCC(NC(=O)OC(C)(C)C)C3)c2c1] |
| 59 | 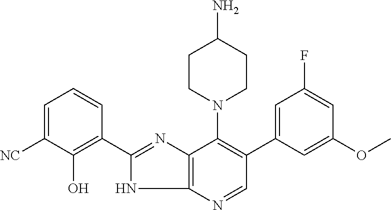 [SMILES: COc1cc(F)cc(-c2cnc3[nH]c(-c4cccc(C#N)c4O)nc3c2N2CCC(N)CC2)c1] | 142 | 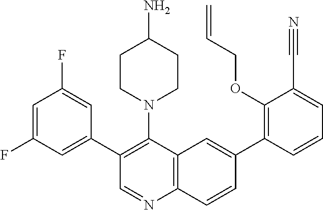 [SMILES: C=CCOc1c(C#N)cccc1-c1ccc2ncc(-c3cc(F)cc(F)c3)c(N3CCC(N)CC3)c2c1] |
| 60 | 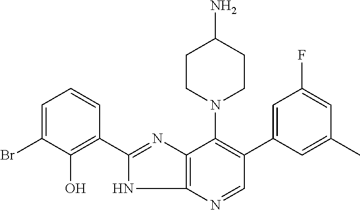 [SMILES: Cc1cc(F)cc(-c2cnc3[nH]c(-c4cccc(Br)c4O)nc3c2N2CCC(N)CC2)c1] | 143 | 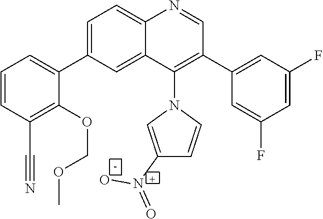 |
| 61 | 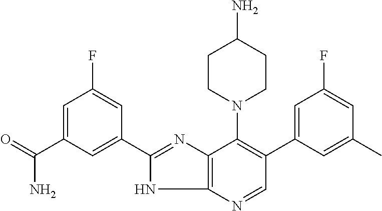 [SMILES: Cc1cc(F)cc(-c2cnc3[nH]c(-c4cc(F)cc(C(N)=O)c4)nc3c2N2CCC(N)CC2)c1] | 144 | 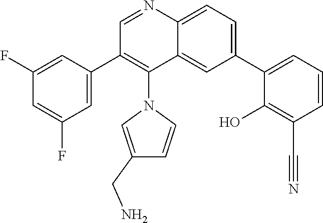 [SMILES: N#Cc1cccc(-c2ccc3ncc(-c4cc(F)cc(F)c4)c(-n4ccc(CN)c4)c3c2)c1O] |
| 62 | 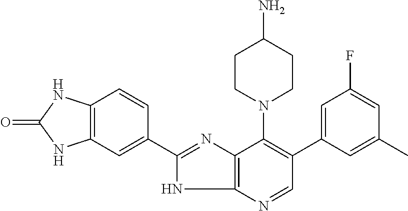 [SMILES: Cc1cc(F)cc(-c2cnc3[nH]c(-c4ccc5[nH]c(=O)[nH]c5c4)nc3c2N2CCC(N)CC2)c1] | 145 | 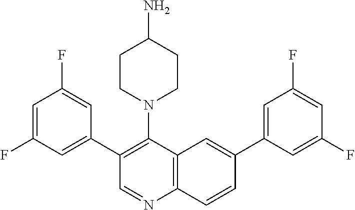 [SMILES: NC1CCN(c2c(-c3cc(F)cc(F)c3)cnc3ccc(-c4cc(F)cc(F)c4)cc23)CC1] |
| 63 | 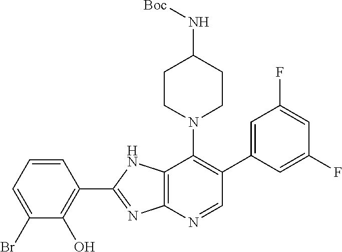 [SMILES: CC(C)(C)OC(=O)NC1CCN(c2c(-c3cc(F)cc(F)c3)cnc3nc(-c4cccc(Br)c4O)[nH]c23)CC1] | 146 | 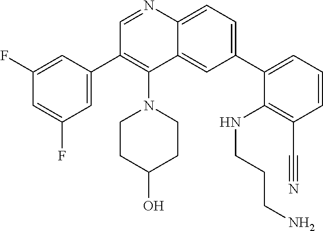 [SMILES: N#Cc1cccc(-c2ccc3ncc(-c4cc(F)cc(F)c4)c(N4CCC(O)CC4)c3c2)c1NCCCN] |
| 64 | 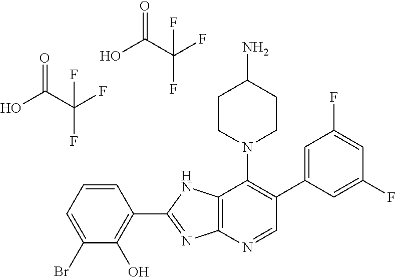 | 147 | 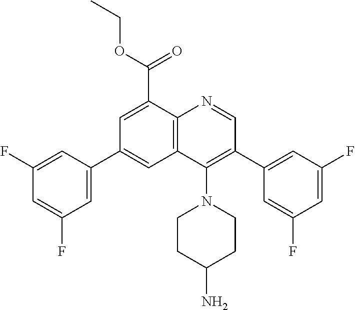 [SMILES: CCOC(=O)c1cc(-c2cc(F)cc(F)c2)cc2c(N3CCC(N)CC3)c(-c3cc(F)cc(F)c3)cnc12] |
| 65 | 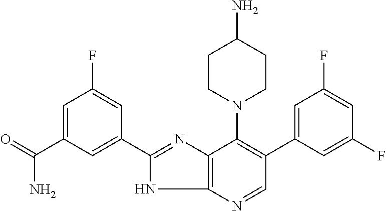 [SMILES: NC(=O)c1cc(F)cc(-c2nc3c(N4CCC(N)CC4)c(-c4cc(F)cc(F)c4)cnc3[nH]2)c1] | 148 | 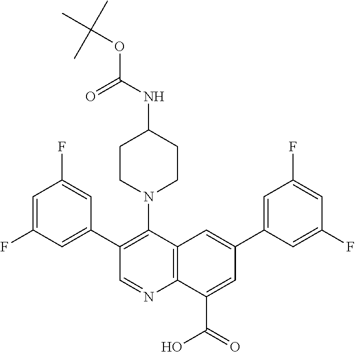 [SMILES: CC(C)(C)OC(=O)NC1CCN(c2c(-c3cc(F)cc(F)c3)cnc3c(C(=O)O)cc(-c4cc(F)cc(F)c4)cc23)CC1] |
| 66 | 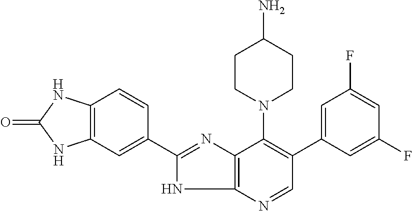 [SMILES: NC1CCN(c2c(-c3cc(F)cc(F)c3)cnc3[nH]c(-c4ccc5[nH]c(=O)[nH]c5c4)nc23)CC1] | 149 | 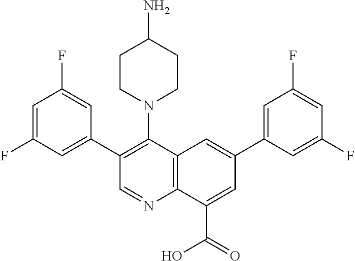 [SMILES: NC1CCN(c2c(-c3cc(F)cc(F)c3)cnc3c(C(=O)O)cc(-c4cc(F)cc(F)c4)cc23)CC1] |
| 67 | 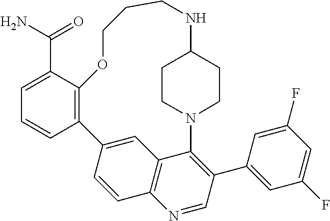 [SMILES: NC(=O)c1cccc2c1OCCCNC1CCN(CC1)c1c(-c3cc(F)cc(F)c3)cnc3ccc-2cc13] | 150 | 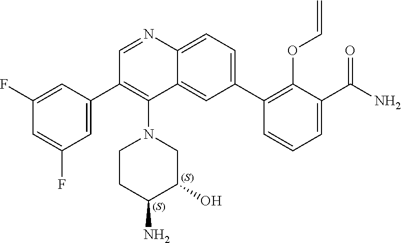 |
| 68 | 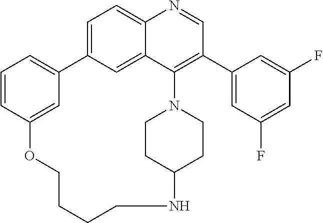 [SMILES: Fc1cc(F)cc(-c2cnc3ccc4cc3c2N2CCC(CC2)NCCCCOc2cccc-4c2)c1] | 151 | 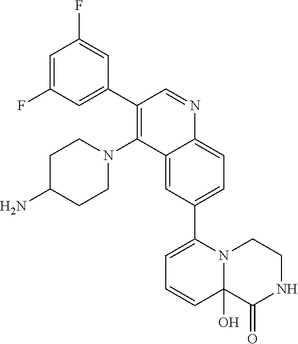 [SMILES: NC1CCN(c2c(-c3cc(F)cc(F)c3)cnc3ccc(C4=CC=CC5(O)C(=O)NCCN45)cc23)CC1] |
| 69 | 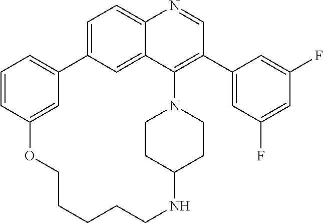 [SMILES: Fc1cc(F)cc(-c2cnc3ccc4cc3c2N2CCC(CC2)NCCCCCOc2cccc-4c2)c1] | 152 | 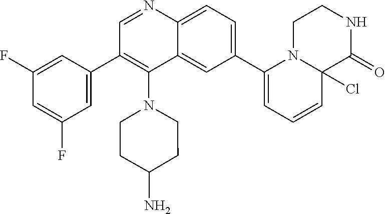 [SMILES: NC1CCN(c2c(-c3cc(F)cc(F)c3)cnc3ccc(C4=CC=CC5(Cl)C(=O)NCCN45)cc23)CC1] |
| 70 | 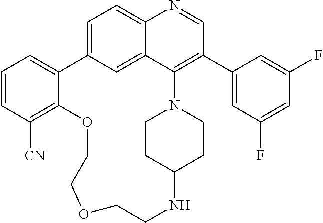 [SMILES: N#Cc1cccc2c1OCCOCCNC1CCN(CC1)c1c(-c3cc(F)cc(F)c3)cnc3ccc-2cc13] | 153 | 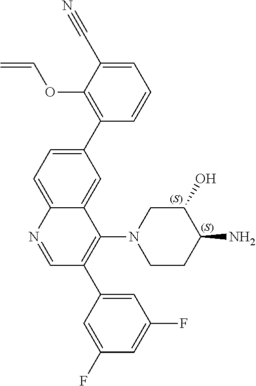 |
| 71 | 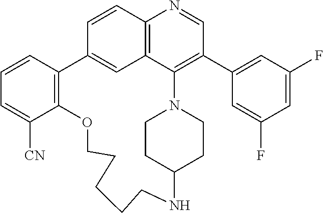 [SMILES: N#Cc1cccc2c1OCCCCCNC1CCN(CC1)c1c(-c3cc(F)cc(F)c3)cnc3ccc-2cc13] | 154 | 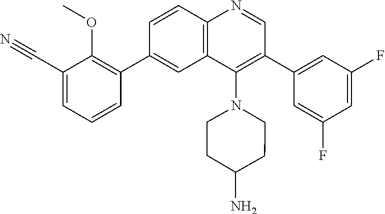 [SMILES: COc1c(C#N)cccc1-c1ccc2ncc(-c3cc(F)cc(F)c3)c(N3CCC(N)CC3)c2c1] |
| 72 | 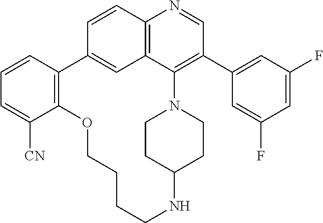 [SMILES: N#Cc1cccc2c1OCCCCNC1CCN(CC1)c1c(-c3cc(F)cc(F)c3)cnc3ccc-2cc13] | 155 | 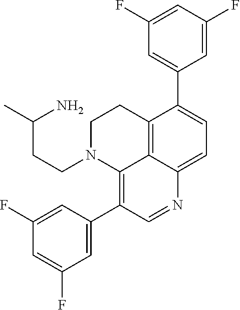 [SMILES: CC(N)CCN1CCc2c(-c3cc(F)cc(F)c3)ccc3ncc(-c4cc(F)cc(F)c4)c1c23] |
| 73 | 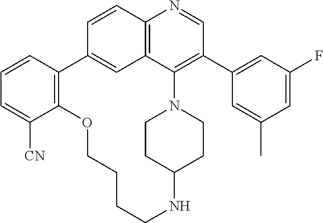 [SMILES: Cc1cc(F)cc(-c2cnc3ccc4cc3c2N2CCC(CC2)NCCCCOc2c(C#N)cccc2-4)c1] | 156 | 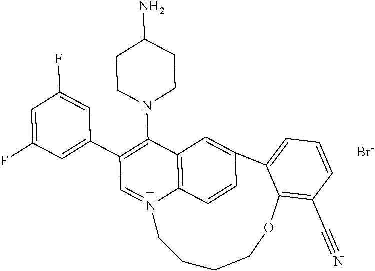 |
| 74 | 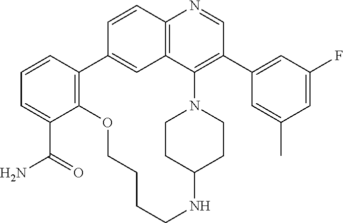 [SMILES: Cc1cc(F)cc(-c2cnc3ccc4cc3c2N2CCC(CC2)NCCCCOc2c(C(N)=O)cccc2-4)c1] | 157 | 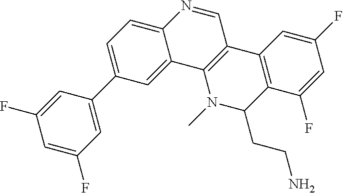 [SMILES: CN1c2c(cnc3ccc(-c4cc(F)cc(F)c4)cc23)-c2cc(F)cc(F)c2C1CCN] |
| 75 | 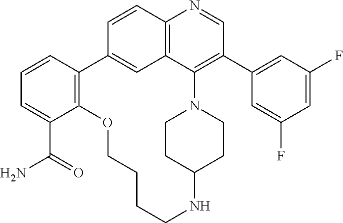 [SMILES: NC(=O)c1cccc2c1OCCCCNC1CCN(CC1)c1c(-c3cc(F)cc(F)c3)cnc3ccc-2cc13] | 158 | 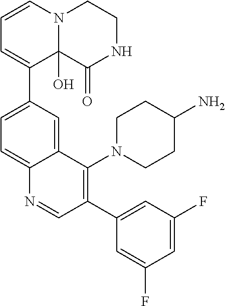 [SMILES: NC1CCN(c2c(-c3cc(F)cc(F)c3)cnc3ccc(C4=CC=CN5CCNC(=O)C45O)cc23)CC1] |
| 76 | 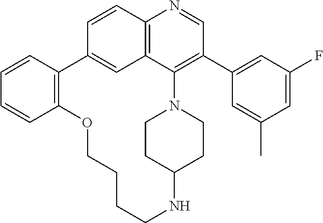 [SMILES: Cc1cc(F)cc(-c2cnc3ccc4cc3c2N2CCC(CC2)NCCCCOc2ccccc2-4)c1] | 159 | 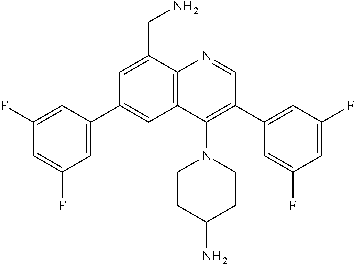 [SMILES: NCc1cc(-c2cc(F)cc(F)c2)cc2c(N3CCC(N)CC3)c(-c3cc(F)cc(F)c3)cnc12] |
| 77 | 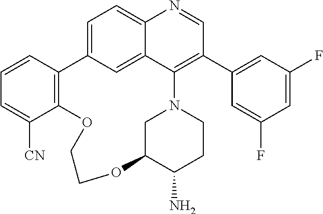 [SMILES: N#Cc1cccc2c1OCCO[C@H]1CN(CC[C@@H]1N)c1c(-c3cc(F)cc(F)c3)cnc3ccc-2cc13] | 160 | 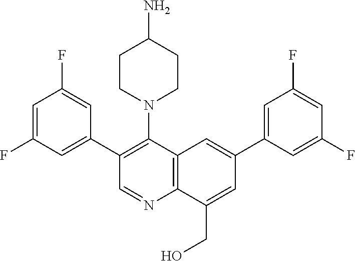 [SMILES: NC1CCN(c2c(-c3cc(F)cc(F)c3)cnc3c(CO)cc(-c4cc(F)cc(F)c4)cc23)CC1] |
| 78 | 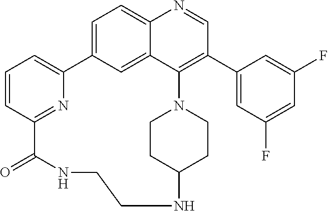 [SMILES: O=C1NCCNC2CCN(CC2)c2c(-c3cc(F)cc(F)c3)cnc3ccc(cc23)-c2cccc1n2] | 161 | 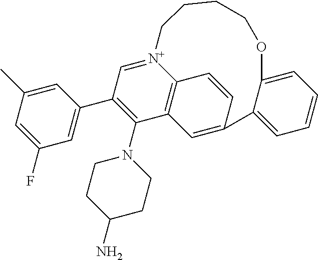 [SMILES: Cc1cc(F)cc(-c2c[n+]3c4ccc(cc4c2N2CCC(N)CC2)-c2ccccc2OCCCC3)c1] |
| 79 | 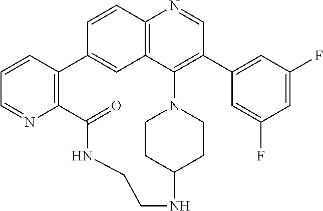 [SMILES: O=C1NCCNC2CCN(CC2)c2c(-c3cc(F)cc(F)c3)cnc3ccc(cc23)-c2cccnc21] | 162 | 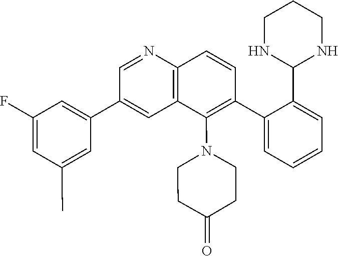 [SMILES: Cc1cc(F)cc(-c2cnc3ccc(-c4ccccc4C4NCCCN4)c(N4CCC(=O)CC4)c3c2)c1] |
| 80 | 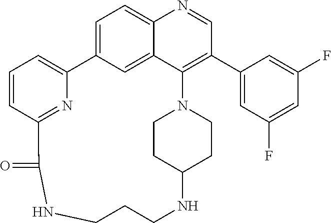 [SMILES: O=C1NCCCNC2CCN(CC2)c2c(-c3cc(F)cc(F)c3)cnc3ccc(cc23)-c2cccc1n2] | 163 | 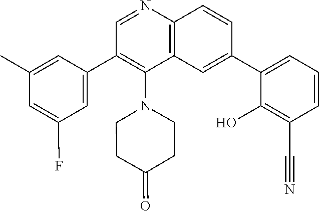 [SMILES: Cc1cc(F)cc(-c2cnc3ccc(-c4cccc(C#N)c4O)cc3c2N2CCC(=O)CC2)c1] |
| 81 | 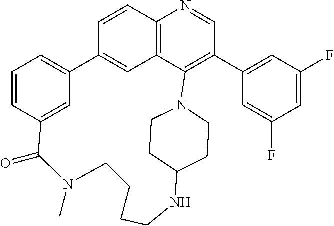 [SMILES: CN1CCCCNC2CCN(CC2)c2c(-c3cc(F)cc(F)c3)cnc3ccc(cc23)-c2cccc(c2)C1=O] | 164 | 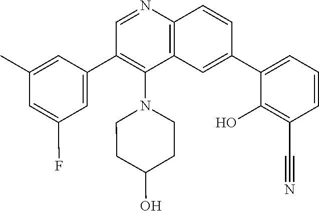 [SMILES: Cc1cc(F)cc(-c2cnc3ccc(-c4cccc(C#N)c4O)cc3c2N2CCC(O)CC2)c1] |
| 82 | 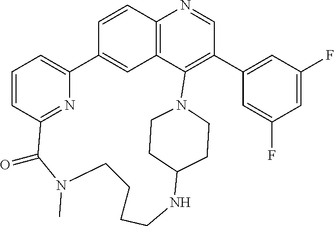 [SMILES: CN1CCCCNC2CCN(CC2)c2c(-c3cc(F)cc(F)c3)cnc3ccc(cc23)-c2cccc(n2)C1=O] | 165 | 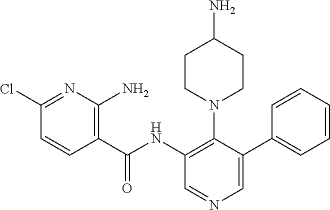 [SMILES: Nc1nc(Cl)ccc1C(=O)Nc1cncc(-c2ccccc2)c1N1CCC(N)CC1] |

or pharmaceutically acceptable salt thereof.

114. A pharmaceutical composition comprising a compound-of claim 3, or pharmaceutically acceptable salt thereof, and a pharmaceutically acceptable excipient.

115. A method of treating a subject afflicted with a disease associated with somatostatin, comprising administering to the subject a compound of claim 3, wherein the disease is diabetes, diarrhea, inflammatory bowel disease, irritable bowel syndrome, cancer, acromegaly, depression, chronic atrophic gastritis, Crohn's disease, ulcerative colitis, retinopathy, arthritis, restenosis, neuroendocrine tumors (NETs), or pain.

116-117. (canceled)
